(19) United States
(12) Patent Application Publication
(10) Pub. No.: US 2021/0161942 A1
(43) Pub. Date: Jun. 3, 2021

(54) GLYCAN COMPOSITIONS AND METHODS OF USE

(71) Applicant: KALEIDO BIOSCIENCES, INC., Lexington, MA (US)

(72) Inventors: Geoffrey A. von Maltzahn, Somerville, MA (US); Jacob Rosenblum Rubens, Cambridge, MA (US)

(21) Appl. No.: 16/630,116

(22) PCT Filed: Jul. 13, 2018
(86) PCT No.: PCT/US2018/042174

§ 371 Date: Jan. 10, 2020

Related U.S. Application Data
(60) Provisional application No. 62/619,084, filed on Jan. 18, 2018.

(30) Foreign Application Priority Data
Jul. 13, 2017 (US) PCT/US2017/042022

(51) Int. Cl. A61K 31/715; A61K 45/06

(52) U.S. Cl. CPC A61K 31/715; A61K 45/06

(57) ABSTRACT

Compositions, e.g., pharmaceutical compositions, nutritional compositions, medical foods, and food ingredients, as well as their methods of use, are provided, for modulating exogenous substances, enzyme activities, and drug activities.

### RELATED APPLICATIONS

This application claims priority to U.S. Application No. 62/619,084, filed Jan. 18, 2018 and International Application No. PCT/US2017/042022, filed Jul. 13, 2017, the entire contents of which are incorporated herein by reference.

### BACKROUND OF THE INVENTION

Maintaining or restoring human health faces a large number of challenges many of which result from the lack of effective treatment options. There is a continued need for novel therapies and treatment regimens.

### SUMMARY OF THE INVENTION

The processing of exogenous substances, such as, e.g., a drug, a drug metabolite, a drug additive, a food, a food additive, an allergen, a toxin or toxicant can be carried out in a subject by mammalian machinery and/or microbial machinery. In some instances, the processing of an exogenous substance can be mediated by the microbial constituents of a subject, such as, e.g., microbes in the gut of the subject. By modulating the processing, e.g., the microbe-mediated processing, of the exogenous substance the effect of the exogenous substance, or its processed forms, on a subject can be altered.

Methods, compositions, kits and the like, described herein are based at least in part on the discovery that a glycan composition can alter the way microbes (e.g., gut microbes) mediate the processing of an exogenous substrate in a subject, e.g., a human subject. In an embodiment, the glycan composition modulates the microbe-mediated processing by increasing or decreasing the number or prevalence of a microbe, e.g., bacterial taxa. In an embodiment, the glycan composition modulates the microbe-mediated processing by increasing or decreasing the activity or level of a constituent or product of the microbe, e.g., an enzyme or a metabolite made by a microbe. In an embodiment, the glycan composition increases or decreases the transcription of an enzyme or other microbial proteins (e.g., one or a plurality of protein constituents of a pathway, such as, e.g., a metabolic pathway) that result in altered activity of the microbe.

In one aspect, the invention features a method for increasing drug activity in a subject comprising:

a) administering a glycan composition in an amount effective and for a time sufficient to increase the drug activity in the subject;

b) administering a glycan composition in an amount effective and for a time sufficient to increase drug activity in the subject, and wherein at the time of administration of the glycan composition, the subject comprises a level of the drug that, in the presence of the administered glycan composition, provides a therapeutic effect;

c) administering the drug, wherein at the time of administration of the drug, the subject has already been administered the glycan composition in an amount effective and for a time sufficient to increase the drug activity in the subject;

d) administering the drug in an amount effective and for a time sufficient to increase the drug activity in the subject, wherein subject has been determined to be in need of the glycan composition, e.g., to increase the activity of the drug; or

e) administering the drug and the glycan composition to the subject, in amounts effective and for times sufficient to increase the drug activity in the subject, wherein administration of the drug and the glycan composition overlap; wherein

i) the glycan preparation comprises glycan polymers that comprise glucose, galactose, arabinose, mannose, fructose, xylose, fucose, or rhamnose glycan units;

ii) the average degree of branching (DB) of the glycan polymers in the glycan preparation is 0, between 0.01 and 0.6, between 0.05 and 0.5, between 0.1 and 0.4, or between 0.15 and 0.4;

iii) at least 50% (at least 60%, 65%, 70%, 75%, 80%, or 85%, or less than 50%) of the glycan polymers in the glycan preparation have a degree of polymerization (DP) of at least 3 and less than 30 glycan units, at least 3 and less than 10 glycan units, at least 5 and less than 25 glycan units, or at least 10 and less than 35 glycan units;

iv) the average DP (mean DP) of the glycan preparation is between about 5 and 8, between about 8 and 13, between about 13 and 25, between about 5 and 15, between about 5 and 20, or between about 5-15;

v) the ratio of alpha- to beta-glycosidic bonds present in the glycan polymers of the glycan preparation is 0, or between about 0.8:1 to about 5:1, between about 1:1 to about 5:1, between about 1:1 to about 3:1, between about 3:2 to about 2:1, or between about 3:2 to about 3:1,

vi) the glycan preparation comprises between 15 mol % and 75 mol % (between 20 mol % and 60 mol %, between 25 mol % and 50 mol %, or between 30 mol % and 45 mol %) 1,6 glycosidic bonds;

vii) the glycan preparation comprises between 1 mol % and 40 mol % (between 1 mol % and 30 mol %, between 5 mol % and 25 mol %, between 10 mol % and 20 mol %) of each at least one, two, or three of 1,2; 1,3; and 1,4 glycosidic bonds;

viii) the glycan preparation has a final solubility limit in water of at least about 50 (at least about 60, 70, at least about 75, or less than 50) Brix at 23° C.; or

ix) the glycan preparation has a dietary fiber content of at least 50% (at least 60%, 70%, 80%, or at least 90%, or less than 50%),

x) any combination of two, three, four, five, six, seven, eight, or nine of i), ii), iii), iv), v), vi), vii), viii), and ix), and

wherein the drug comprises a:

i) cardiac glycoside;

ii) sulfonamide;

iii) nucleoside analogue; or

iv) aminosalicylate; or

wherein the drug is:
a nonsteroidal anti-inflammatory (NSAID) drug;
a chemotherapeutic drug, or generally a drug that is anti proliferative effect on target cells, e.g., cancer cells;
an antibiotic or antibacterial;
an antifungal;
an anti-parasitic agent, e.g., an anti-nematodal;
a hormone;
a sedative;
a heart medication;
a high blood pressure medication;
a colony-stimulating factor;
a dopamine;
an opioid receptor agonist;
a statin;
a CNS stimulant;
a sensitizer/radio-therapy agent;
a narcotic pain reliever;
a hypnotic drug;
an antiacid;
an analgesic;
an uricase inhibitors,
an antipsychotic;
a laxative; or
   a neurotropic agent, e.g., an anticonvulsant.

In another aspect, the invention features a method for increasing an activity of an ingested substance, e.g., a substance in a food, food supplement, or medical food, e.g., phytoestrogen or polyphenol activity, in a subject, e.g., a human subject, comprising:

a) administering a glycan composition in an amount effective and for a time sufficient to increase an activity of the ingested substance, e.g., phytoestrogen or polyphenol, in the subject;

b) administering a glycan composition in an amount effective and for a time sufficient to increase an activity of the ingested substance, e.g., phytoestrogen or polyphenol, in the subject, and wherein at the time of administration of the glycan composition, the subject comprises a level of the ingested substance, e.g., phytoestrogen or polyphenol, that, in the presence of the administered glycan composition, will provide an increase, e.g., a beneficial increase in the ingested substance, e.g., phytoestrogen or polyphenol activity;

c) administering the ingested substance, e.g., phytoestrogen or polyphenol, wherein at the time of administration of the ingested substance, e.g., phytoestrogen or polyphenol, the subject has already been administered the glycan composition in an amount effective and for a time sufficient to increase the activity of the ingested substance, e.g., phytoestrogen or polyphenol, in the subject;

d) administering the ingested substance, e.g., phytoestrogen or polyphenol, wherein subject that has been determined to be in need of the glycan composition; or

e) administering the ingested substance, e.g., phytoestrogen or polyphenol, and the glycan composition to the subject, in amounts effective and for times sufficient to increase the drug activity in the subject, wherein administration of the drug and the glycan composition overlap; wherein:

i) the glycan preparation comprises glycan polymers that comprise glucose, galactose, arabinose, mannose, fructose, xylose, fucose, or rhamnose glycan units;

ii) the average degree of branching (DB) of the glycan polymers in the glycan preparation is 0, between 0.01 and 0.6, between 0.05 and 0.5, between 0.1 and 0.4, or between 0.15 and 0.4;

iii) at least 50% (at least 60%, 65%, 70%, 75%, 80%, or 85%, or less than 50%) of the glycan polymers in the glycan preparation have a degree of polymerization (DP) of at least 3 and less than 30 glycan units, at least 3 and less than 10 glycan units, at least 5 and less than 25 glycan units, or at least 10 and less than 35 glycan units;

iv) the average DP (mean DP) of the glycan preparation is between about 5 and 8, between about 8 and 13, between about 13 and 25, between about 5 and 15, between about 5 and 20, or between about 5-15;

v) the ratio of alpha- to beta-glycosidic bonds present in the glycan polymers of the glycan preparation is 0, or between about 0.8:1 to about 5:1, between about 1:1 to about 5:1, between about 1:1 to about 3:1, between about 3:2 to about 2:1, or between about 3:2 to about 3:1,

vi) the glycan preparation comprises between 15 mol % and 75 mol % (between 20 mol % and 60 mol %, between 25 mol % and 50 mol %, or between 30 mol % and 45 mol %) 1,6 glycosidic bonds;

vii) the glycan preparation comprises between 1 mol % and 40 mol % (between 1 mol % and 30 mol %, between 5 mol % and 25 mol %, between 10 mol % and 20 mol %) of each at least one, two, or three of 1,2; 1,3; and 1,4 glycosidic bonds;

viii) the glycan preparation has a final solubility limit in water of at least about 50 (at least about 60, 70, at least about 75, or less than 50) Brix at 23° C.; or

ix) the glycan preparation has a dietary fiber content of at least 50% (at least 60%, 70%, 80%, or at least 90%, or less than 50%),

x) any combination of two, three, four, five, six, seven, eight, or nine of i), ii), iii), iv), v), vi), vii), viii), and ix).

In another aspect, the invention features a method for decreasing a toxic activity of an ingested substance, e.g., a substance in a food, food supplement, or medical food, e.g., a heterocyclic amine (HCA) or a polycyclic aromatic hydrocarbon (PAH), in a subject, e.g., a human subject, comprising:

a) administering a glycan composition in an amount effective and for a time sufficient to decrease a toxic activity of the ingested substance, e.g., a HCA or PAH, in the subject;

b) administering a glycan composition in an amount effective and for a time sufficient to decrease a toxic activity of the ingested substance, e.g., a HCA or PAH, in the subject, and wherein at the time of administration of the glycan composition, the subject comprises a level of the ingested substance, e.g., a HCA or PAH, that, in the presence of the administered glycan composition, will provide a decrease, e.g., a beneficial decrease, in a toxic activity of the ingested substance, e.g., a HCA or PAH;

c) administering the ingested substance, e.g., a HCA or PAH, wherein at the time of administration of the ingested substance, e.g., A HCA or PAH, the subject has already been administered the glycan composition in an amount effective and for a time sufficient to decrease a toxic activity of the ingested substance, e.g., a HCA or PAH, in the subject;

d) administering the ingested substance, e.g., a HCA or PAH, wherein subject that has been determined to be in need of the glycan composition; or

e) administering the ingested substance, e.g., a HCA or PAH, and the glycan composition to the subject, in amounts effective and for times sufficient to decrease a toxic activity of the ingested substance, e.g., a HCA or PAH, in the subject, wherein administration of the drug and the ingested substance, e.g., a HCA or PAH composition overlap; wherein:

i) the glycan preparation comprises glycan polymers that comprise glucose, galactose, arabinose, mannose, fructose, xylose, fucose, or rhamnose glycan units;

ii) the average degree of branching (DB) of the glycan polymers in the glycan preparation is 0, between 0.01 and 0.6, between 0.05 and 0.5, between 0.1 and 0.4, or between 0.15 and 0.4;

iii) at least 50% (at least 60%, 65%, 70%, 75%, 80%, or 85%, or less than 50%) of the glycan polymers in the glycan preparation have a degree of polymerization (DP) of at least 3 and less than 30 glycan units, at least 3 and less than 10 glycan units, at least 5 and less than 25 glycan units, or at least 10 and less than 35 glycan units;

iv) the average DP (mean DP) of the glycan preparation is between about 5 and 8, between about 8 and 13, between about 13 and 25, between about 5 and 15, between about 5 and 20, or between about 5-15;

v) the ratio of alpha- to beta-glycosidic bonds present in the glycan polymers of the glycan preparation is 0, or between about 0.8:1 to about 5:1, between about 1:1 to about 5:1, between about 1:1 to about 3:1, between about 3:2 to about 2:1, or between about 3:2 to about 3:1,

vi) the glycan preparation comprises between 15 mol % and 75 mol % (between 20 mol % and 60 mol %, between 25 mol % and 50 mol %, or between 30 mol % and 45 mol %) 1,6 glycosidic bonds;

vii) the glycan preparation comprises between 1 mol % and 40 mol % (between 1 mol % and 30 mol %, between 5 mol % and 25 mol %, between 10 mol % and 20 mol %) of each at least one, two, or three of 1,2; 1,3; and 1,4 glycosidic bonds;

viii) the glycan preparation has a final solubility limit in water of at least about 50 (at least about 60, 70, at least about 75, or less than 50) Brix at 23° C.; or

ix) the glycan preparation has a dietary fiber content of at least 50% (at least 60%, 70%, 80%, or at least 90%, or less than 50%),

x) any combination of two, three, four, five, six, seven, eight, or nine of i), ii), iii), iv), v), vi), vii), viii), and ix).

In another aspect, the invention features a method of:

(i) modulating the processing of an exogenous substance (modulating processing) in, or

(ii) modulating an enzyme activity in the gastrointestinal tract (modulating activity) of, a subject comprising:

a) administering a glycan composition in an amount effective and for a time sufficient to modulate processing or modulate activity in the subject;

b) administering a glycan composition in an amount effective and for a time sufficient to to modulate processing or modulate activity in the subject, and wherein at the time of administration of the glycan composition, the subject comprises the exogenous substance or enzyme;

c) administering the exogenous substance, wherein at the time of administration of the exogenous substance, the subject has already been administered the glycan composition in an amount effective and for a time sufficient to modulate the processing of the exogenous substance in the subject;

d) administering the exogenous substance, wherein subject that has been determined to be in need of the glycan composition; or

e) administering the exogenous substance and the glycan composition to the subject, in amounts effective and for times sufficient to increase the modulate processing or modulate activity in the subject, wherein administration of the exogenous substance and the glycan composition overlap; wherein:

i) the glycan preparation comprises glycan polymers that comprise glucose, galactose, arabinose, mannose, fructose, xylose, fucose, or rhamnose glycan units;

ii) the average degree of branching (DB) of the glycan polymers in the glycan preparation is 0, between 0.01 and 0.6, between 0.05 and 0.5, between 0.1 and 0.4, or between 0.15 and 0.4;

iii) at least 50% (at least 60%, 65%, 70%, 75%, 80%, or 85%, or less than 50%) of the glycan polymers in the glycan preparation have a degree of polymerization (DP) of at least 3 and less than 30 glycan units, at least 3 and less than 10 glycan units, at least 5 and less than 25 glycan units, or at least 10 and less than 35 glycan units;

iv) the average DP (mean DP) of the glycan preparation is between about 5 and 8, between about 8 and 13, between about 13 and 25, between about 5 and 15, between about 5 and 20, or between about 5-15;

v) the ratio of alpha- to beta-glycosidic bonds present in the glycan polymers of the glycan preparation is 0, or between about 0.8:1 to about 5:1, between about 1:1 to about 5:1, between about 1:1 to about 3:1, between about 3:2 to about 2:1, or between about 3:2 to about 3:1,

vi) the glycan preparation comprises between 15 mol % and 75 mol % (between 20 mol % and 60 mol %, between 25 mol % and 50 mol %, or between 30 mol % and 45 mol %) 1,6 glycosidic bonds;

vii) the glycan preparation comprises between 1 mol % and 40 mol % (between 1 mol % and 30 mol %, between 5 mol % and 25 mol %, between 10 mol % and 20 mol %) of each at least one, two, or three of 1,2; 1,3; and 1,4 glycosidic bonds;

viii) the glycan preparation has a final solubility limit in water of at least about 50 (at least about 60, 70, at least about 75, or less than 50) Brix at 23° C.; or

ix) the glycan preparation has a dietary fiber content of at least 50% (at least 60%, 70%, 80%, or at least 90%, or less than 50%),

x) any combination of two, three, four, five, six, seven, eight, or nine of i), ii), iii), iv), v), vi), vii), viii), and ix).

In another aspect, the invention features a glycan composition disclosed herein.

In an embodiment, a glycan composition, alters the way microbes (e.g., gut microbes) mediate host processing, e.g., mediate the production, level, structure, distribution, effect, or the activity, of a microbial entity, e.g., an enzyme, that acts on an exogenous substance.

In an embodiment, a glycan composition, alters the way microbes (e.g., gut microbes) mediate the processing by increasing or decreasing the number or prevalence of a microbe, e.g., in the gut of the subject. In an embodiment, the increase or decrease in number or prevalence of a microbe, e.g., bacterial taxa is associated with an increase or decrease in the processing activity, e.g., of an enzyme, that acts on the exogenous substance. In an embodiment, a microbial entity, e.g., an enzyme, alters the processing of an exogenous compound, e.g., a drug, a drug metabolite, a drug additive, a food, a food additive, an allergen, a toxin or toxicant.

In an embodiment, a microbe mediates a change in the production, level, structure, distribution, or activity, of a constituent of the host, e.g., an enzyme (such as a mammalian enzyme), made by the subject. In an embodiment, the host entity, e.g., an enzyme, alters the processing of an exogenous compound, e.g., a drug, a drug metabolite, a drug additive, a food, a food additive, an allergen, a toxin or toxicant.

Methods described herein provide administering, in combination with an exogenous substance, a glycan composition, having a preselected property, e.g., the ability to alter i) the number or prevalence (relative abundance) of a microbe, e.g., a gut microbe, that alters the exogenous substance (e.g., by increasing or decreasing growth of the microbe), ii) the ability of the microbe, e.g., a gut microbe, to provide a processing activity (e.g., in form of a microbial enzyme) that alters the exogenous substance (e.g., by altering the transcription levels/expression level of the microbial enzyme in a microbe), or iii) the ability of the microbe, e.g., a gut microbe, to modulate a host subject response (e.g., increase or decrease a host processing activity (e.g., in form of a host enzyme) that alters the exogenous substance (e.g., by altering metabolite or signaling output by the microbe).

In some embodiments, an enzyme provided by a microbe modifies (e.g., modifies the production, level, structure, distribution, effect, and/or the activity) an exogenous compound, e.g., directly. In some embodiments, an enzyme provided by a microbe modifies (e.g., modifies the production, level, structure, distribution, effect, and/or the activity) an exogenous compound, e.g., indirectly, e.g., the enzyme generates a metabolite that modifies an exogenous compound or mediates host processing in such a way as to modify an exogenous compound. For example, an enzyme provided by a microbe that indirectly modifies an exogenous compound may generate a metabolite that competes with a host enzyme, altering the way the host enzyme interacts with an exogenous compound, thus modifying the exogenous compound. In another example, an enzyme provided by a microbe that indirectly modifies an exogenous compound may activate or inhibit a host enzyme which processes (e.g., metabolizes) the exogenous compound. In embodiments, the increase or decrease in the number or prevalence (e.g., relative abundance) of the microbe, or its ability to provide an enzyme activity, is associated with an increase or decrease in the activity of an enzyme that interacts with the exogenous substance. In an embodiment, the interaction of the enzyme with the exogenous substance, e.g., a drug, can optimize the effect of the drug, e.g., by increasing levels of an active (activated) form or its bioavailability, or decreasing the levels of an inactive (or inactivated) form or the levels of toxic intermediate (e.g., produced through drug metabolization, e.g., by host or microbial enzymes), thus modulating the subsequent effects of the drug on the host (e.g., treatment effects). In an embodiment, the interaction of the enzyme with the exogenous substance, e.g., a toxin or toxicant, can reduce the harmful effects on the subject, e.g., by increasing the processing to non-toxic or less forms or intermediates that are more rapidly excreted from the subject's body (e.g., more soluble, less reactive, etc.).

In an embodiment, a glycan composition that promotes the growth of a Bacteroides sp., Enterococcus faecalis, and/or a Lactobacillus sp, is administered in combination with sulfasalazine to a subject, e.g., a subject with rheumatoid arthritis. Methods are provided (e.g., methods of treatment of rheumatoid arthritis) comprising the administration of a glycan composition described herein to a subject receiving (or about to receive) sulfasalazine in an amount effective to increase conversion of the prodrug sulfasalazine to 5-aminosalicylic acid, e.g., by increasing the level or activity of microbial azoreductase, thereby resulting in increased levels of 5-aminosalicylic acid. See, e.g., Table 1, row 3.

In an embodiment, a glycan composition that decreases growth of a gut microbes (e.g. aerobic enterobacteria or anaerobes such as Clostridium perfringens) or decreases the production of an enzyme that generates p-cresol, e.g., from tyrosine is administered in combination with acetaminophen/paracetamol. Methods are provided comprising the administration of a glycan composition described herein to a subject receiving (or about to receive) acetaminophen/paracetamol in an amount effective to decrease drug-induced toxicity by acetaminophen/paracetamol and or increase activity of acetaminophen/paracetamol, e.g., by decreasing the levels of p-cresol which competes with acetaminophen as a substrate of SILT1A1, thereby decreasing interference of p-cresol with host metabolism of acetaminophen/paracetamol. In an embodiment, a glycan composition that inhibits the growth of pathogenic Firmicutes (e.g., Clostridium difficile), Bacteroidetes, Actinobacteria, and/or Fusobacteria is administered in combination with tyrosine and/or phenylalanine, to a subject, e.g., a subject with pain, fever, or drug-induced toxicity (e.g., from acetaminophen). Methods are provided comprising the administration of a glycan composition described herein to a subject in an amount effective to decrease levels of p-cresol, e.g., by decreasing the level or activity of a microbial enzyme that metabolizes a substrate to p-cresol. See, e.g., Table 1, row 8.

In an embodiment, a glycan composition that promotes the growth of a gut microbe (e.g., Proteobacteria, Firmicutes, or Actinobacteria), is administered in combination with irinotecan to a subject, e.g., a subject with cancer, e.g., colorectal cancer. Methods are provided (e.g., methods of treatment of cancer) comprising the administration of a glycan composition described herein to a subject receiving (or about to receive) irinotecan in an amount effective to decrease the levels of toxic intermediates of irinotecan (such as, e.g., SN-38 glucoronide), e.g., by decreasing the level or activity of microbial beta-glucuronidase. In embodiments, methods are provided to treat side effects associated with irinotecan treatment, e.g. myelosuppresion, diarrhea, and neutropenia. See, e.g., Table 1, row 5.

In an embodiment, a glycan composition that promotes the growth of Eggerthella lenta, e.g., strain DSM2243, is administered in combination with digoxin to a subject, e.g., a subject having a cardiac disease or disorder, e.g., cardiac arrhythmia, or heart failure. Methods are provided (e.g., methods of treatment of cardiac disease or disorder) comprising the administration of a glycan composition described herein to a subject receiving (or about to receive) digoxin in an amount effective to increase drug activity, e.g., by decreasing the level or activity of microbial bacterial reductase. See, e.g., Table 1, row 7.

In an embodiment, a glycan composition that promotes the growth Enterococcus faecium, Lactobacillus mucosae, a Bifidobacterium sp., ora Eggerthella sp is administered in combination with phytoestrogen (e.g., isoflavone or lignan), e.g., a glycosidic isoflavone such as daidzin to a subject, e.g., a subject having or at risk for breast cancer. Methods are provided (e.g., methods of treatment of breast cancer) comprising the administration of a glycan composition described herein to a subject receiving (or about to receive) daidzin in an amount effective to increase levels of equol, e.g., by increasing the level or activity of microbial bacterial reductase that catalyzes the glycosidic cleavage and reduction of an α, β-unsaturated ketone. See, e.g., Table 1, row 14.

In an embodiment, a glycan composition that promotes the growth of Actinobacteria, Bacteroidetes, and/or Firmicutes is administered in combination with phytoestrogen (e.g., isoflavone or lignan), e.g., a glycosidic isoflavone such as daidzin to a subject, e.g., a subject having or at risk for breast cancer. Methods are provided (e.g., methods of treatment of breast cancer) comprising the administration of a glycan composition described herein to a subject receiving (or about to receive) a phytoestrogen in an amount effective to increase levels or enhance activity of a microbial enzyme which boosts metabolism of phytoestrogen to molecules that bind estrogen receptors. See, e.g., Table 1, row 13.

In an embodiment, a glycan composition that inhibits or reduces the growth of bacteria that carry the uidA gene, e.g., Escherichia coli, is administered in combination with a heterocyclic amine (e.g., 2-amino-3-methylimidazo[4,5-f]-quinolone (IQ), 2-amino-l-methyl-6-phenylimidazo[4,5-b]-pyridine (PhIP), 2-amino-3,8-dimethylimidazo[4,5-f]-quinoxaline (MeIQx)) (e.g., formed during the burning of meat), to a subject, e.g., a subject at risk for a cancer. Methods are provided comprising the administration of a glycan composition described herein to a subject receiving (or about to receive), e.g., eating or about to eat, heterocyclic amine (e.g., as a constituent of burned meat) in an amount effective to decrease levels of a toxic compound, e.g., a carcinogen, e.g., by decreasing the level or activity of microbial beta-glucuronidase. See, e.g., Table 1, row 16.

In an embodiment, a glycan composition that inhibits or reduces the growth of a microbe in the colon, e.g., Firmicutes, Proteobacteria, Actinobacteria (e.g., not including Bacteroides), and/or bacteria carrying the choline utilization (cut) gene cluster, is administered in combination with a choline containing compound, e.g., L-carnitine, to a subject, e.g., a subject having or at risk for high cholesterol or a cardiac condition. Methods are provided comprising the administration of a glycan composition described herein to a subject receiving (or about to receive) L-carnitine in an amount effective to decrease levels of a trimethylamine (TMA), e.g., by decreasing the level or activity of microbial glycyl radical enzyme. See, e.g., Table 1, row 17.

In an embodiment, a glycan composition that inhibits the growth of Firmicutes (e.g., Lactobacillus) is administered in combination with taurine-conjugated bile acid (e.g., tauro-beta-muricholic acid), to a subject, e.g., a subject with obesity (e.g., diet-induced obesity). Methods are provided comprising the administration of a glycan composition described herein to a subject in an amount effective to decrease levels of free bile acids and/or increase the levels of taurine-conjugated bile acids (which, e.g., emulsify fats and oils), e.g., by decreasing the level or activity of a microbial bile salt hydrolase. See, e.g., Table 1, row 10.

In an embodiment, a glycan composition that inhibits the growth of Actinobacteria, e.g., Gordonibacter, is administered in combination with ellagitannin to a subject. Methods are provided comprising the administration of a glycan composition described herein to a subject in an amount effective to decrease levels of ellagitannin and/or increase the levels of ellagic acid, e.g., by increasing the level or activity of a microbial enzyme that hydrolyzes ellagitannin to ellagic acid. See, e.g., Table 1, rows 11 and 12.

In an embodiment, a glycan composition that promotes the growth of E. faecalis, E. lenta, Blautia product, Eubacterium limosum, Clostridium scindens, Lactonifactor longoviformis, Clostridium saccharogumia, and/or P. producta is administered in combination with lignan (from plants), e.g., pinoresinol, secoisolariciresinol, to a subject, e.g., a subject having or at risk for breast cancer. Methods are provided (e.g. methods of treating breast cancer) comprising the administration of a glycan composition described herein to a subject in an amount effective to increase levels of enterodiol and/or enterolactone, e.g., by increasing the level or activity of a microbial enzyme which metabolizes pinoresinol and/or secoisolariciresinol to enterodiol and/or enterolactone. See, e.g., Table 1, row 15.

In an embodiment, a glycan composition that inhibits the growth of Enterococcus, Clostridium, Corynebacterium, Campylobacter, and/or Escherichia is administered in combination with a non-caloric artificial sweetener, e.g., cyclamate, xylitol, or saccharin, to a subject. Methods are provided comprising the administration of a glycan composition described herein to a subject in an amount effective to decrease levels of toxic conversion products of sweeteners (e.g., conversion of cyclamate to cyclohexylamine, which can be toxic), e.g., by decreasing the level or activity of a microbial enzyme which metabolizes artificial sweeteners. See, e.g., Table 1, row 18.

In an embodiment, a glycan composition that inhibits or reduces the growth of a gut microbe, e.g., Klebsiella terrigena, is administered in combination with melamine, e.g., food or substance containing melamine, to a subject, e.g., a subject having or at risk for a renal condition (e.g., renal failure). Methods are provided comprising the administration of a glycan composition described herein to a subject in an amount effective to decrease toxic levels of cyanuric acid, e.g., by decreasing the level or activity of a microbial enzyme which metabolizes melamine.

In an embodiment, a glycan composition that increases the growth of a gut microbe, e.g., Bifidobacterium, Lactobacillus, Escherichia, is administered in combination with a conjugated hydroxycinnamate (e.g., found in foods, such as fruits, vegetables, cereals, and coffee), to a subject, e.g., a subject having or at risk for inflammation, e.g., an inflammatory disease. Methods are provided comprising the administration of a glycan composition described herein to a subject in an amount effective to increase levels of anti-inflammatory substances and/or anti-oxidants caffeic acid, ferulic acid, and p-coumaric acid, e.g., by increasing the level or activity of a microbial enzyme processing these substances.

In an embodiment, a glycan composition that inhibits or decreases the growth of a microbe, is administered in combination with a cycasin (e.g., found in some plants), to a subject, e.g., a subject having or at risk for cancer. Methods are provided comprising the administration of a glycan composition described herein to a subject in an amount effective to decrease toxic levels of methylazoxymethanol, a carcinogen, e.g., by decreasing the level or activity of a microbial enzyme processing this substance.

In an embodiment, a glycan composition that increases the growth of a microbe, is administered in combination with an anthocyanin, to a subject, e.g., a subject having or at risk for cancer. Methods are provided comprising the administration of a glycan composition described herein to a subject in an amount effective to increase levels and/or activity of aglycone, which has anticancer properties, e.g., by increasing the level or activity of a microbial enzyme processing this substance.

In an embodiment, a glycan composition that increases the growth of a microbe, e.g., Oxalobacter formigenes, is administered in combination with an oxalate, to a subject, e.g., a subject having or at risk for kidney stones, renal failure, hyperoxaluria, and/or cardiac conduction disorders. Methods are provided comprising the administration of a glycan composition described herein to a subject in an amount effective to decrease levels of oxalate, which is associated with renal toxicity, e.g., by increasing the level or activity of an oxalate-processing microbial enzyme (e.g., oxalate:formate antiporter, formyl-CoA transferase, or oxalyl-CoA decarboxylase).

In an embodiment, a glycan composition that increases the growth of a microbe, e.g., a microbe that upregulates expression of host CYP450 enzyme(s), is administered in combination with a polycyclic aromatic hydrocarbon (PAH), e.g., benzo[a]pyrene, e.g., found in some plant and animal foods, e.g., meats cooked over open flame, to a subject, e.g., a subject having or at risk for cancer. Methods are provided comprising the administration of a glycan composition described herein to a subject in an amount effective to upregulate host CYP450 enzyme(s), thereby conferring protection against carcinogenic PAHs, e.g., by increasing the level or activity of a suitable microbial enzyme.

In an embodiment, a glycan composition that modulates the growth of a microbe, e.g., a microbe that controls expression of host Phase I (CYPs) and Phase II drug metabolizing enzymes (e.g. UGTs, SULTs) which are implicated in the metabolism of drugs can mediate host drug response. Methods are provided comprising the administration of a glycan composition described herein to a subject in an amount effective to modulate levels or activity of a microbe thereby altering host drug metabolizing enzymes, thereby increasing the hosts ability to better respond to a drug.

In an embodiment, a glycan composition that decreases the growth of a microbe, e.g., a microbe which generates a metabolite of sorivudine, e.g., (E)-5-(2-bromovinyl)-uracil (BVU), is administered in combination with sorivudine and 5-fluorouracil (5-FU), to a subject, e.g., a subject having or at risk for a viral infection, e.g., herpes zoster, e.g., a cancer patient having or at risk of having herpes zoster. Methods are provided comprising the administration of a glycan composition described herein to a subject in an amount effective to decrease toxic levels of 5-FU, e.g., by modulating (increasing or decreasing) the level or activity of a suitable microbial enzyme.

In an embodiment, a glycan composition that decreases the growth of an Enterobacteria, e.g., K. pneumoniae, is administered in combination with sorivudine, to a subject, e.g., a subject having or at risk for a viral infection, e.g., herpes zoster or varicella-zoster. Methods are provided comprising the administration of a glycan composition described herein to a subject in an amount effective to decrease inactivation of sorivudine, e.g., by decreasing the levels of or activity of a microbial phosphorylase, e.g., thymidine phosphorylase or uridine phosphorylase. See, e.g., Table 1, row 25.

In an embodiment, a glycan composition that decreases the growth of bacteria, e.g., described herein, e.g., Gram-negative bacteria, e.g., that produce lipopolysaccharide, is administered in combination with CpG-oligonucleotide immunotherapy for cancer, to a subject, e.g., a subject having or at risk of cancer. Methods are provided comprising the administration of a glycan composition described herein to a subject in an amount effective to increase efficacy of CpG-oligonucleotide immunotherapy, e.g., by modulating the levels of or activity of a suitable microbial enzyme. See, e.g., Table 3, row 2.

In an embodiment, a glycan composition that decreases the growth of Bacteroides, e.g., Bacteroides thetaiotaomicron and/or Bacteroides fragilis, is administered in combination with Cytotoxic T lymphocyte protein 4 (CTLA4) inhibitor (e.g., antibody), to a subject, e.g., a subject having or at risk of cancer. Methods are provided comprising the administration of a glycan composition described herein to a subject in an amount effective to increase efficacy of CTLA4 inhibitor, e.g., by modulating the levels of or activity of a suitable microbial enzyme. See, e.g., Table 3, row 6.

In an embodiment, a glycan composition that decreases the growth of Staphylococcus is administered in combination with anti-inflammatory drugs, e.g., to treat inflammatory bowel disease, e.g., tumor necrosis factor (TNF) inhibitors (e.g., antibodies), to a subject, e.g., a subject having or at risk of inflammation or an inflammatory disorder, e.g., inflammatory bowel disease. Methods are provided comprising the administration of a glycan composition described herein to a subject in an amount effective to increase efficacy of the anti-inflammatory drugs, e.g., by modulating the levels of or activity of a suitable microbial enzyme. See, e.g., Table 3, row 7.

In an embodiment, a glycan composition that decreases the growth of Bacteroidetes (e.g., Bacteroidales), and/or mucolytic bacteria such as Ruminococcus gnavus, is administered in combination with an emulsifying agent, e.g., carboxymethylcellulose, polysorbate-80, to a subject, e.g., a subject having or at risk of inflammation, an inflammatory disorder, or metabolic syndrome. Methods are provided comprising the administration of a glycan composition described herein to a subject in an amount effective to increase efficacy of the emulsifying agent, e.g., by modulating the levels of or activity of a suitable microbial enzyme. See, e.g., Table 3, row 8.

Examples of other exogenous substances, enzymes, microbes, and disorders can be found in Tables 1, 2, and 3.

Methods described herein include the administration of a glycan composition to increase the level or prevalence (relative abundance) of a microbe, or increases its ability to make an enzyme that catalyzes the converson of a drug or prodrug into an active form.

Methods described herein include the administration of a glycan composition to decrease the level or prevalence (relative abundance) of a microbe, or decreases its ability to make an enzyme that inhibits the converson of a drug or prodrug into an active form.

Methods described herein include the administration of a glycan composition to decrease the level or prevalence (relative abundance) of a microbe, or decreases its ability to make an enzyme that coverts a drug or prodrug into an undesirable form, e.g., a toxic intermediate/metabolite.

Methods described herein include the administration of a glycan composition to increase the level or prevalence of a microbe, or increases its ability to make an enzyme that inhibits the conversion of a drug or prodrug into an undesirable form, e.g., a toxic intermediate/metabolite.

The glycan compositions can be useful to treat a variety of disorders, as described herein. In addition, the glycan compositions can be used in combination with a substance, e.g., exogenous substance, which is processed by a microbe. Accordingly, provided herein are compositions and methods for modulating the processing of an exogenous substance, modulating an enzyme activity, identifying/selecting a treatment for a subject, reducing toxicity of a substance, increasing drug efficacy, and eliciting specific chemical modifications or reactions in vivo.

### BRIEF DESCRIPTION OF THE FIGURES

FIGS. 1A, 1B, and 1C: A set of graphs showing modification of exogenous substances by glycan-mediated microbiota shifts. (*P<0.05, Welch two sample t-test).

FIG. 2: Box and whisker plots showing the change in abundance of Bacteroidaceae/Bacteroides microbes associated, e.g., with sulfasalazine metabolism in 12 human fecal cultures (from healthy subjects) contacted with various glycan compositions described herein, commercially obtained FOS, and no added carbon control.

FIG. 3: Box and whisker plots showing the change in abundance of Enterococcaceae/Enterococcus microbes associated, e.g., with sulfasalazine metabolism, non-caloric artificial sweetener metabolism, and daidzin metabolism in 12 human fecal cultures (from healthy subjects) contacted with various glycan compositions described herein, commercially obtained FOS, and no added carbon control.

FIG. 4: Box and whisker plots showing the change in abundance of Bacteria/Firmicutes microbes associated, e.g., with irinotecan/SN-38 glucuronide metabolism and tyrosine and/or phenylalanine metabolism in 12 human fecal cultures (from healthy subjects) contacted with various glycan compositions described herein, commercially obtained FOS, and no added carbon control.

FIG. 5: Box and whisker plots showing the change in abundance of Bacteria/Proteobacteria microbes associated, e.g., with irinotecan/SN-38 glucuronide metabolism in 12 human fecal cultures (from healthy subjects) contacted with various glycan compositions described herein, commercially obtained FOS, and no added carbon control.

FIG. 6: Box and whisker plots showing the change in abundance of Bacteria/Actinobacteria microbes associated, e.g., with irinotecan/SN-38 glucuronide metabolism, tyrosine and/or phenylalanine metabolism, and ellagitannin metabolism in 12 human fecal cultures (from healthy subjects) contacted with various glycan compositions described herein, commercially obtained FOS, and no added carbon control.

FIG. 7: Box and whisker plot showing the change in abundance of Eggerthella lenta microbes associated with digoxin metabolism in 12 human fecal cultures (from healthy subjects) contacted with various glycan compositions described herein, commercially obtained FOS, and no added carbon control.

FIG. 8: Box and whisker plots showing the change in abundance of Coriobacteriaceae/Gordonibacter microbes associated, e.g., with ellagitannin metabolism in 12 human fecal cultures (from healthy subjects) contacted with various glycan compositions described herein, commercially obtained FOS, and no added carbon control.

FIG. 9: Box and whisker plots showing the change in abundance of Bacteria/Bacteroidetes microbes associated, e.g., with phytoestrogen metabolism and CpG-oligonucleotide immunotherapy metabolism in 12 human fecal cultures (from healthy subjects) contacted with various glycan compositions described herein, commercially obtained FOS, and no added carbon control.

FIG. 10: Box and whisker plots showing the change in abundance of Bifidobacteriaceae/Bifidobacterium microbes associated, e.g., with daidzin metabolism in 12 human fecal cultures (from healthy subjects) contacted with various glycan compositions described herein, commercially obtained FOS, and no added carbon control.

FIG. 11: Box and whisker plots showing the change in abundance of Lachnospiraceae/Blautia microbes associated, e.g., with lignan metabolism in 12 human fecal cultures (from healthy subjects) contacted with various glycan compositions described herein, commercially obtained FOS, and no added carbon control.

FIG. 12: Box and whisker plots showing the change in abundance of Erysipelotrichaceae/Clostridium_XVIII microbes associated, e.g., with lignan metabolism in 12 human fecal cultures (from healthy subjects) contacted with various glycan compositions described herein, commercially obtained FOS, and no added carbon control.

FIG. 13: Box and whisker plots showing the change in abundance of Lactonifactor/longoviformis microbes associated, e.g., with lignan metabolism in 12 human fecal cultures (from healthy subjects) contacted with various glycan compositions described herein, commercially obtained FOS, and no added carbon control.

FIG. 14: Box and whisker plots showing the change in abundance of Enterobacteriaceae/Escherichia/Shigella microbes associated, e.g., with heterocyclic amine metabolism and non-caloric artificial sweetener metabolism in 12 human fecal cultures (from healthy subjects) contacted with various glycan compositions described herein, commercially obtained FOS, and no added carbon control.

FIG. 15: Box and whisker plots showing the change in abundance of Enterobacteriales/Enterobacteriaceae microbes associated, e.g., with sorivudine metabolism in 12 human fecal cultures (from healthy subjects) contacted with various glycan compositions described herein, commercially obtained FOS, and no added carbon control.

FIG. 16: Box and whisker plots showing the change in abundance of Bacteroides/dorei/fragilis microbes associated, e.g., with cytotoxic T lymphocyte protein 4 (CTLA4) inhibitor metabolism in 12 human fecal cultures (from healthy subjects) contacted with various glycan compositions described herein, commercially obtained FOS, and no added carbon control.

FIG. 17: Box and whisker plots showing the change in abundance of Ruminococcaceae/Ruminococcus microbes associated, e.g., with emulsifying agent metabolism in 12 human fecal cultures (from healthy subjects) contacted with various glycan compositions described herein, commercially obtained FOS, and no added carbon control.

FIGS. 18A and 18B: Graphs of weight loss over time in mice gavaged with glycans from Day -7 to Day 6. Mice were dosed with 200 mg irinotecan per kg body weight at Day 0.

FIG. 19: A graph showing a representative SEC curve between 16 and 20.5 minutes of a glu100 sample showing the average MW and the MW at 10% of maximum absorption on both the leading and trailing edges of the curve.

FIG. 20: A graph showing a representative anomeric region of an ¹H-¹³C HSQC spectrum of a glu100 sample showing the signal distribution of alpha- and beta-glycosidic bonds.

FIG. 21. A graph showing a representative partial assignment of the peaks in the anomeric region of a glu100 sample ¹H-¹³C HSQC spectrum showing the separation between alpha and beta isomers in the ¹H axis, with alpha isomers downfield (¹H>4.8 ppm in this case) and beta isomers upfield (¹H <4.8 ppm in this case). In addition, terminal and internal sugars can be distinguished in the ¹³C axis with terminal sugars upfield (¹³C<94 ppm for alpha and ¹³C<100 ppm for beta in this case) and internal sugars downfield (¹³C>94 ppm for alpha and ¹³C>100 ppm for beta in this case).

FIGS. 22A, 22B, and 23C. Graphs showing representative anomeric region of an ¹H-¹³C HSQC spectrum of glu100 (FIG. 22A), glu50gal50 (FIG. 22B), and gal100 (FIG. 22C) samples, demonstrating the additive effect of the fingerprint peaks.

FIGS. 23A and 23B. Graphs showing the anomeric region of the 1H-13C HSQC spectrum of man100 (FIG. 23A) and the anomeric region of the 1H-13C HSQC spectrum of xyl100 (FIG. 23B).

FIGS. 24A, 24B, and 24C: A series of graphs showing representative GC chromatograms of three representative permethylated and hydrolyzed glycans, glu50gal50 (FIG. 24A), man52glu29gal19 (FIG. 24B), and glu100 (FIG. 24C), showing distribution of regio-chemistry as assigned by comparison to known standards.

FIGS. 25A, 25B, 25C, 25D, 25E, and 25F: are a series of plots depicting the effect of glycans on microbial enzyme expression. Expressed enzymes include N-acetyl transferase (FIG. 25A), beta-glucuronidase (FIG. 25B), thymidine phosphorylase (FIG. 25C), uridine phosphorylase (FIG. 25D), bile acid CoA hydrolase (FIG. 25E), and urease (FIG. 25F).

### DETAILED DESCRIPTION OF THE INVENTION

Described herein are method for modulating the processing of exogenous substances, such as, e.g., a drug, a drug metabolite, a drug additive, a food, a food additive, an allergen, a toxin or toxicant. In embodiments, modulating the processing, e.g., the microbe-mediated processing of the exogenous substance alters the effect of the exogenous substance, or its processed forms, on a subject. Further described herein are glycan compositions for modulating the processing of exogenous substances. In embodiments, glycan compositions are provided as pharmaceutical compositions, medical foods, nutritional compositions, and food ingredients. Further provided are methods, which are effective to treat a number of diseases, disorders or pathological conditions.

### Definitions

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is to be understood to preferably also disclose a group which consists only of these embodiments.

Where an indefinite or definite article is used when referring to a singular noun, e.g. “a”, “an” or “the”, this includes a plural of that noun unless something else is specifically stated.

The terms “obtainable by”, “producible by” or the like are used to indicate that a claim or embodiment refers to compound, composition, product, etc. per se, e.g., that the compound, composition, product, etc. can be obtained or produced by a method which is described for manufacture of the compound, composition, product, etc., but that the compound, composition, product, etc. may be obtained or produced by other methods than the described one as well. The terms “obtained by”, “produced by” or the like indicate that the compound, composition, product, is obtained or produced by a recited specific method. It is to be understood that the terms “obtainable by”, “producible by” and the like also disclose the terms “obtained by”, “produced by” and the like as a preferred embodiment of “obtainable by”, “producible by” and the like.

The wording “compound, composition, product, etc. for treating, modulating, etc.” is to be understood to refer a compound, composition, product, etc. per se which is suitable for the indicated purposes of treating, modulating, etc. The wording “compound, composition, product, etc. for treating, modulating, etc.” additionally discloses that, as a preferred embodiment, such compound, composition, product, etc. is for use in treating, modulating, etc. The wording “compound, composition, product, etc. for use in . . . ” or “use of a compound, composition, product, etc in the manufacture of a medicament, pharmaceutical composition, veterinary composition, diagnostic composition, etc. for . . . ” indicates that such compounds, compositions, products, etc. are to be used in diagnostic or therapeutic methods which may be practiced on the human or animal body. They are considered as an equivalent disclosure of embodiments and claims pertaining to methods of treatment, diagnosis, etc. If an embodiment or a claim thus refers to “a compound for use in treating a human or animal being suspected to suffer from a disease”, this is considered to be also a disclosure of a “use of a compound in the manufacture of a medicament for treating a human or animal being suspected to suffer from a disease” or a “method of treatment by administering a compound to a human or animal being suspected to suffer from a disease”. The wording “compound, composition, product, etc. for treating, modulating, etc.” is to be understood to refer a compound, composition, product, etc. per se which is suitable for the indicated purposes of treating, modulating, etc. The wording “compound, composition, product, etc. for treating, modulating, etc.” additionally discloses that, as a preferred embodiment, such compound, composition, product, etc. is for use in treating, modulating, etc. As used herein, the term “abundance” or “prevalence” as it relates to microbial taxa refers to the presence of one microbial taxa as compared to another microbial taxa in a defined microbial niche, such as the GI tract, or in the entire host organism (e.g., a human or an animal model).

“Acquire” or “acquiring” as the terms are used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing a synthetic or analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., an NMR spectrometer to obtain an NMR spectrum.

As used herein, “antibody” is used in the broadest sense and includes monoclonal antibodies (including full length or intact monoclonal antibodies), polyclonal antibodies, multivalent antibodies, multi-specific antibodies (e.g., bispecific antibodies), and antibody fragments so long as they exhibit the desired activity.

As used herein, the term “cancer” refers to a cell (or cells) that has an aberrant capacity for autonomous growth or replication and an abnormal state or condition (e.g. of a tissue or organ) characterized by proliferative cell growth. “Cancer” as used herein includes any solid or liquid, benign or malignant, non-invasive or invasive cancer or tumor, including hyperplasias, neoplasms, carcinoma, sarcoma, or a hematopoietic neoplastic disorder (e.g., a leukemia) and pre-cancerous or premalignant lesions.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be affected by any appropriate route including oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally. In some embodiments, a combination therapy means that two (or more) different agents or treatments are administered to a subject as part of a defined treatment regimen in response to a condition related to previous administration of one (or more) of the two (or more) different agents. For example, administration of a first agent may produce an undesirable condition in a subject, prompting administration of a combination therapy comprising the first agent and a second (or further) agent (taken/formulated together or separately) which addresses the undesirable condition, e.g., treats, ameliorates, or mitigates the undesirable condition.

“Distinct” as used herein, e.g. with reference to a species in a glycan polymer, is meant to denote that it is chemically and/or structurally different from another. For example, two sugars are “distinct” if they are chemically different, e.g. a fucose and a xylose, or structurally different, e.g. cyclic vs. acyclic, L- vs. D-form. Two dimers are distinct if they consist of the same two monomers but one pair contains alpha-1,4 bond and the other contains a beta-1,6 bond. Distinct entities may have any other suitable distinguishing characteristic or property that can be detected by methods known in the art and/or described herein.

As used herein, “toxin” refers to any compound, naturally occurring or made by humans, e.g., introduced into the environment by human action. Examples of toxins include toxicants, environmental pollutants (e.g., triclosan, TCDD, pesticides, and arsenic), poisons produced by mushrooms, and snake venom. As used herein, an environmental toxin is a toxin commonly encountered by humans in the environment.

As used herein, a “dosage regimen”, “dosing regimen”, or “treatment regimen” is a modality of drug administration that achieves a therapeutic objective. A dosage regimen includes definition of one, two, three, or four of: a route of administration, a unit dose, a frequency of dosage, or a length of treatment.

An “effective amount” and “therapeutically effective amount” as used herein refers to an amount of a pharmaceutical composition or a drug agent that is sufficient to provide a desired effect. In some embodiments, a physician or other health professional decides the appropriate amount and dosage regimen. An effective amount also refers to an amount of a pharmaceutical composition or a drug agent that prevents the development or relapse of a medical condition.

As used herein, “exogenous substance” refers to any substance introduced from or produced outside an organism, cell tissue, or system, e.g., outside a subject. In embodiments, the exogenous substance is introduced into a subject, e.g., orally, nasally, intravenously, intramuscularly. Exogenous substances can include foreign substances, e.g., that do not naturally exist in the subject. Exogenous substances can include substances that naturally exist in some humans, e.g., but may not exist in all humans, can include substance that naturally exist in some humans at some points in time but not others during their lifetime. In some embodiments, an exogenous substance includes a derivative of the exogenous substance, provided that the derivative has not been incorporated into a host macromolecule, e.g., protein, lipid, polysaccharide, or nucleic acid molecules. In some embodiments, the molecular weight of the derivative does not differ from the molecular weight of the exogenous substance by more than 5%, e.g., more than 5%, 10%, 15%, or 20%. In an embodiment, the derivative excludes products of ordinary metabolism, which incorporates atoms from a food or other energy source, vitamin, mineral, or the like. Exogenous substances are described in greater detail herein.

A “glycan unit” as used herein refers to the individual unit of a glycan disclosed herein, e.g., the building blocks from which the glycan is made.

As used herein, an “isolated” or “purified” glycan composition (or component thereof) is substantially pure and free of contaminants, e.g. pathogens or otherwise unwanted biological material, or toxic or otherwise unwanted organic or inorganic compounds. In some embodiments, pure or isolated compounds, compositions or preparations may contain traces of solvents and/or salts (such as less than 10%, 9%, 8%, 7%, 6%, 5%, 4%, 3%, 2%, 1%, less than 0.5% or 0.1% by w/w, w/v, v/v or molar %). Purified compounds or preparations contain at least about 60% (by w/w, w/v, v/v or molar %), at least about 75%, at least about 90%, at least about 95%, at least about 97%, at least about 98%, or at least about 99% by w/w, w/v, v/v or molar % the compound(s) of interest. For example, a purified (substantially pure) or isolated glycan composition is one that is at least 80%, 85%, 90%, 91%, 92%, 93%, 94%, 95%, 98%, 99%, 99.5%, 99.8%, 99.9% or 100% of the glycan therapeutic by w/w, w/v, v/v or molar % (i.e. not including any solvent, such as e.g. water, in which the glycan composition may be dissolved) and separated from the components that accompany it, e.g. during manufacture, extraction/purification and/or processing (e.g. such that the glycan composition is substantially free from undesired compounds). Purity may be measured by any appropriate standard method, for example, by column chromatography (e.g., size-exclusion chromatography (SEC)), thin layer chromatography (TLC), gas chromatography (GC), high-performance liquid chromatography (HPLC) or nuclear magnetic resonance (NMR) spectroscopy. Purified or purity may also define a degree of sterility that is safe for administration to a human subject, e.g., lacking viable infectious or toxic agents.

As used herein, “microbiome” refers to the genetic content of the communities of microbes that live in and on a subject (e.g. a human subject), both sustainably and transiently, including eukaryotes, archaea, bacteria, and viruses (including bacterial viruses (e.g., phage)), wherein “genetic content” includes genomic DNA, RNA such as ribosomal RNA and messenger RNA, the epigenome, plasmids, and all other types of genetic information. In some embodiments, microbiome specifically refers to genetic content of the communities of microorganisms in a niche.

“Microbiota” as used herein refers to the community of microorganisms that occur (sustainably or transiently) in and on a subject (e.g. a human subject), including eukaryotes, archaea, bacteria, and viruses (including bacterial viruses, e.g. phage). In some embodiments, microbiota specifically refers to the microbial community in a niche.

“Modulate the microbiota” or “modulating the microbiota” as used herein refers to changing the state of the microbiota. Changing the state of the microbiota may include changing the structure and/or function of the microbiota. A change in the structure of the microbiota is, e.g., a change in the relative composition of a taxa, e.g., in one or more region of the GI tract such as the cecum, ascending colon, transverse colon, descending colon, sigmoid colon, and/or rectum. In an embodiment, a change in the structure of the microbiota comprises a change in the abundance of a taxa, e.g., relative to another taxa or relative to what would be observed in the absence of the modulation. Modulation of the microbiota may also, or in addition, include a change in a function of the microbiota, such as a change in microbiota gene expression, level of a gene product (e.g., RNA or protein), or metabolic output of the microbiota. Modulation of the structure or function of the microbiota may additionally induce a change in one or more functional pathway of the host (e.g., a change in gene expression, level of a gene product, and/or metabolic output of a host cell or host process) as a result of a change in the microbiota or its function.

As used herein, the term “oligosaccharide” refers to a molecule consisting of multiple (i.e., two or more) individual glycan units linked covalently. Each glycan unit may be linked through a glycosidic bond (e.g., a 1→2 glycosidic bond, a 1→3 glycosidic bond, a 1→4 glycosidic bond, a 1→5 glycosidic bond or a 1→6 glycosidic bond) present in either the alpha or beta configuration.

As used herein, a “pharmaceutical composition” or “pharmaceutical preparation” is a composition or preparation having pharmacological activity or other direct effect in the mitigation, treatment, or prevention of disease, and/or a finished dosage form or formulation thereof and is for human use. A pharmaceutical composition or pharmaceutical preparation is typically produced under good manufacturing practices (GMP) conditions. Pharmaceutical compositions or preparations may be sterile or non-sterile. If non-sterile, such pharmaceutical compositions meet the microbiological specifications and criteria for non-sterile pharmaceutical products as described in the U.S. Pharmacopeia (USP) or European Pharmacopoeia (EP). Pharmaceutical compositions may further comprise or may be co-administered with additional active agents, such as, e.g. additional therapeutic agents. Pharmaceutical compositions may also comprise pharmaceutically acceptable excipients, solvents, carriers, fillers, or any combination thereof.

As used herein, the term “polysaccharide” refers to a polymeric molecule consisting of multiple individual glycan units linked covalently. In some embodiments, a polysaccharide comprises at least 10 or more glycan units (e.g., at least 10, at least 15, at least 20, at least 25, or at least 50, at least 100, at least 250, at least 500, or at least 1000 glycan units). Each glycan unit may be linked through a glycosidic bond (e.g., a 1→2 glycosidic bond, a 1→3 glycosidic bond, a 1→4 glycosidic bond, a 1→5 glycosidic bond and a 1→6 glycosidic bond) present in either the alpha or beta confirguation. In some embodiments, a polysaccharide is a homogenous polymer comprising identical repeating units. In other embodiments, a polysaccharide is a heterogenous polymer comprised of varied repeating units. Polysaccharides may further be characterized by a degree of branching (DB, branching points per residue) or a degree of polymerization (DP).

As used herein, the term “subject” or “patient” generally refers to any human subject. The term does not denote a particular age or gender. Subjects may include pregnant women. Subjects may include a newborn (a preterm newborn, a full-term newborn), an infant up to one year of age, young children (e.g., 1 yr to 12 yrs), teenagers, (e.g., 13-19 yrs), adults (e.g., 20-64 yrs), and elderly adults (65 yrs and older). In general, a subject comprises a host and its corresponding microbiota.

A “substantial decrease” as used herein is a decrease of 5%, 10%, 20%, 30%, 40%, 50%, 60%, 70%, 80%, 90%, 95%, 97%, 98%, 99%, 99.9%, or 100%.

A “substantial increase” as used herein is an increase of 10%, 20%, 30%, 40%, 50%, 60%, 70%, 80%, 90%, 100%, 150%, 200%, 250%, 300%, 350%, 400%, 450%, 500%, 550%, 600%, 650%, 700%, 750%, 800%, 850%, 900%, 950%, 1000%, or more than 1000%.

“Synthetic” as used herein refers to a man-made compound or preparation, such as a glycan composition, that is not naturally occurring. In one embodiment, the polymeric catalyst described herein is used to synthesize the glycans of the preparation under suitable reaction conditions, e.g. by a polymerization reaction that creates oligomers and polymers from individual glycan units that are added to the reaction. In some embodiments, the polymeric catalyst acts as a hydrolysis agent and can break glycosidic bonds. In other embodiments, the polymer catalyst can form glycosidic bonds.

The terms “treating” and “treatment” as used herein refer to the administration of an agent or composition to a subject (e.g., a symptomatic subject afflicted with an adverse condition, disorder, or disease) so as to affect a reduction in severity and/or frequency of a symptom, eliminate a symptom and/or its underlying cause, and/or facilitate improvement or remediation of damage, and/or preventing an adverse condition, disorder, or disease in an asymptomatic subject who is susceptible to a particular adverse condition, disorder, or disease, or who is suspected of developing or at risk of developing the condition, disorder, or disease.

The term “antigen” refers to a substance capable of eliciting an immune response and ordinarily this is also the substance used for detection of the corresponding antibodies by one of the many in vitro and in vivo immunological procedures available for the demonstration of antigen-antibody interactions. Similarly, the term allergen is used to denote an antigen having the capacity to induce and combine with antibodies; however, this definition does not exclude the possibility that allergens may also induce antibodies of classes other than IgE.

As used herein, “derivative” refers to the product of a processed exogenous substance. A derivative can include a metabolite and/or a product of any enzymatic reaction described herein.

As used herein, administered “in combination”, means that two (or more) different treatments, e.g., treatments described herein, are delivered to a subject, e.g., during the course of the subject's affliction with a disorder/condition. For example, the two or more treatments are delivered after the subject has been diagnosed with the disorder/condition and before the disorder/condition has been cured or eliminated or treatment has terminated for other reasons. In embodiments, the delivery of one treatment is still occurring when the delivery of the second commences, i.e., there is overlap in terms of administration. This is sometimes referred to herein as “simultaneous” or “concurrent delivery”. In other embodiments, the delivery of one treatment stops before the delivery of the other treatment starts. In some embodiments of either case, the treatment is more effective because of the combined administration. For example, the second treatment is more effective, e.g., an equivalent effect is seen with less of (e.g., a lower dosage of) the second treatment, or the second treatment reduces symptoms to a greater extent, than would be seen if the second treatment were administered in the absence of the first treatment, or the analogous situation is seen with the first treatment. In some embodiments, delivery of the treatments is such that the reduction in a symptom, or other parameter related to the disorder/condition is greater than what would be observed with one treatment delivered in the absence of the other. In embodiments, the effect of the two treatments can be partially additive, wholly additive, or greater than additive. In embodiments, the delivery can be such that an effect of the first delivered treatment is still detectable when the second treatment is delivered.

“Fructooligosaccharide” or “FOS”, as the terms are used herein, refer to a fructose polymer, optionally comprising terminal glucose, of the following sequence: (Fru)n-Glc consisting of one or more of: beta 2,1, beta 2,6, alpha 1,2 and beta-1,2 glycosidic bonds, wherein n typically is 3-10. Variants include Inulin type β-1,2 and Levan type β-2,6 linkages between fructosyl units in the main chain. In an embodiment, FOS is made from an enzyme from B. macerans, Z. mobilis, L. reutri, A. niger, A. japonicas, A. foetidus, A. sydowi, A. pullans, C. purpurea, F. oxysporum P. citrinum, P. frequentans, P. spinulosum, P. rigulosum, P. parasitica S. brevicaulis, S. cerevisiae, or K. marxianus. In embodiments FOS is produced by enzymatic action of a Fructosyltransferase, β-fructofuranosidase (EC 3.2.1.26), inulosuscrase (EC 2.4.1.9) levansucrase (EC 2.4.1.10), or endoinulinase.

As used herein, a “glycan polymer preparation” (also referred to as a “preparation of glycan polymers”, “glycan preparation”, “glycan composition”, or “glycan polymer”) is a preparation comprising glycan polymers that exhibits a desired effect (e.g., a therapeutic effect or a modulating effect, e.g., with regard to an exogenous substance, or a beneficial effect, e.g., with regard to a subject's health). In some embodiments, preparations of glycan polymers do not contain one or more naturally occurring oligosaccharide, including: glucooligosaccharide, mannanoligosaccharide, inulin, lychnose, maltotretraose, nigerotetraose, nystose, sesemose, stachyose, isomaltotriose, nigerotriose, maltotriose, melezitose, maltotriulose, raffinose, kestose, fructooligosaccharide, 2′-fucosyllactose, galactooligosaccharide, glycosyl, idraparinux, isomaltooligosaccharide, maltodextrin, xylooligosaccharide, agar, agarose, alginic acid, alguronic acid, alpha glucan, amylopectin, amylose, arabioxylan, beta-glucan, callose, capsulan, carrageenan, cellodextrin, cellulin, cellulose, chitin, chitin nanofibril, chitin-glucan complex, chitosan, chrysolaminarin, curdlan, cyclodextrin, alpha-cylcodextrin, dextran, dextrin, dialdehyde starch, ficoll, fructan, fucoidan, galactoglucomannan, galactomannan, galactosamineogalactan, gellan gum, glucan, glucomannan, glucoronoxyland, glycocalyx, glycogen, hemicellulose, hypromellose, icodextrin, kefiran, laminarin, lentinan, levan polysaccharide, lichenin, mannan, mucilage, natural gum, paramylon, pectic acid, pectin, pentastarch, phytoglycogen, pleuran, poligeenan, polydextrose, porphyran, pullulan, schizophyllan, sepharose, sinistrin, sizofiran, sugammadex, welan gum, xantham gum, xylan, xyloglucan, zymosan, and the like. In some embodiments, a glycan polymer exists as a salt, e.g., a pharmaceutically acceptable salt. In some embodiments, glycan preparations do not contain sorbitol. In some embodiments, glycan preparations do not contain citric acid. In some embodiments, glycan preparations do not contain cyclic glycans.

“Increasing drug activity” as that term is used herein, refers to one or more of any of: a) increasing a therapeutic or other beneficial effect, of a drug on a weight, molar, number of molecules, or dosage unit, basis. By way of example, the administration of X mgs of a drug, when the drug activity is increased, has greater activity than the administration of X mgs in the absence of the increase. While not wishing to be bound by mechanism, an increase in drug activity can comprise an increase in conversion from inactive form to active form, e.g., an increase in processing of a drug to a prodrug, a decrease in the conversion of an active form of the drug to an inactive from, a decrease in the elimination of an active form of a drug (e.g., by excretion), etc.; b) increasing the amount of drug that can be administered relative to the amount that can be administered before reaching a non-therapeutic event. Exemplary non-therapeutic events comprise: toxicity, e.g., toxicity of the drug, or of a species arising from metabolism of the drug or off-target activity; c) increasing drug efficacy, wherein drug efficacy refers to the ability of a drug to produce an effect, e.g., a desired effect, e.g., increasing GI motility or lowering cholesterol levels. In embodiments, drug efficacy includes a drug's intrinsic activity, which can be expressed as the amount of a biological effect produced per unit of drug-receptor complex formed. A drug's bioavailability refers to the proportion of the drug that enters the circulation of a subject after administration and is thus able to elicit an effect. Drug potency refers to the measure of drug activity expressed in terms of the amount of drug required to produce an effect with a predetermined intensity. Drug efficacy can be measured qualitatively and/or quantitatively. In embodiments, drug efficacy can be measured by detecting an improvement in (e.g., lack of) one or more symptoms of the disease/disorder that the drug was intended to treat. In embodiments, drug efficacy can be measured in vitro, e.g., in a cell or tissue sample, e.g., cell culture, by determining a functional readout (e.g., protein, e.g., enzyme level or activity, production of a molecule such as a second messenger, posttranslational modification such as phosphorylation of a protein, or changes in gene expression) after incubation of the cell or tissue sample with the drug. In other embodiments, drug efficacy can be measured ex vivo or in vivo, e.g., by determining a functional readout after administration of the drug or incubation ex vivo with a sample from the subject; d) increasing drug potency, wherein drug potency can be measured by determining the drug's EC50 (half maximal effective concentration), which is the concentration of the drug at which the effect is 50% of Emax (the maximum possible effect for the drug). The lower the EC50, the higher the potency of the drug. EC50 of a drug can be determined by standard methods, e.g., measuring a functional readout in a cell or tissue sample, e.g., cell culture, after administration of increasing doses of the drug; or e) increasing drug (bio-) availability, wherein drug bioavailability can be measured by determining the area under the plasma concentration-time curve (AUC), which is directly proportional to the total amount of drug (e.g., unmodified drug) that reaches the systemic circulation of a subject.

### Exogenous Substances

Methods described herein modulate the processing of an exogenous substance. In some embodiments, methods described herein modulate the ability of the microbiome to mediate the processing of an exogenous substance. Exogenous substances can include a variety of agents, e.g., pharmaceutical agents, environmental toxins or toxicants, dietary components, food additives, drug additives, and/or allergens.

Pharmaceutical agents can be, e.g., a protein/peptide/polypeptide (e.g., antibody molecule or fragment thereof), a nucleic acid (e.g., DNA, RNA, and/or inhibitory nucleic acid, e.g., siRNA, RNAi, miRNA), or modified forms thereof, or a small molecule.

Pharmaceutical agents include an enzyme, a receptor, an antibody, or an adaptor protein. In embodiments, a pharmaceutical agent is an FDA approved agent, e.g., approved for preventing or treating a disorder, disease, or condition described herein.

In some embodiments, the pharmaceutical agent is a prodrug, e.g., that requires bioactivation by the subject (e.g., by a microorganism in the subject, e.g., gut microorganism in the subject) to become an active drug form. Examples of prodrugs are irinotecan, protonsil, or sulfasalazine. For example, a prodrug comprises an azo bond, such as, e.g., protonsil and sulfasalazine.

Exemplary classes of pharmaceutical agents include but are not limited to an anti-inflammatory agent, a non-steroidal anti-inflammatory drug (NSAID), a statin, an antioxidant, an antimicrobial (e.g., antibiotic or antifungal), a cancer therapy, an immunotherapy (e.g., for cancer), an antibody therapy, a protein or peptide therapy, a cell-based therapy, a nucleic acid therapy, or a macrolide.

Exemplary pharmaceutical agents (and some exemplary processed/metabolized forms thereof) include 5-aminosalicylic acid, 5-fluorouracil, balsalazide, benzylpenicillin, BILR 355, calcitonin, chloramphenicol, clonazepam, deleobuvir, diclofenac (glucuronide), digoxin, eltrombopag, flucytosine, glyceryl trinitrate, glycyrrhizin, indocine (n-oxide), indomethacin glucuronide, insulin, isosorbide dinitrate, ketoprofen (glucuronide), levamisole, levodopa, loperamide (N-oxide), lovastatin, methamphetamine, methotrexate, metronidazole, misonidazole, morphine (6-glucuronide), neo-prontosil, nitrazepam, nizatidine, olsalazine, omeprazole, phenacetin, potassium oxonate, prontosil, ranitidine, risperidone, sennosides, irinotecan (SN-38G), sodium picosulfate, sorivudine, succinylsulfathiazole, sulfapyridine, sulfasalazine, sulfinpyrazone, sulindac, and/or zonisamide. Additional exemplary pharmaceutical agents are described herein.

Exemplary pharmaceutical agents (and some exemplary processed/metabolized forms thereof) include:
i) Nonsteroidal anti-inflammatory (NSAID) drug, such as, e.g., 5-aminosalicylic acid and derivatives, e.g., balsalazide, olsalazine, sulfasalazine (aminosalicylate anti-inflammatory drug), diclofenac (⇒glucuronide) (for pain, migraines, and arthritis), indocine (⇒N-oxide), indomethacin (⇒glucuronide), ketoprofen (⇒glucuronide) (propionic acid class), sulindac,
ii) Chemotherapeutic drug, such as, e.g., 5-fluorouracil and methotrexate (antimetabolite antineoplastic agents and immunosuppressants for cancer), irinotecan (SN-38G) (colon and rectum cancer)
iii) Antibiotics/Antibacterials, such as, e.g., benzylpenicillin (a penicillin-class antibacterial), chloramphenicol, metronidazole, prontosil, neo-prontosil,
sulfapyridine iv) Antivirals, such as, e.g., BILR 355 and sorivudine (a nucleoside analog/reverse transcriptase inhibitor, e.g., for HIV), deleobuvir (a non-nucleoside polymerase inhibitor for hepatitis C virus)
v) Antifungals, such as, e.g., flucytosine (5-FC) (a Pyrimidine analogue),
vi) Antinematodals (e.g., anti-worm drugs), such as, e.g., levamisole (an immunomodulatory agent for hookworm infections),
vii) Hormones, such as, e.g., calcitonin (a thyroid gland hormone, e.g., for osteoporosis, cancer-related bone pain), insulin (glucose levels)
viii) Sedatives, such as, e.g., clonazepam (a Benzodiazepine for seizures, panic disorder, and anxiety)
ix) Heart medications/high blood pressure medication, such as, e.g., cardiac glycosides, such as, e.g. digoxin (an antiarrhythmic agent for heart failure), glyceryl trinitrate (for heart failure and high blood pressure), isosorbide dinitrate (nitrate) (for chest pain (angina)),
x) Colony-stimulating factors, such as, e.g., eltrombopag (a bone marrow stimulant for thrombocytopenia and aplastic anemia),
xi) Emulsifiers/gel-forming agents, such as, e.g., glycyrrhizin (a saponin, e.g., for foodstuff and cosmetics)
xii) Dopamines, such as, e.g., levodopa (a dopamine precursor for Parkinson's disease and Parkinson's-like symptoms)
xiii) Opioid receptor agonists, such as, e.g., loperamide (⇒N-oxide) (for diarrhea),
xiv) Statins, such as, e.g., lovastatin (for high cholesterol and triglyceride levels),
xv) CNS stimulants, such as, e.g. methamphetamine (for ADHD and recreational drug use)
xvi) Sensitizers/radio-therapy agents, such as, e.g., misonidazole (a nitroimidazole acting as a radiosensitizer in radiation therapy)
xvii) Narcotic pain relievers, such as, e.g., morphine (⇒6-glucuronide),
xviii) Hypnotic drugs, such as, e.g., nitrazepam (benzodiazepine class for anxiety, insomnia, amnestic, anticonvulsant, and skeletal muscle relaxant)
xix) Antiacids/proton-pump inhibitor, such as, e.g., nizatidine, ranitidine and omeprazole (H2 antagonist) (for ulcers, gastroesophageal reflux disease (GERD)),
xx) Analgesics, such as, e.g., phenacetin (pain relief),
xxi) Uricase inhibitors, such as, e.g., potassium oxonate (for inhibition of 5-fluorouracil-induced gastrointestinal toxicity),
xxii) Antipsychotics, such as, e.g., risperidone (for schizophrenia, bipolar disorder, and irritability caused by autism),
xxiii) Laxatives, such as, e.g., sennosides (Senna glycoside) and sodium picosulfate (for constipation)
xxiv) Sulfonamides, such as, e.g., succinylsulfathiazole, sulfapyridine, sulfasalazine,
xxv) Anticonvulsant, such as, e.g., zonisamide (for seizures, epilepsy)
xxvi) Immunotherapy agents, such as, e.g., cytotoxic T-lymphocyte associated antigen 4 (CTLA-4) and CpG oligonucleotides (for cancer)

In embodiments, dietary components can include substances that are diet-derived and that can have bioactivity, e.g., can affect health and/or disease of a subject.

In embodiments, the dietary component comprises caffeic acid, chlorogenic acid, choline, cycasin, ellagic acid, geniposide, 2-amino-3-methylimidazo[4,5-f]quinoline (IQ), quinic acid, an ellagitannin (e.g., punicalagin, pedunculagin), a flavone (e.g., baicalin, catechin/epicatechin, hesperidine, quercetin-3-glucoside), isoflavones (e.g., daidzein, genistein, glycitein), and/or a lignan (e.g., pinoresinol, secoisolariciresinol).

In embodiments, the dietary component comprises a polyphenol, e.g., a polyphenol described herein, e.g., anthocyanin or proanthocyanidin. In embodiments, the dietary component comprises a phytoestrogen, e.g., a phytoestrogen described herein, e.g., isoflavone or lignan. In embodiments, the dietary component comprises a heterocyclic amine. In embodiments, the dietary component comprises a choline-containing compound, e.g., L-carnitine or phosphatidylcholine.

Food additives and/or drug additives can include chemicals that are added to foods and/or drugs, e.g., to enhance their shelf life and/or flavor. Such food additives and drug additives can interact with gut microbes. Examples of food additives include artificial sweeteners (e.g., cyclamate, xylitol, saccharin), emulsifiers (e.g., carboxymethylcellulose or polysorbate-80), and/or contaminants (e.g., melamine). Some contaminants in foods can be toxic, e.g., melamine and processed intermediates thereof. Examles of food and drug additives include acedoben, cyclamate, lactitol, lactulose, melamine, rebaudioside a, and/or stevioside.

Environmental toxins and toxicants can include chemicals that may affect gut microbes, e.g., affect their growth and/or metabolism. Exemplary environmental toxins can include bisphenol A, oxybenzone, fluoride, parabens, phthalates, butylated hydroxyanisole, perfluorooctanoic acid, perchlorate, decabromodiphenyl ether, and asbestos.

Allergens are a type of antigen that produces an abnormal, vigorous immune response to an antigen, which should normally be perceived by the body as harmless. Exemplary classes of allergens include animal components, drugs, foods, insect components, mold spores, chemicals, and plant components. Exemplary animal component allergens include Fel d1 (from cats), animal fur, animal dander, cockroach calyx, wool, and dust mite excretions. Exemlpary drug allergens include penicillin, sulfonamides, and salicylates. Exemplary food allergens include celery, celeriac, corn, maize, eggs (e.g., egg albumin), fruit, legume (e.g., beans, peas, peanuts, soybeans), milk, seafood, sesame, soy, tree nut (e.g., pecan, almond), and wheat. Exemplary insect component allergens include bee sting venom, wasp sting venom, and mosquito stings. Exemlary chemical allergens include nickel sulfate, Balsam of Peru, fragances, quaternium-15, neomycin, latex, and metal. Exemplary plant component allergens include wood, grass (e.g., ryegrass or timothy grass), weed (e.g., ragweed, plantago, nettle, Chenopodium album, sorrel, and Artemisia vulgaris), and trees (e.g., birch, alder, hazel, hornbeam, Aesculus, willow, Platanus, Tilia, Olea, poplar, Ashe juniper, and Alstonia scholaris).

### Processing of Exogenous Substances

### Chemical Reactions

In embodiments, the processing of the exogenous substance comprises modulation of the level of derivatization and/or degradation of the exogenous substance. In an embodiment, the glycan composition modulates the ability of a microbe, e.g., of the gut, to produce an entity, e.g., an enzyme, that processes, e.g., derivatizes and/or degrades, the exogenous substance, e.g., a drug or a drug metabolite or intermediate. In embodiments, the processing of the exogenous substance comprises metabolizing (e.g., generating of one or more metabolites or intermediates, e.g., from the exogenous substance as a starting material).

In embodiments, the processing of the exogenous substance comprises a reaction such as hydrolysis, oxidation, reduction, aromatization, alkylation, acylation, phosphorylation, glycosylation, sulfation, and/or nitrosylation. In an embodiment, the glycan composition modulates the ability of a microbe, e.g., the gut, to produce an enzyme that catalyzes the hydrolysis, oxidation, reduction, aromatization, alkylation, acylation, phosphorylation, glycosylation, sulfation, and/or nitrosylation, the exogenous substance. In embodiments, the exogenous substance is a drug, a drug metabolite, a drug additive, a food, a food additive, an allergen, a toxin or toxicant.

In embodiments, the processing occurs in vivo, e.g., in a host, e.g., subject described herein.

Provided herein are methods of (i) hydroxylating, (ii) methylating, (iii) sulfonating, (iv) hydrolyzing, (v) oxidizing, (vi) reducing, (vii) aromatizing, (viii) alkylating, (ix) acylating, (x) phosphorylating, (xi) glycosylating, (xii) sulfating, and/or (xiii) nitrosylating, an exogenous substance in vivo in a subject, comprising administering a glycan composition to the subject. In an embodiment, the glycan composition modulates the ability of a microbe of the microbiome, e.g., of the gut, to produce an enzyme that i) hydroxylates, (ii) methylates, (iii) sulfonates, (iv) hydrolyzes, (v) oxidizes, (vi) reduces, (vii) aromatizes, (viii) alkylates, (ix) acylates, (x) phosphorylates, (xi) glycosylates, (xii) sulfates, and/or (xiii) nitrosylates, the exogenous substance. In embodiments, the exogenous substance is a drug, a drug metabolite, a drug additive, a food, a food additive, an allergen, a toxin or toxicant.

In some embodiments, modification of the exogenous substance can be detected and/or pharmacokinetic parameters can be determined using mass spectrometry analsysis, e.g., mass spectrometry analsys of blood, fecal, or urine samples taken from a subject. In some embodiments, modification of the exogenous substance can be detected and/or pharmacokinetic parameters can be determined using in vitro tests, e.g., using isolated exogenous substances as substrates and isolated biological samples (e.g., fecal samples, such as fecal slurries), isolated microbes (e.g., isolated bacterial taxa), and/or isolated enzymes or purified enzyme extracts (e.g., microbial enzymes) to modify the exogenous substances in vitro, e.g., in a test vessel, optionally using appropriate solvents, buffers, energy sources, and other suitable reaction conditions and assays that are suitable to detect the modification.

In embodiments, any one of the (i)-(xiii) is performed by a microbe, e.g., bacterial taxa. In embodiments, any one of the (i)-(xiii) is performed by an enzyme, e.g., microbial enzyme. In embodiments, any one of the (i)-(xiii) is not performed by a host enzyme (e.g., a non-microbial, human or mammalian enzyme). In embodiments, any one of the (i)-(xiii) is performed in the gastrointestinal tract, e.g., the stomach, small intestine, and/or large intestine. In embodimetns, any one of the (i)-(xiii) is performed in a region of the small intestine (e.g., the duodenum, jejunum, or ileum). In embodiments, any one of the (i)-(xiii) is performed in a region of the large intestine (e.g., cecum, colon, or rectum). In embodiments, any one of the (i)-(xiii) is substantially performed in the colon.

### Processing Enzymes

In embodiments, the processing of the exogenous substance is performed by an enzyme, e.g., a microbial (e.g., bacterial) enzyme or a host enzyme (e.g., eukaryotic, e.g., mammalian, e.g., human enzyme). In embodiments, the processing includes derivatization and/or degradation. The processing, e.g., the derivatization and/or degradation, can be carried out by an enzyme described herein. Exemplary enzymes include: (i) oxidoreductase (EC 1) (e.g., dehydrogenases, oxidases, catalases), (ii) transferase (EC 2) (e.g., aminotransferases, peptidyltransferases, glycosyltransferases), (iii) hydrolase (EC 3) (e.g., reductases (e.g. metal reductases), aromatase/cyclases, phosphorylases, glycosidases, cellulases, amylases, ureases, lipases, proteases, peptidases, mannanases, pullulanases, xylanases), (iv) lyase (EC 4) (e.g., pectate lyases), (v) isomerase (EC 5) (e.g., epimerases, mutases);(vi) ligase (EC 6) (e.g., synthases);(vii) azoreductase (e.g., arylamine N-acetyltransferase);(viii) beta-glucuronidase (e.g., uridine diphosphate (UDP)-glucuronosyltransferase); and/or (ix) carboxylesterase.

In embodiments, the enzyme acts on one or more of the following types of bonds: (i) ester bonds;(ii) ether bonds;(iii) peptide bonds;(iv) carbon-nitrogen bonds, e.g., other than peptide bonds;(v) acid anhydrides;(vi) carbon-carbon bonds;(vii) halide bonds;(viii) phosphorus-nitrogen bonds;(ix) sulfur-nitrogen bonds;(x) carbon-phosphorus bonds;(xi) sulfur-sulfur bonds; and/or(xii) carbon-sulfur bonds.

In embodiments, the enzyme comprises a reductase, e.g., nitrate/nitrite/nitric oxide reductase, arsenate reductase, an iron/ferric reductase, a chlorate reductase, a fumarate reductase, an aldehyde reductase, a peroxide reductase, a CO₂-reductase, a morphinone reductase, a TMAO reductase, a sulfite reductase, a DMSO reductase, a ribonucleotide reductase, a fatty acid reductase, a xylose reductase, a thioredoxin reductase, a chromium reductase, a perchlorate reductase, or a dihydrofolate reductase.

In embodiments, the enzyme comprises a hydrolase, e.g., a carboxylic-ester hydrolase, a thioester hydrolase, a phosphoric-mono(di-) (tri)ester hydrolase, a sulfuric-ester hydrolase, a diphosphoric-monoester hydrolase, a phosphoric-triester hydrolase, an exodeoxyribonuclease, an exonuclease, an endodeoxyribonuclease, an endoribonuclease, a glycosylase, a glycosidase (O, N, or S glycosidase), a trialkylsulfonium hydrolase, an ether hydrolase, or a peptidase.

In embodiments, the peptidase comprises an a-amino-acyl-peptide hydrolase, a peptidyl-amino-acid hydrolase, a dipeptide hydrolase, a peptidyl peptide hydrolase, an aminopeptidase, a peptidylamino-acid hydrolase, an acylamino-acid hydrolase, a dipeptidase, a dipeptidyl-peptidase, a tripeptidyl-peptidase, a peptidyl-dipeptidase, a serine-type carboxypeptidase, a metallocarboxypeptidase, a cysteine-type carboxypeptidase, an omega peptidase, a serine endopeptidase, a cysteine endopeptidase, an aspartic endopeptidase, a metalloendopeptidase, a threonine endopeptidase, or an endopeptidase.

Enzymes can be produced by any of the exemplary bacterial taxa described herein, e.g., in Tables 1-6.

In embodiments, the enzyme comprises an activity, e.g., action on a substrate, described herein, e.g., in Tables 1-3.

Methods described herein include modifying an enzyme activity, e.g., activity or level of a microbial or mammalian enzyme, using a glycan composition described herein. In an embodiment, the modification of enzyme activity is or includes the increase of activity by an increase in the number or prevalence (relative abundance) of a microbe (e.g., a microbe comprising or capable of producing the enzyme or producing or capable of producing an entity that increases enzyme activity). In an embodiment, the modification of enzyme activity is or includes the decrease of activity by a decrease in the number or prevalence (relative abundance) of a microbe (e.g., a microbe comprising or capable of producing the enzyme or producing or capable of producing an entity that decreases enzyme activity).

Enzyme activity (or enzymatic activity) can include the level of (e.g., expression level of) the enzyme, activity (e.g., specific activity) of the enzyme, and/or availability/bioavailability of the enzyme, e.g, in a host. In some cases, an increase in enzyme activity may be desired, e.g., where the enzyme generates an active drug form from a prodrug, or where the enzyme detoxifies a substrate. In embodiments, the method comprises increasing the enzyme activity, e.g., by at least 5% (e.g., at least 5%, 10%, 15%, 20%, 25%, 30%, 40%, 50%, 60%, 70%, 80%, 90%, 95%, 97%, 98%, 99%, or at least 100%), or at least 1.5-fold, 2-fold, 3-fold, 4-fold, 5-fold, 10-fold, 15-fold, 20-fold, 25-fold, 50-fold, 100-fold, 250-fold, 500-fold, at least 1000-fold, or more), e.g., relative to a reference level (e.g., level of processing that occurs in the subject prior to administration of the glycan composition).

In some cases, a decrease in enzyme activity may be desired, e.g., where the enzyme creates a toxic product, e.g., metabolite or intermediate, or where the enzyme modifies an active drug form such that it is eliminated more rapidly (e.g., excreted), or otherwise decreased in its bioavailability. In embodiments, the method comprises decreasing the processing (e.g., amount of substance processed and/or rate at which the substance is processed), e.g., by at least least 5% (e.g., at least 5%, 10%, 15%, 20%, 25%, 30%, 40%, 50%, 60%, 70%, 80%, 90%, 95%, 97%, 98%, or at least 99%), or at least 1.5-fold, 2-fold, 3-fold, 4-fold, 5-fold, 10-fold, 15-fold, 20-fold, 25-fold, 50-fold, 100-fold, 250-fold, 500-fold, at least 1000-fold, or more), e.g., relative to a reference level (e.g., level of processing that occurs in the subject prior to administration of the glycan composition).

In some embodiments, an enzyme activity is stereospecific, e.g., the enzyme activity will target one stereoisomer of a drug, metabolite, or toxic product but not another stereoisomer of the drug, metabolite, or toxic product. Drugs administered to patients are often mixtures, e.g., racemic mixtures, of stereoisomers. In some embodiments, the enzyme activity, e.g., modulated by increasing or decreasing the prevalence of a bacterial taxa in a method described herein, targets one stereoisomer of a drug, metabolite, or toxic product. In some embodimemts, the stereisomer targeted is the biologically active stereoisomer of the drug, metabolite, or toxic product. In some embodiments, the stereoisomer targeted is the toxic product (e.g., the other stereoisomer is not substantially toxic). Stereoisomers include enantiomers, diastereomers, cis-trans isomers, conformers, anomers, atropisomers, epimers, and configurational stereoisomers.

Bacterial Taxa that Process the Exogenous Substance

In embodiments, the exogenous substance is processed by a bacterium, e.g., bacterial taxa, e.g., an enzyme produced by a bacterial taxa. In embodiments, the exogenous substance is metabolized by the bacterial taxa. In embodiments, the processing comprises decreasing the amount of an exogenous substance or derivative/metabolite/intermeduates thereof that is toxic to the subject.

In embodiments, the processing comprises increasing excretion of the toxic derivative, e.g., decreasing the synthesis of the toxic derivative.

Exemplary bacterial taxa that can process an exogenous substance include those described herein, e.g., in Tables 1-6.

In embodiments, methods described herein comprise increasing the processing (e.g., amount of substance processed and/or rate at which the substance is processed), e.g., least 5% (e.g., at least 5%, 10%, 15%, 20%, 25%, 30%, 40%, 50%, 60%, 70%, 80%, 90%, 95%, 97%, 98%, 99%, or at least 100%), or at least 1.5-fold, 2-fold, 3-fold, 4-fold, 5-fold, 10-fold, 15-fold, 20-fold, 25-fold, 50-fold, 100-fold, 250-fold, 500-fold, at least 1000-fold, or more), e.g., relative to a reference level (e.g., level of processing that occurs in the subject prior to administration of the glycan composition).

In embodiments, methods described herein comprise decreasing the processing (e.g., amount of substance processed and/or rate at which the substance is processed), e.g., by at least least 5% (e.g., at least 5%, 10%, 15%, 20%, 25%, 30%, 40%, 50%, 60%, 70%, 80%, 90%, 95%, 97%, 98%, or at least 99%), or at least 1.5-fold, 2-fold, 3-fold, 4-fold, 5-fold, 10-fold, 15-fold, 20-fold, 25-fold, 50-fold, 100-fold, 250-fold, 500-fold, at least 1000-fold, or more), e.g., relative to a reference level (e.g., level of processing that occurs in the subject prior to administration of the glycan composition).

### Area in Intestine of Targeting/Processing

In embodiments, the exogenous substance is present in, passes through, and/or travels to, the gastrointestinal tract (e.g., the stomach, small intestine, and/or large intestine). In embodiments, the exogenous substance is present in, passes through, and/or travels to, a region of the small intestine (e.g., the duodenum, jejunum, or ileum). In embodiments, the exogenous substance is present in, passes through, and/or travels to, a region of the large intestine (e.g., cecum, colon, or rectum). In embodiments, the exogenous substance is present in, passes through, and/or travels to the colon. In embodiments, the exogenous substance is present systemically in (e.g., is present in, passes through, and/or travels to, the circulation of) the subject. In embodiments, the exogenous substance accummulates locally, e.g., in an organ (e.g., the liver or kidney) in the subject.

In embodiments, the exogenous substance is processed (e.g., as described herein) in the gastrointestinal tract (e.g., the stomach, small intestine, and/or large intestine), e.g., a region of the small intestine (e.g., the duodenum, jejunum, or ileum); or a region of the large intestine (e.g., cecum, colon, or rectum). In embodiments, the exogenous substance is processed (e.g., as described herein) in the colon.

### Exemplary Processes and Effects

Tables 1, 2, 3, 4, 5, and 6 include exemplary processing enzymes, exemplary exogenous substances, exemplary bacterial taxa, examples of the action of the enzymes on the exogenous substances, and/or examples of effects that a glycan composition described herein can have on the enzyme and/or taxa. These effects include desired effects, e.g., to increase drug efficacy (e.g., where the exogenous substance comprises a drug) and/or decrease drug toxicity, and/or reduce exposure to toxic metabolites/substances.

TABLE 1
Processes mediated by microbial enzymes

| | | | | | Desired effect on | Exemplary | |
|---|---|---|---|---|---|---|---|
| | | | | | the enzyme and/or | diseases/ | |
| | | | | | taxa (e.g., in order | conditions | Exemplary glycans |
| Row | | | Substrate (e.g., | Enzyme can be | to increase drug | applicable to | that can be used |
| Num- | | Action of | exogenous | present in the | efficacy or decrease | the enzyme/ | to achieve |
| ber | Enzyme | Enzyme | substance) | following microbes | toxicity)^{1,2} | substrate/taxa | desired effect |
| 1 | Microbial | Inactivates substrate | 5-aminosalicylic acid (the | | Inhibit or reduce | Rheumatoid arthritis | |
| | arylamine N- | | bioactive component of the | | levels/activity | | |
| | acetyltransferase | | drug sulfasalazine) | | | | |
| 2 | Microbial | Activates substrate to | Prodrug, e.g., protonsil, an | | Increase levels/ | Bacterial infection | |
| | azoreductase | active drug form | antibacterial drug | | activity. | | |
| | | (thereby increasing | | | | | |
| | | bioavailability) | | | | | |
| 3 | Microbial | Activates substrate to | Prodrug, e.g., sulfasalazine | Bacteroides sp., | Increase levels/ | Rheumatoid arthritis | man100, glu100, |
| | azoreductase | active drug form | | Enterococcus, | activity (converts | | man75gal25, |
| | | | | faecalis | the prodrug | | man52glu29gal19, |
| | | | | Lactobacillus sp. | protonsil into | | glu50gal50, ara100, |
| | | | | | 5-aminosalicylic | | FOS, gal100, |
| | | | | | acid | | glu60man40 |
| 4 | Beta- | Adds glucuronic acid | Pharmaceutical agents, e.g., | | Inhibit or reduce | Cancer, | |
| | glucuronidase | to substrate (thereby | used to treat cancer or | | levels/activity | inflammation | |
| | (e.g., uridine | interfering with the | inflammation, hormones, | | of the enzyme to | | |
| | diphosphate | biological activity of | bile acids | | decrease | | |
| | (UDP)- | the substrate and/or | | | inactivation | | |
| | glucuronosyl- | increasing their | | | or decrease | | |
| | transferase), | elimination). | | | eliminatinon of | | |
| | e.g., in | | | | the drug | | |
| | microbes | | | | | | |
| 5 | Microbial | Removes sugar | SN-38 glucuronide | Gut microbes (e.g., | Inhibit or reduce | Cancer, e.g., | man100, gal100, |
| | beta- | moiety from SN-38 | | Proteobacteria, | levels/activity of | colorectal cancer, | man52glu29gal19, |
| | glucuronidase | glucuronide, resulting | | Firmicutes, | the enzyme to | drug-induced side | man75gal25, |
| | (e.g., in | in a toxic compound | | or Actinobacteria) | thereby decrease | effects (e.g., | glu33gal33man34, |
| | intestine) | to intestinal epithelial | | | the production of | diarrhea) | glu100, glu50gal50, |
| | | cells (the prodrug is | | | a toxic metabolite | | lactulose, |
| | | irrenotecan) | | | | | glu33gal33ara33, |
| | | | | | | | FOS, glu60man40, |
| | | | | | | | ara100 |
| 6 | Microbial | Liberates the | Glucuronide from an | | Inhibit or reduce | Inflammation, drug- | |
| | beta- | glucuronide, e.g., | NSAID | | levels/activity | induced side effects | |
| | glucuronidase | enabling reabsorption | | | | (e.g., diarrhea) | |
| | (e.g., in | of the aglycone (the | | | | | |
| | intestine) | compound that exists | | | | | |
| | | after the removal of a | | | | | |
| | | glycosyl group) into | | | | | |
| | | enterocytes (where | | | | | |
| | | the NSAIDs are | | | | | |
| | | further metabolized | | | | | |
| | | into reactive | | | | | |
| | | metabolites, which | | | | | |
| | | are toxic). | | | | | |
| 7 | Bacterial | Inactivates the | digoxin | Eggerthella lenta, | Inhibit/reduce | Cardiac | ara100, |
| | reductase | substrate to form | | e.g., strain | levels/activity | diseases/disorders, | gal33man33ara33, |
| | (e.g., cardiac | dihydrodigoxin | | DSM2243 | of the enzyme to | e.g., cardiac | glu33gal33ara33, |
| | glycoside | (which decreases the | | | thereby increase | arrhythmia, heart | glu60man40, |
| | reductase) | bioavailability) | | | the activity of | failure | man75gal25, |
| | | | | | drug | | man100, gal100, |
| | | | | | | | FOS, |
| | | | | | | | glu33gal33man34, |
| | | | | | | | glu100, |
| | | | | | | | man52glu29gal19, |
| | | | | | | | glu50gal50 |
| 8 | Microbial | Metabolizes a | Tyrosine and/or | Firmicutes (e.g., | Inhibit/reduce | Pain, fever, drug- | man100, gal100, |
| | enzyme | substrate to p-cresol | phenylalanine | Clostridium | levels/activity | induced toxicity | man52glu29gal19, |
| | | | | difficile), | (in order to | (e.g., from | man75gal25, |
| | | | | Bacteroidetes, | reduce levels of | acetaminophen) | glu33gal33man34, |
| | | | | Actinobacteria, | p-cresol, which | | glu33gal33ara33, |
| | | | | and/or Fusobacteria | competes with | | gal33man33ara33 |
| | | | | | acetaminophen as | | |
| | | | | | substrates of | | |
| | | | | | SULT1A1) | | |
| 9 | Microbial | | Statin, e.g., simvastatin | Microbes that | Increase levels/ | High cholesterol, | |
| | enzyme | | | metabolize | activity (e.g., | coronary artery | |
| | | | | bile acids | in order to increase | disease | |
| | | | | | absorption/levels | | |
| | | | | | of statins) | | |
| 10 | Microbial bile | Deconjugates | Taurine-conjugated bile | Firmicutes (e.g., | Inhibit/reduce | Obesity, e.g., diet- | ara100, fos, |
| | salt hydrolase | taurine-conjugated | acid (e.g., tauro-beta- | Lactobacillus) | levels/activity | induced obesity | gal100, lactulose, |
| | | bile acids into free | muricholic acid) | | (e.g., which can | | man75gal25, |
| | | bile acids | | | be done with an | | |
| | | | | | antioxidant, e.g., | | |
| | | | | | tempol) | | |
| 11 | microbial | Hydrolyzes | ellagitannin | Actinobacteria, e.g., | Increase levels/ | | man100, gal100, |
| | enzyme | ellagitannin to ellagic | | Gordonibacter | activity | | man75gal25, |
| | | acid | | | | | man52glu29gal19, |
| | | | | | | | glu33gal33man34, |
| | | | | | | | glu33gal33ara33, |
| | | | | | | | glu50gal50, |
| | | | | | | | glu100, lactulose, |
| | | | | | | | glu60man40, FOS, |
| | | | | | | | ara100 |
| 12 | microbial | Metabolizes ellagic | Ellagic acid | Actinobacteria, e.g., | Increase levels/ | | man100, gal100, |
| | enzyme | acid to a urolithin | | Gordonibacter | activity | | man75gal25, |
| | | (which has | | | | | man52glu29gal19, |
| | | antioxidant, | | | | | glu33gal33man34, |
| | | anticancer, anti- | | | | | glu33gal33ara33, |
| | | inflammatory, and/or | | | | | glu50gal50, |
| | | anti-microbial | | | | | glu100, lactulose, |
| | | properties) | | | | | glu60man40, FOS, |
| | | | | | | | ara100 |
| 13 | microbial | Metabolizes | Phytoestrogen (e.g., | Actinobacteria, | Increase levels/ | | gal100, |
| | enzyme | phytoestrogen to | isoflavone, lignan), e.g., a | Bacteroidetes, | activity | | man75gal25, |
| | | molecules that bind | glycosidic isoflavone such | Firmicutes | | | man100, |
| | | estrogen receptors | as daidzin | | | | glu33gal33man34, |
| | | (e.g., may have | | | | | man52glu29gal19, |
| | | protective effects | | | | | glu100, glu50gal50, |
| | | against breast cancer) | | | | | glu33gal33ara33, |
| | | | | | | | glu60man40, |
| | | | | | | | gal33man33ara33 |
| 14 | Glycosidic | Daidzin metabolized | daidzin | Enterococcus faecium, | increase levels/ | Breast cancer | glu60man40, |
| | cleavage and | to equol (which binds | | Lactobacillus mucosae, | activity of the | | gal33man33ara33, |
| | reduction of | estrogen receptor- | | Bifidobacterium sp., | enzyme to thereby | | man75gal25, |
| | an α,β- | beta and may be | | Eggerthella sp. | increase the | | glu50gal50, |
| | unsaturated | associated with lower | | | levels of equol | | man100, FOS, |
| | ketone, e.g., | risk/incidence of | | | | | lactulose |
| | by a microbial | breast cancer) | | | | | |
| | enzyme | | | | | | |
| 15 | Microbial | Metabolizes | Lignan (from plants), e.g., | E. faecalis, E. lenta, | increase levels/ | | glu100, |
| | enzyme | pinoresinol and | pinoresinol, | Blautia product, | activity | | glu60man40, |
| | | secoisolariciresinol to | secoisolariciresinol | Eubacterium limosum, | | | ara100, |
| | | enterodiol and | | Clostridium scindens, | | | gal33man33ara33, |
| | | enterolactone (which | | Lactonifactor | | | glu33gal33ara33, |
| | | may be protective | | longoviformis, | | | glu50gal50, |
| | | against breast cancer) | | Clostridium | | | man75gal25, |
| | | | | saccharogumia, | | | man52glu29gal19, |
| | | | | P. product, | | | glu33gal33man34 |
| 16 | Microbial | Reactivates | Heterocyclic amine (e.g., | Bacteria that carry | Inhibit/reduce | cancer | ara100, |
| | beta- | glucuronidated | formed during the burning | the uidA gene, e.g., | levels/activity | | gal33man33ara33, |
| | glucuronidase | substrate (detoxified | of meat), which can be | Escherichia coli | of the enzyme, | | glu33gal33ara33, |
| | | by hepatic | carcinogenic. E.g., 2- | | thereby reducing | | glu60man40, |
| | | glucuronidation), by | amino-3- | | the generation | | man75gal25, |
| | | removing the | methylimidazo[4,5- | | of a toxic (e.g., | | man100, FOS, |
| | | conjugate, generating | f]quinolone (IQ), 2-amino- | | carcinogenic) | | glu33gal33man34, |
| | | a toxic compound | 1-methyl-6- | | compound | | man52glu29gal19, |
| | | | phenylimidazo[4,5- | | | | gal100, lactulose, |
| | | | b]pyridine (PhIP), 2-amino- | | | | glu100, glu50gal50 |
| | | | 3,8-dimethylimidazo[4,5-f]- | | | | |
| | | | quinoxaline (MeIQx). | | | | |
| 17 | Microbial | Metabolizes choline | Choline containing | Microbes in the colon, | Inhibit/reduce | High cholesterol or | |
| | glycyl radical | containing compound | compound, e.g., L-carnitine | e.g., Firmicutes, | levels/activity | a cardiac condition, | |
| | enzyme | to form | | Proteobacteria, | of the enzyme, | atherosclerosis | |
| | | trimethylamine | | Actinobacteria (e.g., | thereby reducing | | |
| | | (TMA), which is | | not including | levels of TMA | | |
| | | linked to higher | | Bacteroides), and/or | | | |
| | | cholesterol and risk | | bacteria carrying the | | | |
| | | of cardiac conditions | | choline utilization | | | |
| | | | | (cut) gene cluster | | | |
| 18 | Microbial | Converts sweetener | Non-caloric artificial | Enterococcus, | Inhibit/reduce | | ara100, |
| | enzyme | to a compound that | sweetener, e.g., cyclamate, | Clostridium, | levels/activity | | gal33man33ara33, |
| | | can be toxic. | xylitol, saccharin | Corynebacterium, | | | glu33gal33ara33, |
| | | E.g., converts | | Campylobacter, | | | glu60man40, |
| | | cyclamate to | | Escherichia | | | man75gal25, |
| | | cyclohexylamine, | | | | | man100, FOS |
| | | which can be toxic | | | | | glu33gal33man34, |
| | | | | | | | tnan52glu29gal19, |
| | | | | | | | gal100, lactulose, |
| | | | | | | | glu100, glu50gal50 |
| 19 | Microbial | Metabolizes | melamine | Gut microbes, e.g., | Inhibit/reduce | Renal condition, | |
| | enzyme | melamine to cyanuric | | Klebsiella terrigena | levels/activity | e.g., renal failure | |
| | | acid, which can lead | | | | | |
| | | to renal toxicity | | | | | |
| 20 | Microbial | Metabolizes bile acid | Bile acid | Clostridium scindens | Increase levels/ | | |
| | enzyme | to secondary bile | | | activity | | |
| | | acid, which inhibits | | | | | |
| | | growth of | | | | | |
| | | Clostridium difficile | | | | | |
| 21 | Microbial | Hydrolyzes esters of | Conjugated | Gut microbes, e.g., | Increase levels/ | Inflammation | |
| | cinnamoyl | caffeic acid and | hydroxycinnamate (e.g., | Bifidobacterium, | activity, e.g., | | |
| | esterase | ferulic acid to form | found in foods, such as | Lactobacillus, | of the enzyme, | | |
| | | the active | fruits, vegetables, cereals, | Escherichia | thereby increasing | | |
| | | compounds, e.g., | and coffee) | | the levels of the | | |
| | | caffeic acid, ferulic | | | active compounds, | | |
| | | acid, and p-coumaric | | | e.g., caffeic acid, | | |
| | | acid (having anti- | | | ferulic acid, | | |
| | | oxidant and anti- | | | and p-coumaric acid | | |
| | | inflammatory | | | | | |
| | | properties) | | | | | |
| 22 | Microbial | Hydrolyzes cycasin | Cycasin, e.g., found in | microbe | Inhibit/reduce | cancer | |
| | enzyme | into the carcinogenic | some plants | | levels/activity | | |
| | | glycoside, | | | of the enzyme, | | |
| | | methylazoxymethanol | | | thereby decreasing | | |
| | | | | | the levels of | | |
| | | | | | methylazoxymethanol | | |
| 23 | Microbial | Liberates aglycone | anthocyanin | microbe | Increase levels/ | cancer | |
| | enzyme | with anticancer | | | activity, e.g., of | | |
| | | properties from | | | the enzyme, thereby | | |
| | | anthocyanins | | | increasing levels of | | |
| | | | | | aglycones having | | |
| | | | | | anticancer properties | | |
| 24 | Microbial | Detoxify oxalate | Oxalate (a strong chelating | Oxalobacter formigenes | Increase levels/ | kidney stones, renal | |
| | enzyme, e.g., | | agent that binds to free | | activity, e.g., of | failure, | |
| | oxalate:formate | | metal cations, thereby | | the enzyme, thereby | hyperoxaluria, and | |
| | antiporter, | | leading to kidney stones, | | decreasing levels | cardiac conduction | |
| | formyl-CoA | | renal failure, hyperoxaluria, | | of oxalate | disorders | |
| | transferase, | | and cardiac conduction | | | | |
| | oxalyl-CoA | | disorders), e.g., in some | | | | |
| | decarboxylase | | plants | | | | |
| 25 | Microbial | Deglycosylates the | Antiviral agent, sorivudine | Enterobacteria, | Inhibit/reduce | Viral infection, e.g., | ara100, |
| | phosphorylase, | active agent, | (active against herpes | e.g., K. pneumoniae | levels/activity | herpes simplex virus | gal33man33ara33, |
| | e.g., thymidine | sorivudine | simplex virus type 1 and | | of microbe or | type 1 and varicella- | glu60man40, FOS, |
| | phosphorylase | | varicella-zoster virus) | | enzyme(s), thereby | zoster virus | man75gal25, |
| | or uridine | | | | decreasing the | | glu33gal33man34, |
| | phosphorylase | | | | deglycosylation | | glu33gal33ara33, |
| | | | | | (inactivation) of | | man52glu29gal19, |
| | | | | | sorivudine | | man100, gal100, |
| | | | | | | | glu100, lactulose, |
| | | | | | | | glu50gal50 |

¹In embodiments of any method described herein, the method can include achieving one or more of the following effects on the enzyme and/or bacterial taxa listed in this table. In embodiments, provided herein are compositions for use in achieving one or more of the following effects on the enzyme and/or bacterial taxa listed in this table.
²Inhibiting or reducing levels/activity can include inhibiting or reducing the levels/activity by at least 5% (e.g., at least 5%, 10%, 20%, 30%, 40%, 50%, 60%, 70%, 80%, 90%, 95%, 99%, 100%, 1.5-fold, 2-fold, 3-fold, 4-fold, 5-fold, 10-fold, 15-fold, 20-fold, 50-fold, 100-fold, 500-fold, 1000-fold, or more). Increasing levels/activity can include increasing levels/activity by at least 5% (e.g., at least 5%, 10%, 20%, 30%, 40%, 50%, 60%, 70%, 80%, 90%, 95%, 99%, 100%, 1.5-fold, 2-fold, 3-fold, 4-fold, 5-fold, 10-fold, 15-fold, 20-fold, 50-fold, 100-fold, 500-fold, 1000-fold, or more).

TABLE 2
Processes mediated by host enzymes

| | | | | Desired effect on | |
|---|---|---|---|---|---|
| | | | | the enzyme and/or | |
| | | | | taxa (e.g., in order | Exemplary |
| | | | Enzyme can be | to increase drug | diseases/conditions |
| | | Substrate (e.g., | present in the | efficacy or decrease | applicable to the |
| Enzyme | Action of Enzyme | exogenous substance) | following microbes | toxicity)^{1,2} | enzyme/substrate/taxa |
| Host (human) | Converts substrate to | Irinotecan (CPT-11) (a | | Increase levels/ | Cancer, e.g., colorectal |
| carboxylesterase | bioactive compound SN- | prodrug for treatment of | | activity | cancer, drug-induced side |
| | 38 | colorectal cancer) | | | effects (e.g., diarrhea) |
| Host Beta- | Re-activates SN-38 | SN-38 | | Inhibit or reduce | Cancer, e.g., colorectal |
| glucuronidase | (thereby causing | | | levels/activity | cancer, drug-induced side |
| (e.g., in liver) | increased toxicity) | | | | effects (e.g., diarrhea) |
| | Glucuronidates SN-38 to | | | | |
| | SN-38 glucuronide (the | | | | |
| | prodrug is irrenotecan) | | | | |
| Host Beta- | Glucuronidates substrate | NSAID (e.g., | | Inhibit or reduce | Inflammation, drug-induced |
| glucuronidase | to a glucuronide | diclofenac, | | levels/activity | side effects (e.g., diarrhea) |
| (e.g., in liver) | | indomethacin, or | | | |
| | | ketoprofen) | | | |
| Liver cytochrome | | Pentobarbital | | | anesthesia |
| P450s (CYPs) | | | | | |
| Liver enzyme(s) | Metabolizes substrate to | acetaminophen | | | Pain, fever, drug-induced |
| | acetaminophen sulfate | | | | toxicity (e.g., from |
| | (inactive), acetaminophen | | | | acetaminophen) |
| | glucuronide (inactive), | | | | |
| | and N-acetyl-p- | | | | |
| | benzoquinone imine | | | | |
| | (NAPQI) (toxic). | | | | |
| Human cytosolic | Converts p-cresol to p- | p-cresol and | | | Pain, fever, drug-induced |
| sulfotransferase | cresol sulfate; converts | acetaminophen | | | toxicity (e.g., from |
| 1A1 (SULT1A1) | acetaminophen to | | | | acetaminophen) |
| | acetaminophen sulfate | | | | |
| | (inactive/non-toxic), | | | | |
| | which leads to less | | | | |
| | formation of toxic | | | | |
| | NAPQI | | | | |
| Host CYP450 | Confers protection | Polycyclic aromatic | Microbe, which | Increase levels/ | cancer |
| | against carcinogenic | hydrocarbon (PAH), | upregulates expression | activity of microbe, | |
| | PAHs | e.g., benzo[a]pyrene, | of host CYP450 | which thereby | |
| | | e.g., found in some | enzyme(s) | increases host | |
| | | plant and animal foods, | | CYP450 enzyme(s), | |
| | | e.g., meats cooked over | | thereby conferring | |
| | | open flame | | protection against | |
| | | | | carcinogenic PAHs | |
| Host 5-FU | Metabolizes 5-FU, | Antiviral agent, | Microbe, which | Decrease levels of | Herpes zoster, e.g., in a |
| catabolic enzyme, | preventing its toxic | sorivudine, in | generates a metabolite | the microbe, thereby | cancer patient |
| dihydropyrimidine | buildup | combination with 5- | of sorivudine, e.g., | preventing the toxic | |
| dehydrogenase | | fluorouracil (5-FU) | (E)-5-(2- | buildup of 5-FU, | |
| (DPD) | | | bromovinyl)uracil | e.g., in patients | |
| | | | | prescribed both | |
| | | | | sorivudine and 5-FU | |
| | | | (BVU). BVU inhibits | | |
| | | | the 5-FU catabolic | | |
| | | | enzyme, | | |
| | | | dihydropyrimidine | | |
| | | | dehydrogenase (DPD) | | |

¹In embodiments of any method described herein, the method can include achieving one or more of the following effects on the enzyme and/or bacterial taxa listed in this table. In embodiments, provided herein are compositions for use in achieving one or more of the following effects on the enzyme and/or bacterial taxa listed in this table.
²Inhibiting or reducing levels/activity can include inhibiting or reducing the levels/activity by at least 5% (e.g., at least 5%, 10%, 20%, 30%, 40%, 50%, 60%, 70%, 80%, 90%, 95%, 99%, 100%, 1.5-fold, 2-fold, 3-fold, 4-fold, 5-fold, 10-fold, 15-fold, 20-fold, 50-fold, 100-fold, 500-fold, 1000-fold, or more). Increasing levels/activity can include increasing levels/activity by at least 5% (e.g., at least 5%, 10%, 20%, 30%, 40%, 50%, 60%, 70%, 80%, 90%, 95%, 99%, 100%, 1.5-fold, 2-fold, 3-fold, 4-fold, 5-fold, 10-fold, 15-fold, 20-fold, 50-fold, 100-fold, 500-fold, 1000-fold, or more).

TABLE 3
Additional exemplary interactions between microbes and exogenous substances

| | | | | | Desired effect on | Exemplary | |
|---|---|---|---|---|---|---|---|
| | | | | | the enzyme and/or | diseases/ | |
| | | | | | taxa (e.g., in order | conditions | Exemplary glycans |
| Row | | | Substrate (e.g., | Enzyme can be | to increase drug | applicable to | that can be used |
| Num- | | Action of | exogenous | present in the | efficacy or decrease | the enzyme/ | to achieve |
| ber | Enzyme | Enzyme | substance) | following microbes | toxicity)^{1,2} | substrate/taxa | desired effect |
| 1 | z | | Cyclophosphamide (e.g., | Gram-positive | Increase levels/ | cancer | |
| | | | for cancer therapy) | bacteria, e.g., | activity | | |
| | | | | described herein | | | |
| 2 | | | CpG-oligonucleotide | Bacteria, e.g., | Increase levels/ | cancer | FOS, ara100, |
| | | | immunotherapy for | described herein, | activity | | gal33man33ara33, |
| | | | cancer | e.g., gram negative | | | glu60man40, |
| | | | | bacteria, e.g., | | | glu33gal33ara33, |
| | | | | that produce | | | glu50gal50, glu100, |
| | | | | lipopolysaccharide | | | man52glu29gal19, |
| | | | | | | | glu33gal33man34, |
| | | | | | | | man100, |
| | | | | | | | man75gal25, gal100 |
| 3 | | | oxaliplatin | bacteria, e.g., | Increase levels/ | cancer | |
| | | | | described herein | activity | | |
| 4 | | | Programmed death | Bifidobacteria | Increase levels/ | cancer | |
| | | | ligand-1 (PDL1) | | activity | | |
| | | | inhibitor (e.g., | | | | |
| | | | antibody) | | | | |
| 5 | | | Cytotoxic T | Bacteroides, e.g., | Increase levels/ | cancer | Man72gal25, |
| | | | lymphocyte | Bacteroides | activity | | glu33gal33man34, |
| | | | protein 4 (CTLA4) | thetaiotaomicron | | | glu50gal50, |
| | | | inhibitor | and/or Bacteroides | | | man100, glu100, |
| | | | (e.g., antibody) | fragilis | | | man52glu29gal19, |
| | | | | | | | ara100, FOS, |
| | | | | | | | gal100, glu60man40 |
| 6 | | | Anti-inflammatory | Staphylococcus | Increase levels/ | Inflammation, | Man100, |
| | | | drugs to treat | | activity | e.g., | glu60man40 |
| | | | inflammatory bowel | | | inflammatory | |
| | | | disease, e.g., | | | bowel disease | |
| | | | tumor necrosis | | | | |
| | | | factor (TNF) | | | | |
| | | | inhibitors (e.g., | | | | |
| | | | antibodies) | | | | |
| 7 | | | Dietary polyphenol | Akkermansia | Increase levels/ | Metabolic | |
| | | | (e.g., anthocyanin | muciniphila | activity (e.g., in | syndrome, | |
| | | | (ACN) and/or | (thought to preserve | order to reduce | insulin | |
| | | | proanthocyanidin | the integrity of | adiposity, insulin | resistance, diet- | |
| | | | (PAC)) | the gut mucus layer) | resistance, and/or | induced weight | |
| | | | | | inflammation, e.g., | gain, adiposity, | |
| | | | | | in obese subjects) | inflammation, | |
| | | | | | | e.g., intestinal | |
| | | | | | | inflammation, | |
| | | | | | | oxidative stress, | |
| | | | | | | conditions with | |
| | | | | | | the gut mucus | |
| | | | | | | layer | |
| 8 | | | Emulsifying agent, | Bacteroidetes (e.g., | Inhibit/reduce | | ara100, lactulose, |
| | | | e.g., carboxy- | Bacteroidales), | levels/activity, e.g., | | gal33man33ara33, |
| | | | methylcellulose, | mucolytic | in order to decrease | | glu60man40, |
| | | | polysorbate-80 | bacteria such as | risk/incidence of | | man100, |
| | | | | Ruminococcus gnavus | metabolic syndrome, | | glu33gal33ara33, |
| | | | | | inflammation. | | glu33gal33man34, |
| | | | | | | | man75gal25, |
| | | | | | | | glu50gal50, |
| | | | | | | | man52glu29gal19, |
| | | | | | | | gal100, glu100 |
| 9 | | | Tacrolimus | Faecalibacterium | increase levels/ | | |
| | | | | prausnitzii | activity, e.g., in | | |
| | | | | | order to increase | | |
| | | | | | tacrolimus efficacy | | |
| 10 | | Inactivation | Dopamine precursor, | Helicobacter pylori | Inhibit/reduce | Parkinson | |
| | | of L-DOPA | levodopa (L-DOPA) | (which can directly | levels/activity of | disease, peptic | |
| | | and/or | | bind to L-DOPA, | H. pylori or an | ulceration in | |
| | | decrease its | | decreasing its | enzyme or reactive | Parkinson's | |
| | | bio- | | bioavailability) | oxygen species that | patients | |
| | | availability | | | inactivates L- | | |
| | | | | | DOPA, thereby | | |
| | | | | | increasing the | | |
| | | | | | levels of active | | |
| | | | | | L-DOPA and/or | | |
| | | | | | its bioavailability | | |
| 11 | | | Heterocyclic amine | Gut microbes, e.g., | Increase levels of | cancer | |
| | | | (HCA), e.g., formed | Lactic acid bacteria | the microbes, which | | |
| | | | during charring | (which can directly | directly bind to the | | |
| | | | of meat, poultry and/ | bind to a HCA) | HCA, thereby | | |
| | | | or fish, e.g., 2- | | potentially preventing | | |
| | | | amino-1-methyl- | | induction of DNA | | |
| | | | 6-pehylimidazol[4,5- | | damage and | | |
| | | | b]pyridine | | preneoplastic lesions. | | |
| | | | (PhIP), 2-amino-3- | | | | |
| | | | methylimidazo[4,5- | | | | |
| | | | f]quinolone (IQ), | | | | |
| | | | 2-amino-3,8-dimethyl- | | | | |
| | | | imidazo[4,5- | | | | |
| | | | f]quinoxaline | | | | |
| | | | (MeIQx), 3-amino- | | | | |
| | | | 1-methyl-5H- | | | | |
| | | | pyrido(4,3-b)indole | | | | |
| | | | (Trp-P-2), other | | | | |
| | | | diet-derived | | | | |
| | | | mutagens | | | | |

¹In embodiments of any method described herein, the method can include achieving one or more of the following effects on the enzyme and/or bacterial taxa listed in this table. In embodiments, provided herein are compositions for use in achieving one or more of the following effects on the enzyme and/or bacterial taxa listed in this table.
²Inhibiting or reducing levels/activity can include inhibiting or reducing the levels/activity by at least 5% (e.g., at least 5%, 10%, 20%, 30%, 40%, 50%, 60%, 70%, 80%, 90%, 95%, 99%, 100%, 1.5-fold. 2-fold, 3-fold, 4-fold, 5-fold, 10-fold, 15-fold, 20-fold, 50-fold, 100-fold, 500-fold, 1000-fold, or more). Increasing levels/activity can include increasing levels/activity by at least 5% (e.g., at least 5%, 10%, 20%, 30%, 40%, 50%, 60%, 70%, 80%, 90%, 95%, 99%, 100%, 1.5-fold, 2-fold, 3-fold, 4-fold, 5-fold, 10-fold, 15-fold, 20-fold, 50-fold, 100-fold, 500-fold, 1000-fold, or more).

TABLE 4
Genus level Microbial Constituents of the GI tract

| Phylum | Class | Genus |
|---|---|---|
| Actinobacteria | Actinobacteria | Actinomyces, Adlercreutzia, Atopobium, Bifidobacterium, Collinsella, |
| | | Corynebacterium, Eggerthella, Mobiluncus, Propionibacterium, Rothia, Slackia |
| Bacteroidetes | Bacteroidia | Alistipes, Bacteroides, Dysgonomonas, Odoribacter, Parabacteroides, |
| | | Porphyromonas, Prevotella, Tannerella |
| | Flavobacteria | Capnocytophaga |
| Firmicutes | Bacilli | Bacillus, Enterococcus, Gemella, Granulicatella, Lactobacillus, Lactococcus, |
| | | Staphylococcus, Streptococcus, Turicibacter, Weissella |
| | Clostridia | Acidaminococcus, Anaerococcus, Anaerofilum, Anaerofustis, Anaerostipes, |
| | | Anaerotruncus, Anaerovorax, Bacteroides, Bacteroides, Blautia, Clostridium, |
| | | Coprococcus, Dehalobacterium, Dialister, Dorea, Eubacterium, Faecalibacterium, |
| | | Finegoldia, Lachnobacterium, Lachnospira, Megamonas, Megasphaera, Mitsuokella, |
| | | Moryella, Oribacterium, Oscillospira, Peptococcus, Peptoniphilus, |
| | | Peptostreptococcus, Phascolarctobacterium, Pseudobutyrivibrio, Roseburia, |
| | | Ruminococcus, Ruminococcus, Selenomonas, Subdoligranulum, Veillonella |
| Fusobacteria | Fusobacteria | Fusobacterium, Leptotrichia |
| | Betaproteobacteria | Comamonas, Herbaspirillum, Lautropia, Neisseria, Oxalobacter, Sutterella |
| | Deltaproteobacteria | Bilophila, Desulfovibrio, LE30 |
| | Epsilonproteobacteria | Campylobacter, Helicobacter |
| | Gammaproteobacteria | Actinobacillus, Aggregatibacter, Citrcbacter, Escherichia, Haemophilus, Klebsiella, |
| | | Moraxella, Pseudomonas, Raoultella |
| Spirochaetes | Spirochaetes | Treponema |
| Synergistetes | Synergistetia | Cloacibacillus, Synergistes |
| Tenericutes | Erysipelotrichi | Bulleidia, Catenibacterium, Clostridium, Coprobacillus, Holdemania, RFN20 |
| | Mollicutes | Asteroleplasma, Mycoplasma |
| Verrucomicrobia | Verrucomicrobiae | Akkermansia |
| Euryarchaeota | Methanobacteria | Methanobrevibacter |

TABLE 5
Genus level microbial constituents predominant in the large
intestine (compared to small intestine) in healthy humans.

| Phylum | Class | Genus |
|---|---|---|
| Bacteroidetes | Bacteroidia | Bacteroides, Butyricimonas, Odoribacter, |
| | | Parabacteroides, Prevotella |
| Firmicutes | Clostridia | Anaerotruncus, Phascolarctobacterium, Ruminococcus, |
| Proteobacteria | Deltaproteobacteria | Bilophila |
| Verrucomicrobia | Verrucomicrobiae | Akkermansia |

TABLE 6
Genus level microbial constituents predominant in the small
intestine (compared to large intestine) in healthy humans.

| Phylum | Class | Genus |
|---|---|---|
| Actinobacteria | Actinobacteria | Cryocola, Mycobacterium |
| Firmicutes | Bacilli | Enterococcus, Lactococcus, Streptococcus, Turicibacter |
| | Clostridia | Blautia, Coprococcus, Holdemania, Pseudoramibacter |
| | | Eubacterium |
| Proteobacteria | Alphaproteobacteria | Agrobacterium, Sphingomonas |
| | Betaproteobacteria | Achromobacter, Burkholderia, Ralstonia |

### Methods of Affecting Drug Toxicity/Efficacy

Bacterial taxa can process (e.g., using bacterial enzyme(s)) a substance, e.g., a drug, to generate or release a toxic compound/molecule. Bacterial taxa can also process (e.g., using bacterial enzyme(s)) a toxic substance (e.g., a drug or a metabolic intermediate produced by the processing of the drug, e.g., by host cells or cells of other bacterial taxa) such that it is less toxic, e.g., bacterial taxa can detoxify the toxic substance. In other examples, bacterial taxa can process (e.g., using bacterial enzyme(s)) a substance, e.g., prodrug, to convert it to an active form, e.g., thereby increasing its efficacy. In yet other examples, bacterial taxa can process (e.g., using bacterial enzyme(s)) a substance, e.g., a drug, e.g., active drug form, such that it is less active or not active. Methods for modulating microbial taxa and microbial activity toward exogenous substances are provided herein comprising administering a glycan composition described herein in an effective amount to modulate the microbial taxa and/or microbial activity toward exogenous substances. The compositions and methods described herein can modulate one or more bacterial taxa and/or one or more bacterial enzymes such that toxicity from substances such as drugs is reduced, and/or efficacy of drugs is increased. For example, the compositions and methods described herein can decrease the level of bacterial taxa (and/or decrease the activity of bacterial enzyme(s)) that generate and/or release of toxic compounds/molecules. In some examples, the compositions and methods described herein can increase the level of bacterial taxa (and/or increase the activity of bacterial enzyme(s)) that detoxify substances. In other examples, the compositions and methods described herein can increase the level of bacterial taxa (and/or increase the activity of bacterial enzyme(s)) that increase the efficacy of a drug, e.g., that convert a prodrug into active form. In yet other examples, the compositions and methods described herein can decrease the level of bacterial taxa (and/or decrease the activity of bacterial enzyme(s)) that inactivate a drug or convert it into less active (e.g., inactive) form. In yet other examples, the compositions and methods described herein can increase the level of bacterial taxa (and/or increase the activity of bacterial enzyme(s)) that process a toxic substance (e.g., a drug or a metabolic intermediate produced by the processing of the drug, e.g., by host cells or cells of other bacterial taxa) into a less toxic or nontoxic substance. In yet other examples, the compositions and methods described herein can decrease the level of bacterial taxa (and/or decrease the activity of bacterial enzyme(s)) that inhibit detoxification (e.g., processing of toxic substances to be less toxic or removal, e.g., relocalization or excretion, of toxic substances) of toxic substances (e.g., by promoting retaining of the toxic substance or inhibiting other enzymatic activities, e.g., host or other bacterial enzymatic activities, that would promote detoxification).

Provided herein are methods for reducing drug toxicity in a human subject. In embodiments, the methods comprise administering to the subject a glycan compositionin an amount effective to reduce drug toxicity. In embodiments, the subject has previously been administered, is being administered, or will be administered the drug (e.g., the drug associated with the toxicity). In embodiments, the glycan composition is administered in an amount and/or for a time sufficient to reduce the drug toxicity (and related symptoms, such as, e.g., cytotoxicity, diarrhea, constipation, nausea, dizziness, weight loss, etc.) in the subject (e.g., relative to a reference level, e.g., the drug toxicity in the subject prior to administration of the glycan composition). In embodiments, the method further comprises administering the drug to the subject, e.g., in combination with the glycan composition. In embodiments, the drug toxicity is reduced by at least 5% (e.g., at least 5%, 10%, 15%, 20%, 25%, 30%, 40%, 50%, 60%, 70%, 80%, 90%, 95%, 97%, 98%, or at least 99%), or at least 1.5-fold, 2-fold, 3-fold, 4-fold, 5-fold, 10-fold, 15-fold, 20-fold, 25-fold, 50-fold, 100-fold, 250-fold, 500-fold, at least 1000-fold, or more) relative to a reference level (e.g., the drug toxicity in the subject prior to administration of the glycan composition).

Drug toxicity refers to one or more adverse effects in a subject resulting from the administration of a drug to the subject. The adverse effects can range from discomfort (e.g., symptoms, such as, e.g., cytotoxicity, diarrhea, constipation, nausea, dizziness, weight loss, etc.) to death in some cases. Drug toxicity can result from a number of causes including off target activity, on target toxicity, processing of the drug into a toxic metabolite or intermediate, an inappropriate dosage (e.g., too high of a dosage) for a subject, prolonged used of the drug, and/or interaction of the drug with a second drug or substance. The toxicity of a drug depends on a number of factors, including age, preexisting conditions, genetic makeup, and/or presence of other drugs or metabolites thereof in the subject. Mechanistically, drug toxicity can be due to the production of a toxic metabolite or intermediate of the drug, an on target adverse effect (e.g., where the drug binds to the correct target/receptor but is provided at an inappropriate concentration, displays suboptimal kinetics, and/or is used for the wrong indication) or an off target adverse effect (e.g., where the drug binds to the wrong target/receptor).

Drug toxicity can be measured qualitatively and/or quantitatively. In embodiments, a method (e.g., quantitative method) for measuring drug toxicity can include assays (e.g., in vitro assays) for detecting apoptosis and/or necrosis of a host cell, e.g., a host cell that lines the gastrointestinal tract or a section thereof. In embodiments, the presence of apoptosis and/or necrosis in the cell indicates that the drug is toxic. In embodiments, a lesser extent of apoptosis and/or necrosis (e.g., fewer apoptotic/necrotic cells) in the cells indicates reduced drug toxicity, e.g., compared to prior to treatment with a glycan composition described herein.

In embodiments, a method (e.g., quantitative method) for measuring drug toxicity can include measuring the number of or prevalence of particular microbial taxa (e.g., microbial taxa described herein) in a sample from the subject, e.g., a stool sample. In embodiments, a decrease (e.g., loss) in the abundance of microbial taxa and/or a decrease (e.g., loss) in diversity of microbial taxa indicates drug toxicity, e.g., compared to prior to treatment with the drug. In embodiments, an increase in the abundance of microbial taxa and/or a increase in diversity of microbial taxa indicates decreased drug toxicity, e.g., compared to before treatment with a glycan composition described herein.

In embodiments, a method for measuring drug toxicity can include measuring the level of one or more inflammatory markers and/or other biomarker indicative of a response to injury in the subject, e.g., a sample from the subject. The level of inflammatory markers and/or other biomarkers can be measured using standard methods. In embodiments, the inflammatory marker includes one or more pro-inflammatory cytokines, e.g., TNF-α, IL-1, IL-6, and/or IL-10.

In embodiments, a qualitative method for measuring drug toxicity can include detecting one or more symptoms of drug toxicity in the subject, e.g., constipation, diarrhea, inflammation, and/or vomiting.

In embodiments, the methods described herein comprise increasing the maximum tolerated dose of a drug by decreasing drug toxicity by administering a glycan preparation described herein. In embodiments, the methods decssribed herein comprise increasing the dosage at which a non-therapeutic event is reached by decreasing drug toxicity by administering a glycan preparation described herein. Exemplary non-therapeutic events comprise: toxicity, e.g., toxicity of the drug, or of a species arising from metabolism of the drug or off target activity. Symptoms of toxicity include but are not limited to: experiences ranging from discomfort (e.g., symptoms, such as, e.g., cytotoxicity, diarrhea, constipation, nausea, dizziness, weight loss, etc.) to death.

Also provided herein are methods for increasing drug efficacy, potency, and/or bioavailability in a subject comprising administering to the subject a glycan composition. In embodiments, the subject has previously been administered, is being administered, or will be administered the drug. In embodiments, the glycan composition is administered in an effective amount and/or for a sufficient time to increase the drug efficacy, potency, and/or bioavailability in the subject (e.g., relative to a reference level, e.g., the drug toxicity in the subject prior to administration of the glycan composition).

Drug efficacy refers to the ability of a drug to produce an effect, e.g., a desired effect, e.g., increasing GI motility or lowering cholesterol levels. In embodiments, drug efficacy includes a drug's intrinsic activity, which can be expressed as the amount of a biological effect produced per unit of drug-receptor complex formed. A drug's bioavailability refers to the proportion of the drug that enters the circulation of a subject after administration and is thus able to elicit an effect. Drug potency refers to the measure of drug activity expressed in terms of the amount of drug required to produce an effect with a predetermined intensity.

Drug efficacy can be measured qualitatively and/or quantitatively. In embodiments, drug efficacy can be measured by detecting an improvement in (e.g., lack or milder form of) one or more symptoms of the disease/disorder that the drug was intended to treat. In embodiments, drug efficacy can be measured in vitro, e.g., in a cell or tissue sample, e.g., cell culture, by determining a functional readout (e.g., protein, e.g., enzyme level or activity, production of a molecule such as a second messenger, posttranslational modification such as phosphorylation of a protein, or changes in gene expression) after incubation of the cell or tissue sample with the drug. In other embodiments, drug efficacy can be measured ex vivo or in vivo, e.g., by determining a functional readout after administration of the drug or incubation ex vivo with a sample (e.g., a fecal sample) from the subject.

Drug potency can be measured by determining the drug's EC50 (half maximal effective concentration), which is the concentration of the drug at which the effect is 50% of Eₘₐₓ (the maximum possible effect for the drug). The lower the EC50, the higher the potency of the drug. EC₅₀ of a drug can be determined by standard methods, e.g., measuring a functional readout in a cell or tissue sample, e.g., cell culture, after administration of increasing doses of the drug.

Drug bioavailability can be measured by determining the area under the plasma concentration-time curve (AUC), which is directly proportional to the total amount of drug (e.g., unmodified drug) that reaches the systemic circulation of a subject.

In some embodiments, the compositions and methods can achieve one or more desired effects described in Tables 1, 2, or 3. In embodiments, the methods comprise modulating the activity of an enzyme and/or level of a microbe described in Tables 1, 2, or 3, or Tables 4, 5, or 6. In embodiments, the methods comprise administering the composition to a subject that has been, is being, or will be administered an exogenous substance described herein, e.g., in Tables 1, 2, or 3.

Also, provided herein are methods of identifying or selecting a treatment regimen for a subject. In embodiments, the method comprises the steps of: a) acquiring a value for the presence or level of a bacterial taxa or a microbial metabolite or an enzymatic activity in the subject; b) responsive to the value, selecting a glycan composition to treat the subject; and

c) administering the glycan composition in an effective amount and/or for a sufficient time to treat the subject.

In embodiments, the method further comprises a step prior to step (a), of selecting a subject that has been administered, is being administered, or will be administered an exogenous substance, e.g., an environmental toxin or toxicant, a pharmaceutical agent or drug, a dietary component, a food additive, or a drug additive.

Provided herein are also methods of selecting a subject for treatment using a glycan composition described herein. In embodiments, the subject is selected based on his or her exposure to an exogenous substance, e.g., exogenous substance described herein. In embodiments, the subject is selected for treatment if he/she has been administered (has been exposed to or contacted with), is being administered (being exposed to or contacted with), or will be administered (will be exposed to or contacted with) an exogenous substance, e.g., an environmental toxin or toxicant, a pharmaceutical agent or drug, a dietary component, a food additive, a drug additive. In embodiments, the exogenous substance comprises an allergen. In other embodiments, the subject is selected if he/she has a disease/condition, e.g., an immune disease, an infectious disease, a metabolic disease, a neurodegenerative disease, a cancer, an allergy, or another disease or disorder or detrimental condition, including a precondition or predisposition to develop a disease or disorder. In embodiments, the subject is selected if he/she is (e.g., determined to be) deficient in an enzyme activity (e.g., enzyme level and/or enzyme specific activity), e.g., a microbial enzyme activity. In embodiments, the subject is selected if he/she has (e.g., is determined to have) an overabundance of an enzyme and/or an overactive enzyme, e.g., a microbial enzyme. In embodiments, the enzyme is an enzyme described herein, e.g., in Table 1, 2, or 3. In embodiments, the enzyme is an enzyme having an activity or an enzyme catalyzing a reaction described herein, e.g., in the “Processing of exogenous substances” section herein. In embodiments, the subject is selected if he/she is (e.g., determined to be) deficient in bacterial taxa. In embodiments, the subject is selected if he/she has (e.g., is determined to be have) an overabundance of a bacterial taxa. In embodiments, the bacterial taxa comprises a bacterial taxa that is beneficial, e.g., a bacterial taxa for which an increase in levels is desired according to Tables 1, 2, 3, and 4-6. In embodiments, the bacterial taxa comprises a bacterial taxa that is detrimental, e.g., a bacterial taxa for which a decrease in levels is desired according to Table 1, 2, 3, and 4-6.

### Diseases, Disorders, and Unwanted Conditions

Diabetes Mellitus Type 2

In one embodiment, provided is a method of treating diabetes mellitus type 2 (e.g., type 2 diabetes) in a subject in need thereof, by: administering to the subject a composition in an amount effective to reduce signs and symptoms of type 2 diabetes. Type 2 diabetes is a long-term metabolic disorder that may be characterized by high blood sugar, insulin resistance, and relative lack of insulin. Subjects with type 2 diabetes may present with high blood sugar; insulin resistance; lack of insulin; increased thirst (e.g., polydipsia); frequent urination (e.g., polyuria); unexplained weight loss; increased hunger (e.g., polyphagia); feeling tired (e.g., fatigue); blurred vision; itchiness; recurrent vaginal infections; neuropathy; unhealed sores; fasting plasma glucose is greater than or equal to, e.g., 7.0 mmol/l (126 mg/dl); two hours after an oral dose of glucose, plasma glucose is greater than or equal to, e.g., 11.1 mmol/l (200 mg/dl); glycated hemoglobin (HbA1c) of greater than or equal to, e.g., 48 mmol/mol (greater than or equal to, e.g., 6.5 DCCT %); high C-peptide levels (e.g., for diabetes mellitus type 2); and low C-peptide levels (e.g., for diabetes mellitus type 1). Conditions associated with type 2 diabetes include heart disease, stroke, diabetic retinopathy (which can result in blindness), kidney failure, and poor blood flow in the limbs (which may lead to amputations). Diabetes (both type 1 and type 2) may be determined by any appropriate method known in the art, including, fasting glucose test, glucose tolerance test, measuring glycated hemoglobin (HbA1c), and measuring C-peptide level. Standard-of-care treatment for type 2 diabetes include lifestyle interventions (e.g., lowering other cardiovascular risk factors, and maintaining blood glucose levels), metformin, sulfonylureas, thiazolidinediones, dipeptidyl peptidase-4 inhibitors, SGLT2 inhibitors, glucagon-like peptide-1 analogs, and insulin therapy.

Convulsions

In one embodiment, provided is a method of treating convulsions in a subject in need thereof, by: administering to the subject a composition in an amount effective to reduce convulsive events. A convulsion is a medical condition where body muscles contract and relax rapidly and repeatedly, resulting in an uncontrolled shaking of the body. Subjects suffering from convulsions may present with a brief blackout (e.g., loss of consciousness), confusion, drooling, loss of bowel/bladder control, sudden shaking of entire body, uncontrollable muscle spasms, temporary cessation of breathing, nervous system infection, and intracranial bleeding among others. Conditions associated with convulsions include fevers, head trauma, stroke, hypoglycemia, lack of oxygen to the brain, genetic defects, or brain tumors. Convulsions may be determined by blood tests (e.g., testing for electrolytes, blood glucose, and/or blood calcium), lumbar puncture, electroencephalogram, and/or brain imaging (with CT scan or MRI scan). Standard-of-care treatment for convulsion includes benzodiazepine, lorazepam, barbiturates, or propofol.

Hepatitis C

In one embodiment, provided is a method of treating Hepatitis C infection in a subject in need thereof, by: administering to the subject a composition in an amount effective to reduce Hepatitis C levels in a subject. Hepatitis C is an infectious disease caused by the hepatitis C virus (HCV) that primarily affects the liver. Subjects with Hepatitis C may present with a fever, dark urine, abdominal pain, yellow tinged skin, decreased appetite, fatigue, nausea, muscle or joint pain, and weight loss. Conditions associated with Hepatitis C include liver disease, cirrhosis, liver failure, liver cancer, dilated stomach and esophagus blood vessels, lymphocytes within the parenchyma, lymphoid follicles in portal triad, and changes to the bile ducts. Hepatitis C may be determined by HCV antibody enzyme immunoassay or ELISA; recombinant immunoblot assay; quantitative HCV RNA polymerase chain reaction (PCR); blood tests (e.g., to detect: presence of HCV antibodies, degree of liver fibrosis, and/or liver enzyme level); and liver biopsy. Standard-of-care treatment for Hepatitis C infection include elbasvir/grazoprevir, ledipasvir/sofosbuvir, sofosbuvir/velpatasvir, sofosbuvir/daclatasvir, sofosbuvir/simeprevir; ledipasvir; sofosbuvir; velpatasvir; elbasvir; daclatasvir; voxilaprevir; and liver transplantation (e.g., alone or in combination with ribavirin and pegylated interferon).

### HIV

In one embodiment, provided is a method of treating human immunodeficiency virus (HIV) infection in a subject in need thereof, by: administering to the subject a composition in an amount effective to reduce HIV levels in a subject. The human immunodeficiency virus (HIV) is a lentivirus (e.g., retrovirus) that causes HIV infection and over time acquired immunodeficiency syndrome (AIDS) in which progressive failure of the immune system allows life-threatening opportunistic infections and cancers to thrive. Subjects with HIV may present with influenza-like or a mononucleosis-like illness, fever, large tender lymph nodes, throat inflammation, a rash, headache, and/or sores of the mouth and genitals. Subjects with advanced HIV infection may present with CD4+ T cell count below 200 cells per μL, pneumocystis pneumonia, cachexia, esophageal candidiasis, and opportunistic infections. HIV subjects may develope viral-induced cancers, including Kaposi's sarcoma, Burkitt's lymphoma, primary central nervous system lymphoma, and cervical cancer. Additionally, AIDS patients may have systemic symptoms such as prolonged fevers, sweats, swollen lymph nodes, chills, weakness, unintended weight loss, and diarrhea. Conditions associated with HIV infection include toxoplasmosis (e.g., toxoplasmosis of the brain), candidiasis (e.g., candidiasis of the esophagus, trachea, bronchi, or lungs), Kaposi's sarcoma, Burkitt's lymphoma, primary central nervous system lymphoma, cervical cancer, opportunistic infections, cachexia, and pneumocystis pneumonia. HIV infection may be determined using any appropriate method known in the art, including, for example, testing for specific antibodies; HIV RNA or DNA; and/or p24 antigen using one or more of the following: enzyme-linked immunosorbent assay (ELISA); immunoassay combination test (e.g., for HIV-1 and HIV-2 antgibodies and p24 antigen); western blot; polymerase chain reaction (PCR); immunofluorescence assay (IFA); and nucleic acid testing (NAT). Standard-of-care treatment for HIV infection include maraviroc, enfuvirtide, zidovudine, abacavir, lamivudine, emtricitabine, tenofovir, nevirapine, efavirenz, etravirine, rilpivirine, raltegravir, elvitegravir, dolutegravir, lopinavir, indinavir, nelfinavir, amprenavir, ritonavir, darunavir, atazanavir, bevirimat, vivecon, and combinations thereof.

Inflammation

In one embodiment, provided is a method of treating inflammation in a subject in need thereof, by: administering to the subject a composition in an amount effective to reduce inflammation in a subject Inflammation is part of the complex biological response of body tissues to harmful stimuli, such as pathogens, damaged cells, or irritants, and is a protective response involving immune cells, blood vessels, and molecular mediators. Subjects with inflammation may present with heat, pain, redness, swelling, loss of function, increased movement of plasma and leukocytes (especially granulocytes) from the blood into injured tissues, and prescence of mononuclear cells. Conditions associated with inflammation include allergic reactions, myopathies, cancer, atherosclerosis, and ischemic heart disease. Inflammation may be determined using any appropriate method known in the art, including, for example, physical examination, evaluation of non-joint symptoms, medical imaging (e.g., x-rays), blood tests, and other studies. Standard-of-care treatment for inflammation includes physical therapy and exercise; decrease stress on the joints by using braces, splints, or canes as needed; anti-inflammatory pain reliever drugs (e.g., NSAIDs, aspirin, ibuprofen, or Celebrex); corticosteroids (e.g., prednisone); and other medications including chemotherapeutics, disease modifying treatments, biologic therapy, and/or narcotic pain relievers.

Cardiac Arrhythmia

In one embodiment, provided is a method of treating cardiac arrhythmia in a subject in need thereof, by: administering to the subject a composition in an amount effective to reduce cardiac arrhythmia in a subject. Heart arrhythmia, also known as cardiac dysrhythmia or irregular heartbeat, is a group of conditions in which the heartbeat is irregular, too fast, or too slow. Subjects with cardiac arrythmia may present with palpitations or feeling a pause between heartbeats, lightheadedness, passing out (e.g., syncope (fainting)), shortness of breath, chest pain, stroke, heart failure, cardiac arrest, abnormal heartbeat, lower blood pressure, dizziness, or syncope (fainting). Conditions associated with cardiac arrythmia include increased risks of embolisation and stroke, heart failure, and sudden cardiac death. Cardiac arrythmia may be determined using any appropriate method known in the art, including, for example, auscultation of the heartbeat with a stethoscope; feeling for peripheral pulses; electrocardiogram (e.g., ECG or EKG); and transesophageal atrial stimulation (TAS). Standard-of-care treatments for cardiac arrythmia include physical maneuvers; medications (including warfarin, heparins, and anti-platelet drugs such as aspirin); electricity conversion; electrical treatment (including implanted electrodes, permanent pacemaker, and defibrillation or cardioversion with an implantable cardioverter-defibrillator (ICD)); or electro- or cryo-cautery.

Hypertension

In one embodiment, provided is a method of treating hypertension in a subject in need thereof, by: administering to the subject a composition in an amount effective to reduce blood pressure in a subject. Hypertension (HTN or HT), also known as high blood pressure (HBP), is a long-term medical condition in which the blood pressure in the arteries is persistently elevated. Subjects with hypertension may present with thickening of heart muscle (e.g., left ventricular hypertrophy), heart enlargement, heart damage, and elevated resting blood pressure (e.g., consistent systolic blood pressure above 139 mmHg and/or consistent diastolic blood pressure measurement above 89 mmHg). Conditions associated with hypertension include obesity, coronary artery disease, stroke, heart failure, atrial fibrillation, peripheral vascular disease, vision loss, chronic kidney disease, and dementia. Hypertension may be determined using any appropriate method known in the art, including, for example, medical imaging (e.g., chest X-ray), electrocardiogram, measurement of systolic blood pressure, and measurement of diastolic blood pressure. Standard-of-care treatment for hypertension includes lifestyle changes (e.g., dietary changes, physical exercise, weight loss) and medications including thiazide-diuretics, calcium channel blockers, angiotensin converting enzyme inhibitors, and angiotensin receptor blockers.

Glaucoma

In one embodiment, provided is a method of treating glaucoma in a subject in need thereof, by: administering to the subject a composition in an amount effective to reduce vision loss in a subject. Glaucoma is a group of eye diseases which result in damage to the optic nerve and vision loss. Subjects with glaucoma may present with eye pain, blurred vision, suddenly decreased vision, mid-dilated pupil, redness of the eye, optic nerve damage, seeing halos around lights, intraocular pressure (e.g., very high intraocular pressure (>30 mmHg)), nausea and vomiting, and vision loss. Conditions associated with glaucoma include migraines, high blood pressure, and obesity. Glaucoma may be determined using any appropriate method known in the art, including, for example, measurements of the intraocular pressure via tonometry; examination of the optic nerve to look for any visible damage; gonioscopy; optical coherence tomography; scanning laser polarimetry; scanning laser ophthalmoscopy; perimetry; pachymetry; and nerve fiber analysis. Standard-of-care treatments for glaucoma include medications (including: prostaglandin analogs (e.g., latanoprost, bimatoprost and travoprost); topical beta-adrenergic receptor antagonists (e.g., timolol, levobunolol, and betaxolol); alpha2-adrenergic agonists (e.g., brimonidine and apraclonidine); less-selective alpha agonists (e.g., epinephrine), miotic agents (e.g., parasympathomimetics) (e.g., pilocarpine), carbonic anhydrase inhibitors (e.g., dorzolamide, brinzolamide, and acetazolamide), and echothiophate); laser therapy (including: argon laser trabeculoplasty (ALT), selective laser trabeculoplasty (SLT), and Nd:YAG laser peripheral iridotomy (LPI)); and surgery (including canaloplasty and nonpenetrating deep sclerectomy (NPDS).

Bacterial Infection

In one embodiment, provided is a method of treating bacterial infection in a subject in need thereof, by: administering to the subject a composition in an amount effective to reduce bacterial infection in a subject. Infection is the invasion of an organism's body tissues by disease-causing agents, their multiplication, and the reaction of host tissues to the infectious agents and the toxins they produce. Infectious disease, also known as transmissible disease or communicable disease, includes illness resulting from an infection. Bacteria are one of the agents responsible for causing infections. Subjects with bacterial infection may present with inflammation, fatigue, loss of appetite, weight loss, fevers, night sweats, chills, aches and pains, skin rashes, coughing, or a runny nose. Conditions associated with bacterial infection include abscess, respiratory system infection, and diarrheal disease. Bacterial infection may be determined using any appropriate method known in the art, including, for example, microbiological culture of infectious agents, microscopic procedures (e.g., Gram stain and/or acid stain), medical imaging (e.g., X-rays, CAT scans, PET scans or NMR), biochemical tests (e.g., including detection of metabolic or enzymatic products characteristic of a particular infectious agent), serological methods, immunoassays, and polymerase chain reaction (PCR) methods. Standard-of-care treatment for bacterial infections include anti-infective drugs, e.g., antibacterial (antibiotic; including antitubercular) including penicillin, cephalosporins, aminoglycosides, macrolides, quinolones, tetracyclines, and metronidazole.

Viral Infection (e.g., Herpes Simplex Virus Type 1 and Varicella Zoster Virus)

In one embodiment, provided is a method of treating viral infection (e.g., Herpes Simplex Virus Type 1 (HSV1) and/or Varicella Zoster Virus (VZV) in a subject in need thereof, by: administering to the subject a composition in an amount effective to reduce viral load in a subject. HSV1 and VZV are infectious disease caused by viruses in the Herpesvirus Family. Subjects with viral infection (e.g., HSV1 and/or VZV) may present with lesions in the skin or mucous membranes of the mouth, lips, nose, or genitals (e.g., for HSV1) and lesions (e.g., rashes) of the skin (for VZV). Conditions associated with viral infection (e.g., HSV1 and/or VZV) include Alzheimer's disease, Chickenpox (e.g., varicella), Shingles, encephalitis, pneumonia (e.g., viral or secondary bacterial), or bronchitis (e.g., viral or secondary bacterial), postherpetic neuralgia, Mollaret's meningitis, zoster multiplex, brain and/or nerve inflammation, and Reye's syndrome. Viral infection (e.g., HSV1 and/or VZV) may be determined by detection of lesions, viral DNA, viral proteins, or antibodies to viral proteins. Standard-of-care treatment for viral infection (e.g., HSV1 and/or VZV) include aciclovir (e.g., acyclovir), famciclovir, valaciclovir, zoster-immune globulin(ZIG), vidarabine, and VZV immune globulin.

Ebola Virus

In one embodiment, provided is a method of treating Ebola virus disease in a subject in need thereof, by: administering to the subject a composition in an amount effective to reduce Ebola viral load in a subject. Ebola virus disease (EVD), also known as Ebola hemorrhagic fever (EHF) or simply Ebola, is a viral hemorrhagic fever of humans and other primates caused by ebolaviruses. Subjects with Ebola virus disease may present with fever, sore throat, muscular pain, feeling tired, weakness, decreased appetite, muscular pain, joint pain, headaches, vomiting, diarrhea, abdominal pain, rash, decreased liver function, decreased kidney function, internal bleedidng, external bleeding, low platelet count, elevated alanine aminotransferase level, elevated aspartate aminotransferase level, prolonged prothrombin time, and decreased white blood cell count followed by increased white blood cell count. Ebola virus disease may be determined using any appropriate method known in the art, including, for example, blood tests (e.g., for viral RNA, viral antibodies, or the virus itself), cell culture, microscopy (e.g., electron microscopy), enzyme-linked immunosorbsent assay (ELISA), and polymerase chain reaction (e.g., rRT-PCR). Standard-of-care treatment for Ebola virus disease includes adenosine analogs.

Hepatitis B

In one embodiment, provided is a method of treating Hepatitis B in a subject in need thereof, by: administering to the subject a composition in an amount effective to reduce Hepatitis B viral load in a subject. Hepatitis B is an infectious disease caused, e.g., by the hepatitis B virus (HBV) that affects the liver. Subjects with Hepatitis B may present with vomiting, yellowish skin (e.g., jaundice), tiredness, dark urine, abdominal pain, loss of appetite, body aches, fever, itchy skin, elevated serum alanine aminotransferase, elevated liver inflammation, Hepatitis B Virus (HBV) DNA, Hepatitis B surface antigen (HBsAg), and Hepatitis B e antigen (HBeAg). Conditions associated with Hepatitis B include liver damage, cirrhosis, liver cancer, serum-sickness-like syndrome, acute necrotizing vasculitis (e.g., polyarteritis nodose), and membranous glomerulonephritis. Hepatitis B may be determined using any appropriate method known in the art, including, for example, serum test, blood test, and polymerase chain reaction (PCR) test. Standard-of-care treatment for Hepatitis B include antiviral drugs (including lamivudine (Epivir), adefovir (Hepsera), tenofovir (Viread), telbivudine (Tyzeka), and entecavir (Baraclude)) and immune system modulators (including interferon alpha-2a and PEGylated interferon alpha-2a (Pegasys)).

Ulcerative Colitis

In one embodiment, provided is a method of treating ulcerative colitis in a subject in need thereof, by: administering to the subject a composition in an amount effective to reduce ulcerative colitis in a subject. Ulcerative colitis (UC) is a long-term condition that results in inflammation and ulcers of the colon and rectum. Subjects with ulcerative colitis may present with ulcers (e.g., ulcers of the colon and rectum), inflammation (e.g., inflammation of the colon and rectum), abdominal pain, diarrhea (e.g., diarrhea mixed with blood and mucus), weight loss, painful bowel movements, anemia, fever, increased bowel movements, elevated erythrocyte sedimentation rate, elevated C-reactive protein level, loss of vascular appearance of the colon erythema (e.g., redness of the intestinal mucosa), superficial ulceration, and pseudopolyps. Conditions associated with ulcerative colitis include megacolon, inflammation (e.g., inflammation of the eye, joints, or liver), hypokalemia, hypomagnesemia, pre-renal failure, and colon cancer. Ulcerative colitis may be determined using any appropriate method known in the art, including, for example, blood test, electrolyte analysis, liver function test, x-ray imaging, biopsy, and endoscopy (e.g., sigmoidoscopy). Standard-of-care treatment for ulcerative colitis include medications (including corticosteroid (e.g., prednisone), immunosuppressant (e.g., azathioprine), budesonide, cyclosporin, fexofenadine, tacrolimus, tofacitinib, vedolizumab, etrolizumab, and TNF inhibitors (e.g., infliximab, adalimumab, and golimumab) and surgery (e.g., surgical removal of the large intestine and/or ileo-anal pouch procedure).

Crohn's Disease

In one embodiment, provided is a method of treating Crohn's disease in a subject in need thereof, by: administering to the subject a composition in an amount effective to reduce Crohn's disease in a subject. Crohn's disease is a type of inflammatory bowel disease (IBD) that may affect any part of the gastrointestinal tract from mouth to anus. Subject's with Crohn's disease may present with abdominal pain, diarrhea, fever, weight loss, anemia, skin rashes, arthritis, inflammation of the eye, tiredness, fistulae, skin lesion (e.g., erythema nodo sum and pyoderma gangrenosum), abscesses, bowel obstruction, and patchy distribution of inflammation throughout the colon and ileum but not the rectum. Conditions associated with Crohn's disease include gallstones, rheumatologic disease (e.g., seronegative spondyloarthropathy), blood clots, and bowel cancer. Crohn's disease may be determined using any appropriate method known in the art, including, for example, biopsy, CT imaging, MRI imaging, and colonoscopy. Standard-of-care treatment for Crohn's disease include prednisone, hydrocortisone, immunomodulator (e.g., azathioprine), methotrexate, natalizumab, ustekinumab, certolizumab, infliximab, adalimumab, and/or surgery (e.g., surgical removal of obstructions, fistula, or abscess).

Pulmonary Hypertension

In one embodiment, provided is a method of treating pulmonary hypertension in a subject in need thereof, by: administering to the subject a composition in an amount effective to reduce arterial blood pressure in the lungs of a subject. Pulmonary hypertension (PH or PHTN) is a condition of increased blood pressure within the arteries of the lungs. Subjects with pulmonary hypertension may present with shortness of breath, syncope, tiredness, chest pain, swelling of the legs, swelling (e.g., swelling in legs and/or swelling in ankles), cyanosis, a fast heartbeat, pulmonary arterial pressure (PAP) at least 25 mm Hg at rest, and PAP≥25 mm Hg with pulmonary arterial occlusion pressure ≤15 mm Hg. Conditions associated with pulmonary hypertension include blood clots in the lungs, HIV/AIDS, COPD, and sleep apnea. Pulmonary hypertension may be determined using any appropriate method known in the art, including, for example, echocardiography, electrocardiography, x-rays, computed tomography (CT) scan, and pulmonary function tests (e.g., carbon monoxide and arterial blood gas measurements). Standard-of-care treatment for pulmonary hypertension includes epoprostenol, treprostinil, iloprost, ambrisentan, macitentan, sildenafil, remodulin, prostacyclin, prostaglandin I2, tadalafil, endothelin antagonist, prostanoid, phosphodiesterase inhibitor, endothelin receptor antagonist, calcium channel blocker, diuretic, oxygen therapy, atrial septostomy lung transplantation, and pulmonary thromboendarterectomy.

Heart Failure

In one embodiment, provided is a method of treating heart failure in a subject in need thereof, by: administering to the subject a composition in an amount effective to improve heart function. Heart failure (HF), often referred to as congestive heart failure (CHF), occurs when the heart is unable to pump sufficiently to maintain blood flow to meet the body's needs. Subjects with heart failure may present with shortness of breath, excessive tiredness, leg swelling, cardiomegaly, vascular redistribution, Kerley lines, arrhythmia, ischemic heart disease, ventricular hypertrophy, conduction delay/abnormality, elevated B-natriuretic peptide, and reduced ejection fraction. Conditions associated with heart failure include coronary artery disease, high blood pressure, atrial fibrillation, valvular heart disease, cardiomyopathy, obesity, kidney failure, liver problems, anemia, and thyroid disease. Heart failure may be determined using any method known in the art, including, for example, blood tests (e.g., measuring: brain natriuretic peptide, electrolytes, liver function, kidney function, thyroid function, complete blood count (CBC), and/or C-reactive protein), ultrasound (e.g., echocardiography), x-ray imaging, and electrocardiogram. Standard-of-care treatment for heart failure include angiotensin converting enzyme inhibitor (ACEI), angiotensin receptor blocker (ARB), beta blocker, combination of hydralazine and long-acting nitrate (e.g., isosorbide dinitrate), aldosterone antagonist, mineralocorticoid antagonist (e.g., spironolactone), diuretic (e.g., loop diuretics, thiazide-like diuretics, and potassium-sparing diuretic), automatic implantable cardioconverter defibrillator (AICD), cardiac contractility modulation (CCM) (e.g., ventricular assist device (VAD)), and heart transplantation.

Migraines

In one embodiment, provided is a method of treating migraines in a subject in need thereof, by: administering to the subject a composition in an amount effective to reduce frequency and intensity of headaches. A migraine is a neurological disorder typically characterized by recurrent headaches that are moderate to severe. Subjects with migraines may present with recurrent headaches; nausea; vomiting; sensitivity to light (e.g., photophobia), sound (e.g., phonophobia), or smell, fatigue; irritability; tingling in limbs; neck pain; vertigo; weakness; and cognitive symptoms. Migraines may be determined using any appropriate method known in the art, including, for example, detecting the presence of pulsating headaches. Standard-of-care treatment for migraines include ibuprofen, paracetamol (e.g., acetaminophen), metoprolol, valproate, topiramate, ketorolac, ergotamine, dihydroergotamine, metoclopramide, lidocaine, haloperidol, and dexamethasone.

Erectile Dysfunction

In one embodiment, provided is a method of treating migraines in a subject in need thereof, by: administering to the subject a composition in an amount effective to improve erectile function. Erectile dysfunction (ED), also known as impotence, is a type of sexual dysfunction characterized by the inability to develop or maintain an erection of the penis during sexual activity. Subjects with erectile dysfunction may present with impotence. Conditions associated with erectile dysfunction include diabetes, cardiovascular disease, hormonal insufficiency (e.g., hypogonadism), trauma, coronary artery disease, and peripheral vascular disease. Erectile dysfunction may be determined using any appropriate method known in the art, including, for example, duplex ultrasound, bulbocavernous reflex test, penile biothesiometry, dynamic infusion cavernosometry, cavernosography, digital subtraction angiography, and MRI. Standard-of-care treatment for erectile dysfunction includes sildenafil, vardenafil, tadalafil, alprostadil (e.g., alprostadil with permeation enhancer DDAIP), papaverine, phentolamine, and prostaglandin E1, vacuum erection device, and penile implant.

Benign Prostatic Hyperplasia

In one embodiment, provided is a method of treating benign prostate hyperplasia in a subject in need thereof, by: administering to the subject a composition in an amount effective to reduce prostate hyperplasia. Benign prostatic hyperplasia (BPH), also called prostate enlargement, is a non-cancerous increase in size of the prostate. Subjects with benign prostate hyperplasia may present with inability to urinate, loss of bladder control, abdominal pain, dysuria, urinary hesitancy, urinary intermittency, nocturia, continuous feeling of full bladder, frequent urination, acute urinary retention, slow urine flow, bladder outlet obstruction, and elevated prostate specific antigen (PSA). Conditions associated with benign prostate hyperplasia include obesity, erectile dysfunction, urinary tract infections, bladder stones, chronic kidney problems, prostate cancer, kidney disease, and diabetes. Benign prostate hyperplasia may be determined using any appropriate method known in the art, including, for example, digital rectal exam, urinalysis, kidney function test, prostate specific antigen screening (including free PSA and PSA density), and ultrasound (e.g., trans-rectal ultrasonography). Standard-of-care treatment for benign prostate hyperplasia includes alpha blocker (e.g., alfuzosin, doxazosin, silodosin, and terazosin), 5α-reductase inhibitor (e.g., finasteride and dutasteride), antimuscarinic (e.g., tolterodine), phosphodiesterase-5 inhibitor (e.g., sildenafil and tadalafil), catheterization (e.g., intermittent urinary catheterization), open prostatectomy, transurethral resection of the prostate, transurethral incision of the prostate, and photoselective vaporization of the prostate for benign prostate hyperplasia.

Cancer

In one embodiment, provided is a method of treating cancer in a subject in need thereof, by: administering to the subject a composition in an amount effective to reduce cancer (e.g., induction, growth, and/or metastatic spread). Cancer is a group of diseases involving abnormal cell growth, and frequently invades or spreads to other parts of the body. Subjects with cancer may present with a lump, abnormal bleeding, prolonged cough, unexplained weight loss, a change in bowel movements, genetic mutations, fusion genes, and numerical chromosome changes. Cancer may be determined using any appropriate method known in the art, including, for example, biopsy, blood test, x-ray imaging, CT scan, endoscopy, cytogenetics, and immunohistochemistry. Standard-of-care treatment for cancer includes deoxycytidine analogues (e.g. cytarabine, gemcitabine); pyrimidine analogues (e.g., 5-Fluorouracil (5FU), floxuridine (FUDR), cytarabine (Cytosine arabinoside), 6-azauracil (6-AU)); purine analogs (e.g., mercaptopurine, thiopurines, fludarabine, pentostatin); radiation therapy; laser therapy (e.g., laser-induced interstitial thermotherapy (LITT) (e.g., interstitial laser photocoagulation)); surgery; antibody; and adoptive cell transfer.

### Glycan Polymer Compositions and Manufacture Thereof

Glycan compositions can comprise the glycans described herein, dietary fibers, such as, e.g., FOS (fructooligosaccharide), other sugars (e.g., monomers, dimers, such as, e.g., lactulose) and sugar alcohols, and optionally other components, such as, e.g., polyphenols, fatty acids, peptides, micronutrients, etc., such as those described in WO 2016/172658, “MICROBIOME REGULATORS AND RELATED USES THEREOF”, and microbes, such as bacteria.

Glycan preparations described in WO 2016/122889 “GLYCAN THERAPEUTICS AND RELATED METHODS THEREOF” and WO 2016/172657, “GLYCAN THERAPEUTICS AND METHODS OF TREATMENT”, which in their entirety are hereby incorporated by reference, are suitable for in the methods and compositions described herein.

Preparations comprising glycans can be generated using a non-enzymatic catalyst, e.g., the polymeric catalyst described in WO 2012/118767, “POLYMERIC ACID CATALYSTS AND USES THEREOF” or by other suitable methods. Methods to prepare the polymeric and solid-supported catalysts described herein can be found in WO 2014/031956, “POLYMERIC AND SOLID-SUPPORTED CATALYSTS, AND METHODS OF DIGESTING CELLULOSIC MATERIALS USING SUCH CATALYSTS.” The glycans generated, e.g., by using the catalyst, for example as described in WO 2016/007778, “OLIGOSACCHARIDE COMPOSITIONS AND METHODS FOR PRODUCING THEREOF” are suitable for the methods and compositions described herein. All patent applications are incorporated herein by reference in their entirety.

In one embodiment, glycan preparations are generated as described herein. Glycan units and catalyst (e.g., polymeric catalyst or solid-supported catalyst) are allowed to react for at least 1 hour, up to 24 hours. In certain variations, glycan and catalyst react for up to 48 hours. The degree of polymerization (DP) of the glycan preparation produced according to the methods described herein can be regulated by the reaction time. The reaction temperature is maintained in a range of about 25° C. to about 150° C. The dry solids content of the glycan unit is between about 5 wt % to about 95 wt % (e.g., about 50 wt % to about 95 wt%). The weight ratio of the catalyst to the glycan unit(s) is about 0.01 g/g to about 0.2 g/g. In certain variations, the weight ratio of the catalyst to the glycan unit(s) is about 0.01 g/g to about 50 g/g. Synthesis of the glycans (e.g., oligosaccharides) using the polymeric catalyst (e.g., Dow Marathon C) is carried out in an aqueous environment wherein the aqueous solvent is water at a resistivity of at least 10 megaohm-centimeters. Heating and/or evaporation is used to remove water in the reaction mixture. In certain variations, at least a portion of water is removed to maintain a water content in the reaction mixture of less than 60%, less than 50%, less than 40%, less than 30%, less than 20%, less than 10%, less than 5%, or less than 1% by weight. The degree of polymerization of the glycan preparation is increased by decreasing the water concentration; the water content of the reaction is adjusted during the reaction to regulate the degree of polymerization of the glycan preparation produced. A majority, e.g. about 55%, 60%, 65%, 70%, or about 75% of the glycan therapeutic preparation has a DP of between about 2 and about 25. In certain variations, about 80%, 85%, 90% or about 95% of the glycan preparation has a DP of between 2 and 30. In one embodiment, 0.1-4 equivalents of water are added to one or more glycan units in a round bottom flask equipped with an overhead stirrer and a jacketed short-path condenser. The mixture is heated to 75-165° C. at 1000 mbar vacuum pressure prior to adding 1-10% or 1-20%, by dry weight of one or more catalysts (e.g., Dow Marathon C). The reaction may be stirred for 30 minutes to 8 hours. The yield of conversion to a glycan therapeutic preparation with DP>1 after combining one or more glycan units with the catalyst (e.g., at 2, 3, 4, 8 hours after combining the one or more glycan units with the catalyst (e.g., Dow Marathon C catalyst)) is greater than about 80%-95%. The yield of conversion to a glycan therapeutic preparation with >DP2 is greater than about 70%-85%. The yield of conversion to a glycan therapeutic preparation with >DP3 after is greater than about 35%-70%. Optionally, the glycan preparation produced may undergo additional processing steps, such as purification steps. Purification steps may include, for example, separation, dilution, concentration, filtration, desalting or ion-exchange, chromatographic separation, or decolorization, or any combination thereof. The glycan preparation produced may undergo a decolorization step using any method known in the art, including, for example, treatment with an absorbent, activated carbon, hydrogenation, and/or filtration (e.g., microfiltration). In certain embodiments, the glycan preparations are contacted with a color absorbing material for less than 1 hour, or less than 30 minutes. The glycan preparations are contacted with a color absorbing material at a temperature from about 30 to 80 degrees Celsius. In certain embodiments, the color absorbing material is activated carbon (e.g., powdered activated carbon). Optionally, decolorization may not require a separate processing step. In some embodiments, the methods described herein further include isolating the glycan preparation produced, using any method known in the art, including, for example, centrifugation, filtration (e.g., vacuum filtration, membrane filtration), gravity settling, filtration (e.g., membrane filtration), chromatography (e.g., chromatographic fractionation), differential solubility, and centrifugation (e.g., differential centrifugation). Glycan species (e.g., oligosaccharides) may be separated (e.g., fractionated) by molecular weight using any method known in the art, including, for example, high-performance liquid chromatography, adsorption/desorption (e.g. low-pressure activated carbon chromatography), or filtration (for example, ultrafiltration or diafiltration) into pools representing short (about DP1-2), medium (about DP3-10), long (about DPI 1-18), or very long (about DP>18) species. Furthermore, in an alternative embodiment, fractionation is not performed for some glycan preparations.

In another embodiment, glycan preparations are generated as described herein. Glycan units and catalyst (e.g., polymeric catalyst or solid-supported catalyst) are allowed to react for at least 2 hours, (e.g., at least 2 hours, at least 3 hours, at least 4 hours, at least 6 hours, at least 8 hours) up to 12 hours. The degree of polymerization (DP) of the glycan preparation produced according to the methods described herein can be regulated by the reaction time. The reaction temperature is maintained in a range of about 125° C. to about 150° C. In some embodiments, the reaction does not initiate until the conditions of 120° C. at atmospheric pressure, while a lower temperature is possible while maintaining the reaction under a vacuum. The dry solids content of the glycan unit is between about 90 wt % to about 95 wt %. In some embodiments, the reaction may proceed at a slow rate until the dry solid weight is 90-94%. In some embodiments, a dilute reaction mixture may cause the reaction to reverse and break down glycan oligomers into monomeric sugars. The weight ratio of the catalyst to the glycan unit(s) is about 0.01 g/g to about 5 g/g, about 0.05 g/g to about 1.0 g/g, or about 0.05 g/g to about 0.5 g/g. Synthesis of the glycans (e.g., oligosaccharides) using the polymeric catalyst is carried out in an aqueous environment wherein the aqueous solvent is water at a resistivity of about 0.1 megaohm-centimeters or less. The method further includes removing at least a portion of water produced in the reaction mixture (e.g., by removing at least about any of 10%, 20%, 30%, 40%, such as by vacuum filtration). For example, if the starting material is a dilute syrup of 70% solution and 30% water, up to 30% of the water may be removed. In certain variations, at least a portion of water is removed to maintain a water content in the reaction mixture of less than 99%, less than 90%, less than 80%, less than 70%, less than 60%, less than 50%, less than 40%, less than 30%, less than 20%, less than 10%, or less than 5% by weight. The water content of the reaction is adjusted during the reaction to regulate the degree of polymerization of the glycan preparation produced. For example, a majority, e.g. about 97% of the glycan therapeutic preparation has a DP of between 2 and 25. The solid mass obtained by the process can be dissolved in a volume of water sufficient to create a solution of approximately 50% solids (grams sugar per 100 g solution). Once dissolution is complete, the solid catalyst can be removed by filtration. The solution comprising therapeutic glycans can be concentrated to about 30-50% solids, e.g., by rotary evaporation. Optionally, an organic solvent is used and water immiscible solvents are removed by biphasic extraction and water miscible solvents are removed, e.g., by evaporation (e.g., rotary evaporation or wiped film evaporation) concomitant to the concentration step. Optionally, the glycan preparation produced may undergo additional processing steps. Additional processing steps may include, for example, purification steps. Purification steps may include, for example, separation, dilution, concentration, filtration, desalting or ion-exchange, chromatographic separation, or decolorization, or any combination thereof. The glycan preparation produced may undergo a decolorization step using any method known in the art, including, for example, chromatography (e.g., using ion exchange resin). In certain embodiments, the glycan preparations are contacted with a color absorbing material (e.g., Dowex Optipore SD-2) for less than 10 hours, (e.g., less than 5 hours). The glycan preparations are contacted with a material to remove salts, minerals, and/or other ionic species. The glycan preparations are flowed through an anionic/cationic exchange column pair. The anionic exchange column contains a weak base exchange resin in a hydroxide form and the cationic exchange column contains a strong acid exchange resin in a protonated form. The methods may further include a concentration step. The isolated glycan preparations undergo a spray drying step to produce a powdered glycan preparation. Optionally, the isolated glycan preparations undergo both an evaporation step and a spray drying step. Glycan therapeutic preparations (e.g., oligosaccharides) are created that are polydisperse, exhibiting a range of degrees of polymerization. The methods described herein may further include a fractionation step. Glycan species (e.g., oligosaccharides) may be separated by molecular weight using any method known in the art, including, for example, filtration. Low molecular weight materials may be removed by dialysis, ultrafiltration, diafiltration, or tangential flow filtration.

In some embodiments, glycan polymers are made using a glycosidase enzyme molecule under conditions suitable to generate glycan polymers.

In some embodiments, glycan polymers are made using solid-phase oligosaccharide synthesis, e.g., using a variety of protection groups to accomplish glycan polymer synthesis. Examplary methods are described in “Solid-Phase Oligosaccharide Synthesis and Combinatorial Carbohydrate Libraries”, Peter H. Seeberger and Wilm-Christian Haase, American Chemical Society, 2000; and “Opportunities and challenges in synthetic oligosaccharide and glycoconjugate research”, Thomas J. Boltje et al., Nat Chem. 2009 November 1; 1(8): 611-622.

Glycan Preparation Properties

Glycan may have any one or more of the characterisitics and properties disclosed in WO2016/122889, WO2016/172657, WO 2016/007778, and WO2016/172658, each of which is incorporated herein by reference in its entirety, and any characterisitics and properties disclosed herein.

The glycan polymers produced by the methods described herein may comprise oligosaccharides. In some embodiments, the glycan polymers comprise homo-oligosaccharides (or homoglycans), wherein all the monosaccharides in a polymer are of the same type.

In some embodiments, the glycan polymers comprise hetero-oligosaccharides (or heteroglycans), wherein more than one type of monosaccharide is present in the polymer. In some embodiments, the glycan polymers have one or more of the properties described herein. In some embodiments, the glycan polymer preparation has one or more of the bulk properties described herein.

Degree of Polymerization (DP)

In some embodiments, glycan polymer preparations are produced, e.g., using a method described herein, that are polydisperse, exhibiting a range of degrees of polymerization.

Optionally, the preparations may be fractionated, e.g. representing 60%, 65%, 70%, 75%, 80%, 85%, 90%, 95%, 97%, 98%, or greater than 98% short (about DP1-2), medium (about DP3-10), long (about DP11-18), or very long (about DP>18) species. In one embodiment, a polydisperse, fractionated glycan polymer preparation is provided comprising at least 85%, 90%, or at least 95% medium-length species with a DP of about 3-10. In one embodiment, a polydisperse, fractionated glycan polymer preparation is provided comprising at least 85%, 90%, or at least 95% long-length species with a DP of about 11-18. In one embodiment, a polydisperse, fractionated glycan polymer preparation is provided comprising at least 85%, 90%, or at least 95% very long-length species with a DP of about 18-30.

Optionally, the preparations may be fractionated, e.g. representing 60%, 65%, 70%, 75%, 80%, 85%, 90%, 95%, 97%, 98%, or greater than 98% short (about DP1-2) or medium (about DP3-10) glycans in the preparation. Alternatively, or in addition to fractionation, the small DP fraction (e.g. monomers and dimers) are subjected to enzymatic fermentation, e.g. with suitable yeasts to break down these sugars. In one embodiment, a polydisperse, fractionated glycan polymer preparation is prepared using a method described herein, comprising at least 85%, 90%, or at least 95% of glycans with a DP of about 3-10.

In some embodiments, about 55%, 60%, 65%, 70%, 75%, 80%, 85%, 90%, 95%, or about 97% of the glycan polymers of the glycan preparation have a DP of at least DP3, DP4, DP5, DP6 or DP7. In some embodiments, about 55%, 60%, 65%, 70%, 75%, 80%, 85%, 90%, 95%, or about 97% of the glycan polymers of the glycan preparation have a DP from about DP3 to about DP10, from about DP3 to about DP8, from about DP3 to about DP6, from about DP3 to about DP5, from about DP3 to about DP4, from about DP2 to about DP4, from about DP2 to about DP5, from about DP2 to about DP6, from about DP2 to about DP8, or from about DP2 to about DP10. In some embodiments, less than 1%, 2%, 3%, 5%, 10%, 15%, 20%, 25%, 30%, 40%, or less than 50% of the glycan polymers of the glycan preparation have a DP of DP2 or less.

In some embodiments, about 55%, 60%, 65%, 70%, 75%, 80%, 85%, 90%, 95%, or about 97% of the glycan polymer preparation has a DP of between 2 and 25, between 3 and 25, between 4 and 25, between 5 and 25, between 6 and 25, between 7 and 25, between 8 and 25, between 9 and 25, between 10 and 25, between 2 and 30, between 3 and 30, between 4 and 30, between 5 and 30, between 6 and 30, between 7 and 30, between 8 and 30, between 9 and 30, or between 10 and 30. In one embodiment, the glycan polymer preparation has a degree of polymerization (DP) of at least 3 and less than 30 glycan units.

In some embodiments, about 55%, 60%, 65%, 70%, 75%, 80%, 85%, 90%, 95%, or about 97% of the glycan polymer preparation has a DP of at least 5 and less than 30 glycan units. In some embodiments, about 55%, 60%, 65%, 70%, 75%, 80%, 85%, 90%, 95%, or about 97% of the glycan polymer preparation has a DP of at least 8 and less than 30 glycan units. In some embodiments, about 55%, 60%, 65%, 70%, 75%, 80%, 85%, 90%, 95%, or about 97% of the glycan polymer preparation has a DP of at least 10 and less than 30 glycan units. In some embodiments, about 55%, 60%, 65%, 70%, 75%, 80%, 85%, 90%, 95%, or about 97% of the glycan polymer preparation has a DP of between 3, 4, 5, 6, 7, 8 and 10, 11, 12, 13, 14, 15, 16, 17, 18, 19, 20 glycan units. In some embodiments, about 55%, 60%, 65%, 70%, 75%, 80%, 85%, 90%, 95%, or about 97% of the glycan polymer preparation has a DP of between 10, 11, 12, 13, 14, 15, 16, 17, 18, 19 and 20, 21, 22, 23, 24, 25, 26, 27, 28, 29, 30 glycan units. In some embodiments, about 55%, 60%, 65%, 70%, 75%, 80%, 85%, 90%, 95%, or about 97% of the glycan polymer preparation has a DP of between 3, 4, 5, 6, 7, 8, 9, 10 and 20, 21, 22, 23, 24, 25, 26, 27, 28 glycan units. In one embodiment, about 55%, 60%, 65%, 70%, 75%, 80%, 85%, 90%, 95%, or about 97% of the glycan polymer preparation has a DP of at least 2. In one embodiment, about 55%, 60%, 65%, 70%, 75%, 80%, 85%, 90%, 95%, or about 97% of the glycan polymer preparation has a DP of at least 3.

Average DP

In some embodiments, the glycan polymer preparation has an average degree of polymerization (average DP) of about DP2, DP3, DP4, DPS, DP6, DP7, DP8, or DP9. In some embodiments, the glycan polymer preparation has an average degree of polymerization (average DP) of between about 2 and about 10, between about 2 and about 8, between about 2 and about 6, between about 2 and about 4, between about 3 and about 10, between about 3 and about 8, between about 3 and about 6, or between about 3 and about 4.

In some embodiments, about 55%, 60%, 65%, 70%, 75%, 80%, 85%, 90%, 95%, or about 97% of the glycan polymer preparation has an average degree of polymerization (DP) of about DP5, DP6, DP7, DP8, DP9, DP10, DP11, or DP12. In some embodiments, the average DP of the glycan polymer preparation is between about DP5 and DP10, between about DP6 and DP10, between about DP6 and DP12, between about DP6 and DP14, between about DP8 and DP12, between about DP8 and DP14, between about DP8 and DP16, between about DP10 and DP16 between about DP10 and DP18, between about DP4 and DP18, between about DP6 and DP18, or between about DP8 and DP18.

Average Molecular Weight

In some embodiments, about 55%, 60%, 65%, 70%, 75%, 80%, 85%, 90%, 95%, or about 97% of the glycan polymers of the preparation have an average molecular weight of about 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800 g/mol and less than 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, and 5000 g/mol.

Degree of Branching (DB)

In some embodiments, the glycan preparations range in structure from linear to branched. Branched glycans may contain at least one glycan subunit being linked via an alpha or a beta glycosidic bond so as to form a branch. The branching rate or degree of branching (DB) may vary, such that the glycan polymers of a preparation comprise at least 1, at least 2, at least 3, at least 4, at least 5, or at least about 6 branching points in the glycan polymer. In some embodiments, the glycan polymers of the glycan preparation are unbranched (DB=0).

In some embodiments, the glycan preparations (e.g. oligo- or polysaccharides) range in structure from linear to highly branched. Unbranched glycans may contain only alpha linkages or only beta linkages. Unbranched glycans may contain at least one alpha and at least one beta linkage. Branched glycans may contain at least one glycan unit being linked via an alpha or a beta glycosidic bond so as to form a branch. The branching rate or degree of branching (DB) may vary, such that about every 2^{nd}, 3^{rd}, 4^{th}, 5^{th}, 6^{th}, 7^{th}, 8^{th}, 9^{th}, 10^{th}, 15^{th}, 20^{th}, 25^{th}, 30^{th}, 35^{th}, 40^{th}, 45^{th}, 50^{th}, 60^{th}, or 70^{th} unit comprises at least one branching point. For example, animal glycogen contains a branching point approximately every 10 units.

In some embodiments, preparations of glycan polymer are provided, wherein the preparation comprises a mixture of branched glycans, wherein the average degree of branching (DB, branching points per residue) is 0, 0.01. 0.02, 0.03, 0.04, 0.05, 0.06, 0.07, 0.08, 0.09, 0.1, 0.2, 0.3, 0.4, 0.5, 0.6, 0.7, 0.8, 0.9, 0.95, 0.99, 1, or 2. In some embodiments, preparations of glycan polymers are provided, wherein the avarage degree of branching is at least 0.01, 0.05, 0.1, 0.2, 0.3, or at least 0.4. In some embodiments, preparations of glycan polymers are provided, wherein the avarage degree of branching is between about 0.01 and 0.1, 0.01 and 0.2, 0.01 and 0.3, 0.01 and 0.4, 0.01 and 0.5, 0.01 and 0.6, or between about 0.01 and 0.7. In some embodiments, preparations of glycan polymers are provided, wherein the avarage degree of branching is between about 0.05 and 0.1, 0.05 and 0.2, 0.05 and 0.3, 0.05 and 0.4, 0.05 and 0.5, 0.05 and 0.6, or between about 0.05 and 0.7. In some embodiments, preparations of glycan polymers are provided, wherein the avarage degree of branching is not 0. In some embodiments, preparations of glycan polymers are provided, wherein the avarage degree of branching is not between at least 0.1 and less than 0.4 or at least 0.2 and less than 0.4. In some embodiments, the preparations of glycan polymers comprise linear glycans. In some embodiments, the preparations of glycan polymers comprise glycans that exhibit a branched or branch-on-branch structure.

In some embodiments, preparations of glycan polymers are provided wherein the avarage degree of branching (DB) is not 0, but is at least 0.01, 0.05, 0.1, or at least 0.2, or ranges between about 0.01 and about 0.2 or between about 0.05 and 0.1.

Glycosidic Bonds and Linkages

Linkages between the individual glycan subunits found in preparations of glycan polymers may include alpha 1→2, alpha 1→3, alpha 1→4, alpha 1→5, alpha 1→6, alpha 2→1, alpha 2→3, alpha 2→4, alpha 2→6, beta 1→2, beta 1→3, beta 1→4, beta 1→5, beta 1→6, beta 2→1, beta 2→3, beta 2→4, and beta 2→6.

In some embodiments, the glycan polymer preparations comprise only alpha linkages. In some embodiments, the glycan polymers comprise only beta linkages. In some embodiments, the glycan polymers comprise mixtures of alpha and beta linkages.

In some embodiments, the alpha:beta glycosidic bond ratio in a preparation is about 1:1, 2:1, 3:1, 4:1, or 5:1. . In some embodiments, the beta:alpha glycosidic bond ratio in a preparation is about 1:1, 2:1, 3:1, 4:1, or 5:1.

In some embodiments, the alpha:beta glycosidic bond ratio in a preparation is about 0.1:1, 0.2:1, 0.3:1, 0.4:1, 0.5:1, 0.6:1, 0.7:1, 0.8:1, 0.9:1, 1:1, 1.2:1, 1.5:1, 1.7:1, 2:1, 2.2:1, 2.5:1, 2.7:1, 3:1, 4:1, 5:1, 6:1, 7:1, 8:1, 9:1, or about 10:1.

In some embodiments, the glycan polymers of the glycan polymer preparation comprise both alpha- and beta-glycosidic bonds selected from the group consisting of 1→2 glycosidic bond, a 1→3 glycosidic bond, a 1→4 glycosidic bond, a 1→5 glycosidic bond and a 1→6 glycosidic bond. In some embodiments, the glycan polymer preparation comprises at least two or at least three alpha and beta 1→2 glycosidic bonds, alpha and beta 1→3 glycosidic bonds, alpha and beta 1→4 glycosidic bonds, alpha and beta 1→5 glycosidic bonds, and/or alpha and beta 1→6 glycosidic bonds.

In some embodiments, the glycan polymers of the glycan preparation comprise substantially all alpha- or beta configured glycan subunits, optionally comprising about 1%, 2%, 3%, 4% 5%, 6%, 7%, 8%, 9%, 10%, 11%, 12%, 13%, 14%, 15%, 16%, 17%, 18%, 19%, or 20% of the respective other configuration.

In some embodiments, the preparations of glycan polymers comprise at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 98%, 99%, at least 99.9% or even 100% glycans with alpha glycosidic bonds. In some embodiments, the preparations of glycan polymers comprise at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 98%, 99%, at least 99.9% or even 100% glycans with beta glycosidic bonds. In some embodiments, preparations of glycan polymers are provided, wherein at least 10%, 15%, 20%, 25%, 30%, 35%, 40%, 45%, 50%, 55%, 60%, 65%, 70%, 75%, 80%, or at least 85% of glycans with glycosidic bonds that are alpha glycosidic bonds, at least 10%, 15%, 20%, 25%, 30%, 35%, 40%, 45%, 50%, 55%, 60%, 65%, 70%, 75%, 80%, or at least 85% of glycans with glycosidic bonds that are beta glycosidic bonds, and wherein the percentage of alpha and beta glycosidic bonds does not exceed 100%.

In some embodiments, preparations of glycan polymers are provided, wherein at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 98%, 99%, at least 99.9% or even 100% of glycan glycosidic bonds are one or more of: 1→2 glycosidic bonds, 1→3 glycosidic bonds, 1→4 glycosidic bonds, and 1→6 glycosidic bonds. In some embodiments, preparations of glycan polymers are provided, wherein at least 1%, 2%, 3%, 4%, 5%, 6%, 7%, 8%, 9%, 10%, 15%, at least 20%, or 25% each of glycan glycosidic bonds are 1→2, 1→3, 1→4, and 1→6 glycosidic bonds. Optionally, the preparations of glycan polymers further comprise at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, or at least 85% of glycan glycosidic bonds that are selected from the group consisting of : alpha 2→1, alpha 2→3, alpha 2→4, alpha 2→6, beta 2→1, beta 2→3, beta 2→4, and beta 2→6, glycosidic bonds.

In some embodiments, the glycan polymers of the glycan preparation comprise at least two glycosidic bonds selected from the group consisting of alpha 1→2 and alpha 1→3, alpha 1→2 and alpha 1→4, alpha 1→2 and alpha 1→6, alpha 1→2 and beta 1→2, alpha 1→2 and beta 1→3, alpha 1→2 and beta 1→4, alpha 1→2 and beta 1→6, alpha 1→3 and alpha 1→4, alpha 1→3 and alpha 1→6, alpha 1→3 and beta 1→2, alpha 1→3 and beta 1→3, alpha 1→3 and beta 1→4, alpha 1→3 and beta 1→6, alpha 1→4 and alpha 1→6, alpha 1→4 and beta 1→2, alpha 1→4 and beta 1→3, alpha 1→4 and beta 1→4, alpha 1→4 and beta 1→6, alpha 1→6 and beta 1→2, alpha 1→6 and beta 1→3, alpha 1→6 and beta 1→4, alpha 1→6 and beta 1→6, beta 1→2 and beta 1→3, beta 1→2 and beta 1→4, beta 1→2 and beta 1→6, beta 1→3 and beta 1→4, beta 1→3 and beta 1→6, and beta 1→4 and beta 1→6.

L- and D-Forms

In some embodiments, preparations of glycan polymers are provided, wherein at least one glycan subunit is a sugar in L-form. In some embodiments, preparations of glycans are provided, wherein at least one glycan subunit is a sugar in D-form. In some embodiments, preparations of glycans are provided, wherein the glycan subunits are sugars in L- or D-form as they naturally occur or are more common (e.g. D-glucose, D-xylose, L-arabinose).

In some embodiments, the preparation of glycan polymers (e.g. oligosaccharides and polysaccharides) comprises a desired mixture of L- and D-forms of glycan subunits, e.g. of a desired ratio, such as: 1:1, 1:2, 1:3, 1:4, 1:5 L- to D-forms or D- to L-forms.

In some embodiments, the preparation of glycan polymers comprises a desired mixture of L- and D-forms of glycan units, e.g. of a desired ratio, such as: 1:1, 1:2, 1:3, 1:4, 1:5, 1:6, 1:7, 1:8, 1:9, 1:10, 1:12, 1:14, 1:16, 1:18, 1:20, 1:25, 1:30, 1:35, 1:40, 1:45, 1:50, 1:55, 1:60, 1:65, 1:70, 1:75, 1:80, 1:85, 1:90, 1:100, 1:150 L- to D-forms or D- to L-forms.

In some embodiments, the preparation of glycan polymers comprises glycans with substantially all L- or D-forms of glycan subunits, optionally comprising about 1%, 2%, 3%, 4% 5%, 6%, 7%, 8%, 9%, 10%, 11%, 12%, 13%, 14%, 15%, 16%, 17%, 18%, 19%, or 20% of the respective other form.

Glycan Unit Content

In some embodiments, preparations of glycan polymers are provided, wherein at least one glycan subunit is a tetrose, a pentose, a hexose, or a heptose. Optionally, the glycan subunits involved in the formation of the glycans of the glycan polymer preparation are varied. Examples of monosaccharide glycan subunits include hexoses, such as glucose, galactose, and fructose, and pentoses, such as xylose. Monosaccharides generally have the chemical formula: Cₓ(H₂O)_{y}, where conventionally x≥3. Monosaccharides can be classified by the number x of carbon atoms they contain, for example: diose (2) triose (3) tetrose (4), pentose (5), hexose (6), and heptose (7). The monosaccharide glycan subunits may exist in an acyclic (open-chain) form. Open-chain monosaccharides with same molecular graph may exist as two or more stereoisomers. The monosaccharides may also exist in a cyclic form through a nucleophilic addition reaction between the carbonyl group and one of the hydroxyls of the same molecule. The reaction creates a ring of carbon atoms closed by one bridging oxygen atom. In these cyclic forms, the ring usually has 5 (furanoses) or 6 atoms (pyranoses).

In some embodiments, the preparation of glycan polymers comprises a desired mixture of different monosaccharide glycan subunits, such as a mixture of a diose (2), a triose (3), tetrose (4), pentose (5), hexose (6), or heptose (7). In some embodiments, the glycan polymers of the glycan polymer preparation comprise a desired mixture of a pentose (5) and a hexose (6).

In some embodiments, the preparation of glycan polymers comprises a desired mixture of two, three, four or five different glycan subunits, such as a mixture of, e.g., i) one or more glycan subunits selected from monosaccharides, selected from glucose, a galactose, an arabinose, a mannose, a fructose, a xylose, a fucose, and a rhamnose; ii) one or more glycan subunits selected from disaccharides selected from acarviosin, n-acetyllactosamine, allolactose, cellobiose, chitobiose, glactose-alpha-1,3-galactose, gentiobiose, isomalt, isomaltose, isomaltulose, kojibiose, lactitol, lactobionic acid, lactose, lactulose, laminaribiose, maltitol, maltose, mannobiose, melibiose, melibiulose, neohesperidose, nigerose, robinose, rutinose, sambubiose, sophorose, sucralose, sucrose, sucrose acetate isobutyrate, sucrose octaacetate, trehalose, turanose, vicianose, and xylobiose; iii) one or more glycan subunits selected from amino sugars selected from acarbose, N-acetylemannosamine, N-acetylmuramic acid, N-acetylnueraminic acid, N-acetyletalosaminuronic acid, arabinopyranosyl-N-methyl-N-nitrosourea, D-fructose-L-histidine, N-glycolyneuraminic acid, ketosamine, kidamycin, mannosamine, 1B-methylseleno-N-acetyl-D-galactosamine, muramic acid, muramyl dipeptide, phosphoribosylamine, PUGNAc, sialyl-Lewis A, sialyl-Lewis X, validamycin, voglibose, N-acetylgalactosamine, N-acetylglucosamine, aspartylglucosamine, bacillithiol, daunosamine, desosamine, fructosamine, galactosamine, glucosamine, meglumine, and perosamine; iv) one or more glycan subunits selected from deoxy sugars selected from 1-5-ahydroglucitol, cladinose, colitose, 2-deoxy-D-glucose, 3-deoxyglucasone, deoxyribose, dideoxynucleotide, digitalose, fludeooxyglucose, sarmentose, and sulfoquinovose; v) one or more glycan subunits selected from imino sugars selected from castanospermine, 1-deoxynojirimycin, iminosugar, miglitol, miglustat, and swainsonine; one or more glycan subunits selected from sugar acids selected from N-acetylneuraminic acid, N-acetyltalosamnuronic acid, aldaric acid, aldonic acid, 3-deoxy-D-manno-oct-2-ulosonic acid, glucuronic acid, glucosaminuronic acid, glyceric acid, N-glycolylneuraminic acid, iduronic acid, isosaccharinic acid, pangamic acid, sialic acid, threonic acid, ulosonic acid, uronic acid, xylonic acid, gluconic acid, ascorbic acid, ketodeoxyoctulosonic acid, galacturonic acid, galactosaminuronic acid, mannuronic acid, mannosaminuronic acid, tartaric acid, mucic acid, saccharic acid, lactic acid, oxalic acid, succinic acid, hexanoic acid, fumaric acid, maleic acid, butyric acid, citric acid, glucosaminic acid, malic acid, succinamic acid, sebacic acid, and capric acid; vi) one or more glycan subunits selected from short-chain fatty acids selected from formic acid, acetic acid, propionic acid, butryic acid, isobutyric acid, valeric acid, and isovaleric acid; and vii) one or more glycan subunits selected from sugar alcohols selected from methanol, ethylene glycol, glycerol, erythritol, threitol, arabitol, ribitol, xylitol, mannitol, sorbitol, galactitol, iditol, volemitol, fucitol, inositol, maltotritol, maltotetraitol, and polyglycitol.

Exemplary glycans are described by a three-letter code representing the monomeric sugar component followed by a number out of one hundred reflecting the percentage of the material that monomer constitutes. Thus, ‘glu100’ is ascribed to a glycan generated from a 100% D-glucose (glycan unit) input and ‘glu50gal50’ is ascribed to a glycan generated from 50% D-glucose and 50% D-galactose (glycan units) input or, alternatively from a lactose dimer (glycan unit) input. As used herein: xyl=D-xylose; ara=L-arabinose; gal=D-galactose; glu=D-glucose; rha=L-rhamnose; fuc=L-fucose; man=D-mannose; sor=D-sorbitol; gly=D-glycerol; neu=NAc-neuraminic acid.

In some embodiments, the preparation of glycan polymers comprises one glycan unit A selected from i) to vii) above, wherein glycan unit A comprises 100% of the glycan unit input. For example, in some embodiments, the glycan polymer preparation is selected from the homo-glycans xyl100, rha100, ara100, gal100, glu100, and man100. In some embodiments, the glycan polymer preparation is selected from the homo-glycans fuc100 and fru100.

Provided herein are glycan preparations (as described herein, e.g., having any DP, DB, alpha:beta glycosidic bond ratio, number of glycosidic bonds, bond regiochemistry and bond stereochemistry, and other characteristics (e.g., solubility, fermentability, viscosity, sweetness, etc.) described herein), comprising glycans comprising:
1) a glucose glycan unit, optionally wherein the glycan preparation comprises any amount of glucose between 1% and 100%, further optionally wherein the glycan preparation comprises a second, third, fourth or fifth glycan unit (optionally, independently selected from xylose, arabinose, galactose, mannose, rhamnose, fructose, or fucose), further optionally, wherein the glycan preparation is one of: gal50glu25fru25, gal57glu43, gal57glu43, glu100, glu10gal10man80, glu10gal45man45, glu10gal80man10, glu20ara80, glu20gal20man20xyl20ara20, glu20gal20man60, glu20gal40man40, glu20gal60man20, glu20gal80, glu20xyl80, glu25gal25man25ara25, glu25gal25man25xyl25, glu25gal25xyl25ara25, glu25man25xyl25ara25, glu30gal30man40, glu30gal40man30, glu33gal33ara33, glu33gal33fuc33, glu33gal33man33, glu33gal33xyl33, glu33man33ara33, glu33man33xyl33, glu33xyl33ara33, glu40ara60, glu40gal20man40, glu40gal30man30, glu40gal40man20, glu40gal60, glu40xyl60, glu45gal10man45, glu45gal45man10, glu50gal50, glu5gal5man90, glu5gal90man5, glu60ara40, glu60gal20man20, glu60gal40, glu60man40, glu60xyl40, glu66fru33, glu80ara20, glu80gal10man10, glu80gal20, glu80man20, glu80man20, glu80xyl20, glu90gal5man5, man52glu29gal19, man60glu40, man62glu38, man80glu20, xyl33glu33gal33, or xyl75glu12gal12;
2) a galactose glycan unit, optionally wherein the glycan preparation comprises any amount of galactose between 1% and 100%, further optionally wherein the glycan preparation comprises a second, third, fourth or fifth glycan unit (optionally, independently selected from xylose, arabinose, glucose, mannose, rhamnose, fructose, or fucose), further optionally, wherein the glycan preparation is one of: ara50gal50, gal100, gal20ara80, gal20xyl80, gal25man25xyl25ara25, gal33man33ara33, gal33man33xyl33, gal33xyl33ara33, gal40ara60, gal40man60, gal40xyl60, gal50glu25fru25, gal57fru43, gal57glu43, gal60ara40, gal60man40, gal60xyl40, gal75xyl25, gal80ara20, gal80man20, gal80xyl20, glu10gal10man80, glu10gal45man45, glu10gal80man10, glu20gal20man20xyl20ara20, glu20gal20man60, glu20gal40man40, glu20gal60man20, glu20gal80, glu25gal25man25ara25, glu25gal25man25xyl25, glu25gal25xyl25ara25, glu30gal30man40, glu30gal40man30, glu33gal33ara33, glu33gal33fuc33, glu33gal33man33, glu33gal33xyl33, glu40gal20man40, glu40gal30man30, glu40gal40man20, glu40gal60, glu45gal10man45, glu45gal45man10, glu50gal50, glu5gal5man90, glu5gal90man5, glu60gal20man20, glu60gal40, glu80gal10man10, glu80gal20, glu90gal5man5, man52glu29gal19, man66gal33, man75gal25, man80gal20, xyl33glu33gal33, xyl75gal25, or xyl75glu12gal12;
3) a mannose glycan unit, optionally wherein the glycan preparation comprises any amount of mannose between 1% and 100%, further optionally wherein the glycan preparation comprises a second, third, fourth or fifth glycan unit (optionally, independently selected from xylose, arabinose, glucose, galactose, rhamnose, fructose, or fucose), further optionally, wherein the glycan preparation is one of: gal25man25xyl25ara25, gal33man33ara33, gal33man33xyl33, gal40man60, gal60man40, gal80man20, glu10gal10man80, glu10gal45man45, glu10gal80man10, glu20gal20man20xyl20ara20, glu20gal20man60, glu20gal40man40, glu20gal60man20, glu25gal25man25ara25, glu25gal25man25xyl25, glu25man25xyl25ara25, glu30gal30man40, glu30gal40man30, glu33gal33man33, glu33man33ara33, glu33man33xyl33, glu40gal20man40, glu40gal30man30, glu40gal40man20, glu45gal10man45, glu45gal45man10, glu5gal5man90, glu5gal90man5, glu60gal20man20, glu60man40, glu80gal10man10, glu80man20, glu80man20, glu90gal5man5, man100, man20ara80, man20xyl80, man33xyl33ara33, man40ara60, man40xyl60, man52glu29gal19, man60ara40, man60glu40, man60xyl40, man62glu38, man66gal33, man75gal25, man80ara20, man80gal20, man80glu20, or man80xyl20;
4) an arabinose glycan unit, optionally wherein the glycan preparation comprises any amount of arabinose between 1% and 100%, further optionally wherein the glycan preparation comprises a second, third, fourth or fifth glycan unit (optionally, independently selected from xylose, glucose, galactose, mannose, rhamnose, fructose, or fucose), further optionally, wherein the glycan preparation is one of: ara100, ara50gal50, ara50xyl50, ara60xyl40, ara80xyl20, gal20ara80, gal25man25xyl25ara25, gal33man33ara33, gal33xyl33ara33, gal40ara60, gal60ara40, gal80ara20, glu20ara80, glu20gal20man20xyl20ara20, glu25gal25man25ara25, glu25gal25xyl25ara25, glu25man25xyl25ara25, glu33gal33ara33, glu33man33ara33, glu33xyl33ara33, glu40ara60, glu60ara40, glu80ara20, man20ara80, man33xyl33ara33, man40ara60, man60ara40, man80ara20, xyl60ara40, xyl75ara25, or xyl80ara20;
5) a xylose glycan unit, optionally wherein the glycan preparation comprises any amount of xylose between 1% and 100%, further optionally wherein the glycan preparation comprises a second, third, fourth or fifth glycan unit (optionally, independently selected from arabinose, glucose, galactose, mannose, rhamnose, fructose, or fucose), further optionally, wherein the glycan preparation is one of: ara50xyl50, ara60xyl40, ara80xyl20, gal20xyl80, gal25man25xyl25ara25, gal33man33xyl33, gal33xyl33ara33, gal40xyl60, gal60xyl40, gal75xyl25, gal80xyl20, glu20gal20man20xyl20ara20, glu20xyl80, glu25gal25man25xyl25, glu25gal25xyl25ara25, glu25man25xyl25ara25, glu33gal33xyl33, glu33man33xyl33, glu33xyl33ara33, glu40xyl60, glu60xyl40, glu80xyl20, man20xyl80, man33xyl33ara33, man40xyl60, man60xyl40, man80xyl20, xyl100, xyl33glu33gal33, xyl60ara40, xyl75ara25, xyl75gal25, xyl75glu12gal12, or xyl80ara20;
6) a fructose glycan unit, optionally wherein the glycan preparation comprises any amount of fructose between 1% and 100%, further optionally wherein the glycan preparation comprises a second, third, fourth or fifth glycan unit (optionally, independently selected from xylose, arabinose, glucose, galactose, mannose, rhamnose, or fucose), further optionally, wherein the glycan preparation is one of: fru100, gal50glu25fru25, gal57fru43, or glu66fru33;
7) a fucose glycan unit, optionally wherein the glycan preparation comprises any amount of fucose between 1% and 100%, further optionally wherein the glycan preparation comprises a second, third, fourth or fifth glycan unit (optionally, independently selected from xylose, arabinose, glucose, galactose, mannose, rhamnose, or fructose), further optionally, wherein the glycan preparation is one of: glu33gal33fuc33;
8) a rhamnose glycan unit, optionally wherein the glycan preparation comprises any amount of rhamnose between 1% and 100%, further optionally wherein the glycan preparation comprises a second, third, fourth or fifth glycan unit (optionally, independently selected from xylose, arabinose, glucose, galactose, mannose, fructose, or fucose), further optionally, wherein the glycan preparation is rha100; and further, optionally, wherein the glycan preparation comprises one or more (e.g., two, three, four, five, six, seven, eight, or nine) of the following properties (including bulk properties):

i) the glycan preparation comprises glycans that comprise glucose, galactose, arabinose, mannose, fructose, xylose, fucose, or rhamnose glycan units;

ii) the average degree of branching (DB) of the glycans in the glycan preparation is 0, between 0.01 and 0.6, between 0.05 and 0.5, between 0.1 and 0.4, or between 0.15 and 0.4;

iii) at least 50% (at least 60%, 65%, 70%, 75%, 80%, or 85%, or less than 50%) of the glycans in the glycan preparation have a degree of polymerization (DP) of at least 3 and less than 30 glycan units, at least 2 and less than 10 glycan units, at least 5 and less than 25 glycan units, or at least 10 and less than 35 glycan units (optionally, wherein the glycan unit is a monomer, e.g., a monosugar);

iv) the average DP (mean DP) of the glycan preparation is between about 2 and 5, between about 5 and 8, between about 8 and 13, between about 13 and 25, between about 5 and 15, between about 5 and 20, or between about 5-15;

v) the ratio of alpha- to beta-glycosidic bonds present in the glycans of the glycan preparation is 0, or between about 0.8:1 to about 5:1, between about 1:1 to about 5:1, between about 1:1 to about 3:1, between about 3:2 to about 2:1, or between about 3:2 to about 3:1,

vi) the glycan preparation comprises between 15 mol % and 75 mol % (between 20 mol % and 60 mol %, between 25 mol % and 50 mol %, or between 30 mol % and 45 mol %) 1,6 glycosidic bonds;

vii) the glycan preparation comprises between 1 mol % and 40 mol % (between 1 mol % and 30 mol %, between 5 mol % and 25 mol %, between 10 mol % and 20 mol %) of at least one, two, or three of 1,2; 1,3; and 1,4 glycosidic bonds;

viii) the glycan preparation has a final solubility limit in water of at least about 50 (at least about 60, 70, at least about 75, or less than 50) Brix at 23° C.; or

ix) the glycan preparation has a dietary fiber content (e.g., as measured by AOAC 2009.01) of at least 50% (at least 60%, 70%, 80%, or at least 90%, or less than 50%),

x) any combination of:
two of: i), ii), iii), iv), v), vi), vii), viii), and ix);
three of: i), ii), iii), iv), v), vi), vii), viii), and ix);
four of: i), ii), iii), iv), v), vi), vii), viii), and ix);
five of: i), ii), iii), iv), v), vi), vii), viii), and ix);
six of: i), ii), iii), iv), v), vi), vii), viii), and ix);
seven of: i), ii), iii), iv), v), vi), vii), viii), and ix);
eight of: i), ii), iii), iv), v), vi), vii), viii), and ix); or
all of: i), ii), iii), iv), v), vi), vii), viii), and ix), and as exemplified, e.g., in Table 10.

In some embodiments, the preparation of glycan polymers comprises a mixture of two glycan units A and B selected independently from i) to vii) above, wherein A and B may be selected from the same or a different group i) to vii) and wherein A and B may be selected in any desired ratio (e.g. anywhere from 1-99% A and 99-1% B, not exceeding 100%).

For example, in some embodiments, the glycan polymer preparation is selected from the hetero-glycans ara50gal50, ara50gal50, xyl75gal25, ara80xyl20, ara60xyl40, ara50xyl50, glu80man20, glu60man40, man80glu20, man60glu40, xyl75ara25, gal75xyl25, Man80gal20, gal75xyl25, Man66gal33, Man75gal25, glu80gal20, glu60gal40, glu40gal60, glu20gal80, gal80man20, gal60man40, gal40man60, glu80xyl20, glu60xyl40, glu40xyl60, glu20xyl80, glu80ara20, glu60ara40, glu40ara60, glu20ara80, gal80xyl20, gal60xyl40, gal40xyl60, gal20xyl80, gal80ara20, gal60ara40, gal40ara60, gal20ara80, man80xyl20, man60xyl40, man40xyl60, man20xyl80, man80ara20, man60ara40, man40ara60, man20ara80, xyl80ara20, xyl60ara40, glu50gal50, and man62glu38.

In some embodiments, the preparation of glycan polymers comprises a mixture of three glycan units A, B and C selected independently from i) to vii) above, wherein A, B and C may be selected from the same or a different group i) to vii) and wherein A, B and C may be selected in any desired ratio (e.g. anywhere from 1-99% A, 1-99% B, 1-99% C, not exceeding 100%).

For example, in some embodiments, the glycan polymer preparation is selected from the hetero-glycans xyl75glu12gal12, xyl33glu33gal33, xyl75glu12gal12, glu33gal33fuc33, glu33gal33nman33, glu33gal33xyl33, glu33gal33ara33, gal33man33xyl33, gal33man33ara33, man52glu29gal19, Glu33Man33Xyl33, Glu33Man33Ara33, Glu33Xyl33Ara33, Gal33Man33Xyl33, Gal33Man33Ara33, Gal33Xyl33Ara33, Man33Xyl33Ara33, Glu90Gal5Man5, Glu80Gal10Man10, Glu60Gal20Man20, Glu40Gal30Man30, Glu20Gal40Man40, Glu10Gal45Man45, Glu5Gal90Man5, Glu10Gal80Man10, Glu20Gal60Man20, Glu30Gal40Man30, Glu40Gal20Man40, Glu45Gal10Man45, Glu5Gal5Man90, Glu10Gal10Man80, Glu20Gal20Man60, Glu30Gal30Man40, Glu40Gal40Man20, and Glu45Gal45Man10.

In some embodiments, the preparation of glycan polymers comprises a mixture of four glycan units A, B, C and D selected independently from i) to vii) above, wherein A, B, C and D may be selected from the same or a different group i) to vii) and wherein A, B, C and D may be selected in any desired ratio (e.g. anywhere from 1-99% A, 1-99% B, 1-99% C, 1-99% D, not exceeding 100%).

In some embodiments, the preparation of glycan polymers comprises a mixture of five glycan units A, B, C, D and E selected independently from i) to vii) above, wherein A, B, C, D and E may be selected from the same or a different group i) to vii) and wherein A, B, C, D and E may be selected in any desired ratio (e.g. anywhere from 1-99% A, 1-99% B, 1-99% C, 1-99% D, 1-99% E, not exceeding 100%).

In some embodiments, preparations of glycan polymers are provided, wherein at least one glycan subunit is selected from the group consisting of a glucose, a galactose, an arabinose, a mannose, a fructose, a xylose, a fucose, and a rhamnose.

In some embodiments, the preparation of glycan polymers comprises a desired mixture of two different monosaccharide glycan subunits, such as a mixture of, e.g., glucose and galactose, glucose and arabinose, glucose and mannose, glucose and fructose, glucose and xylose, glucose and fucose, glucose and rhamnose, galactose and arabinose, galactose and mannose, galactose and fructose, galactose and xylose, galactose and fucose, and galactose and rhamnose, arabinose and mannose, arabinose and fructose, arabinose and xylose, arabinose and fucose, and arabinose and rhamnose, mannose and fructose, mannose and xylose, mannose and fucose, and mannose and rhamnose, fructose and xylose, fructose and fucose, and fructose and rhamnose, xylose and fucose, xylose and rhamnose, and fucose and rhamnose, e.g. a in a ratio of 1:1, 1:2, 1:3, 1:4, or 1:5 or the reverse ratio thereof, or a in a ratio of 1:1, 1:2, 1:3, 1:4, 1:5, 1:6, 1:7, 1:8, 1:9, 1:10, 1:12, 1:14, 1:16, 1:18, 1:20, 1:25, 1:30, 1:35, 1:40, 1:45, 1:50, 1:55, 1:60, 1:65, 1:70, 1:75, 1:80, 1:85, 1:90, or 1:100 or the reverse ratio thereof.

In some embodiments, the preparation of glycan polymers comprises a desired mixture of three different monosaccharide glycan subunits, such as a mixture of, e.g. for glucose-containing glycan preparations, glucose, galactose and arabinose; glucose, galactose and mannose; glucose, galactose and fructose; glucose, galactose and xylose; glucose, galactose and fucose, glucose, galactose and rhamnose; glucose, arabinose, and mannose; glucose, arabinose and fructose; glucose, arabinose and xylose; glucose, arabinose and fucose; glucose, arabinose and rhamnose; glucose, mannose and fructose; glucose, mannose and xylose; glucose, mannose and fucose; glucose, mannose rhamnose; glucose, fructose and xylose; glucose, fructose and fucose; glucose, fructose and rhamnose; glucose, fucose and rhamnose, e.g. a in a ratio of 1:1:1, 1:2:1, 1:3:1, 1:4:1, 1:5:1, 1:1:2, 1:2:2, 1:3:2, 1:4:2, 1:1:3, 1:2:3, 1:3:3, 1:1:4, 1:2:4, 1:1:5, 1:2:5, etc., or a in a ratio of 1:1:1, 1:2:1, 1:3:1, 1:4:1, 1:5:1, 1:6:1, 1:7:1, 1:8:1, 1:9:1, 1:10:1, 1:12:1, 1:14:1, 1:16:1, 1:18:1, 1:20:1, 1:1:2, 1:2:2, 1:3:2, 1:4:2, 1:5:2, 1:6:2, 1:7:2, 1:8:2, 1:9:2, 1:10:2, 1:1:3, 1:2:3, 1:3:3, 1:4:3, 1:5:3, 1:6:3, 1:7:3, 1:8:3, 1:9:3, 1:10:3, 1:1:4, 1:2:4, 1:3:4, 1:4:4, 1:5:4, 1:6:4, 1:7:4, 1:8:4, 1:9:4, 1:10:4, 1:1:5, 1:2:5, 1:3:5, 1:4:5, 1:5:5, 1:6:5, 1:7:5, 1:8:5, 1:9:5, 1:10:5, etc.

In some embodiments, the preparation of glycan polymers does not comprise N-acetylgalactosamine or N-acetylglucosamine. In some embodiments, the preparation of glycans does not comprise sialic acid. In some embodiments, the preparation of glycan polymers does not comprise a lipid and fatty acid. In some embodiments, the preparation of glycan polymers does not comprise an amino acid.

Furanose: Pyranose

In some embodiments, preparations of glycan polymers are provided, wherein at least one glycan subunit is a furanose sugar. In some embodiments, preparations of glycans are provided, wherein at least one glycan subunit is a pyranose sugar. In some embodiments, glycan polymers comprise mixtures of furanose and pyranose sugars. In some embodiments, the furanose: pyranose sugar ratio in a preparation is about 0.1:1, 0.2:1, 0.3:1, 0.4:1, 0.5:1, 0.6:1, 0.7:1, 0.8:1, 0.9:1, 1:1, 1.2:1, 1.5:1, 1.7:1, 2:1, 2.2:1, 2.5:1, 2.7:1, 3:1, 4:1, 5:1, or about 6:1 or the furanose: pyranose sugar ratio in a preparation is about 7:1, 8:1, 9:1, or about 10:1.

In some embodiments, the preparation of glycan polymers comprises substantially all furanose or pyranose sugar, optionally comprising 1%, 2%, 3%, 4% 5%, 6%, 7%, 8%, 9%, 10%, 11%, 12%, 13%, 14%, 15%, 16%, 17%, 18%, 19%, or 20% of the respective other sugar.

In some embodiments, the preparation of glycan polymers comprises substantially all pyranose sugar and no more than about 0.1%, 02%, 0.5%, 1%, 2%, 3%, 4%, or no more than 5% of glycan units in the preparation in furanose form. In some embodiments, no more than 3%, 2% or no more than 1% of monomeric glycan units in the preparation are in furanose form.

Salts

In some embodiments, the preparation of glycan polymers comprises a glycan subunit or plurality of glycan subunits present in a salt form (e.g., a pharmaceutically acceptable salt form), such as, e.g., a hydrochlorate, hydroiodate, hydrobromate, phosphate, sulfate, methanesulfate, acetate, formate, tartrate, malate, citrate, succinate, lactate, gluconate, pyruvate, fumarate, propionate, aspartate, glutamate, benzoate, ascorbate salt.

Derivatization

If desired, the monosaccharide or oligosaccharide glycan subunits of the glycans are further substituted or derivatized, e.g., hydroxyl groups can be etherified or esterified. For example, the glycans (e.g. oligo- or polysaccharide) can contain modified saccharide units, such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′-fluororibose, deoxyribose, and hexose). The degree of substitution (DS, average number of hydroxyl groups per glycosyl unit) can be 1, 2, or 3, or another suitable DS. In some embodiments, 1%, 2%, 3%, 4% 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 80%, 85%, 90%, 95%, 96%, 97%, 98%, 99%, or 100% of glycan subunits are substituted or derivatized. In some embodiments, the degree of substitution varies between subunits, e.g., a certain percentage is not derivatized, exhibits a DS of 1, exhibits a DS of 2, or exhibits a DS of 3. Any desired mixture can be generated, e.g. 0-99% of subunits are not derivatized, 0-99% of subunits exhibit a DS of 1, 0-99% of subunits exhibit a DS of 2, and 0-99% of subunits exhibit a DS of 3, with the total making up 100%. The degree of substitution can be controlled by adjusting the average number of moles of substituent added to a glycosyl moiety (molar substitution (MS)). The distribution of substituents along the length of the glycan oligo- or polysaccharide chain can be controlled by adjusting the reaction conditions, reagent type, and extent of substitution. In some embodiments, the monomeric subunits are substituted with one or more of an acetate ester, sulfate half-ester, phosphate ester, or a pyruvyl cyclic acetal group.

Solubility

In some embodiments, the glycan polymers in a preparation are highly soluble. In some embodiments, glycan polymer preparations can be concentrated to at least to 55 Brix, 65 Brix, 60 Brix, 65 Brix, 70 Brix, 75 Brix, 80 Brix, or at least 85 Brix without obvious solidification or crystallization at 23° C. (final solubility limit). In some embodiments, glycan polymer preparations are concentrated to at least about 0.5 g/ml, 1 g/ml, 1.5 g/ml, 2 g/ml, 2.5 g/ml, 3 g/ml, 3.5 g/ml or at least 4 g/ml without obvious solidification or crystallization at 23° C. (final solubility limit).

In some embodiments, the glycan polymer preparations (e.g. oligosaccharides) are branched, e.g. have an average DB of at least 0.01, 0.05, or 0.1 and has a final solubility limit in water of at least about 70 Brix, 75 Brix, 80 Brix, or at least about 85 Brix at 23° C. or is at least about 1 g/ml, 2 g/ml or at least about 3 g/ml.

In some embodiments, the preparation of glycan polymers has a final solubility limit of at least 0.001 g/L, 0.005 g/L, 0.01 g/L, 0.05 g/L, 0.1 g/L, 0.2 g/L, 0.3 g/L, 0.4 g/L, 0.5 g/L, 0.6 g/L, 0.7 g/L, 0.8 g/L, 0.9 g/L, lg/L, 5 g/L, 10 g/L, 20 g/L, 30 g/L, 40 g/L, 50 g/L, 100 g/L, 200 g/L, 300 g/L, 400 g/L, 500 g/L, 600 g/L, 700 g/L, 800 g/L, 900 g/L, 1000 g/L in deionized water, or in a suitable buffer such as, e.g., phosphate-buffered saline, pH 7.4 or similar physiological pH) and at 20° C. In some embodiments, the preparation of glycan polymers is greater than 50%, greater than 60%, greater than 70%, greater than 80%, greater than 90%, greater than 95%, greater than 96%, greater than 97%, greater than 98%, greater than 99%, or greater than 99.5% soluble with no precipitation observed at a concentration of greater than 0.001 g/L, 0.005 g/L, 0.01 g/L, 0.05 g/L, 0.1 g/L, 0.2 g/L, 0.3 g/L, 0.4 g/L, 0.5 g/L, 0.6 g/L, 0.7 g/L, 0.8 g/L, 0.9 g/L, 1 g/L, 5 g/L, 10 g/L, 20 g/L, 30 g/L, 40 g/L, 50 g/L, 100 g/L, 200 g/L, 300 g/L, 400 g/L, 500 g/L, 600 g/L, 700 g/L, 800 g/L, 900 g/L, 1000 g/L in deionized water, or in a suitable buffer such as, e.g., phosphate-buffered saline, pH 7.4 or similar physiological pH) and at 20° C.

Sweetness

In some embodiments, the preparation of glycan polymers has a desired degree of sweetness. For example, sucrose (table sugar) is the prototype of a sweet substance. Sucrose in solution has a sweetness perception rating of 1, and other substances are rated relative to this (e.g., fructose, is rated at 1.7 times the sweetness of sucrose). In some embodiments, the sweetness of the preparation of glycan polymers ranges from 0.1 to 500,000 relative to sucrose.

In some embodiments, the relative sweetness is 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 80, 90, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, 10000, 25000, 50000, 75000, 100000, 150000, 200000, 250000, 300000, 350000, 40000, 450000, 500000, or more than 500,000 relative to sucrose (with sucrose scored as one). In some embodiments, the preparation of glycan polymers is mildly sweet, or both sweet and bitter.

In some embodiments, the preparation of glycan polymers, e.g. a preparation that is substantially DP2+ or DP3+ (e.g. at least 80%, 90%, or at least 95%, or a fractionated preparation of DP2+ or DP3+), is substantially imperceptible as sweet and the relative sweetness is about 0, 0.0001, 0.001, 0.005, 0.01, 0.05, 0.1, 0.2, 0.3, 0.4, 0.5, 0.6, 0.7, or about 0.8 relative to sucrose (with sucrose scored as one).

Glycan polymer preparations can be characterized by any suitable methods including those described in WO2016/122889, WO2016/172657, WO 2016/007778, and WO2016/172658, incorporated herein by reference.

In embodiments, glycan compositions and glycan preparations may comprise one or more (e.g., two, three, four, five, six or more) of the following properties (including bulk properties):
a) the glycan polymer comprising at least one of glucose, galactose, arabinose, mannose, fructose, xylose, fucose, or rhamnose,
b) a high degree of polymerization (DP), e.g. at least about 50%, 60%, 70%, 80%, 90%, 95%, 98%, 99% of polymers range in DP from about 30-100,000, about 30-50,000, about 30-10,000, about 30-5,000, about 30-1,000, about 30-500, about 30-200, about 30-100, or about 3-50,
c) a low degree of polymerization, e.g. at least about 50%, 60%, 70%, 80%, 90%, 95%, 98%, 99% of polymers range in DP from about 2-29, about 2-25, about 2-20, about 2-15, about 2-10, about 2-8, about 2-6, about 3-8, or about 4-8,
d) a high viscosity e.g., ranging from about 100-10,000 mPas, 100-5,000 mPas, 100-1,000 mPas, 100-500 mPas, in water at 20° C.,
e) a low viscosity, e.g., ranging from about 1-99 mPas, 1-50 mPas, 1-10 mPas, 1-5 mPas, 25-75 mPas, or 10-50 mPas, in water at 20° C.,
f) a high final solubility limit in water of at least about 60, 70, or at least about 75 Brix at 23° C.,
g) a low final solubility limit in water of no more than 5, 10, 20, 30, 40, 50 Brix at 23° C., or insolubility (e.g. no more than 0.1 Brix)
h) a caloric value of about 0.1 cal/g to 3 cal/g, 0.1 cal/g to 2 cal/g, 0.1 cal/g to 1.5 cal/g, 0.1 cal/g to 1 cal/g, 0.1 cal/g to 0.5 cal/g,
i) a non-caloric value (e.g., about 0 cal/g to 0.09 cal/g, 0 cal/g to 0.05 cal/g or about 0 cal/g to 0.01 cal/g
j) a low degree of digestibility, wherein no more than about 30%, 20%, 10%, 5%, 1%, 0.5% of the glycan polymer is digestible by a human glycosidase (e.g., alpha-amylase)
k) a high degree of digestibility, wherein at least 50%, 60%, 70%, 80%, 90%, 95% of the glycan polymer is digestible by a human glycosidase (e.g., alpha-amylase)
l) a low degree of fermentability, wherein no more than about 40%, 30%, 20%, 10%, 5%, 1%, 0.5% of the glycan polymer is fermentable by a human (e.g., colonic) microbial community or a single bacterial strain,
m) a high degree of fermentability, wherein at least 50%, 60%, 70%, 80%, 90%, 95% of the glycan polymer is fermentable by a human (e.g. colonic) microbial community or a single bacterial strain,
n) a slow rate of fermentation, wherein no more than about 0.5%, 1%, 2%, 5%, 10%, or 15% of the glycan polymer is fermented by a human (e.g., colonic) microbial community or a single bacterial strain in 12-24 hours,
o) a fast rate of fermentation, wherein at least about 15%, 20%, 30%, 40%, or 50% of the glycan polymer is fermented by a human (e.g. colonic) microbial community or a single bacterial strain in 12-24 hours,
p) a high degree of gastrointestinal tolerance (e.g., is tolerated by a subject in high daily doses, e.g. at least about 5 g/day, 10 g/day, 15 g/day, 20 g/day, 30 g/day, 40 g/day, 50 g/day, 60 g/day, or 70 g/day without substantial side effects, e.g. such as bloating, excess gas, GI discomfort, diarrhea or constipation);
q) any combination of:
   two of: a), b), c), d), e), f), g), h), i), j), k), l), m), n), o), p);
   three of: a), b), c), d), e), f), g), h), i), j), k), l), m), n), o), p);
   four of: a), b), c), d), e), f), g), h), i), j), k), l), m), n), o), p);
   five of: a), b), c), d), e), f), g), h), i), j), k), l), m), n), o), p);
      six of: a), b), c), d), e), f), g), h), i), j), k), l), m), n), o), p);
   seven of: a), b), c), d), e), f), g), h), i), j), k), l), m), n), o), p);
   eight of: a), b), c), d), e), f), g), h), i), j), k), l), m), n), o), p);
   nine of: a), b), c), d), e), f), g), h), i), j), k), l), m), n), o), p);
   ten of: a), b), c), d), e), f), g), h), i), j), k), l), m), n), o), p); or
   all of: a), b), c), d), e), f), g), h), i), j), k), l), m), n), o), p).

In embodiments, glycan compositions and glycan preparations may comprise one or more (e.g., two, three, four, five, six or more) of the following properties (including bulk properties):

i) the glycan preparation comprises glycans that comprise glucose, galactose, arabinose, mannose, fructose, xylose, fucose, or rhamnose glycan units;

ii) the average degree of branching (DB) of the glycans in the glycan preparation is 0, between 0.01 and 0.6, between 0.05 and 0.5, between 0.1 and 0.4, or between 0.15 and 0.4;

iii) at least 50% (at least 60%, 65%, 70%, 75%, 80%, or 85%, or less than 50%) of the glycans in the glycan preparation have a degree of polymerization (DP) of at least 3 and less than 30 glycan units, at least 2 and less than 10 glycan units, at least 5 and less than 25 glycan units, or at least 10 and less than 35 glycan units (optionally, wherein the glycan unit is a monomer, e.g., a monosugar);

iv) the average DP (mean DP) of the glycan preparation is between about 2 and 5, between about 5 and 8, between about 8 and 13, between about 13 and 25, between about 5 and 15, between about 5 and 20, or between about 5-15;

v) the ratio of alpha- to beta-glycosidic bonds present in the glycans of the glycan preparation is 0, or between about 0.8:1 to about 5:1, between about 1:1 to about 5:1, between about 1:1 to about 3:1, between about 3:2 to about 2:1, or between about 3:2 to about 3:1,

vi) the glycan preparation comprises between 15 mol % and 75 mol % (between 20 mol % and 60 mol %, between 25 mol % and 50 mol %, or between 30 mol % and 45 mol %) 1,6 glycosidic bonds;

vii) the glycan preparation comprises between 1 mol % and 40 mol % (between 1 mol % and 30 mol %, between 5 mol % and 25 mol %, between 10 mol % and 20 mol %) of at least one, two, or three of 1,2; 1,3; and 1,4 glycosidic bonds;

viii) the glycan preparation has a final solubility limit in water of at least about 50 (at least about 60, 70, at least about 75, or less than 50) Brix at 23° C.; or

ix) the glycan preparation has a dietary fiber content (e.g., as measured by AOAC 2009.01) of at least 50% (at least 60%, 70%, 80%, or at least 90%, or less than 50%),

x) any combination of two, three, four, five, six, seven, eight, or nine of i), ii), iii), iv), v), vi), vii), viii), and ix), and as exemplified, e.g., in Table 10.

Glycan compositions described herein can comprise one or more sugars and/or sugar alcohols._{—} Compositions can comprise a simple sugar (such as a monosaccharide, a disaccharide, a trisaccharide, a tetrasacchaaride or a pentasaccharide), a sugar alcohol, or any combination thereof. In some embodiments, composition comprises a metabolizable sugar or metabolizable sugar alcohol, wherein the sugar or sugar alcohol is metabolized in the gastrointestinal tract of the host. The sugars, and sugar alcohols disclosed in WO 2016/172658, which is hereby incorporated by reference, are suitable for use in methods and compositions described herein. In embodiments, a composition described herein, e.g., glycan composition described herein, can comprise polyphenols, fatty acids (e.g., short chain fatty acids), amino acids, peptides, and micronutrients, e.g., as described herein and in WO 2016/172658 hereby incorporated by reference and in Table 7.

TABLE 7
Exemplary constituents of glycan compositions: Sugars, Sugar Alcohols,
Amino Acids, Vitamins, Minerals, Fatty Acids, and Polyphenols

| Compound | Examples |
|---|---|
| Sugar | glucose, galactose, N-acetylglucosamine, N-acetylgalactosamine, fructose, |
| | fucose, mannose, N-acetylmannosamine, glucuronic acid, N-acetylglucuronic |
| | acid, galactosuronic acid, N-acetylgalactosuronic acid, xylose, arabinose, |
| | rhamnose, ribose, sucrose, sorbose, lactose, maltose, lactulose, tagatose, |
| | kojibiose, nigerose, isomaltose, trehalose, sophorose, laminaribiose, gentiobiose, |
| | turanose, maltulose, palatinose, gentiobiulose, mannobiose, melibiulose, |
| | rutinulose, xylobiose |
| Sugar Alcohol | sorbitol, mannitol, lactitol, erythritol, glycerol, arabitol, maltitol, xylitol, ribitol, |
| | threitol, galactitol, fucitol, iditol, inositol |
| Amino Acid | alanine, arginine, asparagine, aspartic acid, cysteine, glutamic acid, glutamine, |
| | glycine, histidine, isoleucine, leucine, lysine, methionine, phenylalanine, proline, |
| | serine, threonine, tryptophan, tyrosine, valine |
| Vitamin | pantothenate, thiamine, riboflavin, niacin, pyridoxol, biotin, folate, 4- |
| | aminobenzoate, cobinamide, phenyolyl cobamide, 5-methylbenzimidazolyl |
| | cobamide, cobalamin, pyridoxine, pyridoxamine, ergadenylic acid, |
| | cyanocobalamin, choline, retinol, a carotenoid, zeaxanthin |
| Element/Mineral | chloride, sodium, calcium, magnesium, nitrogen, potassium, manganese, iron, |
| | zinc, nickel, copper, cobalt |
| Fatty Acid | acetic acid, propionic acid, butryic acid, isobutyric acid, valeric acid, isovaleric |
| | acid, hexanoic acid, octanoic acid, formic acid, oxalic acid, glyoxylic acid, |
| | glycolic acid, acrylic acid, malonic acid, pyruvic acid, lactic acid, succinic acid, |
| | acetoacetic acid, fumaric acid, maleic acid, oxaloacetic acid, malic acid, tartaric |
| | acid, crotonic acid, glutaric acid, alpha-ketoglutaric acid, caproic acid, adipic |
| | acid, citric acid, aconitic acid, isocitric acid, sorbic acid, enanthic acid, pimelic |
| | acid, benzoic acid, salicylic acid, caprylic acid, phthalic acid, pelargonic acid, |
| | trimesic acid, cinnamic acid, capric acid, sebacic acid, stearic acid, oleic acid, |
| | linoleic acid, α-linolenic acid, γ-linolenic acid, stearidonic acid |
| Polyphenol | Anthocyanins, Chalcones, Dihydro-chalcones, Dihydro-flavonols, Flavanols, |
| | Flavanones, Flavones, Flavonols, Isoflavonoids, Lignans, Non-phenolic |
| | metabolites, Alkylmethoxy-phenols, Alkylphenols, Betacyanins, Capsaicinoids, |
| | Curcuminoids, Dihydro-capsaicins, Furano-coumarins, Hydroxy-benzaldehydes, |
| | Hydroxy-benzoketones, Hydroxycinnam-aldehydes, Hydroxy-coumarins, |
| | Hydroxyphenyl-alcohols, Hydroxy-phenylpropenes, Methoxyphenols, |
| | Naphtoquinones, Phenolic terpenes, Tyrosols, Hydroxybenzoic acids, Hydroxy- |
| | cinnamic acids, Hydroxy-phenylacetic acids, Hydroxy-phenylpropanoic acids, |
| | Hydroxy-phenylpentanoic acids, Stilbenes, catechin, ellagitannin, isoflavone, |
| | flavonol, flavanone, anthocyanin, lignin, alkylmethoxyphenol, alkylphenol, |
| | curcuminoid, furanocoumarin, hydroxybenzaldehyde, hydroxybenzoketone, |
| | hydroxycinnamaldehyde, hydroxycoumarin, hydroxyphenylpropene, |
| | methoxyphenol, naphtoquinone, phenolic terpenes, tyrosols |

The glycan preparations have been described above with respect to different parameters including inter alia the degree of polymerization (DP), average DP, glycosidid bonds and glycan unit content, etc. It needs to be understood that the present disclosure in particular contemplates glycan preparations with combinations of the parameters mentioned above as they are supported by the examples. The examples in this context served as general guidance on how these properties can be combined and generalized.

Simply for exemplary purposes, reference is made to Table 10. This table highlights some of the above discussed parameters for glycan preparations having different amounts of glycan units as well as for different batches of the same glycan preparations having the same amounts of glycan units. From the indicated parameters it is for example apparent that different batches of the same glycan preparations have comparable properties for the indicated parameters.

For example, on the basis of Table 10, a Glu50Gal50 glycan preparation (e.g. Glu50Gal50-11, Glu50Gal50-32, Glu50Gal50-14, Glu50Gal50-27, Glu50Gal50-23, Glu50Gal50-2) obviously seems to have a total molar incidence of 1,2-bonds of between about 10% and about 30%. Some of the batches seem to have an even narrower distribution of between about 15% and about 20%. The same can be said for the total molar incidence 1,3 bonds. The 1,4-bond molar incidence is between about 15% and about 30%. A narrower distribution seems to be between about 20% and about 30%. The 1,6-bond molar incidence differs a bit in that it is between about 35% and about 55%. A narrower range seems to be between about 40% and about 50%. The ratio of alpha/beta bonds seems to vary by between 1.5:1-2.5:1. DP2+ seems to be across all batches >90%, with most of the batches being 95%. It is on the basis of this data that one will consider combinations of the parameters set out above as well as the values indicated for these parameters to characterize a Glu50Gal50 glycan preparation disclosed herein as having for example a total molar incidence of 1,2 bonds of between about 10% to about 30%, of 1,3 bonds of between about 10% to about 30%, of 1,4 bonds of between about 15% to about 30% and of 1,6 bonds of between about 35% to about 55%. Such glycan compositions can be further characterized by an alpha/beta-ratio of 1.5:1-2.5:1, etc. For the considerations outlaid above, one would also contemplate the indicated narrow ranges.

Similar considerations apply to other glycan compositions. As can be taken from Table 10, a Glu100 composition may be substantially linear or branched. As far as branched Glu100 compositions are concerned, it can be taken from Table 10 that Glu100 seems to have a total molar incidence of 1,2-bonds of between about 10% and about 30%. Some of the batches seem to have an even narrower distribution of between about 15% and about 25%. The same can be said for the total molar incidence 1,3 bonds. The 1,4 bond molar incidence is also between about 10% and about 30%. A narrower distribution seems to be between about 20% and about 30%. The 1,6 bond molar incidence differs a bit in that it is between about 35% and about 55%. A narrower range seems to be between about 40% and about 50%. The ratio of alpha/beta bonds seems to vary by between 1.5:1-2.5:1. DP2+ seems to be across all batches >95%, with most of the batches being 98%. It is on the basis of this data that one will consider combinations of the parameters set out above as well as the values indicated for these parameters to characterize a Glu100 glycan preparation disclosed herein as having for example a total molar incidence of 1,2 bonds of between about 10% to about 30%, of 1,3 bonds of between about 10% to about 30%, of 1,4 bonds of between about 10% to about 30% and of 1,6 bonds of between about 35% to about 55%. Such glycan compositions can be further characterized by an alpha/beta-ratio of 1.5:1-2.5:1, etc. For the considerations outlaid above, one would also contemplate the indicated narrow ranges.

As far as Ara100 compositions are concerned, it can be taken from Table 10 that Ara100 seems to have a total molar incidence of 1,2-bonds of between about 15% and about 30%. Some of the batches seem to have an even narrower distribution of between about 20% and about 30%. The 1,3 bond molar incidence seems to be about 30% and about 50%. A narrower distribution seems to be between about 30% and about 50%. The 1,4 bond molar incidence seems to be about 20% and about 40%. A narrower distribution seems to be between about 25% and about 35%. The ratio of alpha/beta bonds seems to vary by between 2:1-4:1 and approxiamtely 3:1. It is on the basis of this data that one will consider combinations of the parameters set out above as well as the values indicated for these parameters to characterize a Ara100 glycan preparation disclosed herein as having for example a total molar incidence of 1,2 bonds of between about 15% to about 30%, of 1,3 bonds of between about 30% to about 40%, and of 1,4 bonds of between about 20% to about 40%. Such glycan compositions can be further characterized by an alpha/beta-ratio of 2:1-4:1, etc. For the considerations outlaid above, one would also contemplate the indicated narrow ranges.

As far as Gal100 compositions are concerned, it can be taken from Table 10 that Gal100 seems to have a total molar incidence of 1,2-bonds of between about 10% and about 30%. Some of the batches seem to have an even narrower distribution of between about 10% and about 20%. The 1,3 bond molar incidence seems to be between about 10% and about 30%. A narrower distribution seems to be between about 15% and about 25%. The 1,4 bond molar incidence seems to be about 15% and about 35%. A narrower distribution seems to be between about 25% and about 35%. The 1,6 bond molar incidence seems to be about 35% and about 55%. A narrower distribution seems to be between about 35% and about 45%. The ratio of alpha/beta bonds seems to vary by between 2:1-4:1 and approxiamtely 3:1. It is on the basis of this data that one will consider combinations of the parameters set out above as well as the values indicated for these parameters to characterize a Gal100 glycan preparation disclosed herein as having for example a total molar incidence of 1,2 bonds of between about 10% to about 30%, of 1,3 bonds of between about 10% to about 30%, of 1,4 bonds of between about 15% to about 35%, and of 1,6 bonds of between about 35% to about 55%. Such glycan compositions can be further characterized by an alpha/beta-ratio of 2:1-4:1, etc. For the considerations outlaid above, one would also contemplate the indicated narrow ranges.

As far as Man100 compositions are concerned, it can be taken from Table 10 that Man100 seems to have a total molar incidence of 1,2-bonds of between about 10% and about 30%. Some of the batches seem to have an even narrower distribution of between about 10% and about 20%. The 1,3 bond molar incidence seems to be between about 10% and about 30%. A narrower distribution seems to be between about 20% and about 30%. The 1,4 bond molar incidence seems to be about 10% and about 30%. A narrower distribution seems to be between about 20% and about 30%. The 1,6 bond molar incidence seems to be about 25% and about 45%. A narrower distribution seems to be between about 30% and about 40%. The ratio of alpha/beta bonds seems to vary by between 1:1-2:1 and approxiamtely 1.3:1. It is on the basis of this data that one will consider combinations of the parameters set out above as well as the values indicated for these parameters to characterize a Man100 glycan preparation disclosed herein as having for example a total molar incidence of 1,2 bonds of between about 10% to about 30%, of 1,3 bonds of between about 10% to about 30%, of 1,4 bonds of between about 10% to about 30%, and of 1,6 bonds of between about 25% to about 45%. Such glycan compositions can be further characterized by an alpha/beta-ratio of 1:1-3:1, etc. For the considerations outlaid above, one would also contemplate the indicated narrow ranges.

As far as Glu60Man40 compositions are concerned, it can be taken from Table 10 that Glu60Man40 seems to have a total molar incidence of 1,2-bonds of between about 5% and about 25%. Some of the batches seem to have an even narrower distribution of between about 5% and about 15%. The 1,3 bond molar incidence seems to be between about 10% and about 30%. A narrower distribution seems to be between about 20% and about 30%. The 1,4 bond molar incidence seems to be about 10% and about 30%. A narrower distribution seems to be between about 20% and about 30%. The 1,6 bond molar incidence seems to be about 30% and about 50%. A narrower distribution seems to be between about 40% and about 50%. The ratio of alpha/beta bonds seems to vary by between 3:1-5:1 and approxiamtely 4:1. It is on the basis of this data that one will consider combinations of the parameters set out above as well as the values indicated for these parameters to characterize a Glu60Man40 glycan preparation disclosed herein as having for example a total molar incidence of 1,2 bonds of between about 5% to about 25%, of 1,3 bonds of between about 10% to about 30%, of 1,4 bonds of between about 10% to about 30%, and of 1,6 bonds of between about 30% to about 50%. Such glycan compositions can be further characterized by an alpha/beta-ratio of 3:1-5:1, etc. For the considerations outlaid above, one would also contemplate the indicated narrow ranges.

One will consider such parameter values and considerations also for the other glycan compositions mentioned in Table 10 as well as for the glycan compositions mentioned in e.g. Tables 7, 8, and 9.

It is also to be understood that even though glycan of defined composition have been tested in the examples it seems reasonable to assume that if e.g. a Glu50Gal50 glycan composition is effective in increasing the effect of cardiac glycosides other glycan compositions with comparable relative monomeric composition such as Glu45Gal55 or Glu55Gal45 will have a similar activity.

The present disclosure thus describes numerous pointers in terms of the tables and data presented herein which allows one to select parameter combinations for specific glycan preparations with respect to a specific therapeutic purpose.

### Glycan Polymer Compositions and Use Thereof

One will further consider the disclosure on glycan compositions together with the examples that address the change of the abundance of certain micro-organisms and associated enzymatic activities as a consequence of administration of glycan compositions. This will allow to understant which glycan compositions can used for treatment of diseases by maintaning and/or increasing the activity of drugs commonly used for the treatment of such disease.

Simply for exemplary purposes it is pointed out that Examples 9, 10, and 19 describe how administration of certain glycans allow to increase the activity of cardiac glycosides (Example 9), sulfonamides (Example 10) and nucleoside analogues (Example 19).

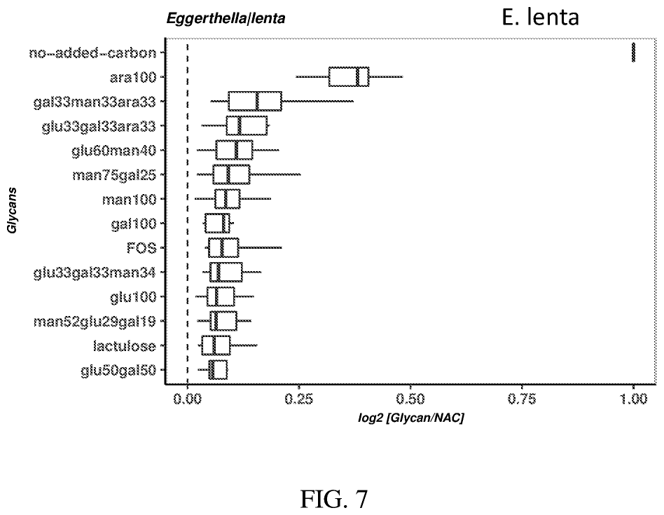

As can be taken e.g. from FIG. 7/Example 9 Glu50Gal50 is particularly effective in reducing Eggerthella lenta thereby potentially avoiding degradation of digoxin. However, FIG. 7 indicates that Glu100 and Man52Glu29Gal19 are also effective. It is noted that Man52Glu29Gal19 is mentioned as well in Table 10. With the information provided e.g. in Table 10 one will thus also consider Man52Glu29Gal19 and Man52Glu29Gal19 glycan compositions being defined by such parameters as e.g. described above compositions as well as Glu100 glycan compositions being defined by such parameter as e.g. described above for use in maintaining the activity of cardiac glycosides thereby allowing e.g. treatment of heart failure.

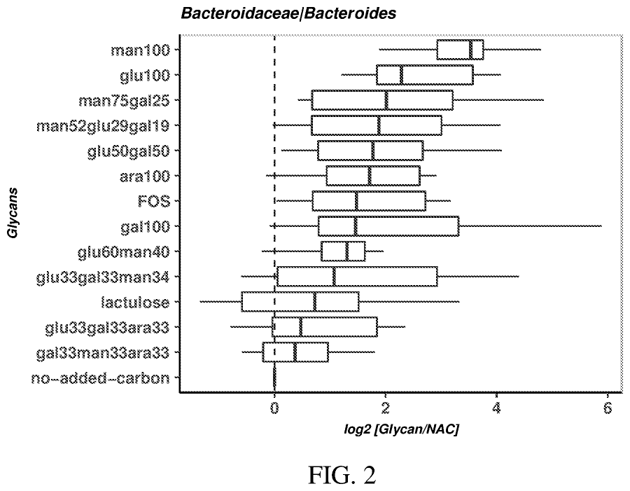

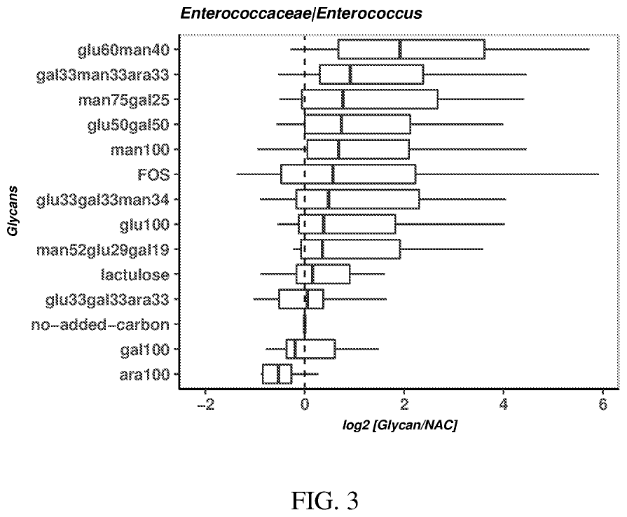

Similar considerations apply to e.g. Example 10 for which it can be taken from FIG. 2 and FIG. 3 that Man100 is particularly effective in increasing Bacteroidaceae and that Glu60man40 is particularly effective in increasing Enterococcaceaea which may increase the activity of microbial azoreductase and thereby increase the levels of sulfasalazine. However, as it can be taken from FIG. 2 and FIG. 3 Man75Gal25 and Gal33Man33Ara33 may also have positive effects in this regard. With the information provided e.g. in Table 10 one will thus also consider parameter combinations for Man75Gal25 and Gal33Man33Ara33 and Man75Gal25 and Gal33Man33Ara33 as e.g. described above will be considered as well as Man100 or Glu60Man40 as e.g. described above for use in maintaining the activity of sulfonamides thereby allowing e.g. treatment of inflammatory disease or other of the below listed disease depending on which sulfonamid is applied.

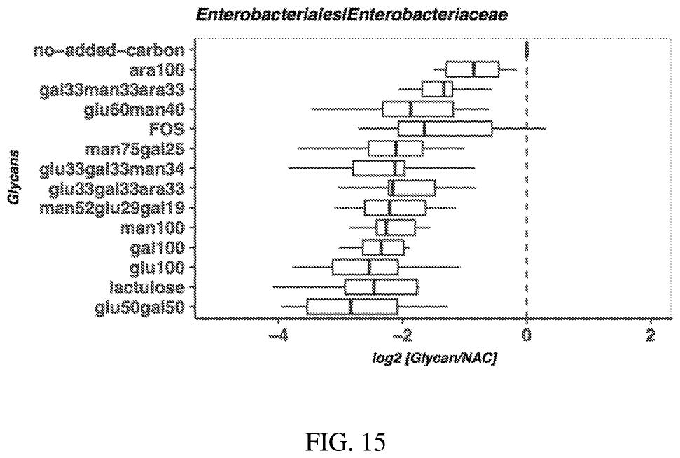

Similar considerations apply to e.g. Example 19 for which it can be taken from FIG. 15 that Glu50Gal50 is particularly effective in decreasing Enterobacteriales thereby potentially avoiding deglycosylation of soriudine. However, as it can be taken from FIG. 15 Man75Gal25 and Man52Glu29Gal19 may also have positive effects in this regard. With the information provided in e.g. Table 10 one will thus also consider parameter combinations for Man52Glu29Gal19 and Man52Glu29Gal19 as e.g. described above as well as Glu50Gal50 as e.g. described above for use in maintaining the activity of nucleoside analogues thereby allowing e.g. treatment of cancer.

Even though the preceding paragraphs, which aim at pointing out how the information provided in e.g. Table 10, the data in the embodiment examples, etc. can be considered as a pointer for combining glycan parameters that have been disclosed in general above, have used the examples pertaining to cardiac glycosides, sulfonamides and nucleoside analogues for explanatory purposes, it must be understood that similar considerations apply to the other drugs and conditions mentioned herein. For example, Example 3 describes how Glu80Man20 is particularly effective in promoting growth of strains metabolizing phytoestrogens which have an protective effect against breast cancer. With the information provided e.g. in Table 10 one will thus also consider parameter combinations for Glu80Man20 and Glu80Man20 as e.g. described above for increasing the the activity of an ingested substance such as a phytoestrogen for e.g. treatment for breast cancer.

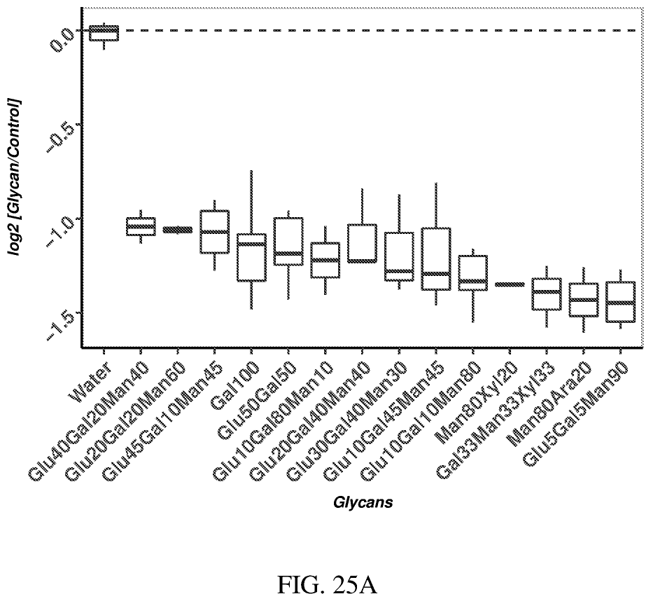

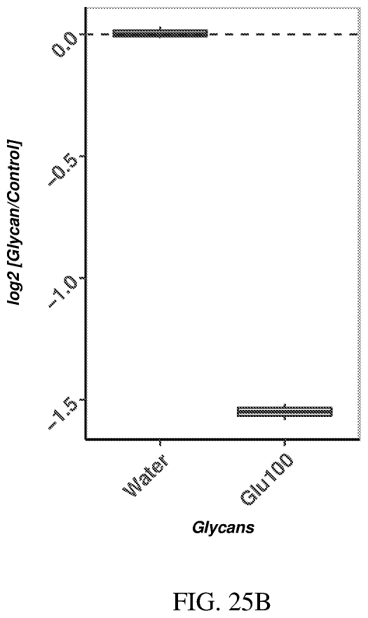

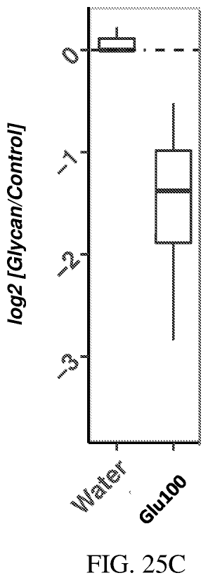

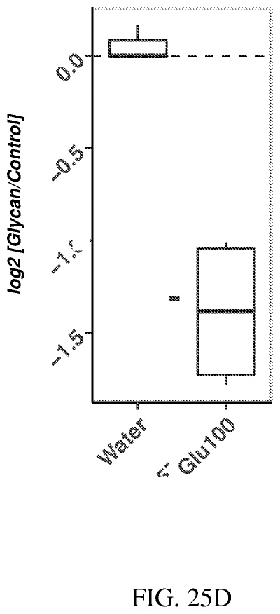

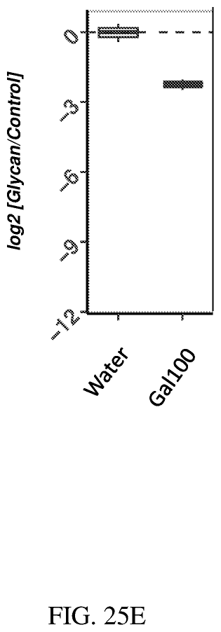

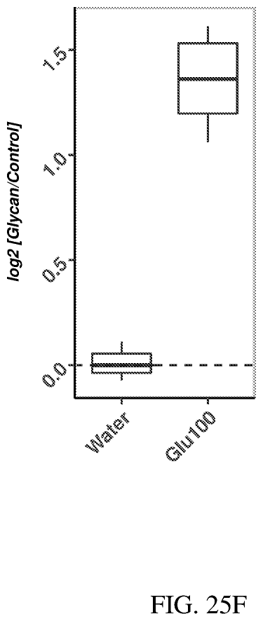

One will also consider combining the information of e.g. FIGS. 25A-25F which show the change in expression for certain enzymes in relation to glycan preparations, the information in e.g. Table 10 providing information on parameters for representative glycan preparations and the information in e.g. Tables 1 through 5 which allow linking microbial enzymatic activities with potential effects on the activity of drugs when considering glycan compositions being defined by specific parameters for the various embodiments described herein, for example embodiments being concerned with increasing the activity of cardiac glycosides, sulfonamides, nucleoside analogues or aminosalicylates but also for the other embodiments described herein such as the impact of glycans on the activity of ingested substances such as phytoestrogens. For example, given the effect of e.g. Man100, Glu100, Man75Gal25, etc. on Bacteroides leading to increased levels of sulfalazine (See Table 1, row 3), one will consider consider these oligosaacharides for influencing other drugs and aspects being influenced by Bacteroides such as reduction of p-cresol levels.

One will further understand that the glycan preparations described herein which can be employed, e.g. for maintaining or increasing the activity of drugs such as cardiac glycosides, sulfonamides, nucleoside analogues or aminosalicylates by these properties can be used for treating disease which are known to respond to these classes of drugs.

For example, as it is explained above Diabetes mellitus, convulsions, Hepatitis C, HIV, inflammation, cardiac arrythmia, hypertension, glaucoma, bacterial infections, Ebola virus infections, Hepatitis B, pulmonary hypertension, migraine, erectile dysfunction and benign prostate hyperplasia can be treated by sulfonamides. Glycan preparations described herein may thus be used in addition to sulfonamides to treat the respective disease.

More specifically, sulfonylureas used to treat diabetes include Acetohexamide, Carbutamide, Chlorpropamide, Glibenclamide (glyburide), Glibornuride, Gliclazide, Glyclopyramide, Glimepiride, Glipizide, Gliquidone, Glisoxepide, Tolazamide, or Tolbutamide. Sulfonamides used to treat convulsions include Ethoxzolamide, Sultiame, Topiramate, and Zonisamide. In some embodiments, glycans of the present invention may be administered to improve the effectiveness of sulfonamides as antiviral drugs.

Sulfonamides used to treat convulsions include Ethoxzolamide, Sultiame, Topiramate, and Zonisamide. In some embodiments, glycans of the present invention may be administered to improve the effectiveness of sulfonamide anticonvulsants.

Sulfonamides used to treat Hepatitis C include Asunaprevir (or other NS3/4A protease inhibitor), Beclabuvir (or other NS5B RNA polymerase inhibitor), Dasabuvir, Grazoprevir, Paritaprevir, and Simeprevir. In some embodiments, glycans of the present invention may be administered to improve the effectiveness of sulfonamides as antiviral drugs.

Sulfonamides used to treat HIV include Amprenavir (or other HIV protease inhibitor), Darunavir, Delavirdine (or other non-nucleoside reverse transcriptase inhibitor), Fosamprenavir, and Tipranavir. Nucleoside analogue sulfonamides may also be used to treat HIV. Sulfonamides used to treat HIV alsoinclude deoxyadenosine analogues: didanosine (ddI), vidarabine (antiviral); deoxycytidine analogues: emtricitabine (FTC), lamivudine (3TC), zalcitabine (ddC); guanosine and deoxyguanosine analogues: abacavir; and thymidine and deoxythymidine analogues: stavudine (d4T), zidovudine (azidothymidine, or AZT). In some embodiments, glycans of the present invention may be administered to improve the effectiveness of sulfonamides as antiretroviral drugs.

Sulfonamides used to treat inflammation include Apricoxib (COX-2 inhibitor), Celecoxib (COX-2 inhibitor), Parecoxib (COX-2 inhibitor), and Sulfasalazine (anti-inflammatory agent and a DMARD). In some embodiments, glycans of the present invention may be administered to improve the effectiveness of sulfonamides as anti-inflammatory drugs.

Sulfonamides used to treat arrhythmias include Dofetilide (class III antiarrhythmic), Dorzolamide (anti-glaucoma carbonic anhydrase inhibitor), Dronedarone (class III antiarrhythmic), and Ibutilide (class III antiarrhythmic). In some embodiments, cardiac glycoside sulfonamides used to treat arrhythmias include digoxin, digitoxin, convallotoxin, antiarin, and oleandrin. In some embodiments, glycans of the present invention may be administered to improve the effectiveness of sulfonamides as anti-arrhythmia drugs.

Sulfonamides used to treat hypertension include Acetazolamide, Bumetanide, Chlorthalidone, Clopamide, Furosemide, Hydrochlorothiazide, Indapamide, Mefruside, Metolazone, and Xipamide. In some embodiments, glycans of the present invention may be administered to improve the effectiveness of sulfonamides as anti-hypertensive drugs.

Sulfonamides may be used to treat glaucoma. Sulfonamides used to treat glaucoma include Brinzolamide (carbonic anhydrase inhibitor for glaucoma), Dorzolamide (anti-glaucoma carbonic anhydrase inhibitor) and Acetazolamide. In some embodiments, glycans of the present invention may be administered to improve the effectiveness of sulfonamides as anti-glaucoma drugs.

Sulfonamides used to treat bacterial infection include Sulfafurazole, Sulfacetamide, Sulfadiazine, Sulfadimidine, Sulfafurazole (sulfisoxazole), Sulfisomidine (sulfaisodimidine), Sulfadoxine, Sulfamethoxazole, Sulfamoxole, Sulfanitran, Sulfadimethoxine, Sulfamethoxypyridazine, Sulfametoxydiazine, Sulfadoxine, Sulfametopyrazine, and Terephtyl. In some embodiments, glycans of the present invention may be administered to improve the effectiveness of sulfonamides as anti-microbial drugs.

Sulfonamides used to treat Ebola infection include adenosine analogs, e.g., BCX4430. In some embodiments, glycans of the present invention may be administered to improve the effectiveness of sulfonamides as anti-Ebola virus drugs.

Sulfonamides used to treat Hepatitis B include deoxycytidine analogues: lamivudine (3TC); guanosine and deoxyguanosine analogues: entecavir; and thymidine and deoxythymidine analogues: telbivudine. In some embodiments, glycans of the present invention may be administered to improve the effectiveness of sulfonamides as antiviral drugs.

Sulfonamides used to treat pulmonary hypertension include Bosentan and Udenafil. In some embodiments, glycans of the present invention may be administered to improve the effectiveness of sulfonamides as pulmonary hypertensive drugs.

Sulfonamides used to treat migraines include Sumatriptan. In some embodiments, glycans of the present invention may be administered to improve the effectiveness of sulfonamides as migraine drugs.

Sulfonamides used to treat erectile dysfunction include Udenafil. In some embodiments, glycans of the present invention may be administered to improve the effectiveness of sulfonamides as erectile dysfunction drugs.

Sulfonamides used to treat benign prostatic hyperplasia include Tamsulosin and Udenafil. In some embodiments, glycans of the present invention may be administered to improve the effectiveness of sulfonamides as benign prostatic hyperplasia drugs.

Further, as is explained above ulcerative colitis and Crohn's disease can be treatetd by aminosalicylates. Glycan preparations described herein may thus be used in addition to aminosalicylates to treat the respective diseases.

Aminosalicylates used to treat ulcerative colitis include 4-Aminosalicylic acid, Balsalazide, Olsalazine, Sulfasalazine, or Mesalazine (5-Aminosalicylic acid). Nucleoside analogs may be used to treat ulcerative colitis. Nucleoside analogs used to treat ulcerative colitis include Azathioprine, Mercaptopurine, and Thiopurines. In some embodiments, glycans of the present invention may be administered to improve the effectiveness of aminosalicylates and nucleoside analogs as anti-ulcerative colitis drugs.

Aminosalicylates used to treat Crohn's disease include 4-Aminosalicylic acid, Balsalazide, Olsalazine, Sulfasalazine, or Mesalazine (5-Aminosalicylic acid). Nucleoside analogs may be used to treat Crohn's disease. Nucleoside analogs used to treat Crohn's disease include Azathioprine, Mercaptopurine, and Thiopurine. In some embodiments, glycans of the present invention may be administered to improve the effectiveness of Aminosalicylates and nucleoside analogs as anti-Crohn's disease drugs.

Further, as is explained above heart failure can be treatetd by cardia glycosides. Glycan preparations described herein may thus be used in addition to cardia glycosides to treat heart failure.

Cardiac glycosides used to treat heart failure include digoxin, digitoxin, convallotoxin, antiarin, and oleandrin. In some embodiments, glycans of the present invention may be administered to improve the effectiveness of cardiac glycosides as heart failure drugs.

Further, as is explained above cancer can be treatetd by cardia glycosides. Glycan preparations described herein may thus be used in addition to cardia glycosides to treat the rcancer.

Nucleoside analogs used to treat cancer include deoxycytidine analogues: cytarabine (chemotherapy), gemcitabine; pyrimidine analogues: 5-Fluorouracil (5FU), Floxuridine (FUDR), Cytarabine (Cytosine arabinoside), 6-azauracil (6-AU); and purine analogs: Mercaptopurine, Thiopurines, Fludarabine, Pentostatin. In some embodiments, glycans of the present invention may be administered to improve the effectiveness of nucleoside analogs as anti-cancer drugs.

Is further to be understood that disclosure relating to glycan preparations described herein which can be employed e.g. for maintaining or increasing the activity of drugs such as sulfonamides when treating patients suffering from a disease which is treated by this class of drugs, is considered tantamount to a disclosure of method of treating a disease such as e.g. an inflammatory disease by administering a glycan preparation described herein in addition to a drug which is used to treat said disease such as e.g a sulfonamide, for example sulfasalazine without necessarily mentioning that the glycan composition will maintain or increase the activity of the respective sulfonamide. By way of example: the disclosure of a method of increasing the activity of sulfasalazine by administering a glycan composition described herein to a patient suffering from an inflammatory disease and being treated with sulfasalazine is considered equivalent to a method of treating a patient suffering from am inflammatory disease by administering to said patient a glycan preparation described herein in addtion to sulfasalazine.

It is against this background that the present disclosure further considers for all of the embodiments described herein, e.g. for maintaining or increasing the activity of cardiac glycosides, sulfonamides, nucleoside analogues or aminosalicylates to first assess a patient suffering from a disease as to the composition of its microbiome to determine whether the patient could benefit from administration of a specific glycan considering the information in the examples on how certain glycans influence the abundance of specific microorganisms. For example, a patient which suffers from an inflammatory disease such as Rheumathoid Arthritis and for which it is shown that it contains Bacteroidaceae and/or Enterococcaceaea in its microflora, may qualify for administration of Man100 as e.g. Example 10 establishes that such a glycan preparation supports growth of these microorganisms and through enzymatic conversion allows to increase the level of sulfasalazine. Similarly, a patient for which it is shown that it contains Eggerthella lenta in its microflora may qualify for administration of Glu50Gal50 in order to increase the activity of cardiac glycosides as this glycan preparation is shown by Example 9 to relatively reduce the growth of Eggerthella lenta.

### Probiotics

In embodiments, a composition described herein, e.g., glycan composition described herein, can comprise commensal or probiotic bacterial taxa, e.g., those described in Tables 4-6, and bacteria that are generally recognized as safe (GRAS) or known commensal or probiotic microbes. In embodiments, a composition described herein, e.g., glycan composition described herein, can comprise a bacterial taxa described in Tables 1-3. In some embodiments, probiotic or commensal bacterial taxa (or preparations thereof) may be administered to a subject receiving the glycan preparations.

In some embodiments, the composition further comprises at least about 1% (w/w) of a probiotic or commensal bacterium or a combination thereof (e.g., at least about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or more).

Probiotic microorganisms may also be included in the glycan compositions or used in combination with a glycan composition described herein. A probiotic microorganism is also referred to a probiotic. Probiotics can include the metabolites generated by the probiotic microorganisms during fermentation. These metabolites may be released to the medium of fermentation, e.g., into a host organism (e.g., subject), or they may be stored within the microorganism. Probiotic microorganism includes bacteria, bacterial homogenates, bacterial proteins, bacterial extracts, bacterial ferment supernatants and combinations thereof, which perform beneficial functions to the host animal, e.g., when given at a therapeutic dose.

Useful probiotic microorganisms include at least one lactic acid and/or acetic acid and/or propionic acid producing bacteria, e.g., microbes that produce lactic acid and/or acetic acid and/or propionic acid by decomposing carbohydrates such as glucose and lactose. Preferably, the probiotic microorganism is a lactic acid producing bacteria. In embodiments, lactic acid bacteria include Lactobacillus, Leuconostoc, Pediococcus, Streptococcus, and Bifidobacterium. Suitable probioitc microorganisms can also include other microorganisms which beneficially affect a host by improving the hosts intestinal microbial balance, such as, but not limited to yeasts such as Saccharomyces, Debaromyces, Candida, Pichia and Torulopsis, molds such as Aspergillus, Rhizopus, Mucor, and Penicillium and Torulopsis, and other bacteria such as but not limited to the genera Bacteriodes, Clostridium, Fusobacterium, Melissococcus, Propionibacterium, Enterococcus, Lactococcus, Staphylococcus, Peptostreptococcus, Bacillus, Pediococcus, Micrococcus, Leuconostoc, Weissella, Aerococcus, and Oenococcus, and combinations thereof.

Non-limiting examples of lactic acid bacteria useful in the disclosure herein include strains of Streptococcus lactis, Streptococcus cremoris, Streptococcus diacetylactis, Streptococcus thermophilus, Lactobacillus bulgaricus, Lactobacillus acidophilus, Lactobacillus helveticus, Lactobacillus bifidus, Lactobacillus casei, Lactobacillus lactis, Lactobacillus plantarum, Lactobacillus rhamnosus, Lactobacillus delbruekii, Lactobacillus thermophilus, Lactobacillus fermentii, Lactobacillus salivarius, Lactobacillus paracasei, Lactobacillus brevis, Bifidobacterium longum, Bifidobacterium infantis, Bifidobacterium bifidum, Bifidobcterium animalis, Bifidobcterium lactis, Bifidobcterium breve, Bifidobcterium adolescentis, and Pediococcus cerevisiae and combinations thereof, in particular Lactobacillus, Bifidobacterium, and combinations thereof.

Probiotic microorganisms which are particularly useful in the present disclosure include those which (for human administration) are of human origin (or of the origin of the mammal to which the probiotic microorganism is being administered), are non-pathogenic to the host, resist technological processes (i.e. can remain viable and active during processing and in delivery vehicles), are resistant to gastric acidity and bile toxicity, adhere to gut epithelial tissue, have the ability to colonize the gastrointestinal tract, produce antimicrobial substances, modulate immune response in the host, and influence metabolic activity (e.g. cholesterol assimilation, lactase activity, vitamin production).

The probiotic microorganism can be included in the glycan preparations as a single strain or a combination of multiple strains, wherein the total number of bacteria in a dose of probiotic microorganism is from about 1×10³ to about 1×10¹⁴, or from about 1×10 to about 1×10¹², or from about 1×10⁷ to about 1×10¹¹ CFU per dose.

The probiotic microorganisms can be incorporated into the glycan preparations while the probiotic microorganism is alive but in a state of “suspended animation” or somnolence. Once freeze-dried, the viable cultures(s) of probiotic microorganism are handled so as to minimize exposure to moisture that would reanimate the cultures because, once reanimated, the cultures can experience high rates of morbidity unless soon cultured in a high moisture environment or medium. Additionally, the cultures are handled to reduce possible exposure to high temperatures (particularly in the presence of moisture) to reduce morbidity.

The probiotic microorganisms can be used in a powdered, dry form. The probiotic microorganisms can also be administered in the glycan preparation or in a separate glycan preparation, administered at the same time or different time as the glycan preparations.

Examples of probiotics include, but are not limited to, those that acidify the colon such as those from the genera Lactobacillus or Bifidobacterium, which are thought to maintain a healthy balance of intestinal microbiota by producing organic acids (lactic & acetic acids), hydrogen peroxide, and bacteriocins which are documents to inhibit enteric pathogens.

Other Lactobacillus bacteria which can be employed include, but are not limited to, L. crispatus, L. casei, L. rhamnosus, L. reuteri, L. fermentum, L. plantarum, L. sporogenes, and L. bulgaricus. Other probiotic bacteria suitable for the glycan compositions include Bifidobacterium lactis, B. animalis, B. bifidum, B. longum, B. adolescentis, and B. infantis.

In embodiments, a commensal bacterial taxa that can be used in and/or in combination with a composition described herein comprises Akkermansia, Anaerococcus, Bacteroides, Bifidobacterium (including Bifidobacterium lactis, B. animalis, B. bifidum, B. longum, B. adolescentis, B. breve, and B. infantis), Blautia, Clostridium, Corynebacterium, Dialister, Eubacterium, Faecalibacterium, Finegoldia, Fusobacterium, Lactobacillus (including, L. acidophilus, L. helveticus, L. bifidus, L. lactis, L. fermentii, L. salivarius, L. paracasei, L. brevis, L. delbruekii, L. thermophiles, L. crispatus, L. casei, L. rhamnosus, L. reuteri, L. fermentum, L. plantarum, L. sporogenes, and L. bulgaricus), Peptococcus, Peptostreptococcus, Peptoniphilus, Prevotella, Roseburia, Ruminococcus, Staphylococcus, and/or Streptococcus (including S. lactis, S. cremoris, S. diacetylactis, S. thermophiles).

In embodiments, a commensal bacterial taxa, e.g., GRAS strain, that can be used in and/or in combination with a composition described herein comprises Bacillus coagulans GBI-30, 6086; Bifidobacterium animalis subsp. Lactis BB-12; Bifidobacterium breve Yakult; Bifidobacterium infantis 35624; Bifidobacterium animalis subsp. Lactis UNO 19 (DR10); Bifidobacterium longum BB536; Escherichia coli M-17; Escherichia coli Nissle 1917; Lactobacillus acidophilus DDS-1; Lactobacillus acidophilus LA-5; Lactobacillus acidophilus NCFM; Lactobacillus casei DN 114-001 {Lactobacillus casei Immunitas(s)/Defensis); Lactobacillus casei CRL431; Lactobacillus casei F19; Lactobacillus paracasei Stl l (or NCC2461); Lactobacillus johnsonii Lai (Lactobacillus LCI, Lactobacillus johnsonii NCC533); Lactococcus lactis L1A; Lactobacillus plantarum 299V; Lactobacillus reuteri ATTC 55730 (Lactobacillus reuteri SD2112); Lactobacillus rhamnosus ATCC 53013; Lactobacillus rhamnosus LB21; Saccharomyces cerevisiae {boulardii) lyo; mixture of Lactobacillus rhamnosus GR-1 and Lactobacillus reuteri RC-14; mixture of Lactobacillus acidophilus NCFM and Bifidobacterium lactis BB-12 or BL-04; mixture of Lactobacillus acidophilus CL1285 and Lactobacillus casei; and a mixture of Lactobacillus helveticus R0052, Lactobacillus rhamnosus R0011, and/or Lactobacillus rhamnosus GG (LGG).

### Synbiotics

Provided herein are combinations of microbes (e.g., bacterial taxa) with glycan compositions disclosed herein which can, e.g., be utilized by the microbes as their substrate for growth. Exogenously introduced microbes can provide a number of beneficial effects, such as, e.g., those described in Tables 1-3. This may occur by promoting the growth of the microbes (using the glycans), thereby allowing the microbes to outgrow other bacteria at the site of colonization.

Methods provided herein include administering one or more (e.g., one or more, two or more, three or more, four or more, and so on) bacterial taxa, such as those listed in Tables 1-3 or Tables 4-6 to a subject in combination with a glycan composition. Such a combination can increase, suppress, and/or alter certain bacterial taxa. Methods are provided herein to modulate the processing of an exogenous substance described herein, comprising administering one or more (e.g., one or more, two or more, three or more, four or more, and so on) bacterial taxa to a subject in combination with a glycan described herein to a subject. The subject can include a subject that has taken, is taking or will be taking an antibiotic. The subject can include a subject that is not taking or has not taken an antibiotic.

### Prebiotics

In some embodiments, the glycan compositions comprise a prebiotic substance. In some embodiments, prebiotics may be administered to a subject receiving the glycan preparations. Prebiotics are substantially non-digestible substances by the host that when consumed may provide a beneficial physiological effect on the host by selectively stimulating the favorable growth or activity of a limited number of indigenous bacteria in the gut (Gibson G R, Roberfroid M B. J Nutr. (1995) 125:1401-12.). A prebiotic such as a dietary fiber or prebiotic oligosaccharide (e.g. crystalline cellulose, wheat bran, oat bran, cone fiber, soy fiber, beet fiber and the like) may further encourage the growth of probiotic and/or commensal bacteria in the gut by providing a fermentable dose of carbohydrates to the bacteria and increase the levels of those microbial populations (e.g. lactobacilli and bifidobacteria) in the gastrointestinal tract.

Prebiotics may include, but are not limited to, various galactans and carbohydrate based gums, such as psyllium, guar, carrageen, gellan, lactulose, and konjac. In some embodiments, the prebiotic is one or more of galactooligosaccharides (GOS), lactulose, raffinose, stachyose, lactosucrose, fructo-oligosaccharides (FOS, e.g. oligofructose or oligofructan), inulin, isomalto-oligosaccharide, xylo-oligosaccharides (XOS), paratinose oligosaccharide, isomaltose oligosaccharides (IMOS), transgalactosylated oligosaccharides (e.g. transgalacto-oligosaccharides), transgalactosylate disaccharides, soybean oligosaccharides (e.g. soyoligosaccharides), chitosan oligosaccharide (chioses), gentiooligosaccharides, soy- and pectin-oligosaccharides, glucooligosaccharides, pecticoligosaccharides, palatinose polycondensates, difructose anhydride III, sorbitol, maltitol, lactitol, polyols, polydextrose, linear and branched dextrans, pullalan, hemicelluloses, reduced paratinose, cellulose, beta-glucose, beta-galactose, beta-fructose, verbascose, galactinol, xylan, inulin, chitosan, beta-glucan, guar gum, gum arabic, pectin, high sodium alginate, and lambda carrageenan, or mixtures thereof.

Prebiotics can be found in certain foods, e.g. chicory root, Jerusalem artichoke, Dandelion greens, garlic, leek, onion, asparagus, wheat bran, wheat flour, banana, milk, yogurt, sorghum, burdock, broccoli, Brussels sprouts, cabbage, cauliflower, collard greens, kale, radish and rutabaga, and miso. In some embodiments, the microbiome regulators described herein are administered to a subject in conjunction with a diet that includes foods rich in prebiotics. Suitable sources of soluble and insoluble fibers are commercially available.

In some embodiments, a glycan composition comprises at least about 1% (w/w) of a prebiotic substance (e.g., at least about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or more). In embodiments, the glycan composition comprises FOS. In embodiments, the glycan composition comprises lactulose.

Changes in bacterial populations can be measured by the “prebiotic index.” The prebiotic index considers increases in the growth rate of bifidobacteria, eubacteria, and lactobacilli as positive effects and increases in Clostridia, bacteriodes, sulphate-reducing bacteria, and Escherichia coli as negative effects. The prebiotic index (PI) relates to the sum of: (Bifidobacteria/total bacteria)+(Lactobacilli/total bacteria)−(Bacteroides/total bacteria)−(Clostridia/total bacteria), (see Palframan et al, 2003, Lett Appl Microbiol 37:281-284). In embodiments, administration of the glycan composition to a subject may result in an increased prebiotic index. Administration of a glycan composition to a subject may result in an increase in: Bacteroides, Blautia, Clostridium, Fusobacterium, Eubacterium, Ruminococcus, Peptococcus, Peptostreptococcus, Akkermansia, Faecalibacterium, Roseburia, Prevotella, Bifidobacterium, Lactobacilli, Christensenella minuta, or a Christensenellaceae.

In some embodiments, the glycan composition comprises an antibiotic, an antifungal agent, an antiviral agent, or an anti-inflammatory agent (e.g. a cytokine, hormone, etc.).

In some embodiments, the glycan compositions further comprise a second therapeutic agent or preparation thereof, such as a drug.

For example, the second therapeutic agent is an anti-cancer drug. Examples of anti-cancer drugs include: checkpoint inhibitors (such as, e.g., anti-PD-1, anti-PD-L1, anti-CTLA4, anti-TIM-3, anti-LAG-3); vaccines (such as, e.g., autologous cancer vaccines, allogeneic cancer vaccines, neoantigen cancer vaccines, shared antigen cancer vaccines (e.g. NY-ESO-1)); targeted kinase inhibitors (such as, e.g., Imatinib mesylate, Ibrutinib, Neratinib, Palpociclib, Erlotinib, Lapatinib); antibodies (such as, e.g., Bev acizumab, Trastuzumab, Rituximab, Cetuximab); chemotherapeutics (such as, e.g., irinotecan, 5-flurouracil, lenalidomide, capecitabine, docetaxel), antibody-drug conjugates (e.g. ado-trastuzumab emtansine), and any other anti-cancer drug mentioned elsewhere herein.

For example, the second therapeutic agent is a pain-management drug. In some embodiments, the pain-management drug is an opioid, such as, e.g., codeine, fentanyl, hydrocodone, hydrocodone/acetaminophen, hydromorphone, meperidine, methadone, morphine, oxycodone, oxycodone and acetaminophen, or oxycodone and naloxone. In other embodiments, the pain-management drug is a non-opioid, such as, e.g., acetaminophen or nonsteroidal anti-inflammatory drugs (NSAIDs), such as aspirin and ibuprofen.

For example, the second therapeutic agent is a cardiac glycoside, sulfonamide (sulfa drug), nucleoside analogue, or aminosalicylate.

In some embodiments, the cardiac glycoside is digoxin, digitoxin, convallotoxin, antiarin, or oleandrin.

In some embodiments, the sulfonamide (sulfa drug) is an antimicrobial, e.g., a short acting antimicrobial, e.g., Sulfafurazole, Sulfacetamide, Sulfadiazine, Sulfadimidine, Sulfafurazole (sulfisoxazole), Sulfisomidine (sulfaisodimidine). In some embodiments, the sulfonamide (sulfa drug) is an antimicrobial, e.g., an intermediate-acting microbial, e.g., Sulfadoxine, Sulfamethoxazole, Sulfamoxole, or Sulfanitran. In some embodiments, the sulfonamide (sulfa drug) is an antimicrobial, e.g., a long-acting antimicrobial, e.g., Sulfadimethoxine, Sulfamethoxypyridazine, orSulfametoxydiazine. In some embodiments, the sulfonamide (sulfa drug) is an antimicrobial, e.g., an ultra-long-acting antimicrobial, e.g., Sulfadoxine, Sulfametopyrazine, or Terephtyl. In some embodiments, the sulfonamide (sulfa drug) is a Sulfonylurea, e.g., a anti-diabetic agents, e.g., Acetohexamide, Carbutamide, Chlorpropamide, Glibenclamide (glyburide), Glibornuride, Gliclazide, Glyclopyramide, Glimepiride, Glipizide, Gliquidone, Glisoxepide, Tolazamide, or Tolbutamide. In some embodiments, the sulfonamide (sulfa drug) is a diuretic, e.g., Acetazolamide, Bumetanide, Chlorthalidone, Clopamide, Furosemide, Hydrochlorothiazide, Indapamide, Mefruside, Metolazone, or Xipamide. In some embodiments, the sulfonamide (sulfa drug) is an anticonvulsant, e.g., Ethoxzolamide, Sultiame, Topiramate, or Zonisamide. In some embodiments, the sulfonamide (sulfa drug) is an antiretrovirals, e.g., Amprenavir (HIV protease inhibitor), Darunavir (HIV protease inhibitor), Delavirdine (non-nucleoside reverse transcriptase inhibitor), Fosamprenavir (HIV protease inhibitor), or Tipranavir (HIV protease inhibitor). In some embodiments, the sulfonamide (sulfa drug) is a Hepatitis C antiviral, e.g., Asunaprevir (NS3/4A protease inhibitor), Beclabuvir (NS5B RNA polymerase inhibitor), Dasabuvir (NS5B RNA polymerase inhibitor), Grazoprevir (NS3/4A protease inhibitor), Paritaprevir (NS3/4A protease inhibitor), or Simeprevir (NS3/4A protease inhibitor). In some embodiments, the sulfonamide (sulfa drug) is e.g., Apricoxib (COX-2 inhibitor), Bosentan (endothelin receptor antagonist), Brinzolamide (carbonic anhydrase inhibitor for glaucoma), Celecoxib (COX-2 inhibitor), Dofetilide (class III antiarrhythmic), Dorzolamide (anti-glaucoma carbonic anhydrase inhibitor), Dronedarone (class III antiarrhythmic), Ibutilide (class III antiarrhythmic), Parecoxib (COX-2 inhibitor), Probenecid (uricosuric), Sotalol (0 blocker), Sulfasalazine (anti-inflammatory agent and a DMARD), Sumatriptan (antimigraine triptan), Tamsulosin (a blocker), or Udenafil (PDE5 inhibitor).

In some embodiments, the nucleoside analogues are deoxyadenosine analogues, e.g., didanosine (ddl)(HIV) or vidarabine (antiviral). In some embodiments, the nucleoside analogue is an adenosine analogue, e.g., BCX4430 (Ebola). In some embodiments, the nucleoside analogue is a deoxycytidine analogue, e.g., cytarabine (chemotherapy), gemcitabine (Chemotherapy), emtricitabine (FTC)(HIV), lamivudine (3TC)(HIV, hepatitis B), or zalcitabine (ddC)(HIV). In some embodiments, the nucleoside analogue is a guanosine or deoxyguanosine analogue, e.g., abacavir (HIV), acyclovir, or entecavir (hepatitis B). In some embodiments, the nucleoside analogue is a thymidine or deoxythymidine analogue, e.g., stavudine (d4T), telbivudine (hepatitis B) or zidovudine (azidothymidine, or AZT)(HIV). In some embodiments, the nucleoside analogue is a deoxyuridine analogue, e.g., idoxuridine or trifluridine. In some embodiments, the nucleoside analogue is a Pyrimidine analogue, e.g., 5-Fluorouracil (5FU), Floxuridine (FUDR), Cytarabine (Cytosine arabinoside), or 6-azauracil (6-AU). In some embodiments, the nucleoside analogue is a purine analog, e.g., Azathioprine, Mercaptopurine, Thiopurines, Fludarabine, or Pentostatin. In some embodiments, the aminosalicylate is 4-Aminosalicylic acid, Balsalazide, Olsalazine, Sulfasalazine, or Mesalazine (5-Aminosalicylic acid).

For example, the second therapeutic agent is an anti-proliferative, anti-neoplastic or anti-tumor drugs or treatments. In some embodiments, such drugs or treatments include chemotherapeutic drugs, e.g., cytotoxic drugs (e.g., alkylating agents, antimetabolites, anti-tumor antibiotics, topoisomerase inhibitors, mitotic inhibitors, corticosteroids); cancer growth blockers such as tyrosine kinase inhibitors and proteasome inhibitors; other chemical drugs such as L-asparaginase and bortezomib (Velcade®), anti-cancer drugs, e.g., checkpoint inhibitors (such as, e.g., anti-PD-1, anti-PD-L1, anti-CTLA4, anti-TIM-3, anti-LAG-3); vaccines (such as, e.g., autologous cancer vaccines, allogeneic cancer vaccines, neoantigen cancer vaccines, shared antigen cancer vaccines (e.g. NY-ESO-1)); targeted kinase inhibitors (such as, e.g., Imatinib mesylate, Ibrutinib, Neratinib, Palpociclib, Erlotinib, Lapatinib); or antibodies (such as, e.g., Bevacizumab, Trastuzumab, Rituximab, Cetuximab). Hormone therapies (or anti-hormone therapies) may be used, e.g., for hormone-sensitive cancers.

For example, the second therapeutic agent is a drug that is known to induce diarrhea or a drug that is known to induce constipation. In some embodiments the drugs known to induce diarrhea include 5-fluorouracil (5-FU), methotrexate, irinotecan, taxanes, monoclonal antibodies, and hormonal agents. In some embodiments the drugs known to induce constipation include vinca alkaloids, platinums (e.g., cisplatin), thalidomide and hormonal agents.

### Pharmaceutical Compositions, Medical Foods, Supplements, Food Ingredients, and Unit Dosage Form

Provided herein are pharmaceutical compositions comprising glycan compositions. Further provided herein are medical foods comprising glycan compositions. Still further provided herein are dietary supplements comprising glycan compositions. Still further provided herein are food ingredients comprising glycan compositions.

Optionally, the compositions comprise one or more of the following: i) a prebiotic substance, such as, e.g., a dietary fiber; ii) a bacterial taxa, such as, e.g., a probiotic bacterium; iii) a micronutrient, such as, e.g., a vitamin, mineral or polyphenol compound, iv) a therapeutic drug, such as, e.g., an anti-cancer drug, a pain management drug, a drug that manages treatment side-effects, a drug that manages metabolism, an anti-inflammatory drug, or an anti-microbial agent.

Pharmaceutical compositions, medical foods, supplements and unit dosage forms suitable for use in the methods and compositions described herein can be found in WO 2016/122889, WO 2016/172657, and WO 2016/172658, which are hereby incorporated by reference.

In some embodiments, the glycan compositions do not contain a prebiotic substance. In some embodiments glycan compositions do not contain a probiotic bacterium. In some embodiments, glycan compositions comprie one or more of glycan preparations described herein.

The glycan polymer preparations described herein may be formulated into any suitable dosage form, e.g. for nasal, oral, rectal or gastric administration. In some embodiments, the glycan polymer preparations described herein may be formulated for enteral administration. In some embodiments, the glycan polymer preparations described herein may be formulated for tube feeding (e.g. naso-gastric, oral-gastric or gastric feeding). The dosage forms described herein can be manufactured using processes that are known to those of skill in the art.

The dosage form may be a packet, such as any individual container that contains a glycan polymer preparation in the form of, e.g., a liquid (wash/rinse), a gel, a cream, an ointment, a powder, a tablet, a pill, a capsule, a depository, a single-use applicator or medical device (e.g. a syringe). For example, provided is also an article of manufacture, such as a container comprising a unit dosage form of the glycan polymer preparation, and a label containing instructions for use of such glycan polymer.

Forms of the compositions that can be used orally include tablets, push-fit capsules made of gelatin, as well as soft, sealed capsules made of gelatin and a plasticizer, such as glycerol or sorbitol. Tablets can be made by compression or molding, optionally with one or more accessory ingredients. Compressed tablets can be prepared by compressing in a suitable machine the active ingredient in a free-flowing form such as a powder or granules, optionally mixed with binders (e.g., povidone, gelatin, hydroxypropylmethyl cellulose), inert diluents, preservative, antioxidant, disintegrant (e.g., sodium starch glycolate, cross-linked povidone, cross-linked sodium carboxymethyl cellulose) or lubricating, surface active or dispersing agents. Molded tablets can be made by molding in a suitable machine a mixture of the powdered compound moistened with an inert liquid diluent. The tablets can optionally be coated or scored and can be formulated so as to provide slow or controlled release of the active ingredient therein. Tablets can optionally be provided with an enteric coating, to provide release in parts of the gut (e.g., colon, lower intestine) other than the stomach. All formulations for oral administration can be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds and/or other agents (e.g., prebiotics or probiotics) can be dissolved or suspended in suitable liquids, such as fatty oils, liquid paraffin, or liquid polyethylene glycols. In addition, stabilizers can be added. Dragee cores are provided with suitable coatings. For this purpose, concentrated sugar solutions can be used, which can optionally contain gum arabic, talc, polyvinyl pyrrolidone, carbopol gel, polyethylene glycol, or titanium dioxide, lacquer solutions, and suitable organic solvents or solvent mixtures. Dyestuffs or pigments can be added to the tablets or dragee coatings for identification or to characterize different combinations of active compound doses.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethylene glycol or an oil medium, for example peanut oil, liquid paraffin, or olive oil.

In one embodiment, a provided glycan polymer preparation includes a softgel formulation. A softgel can contain a gelatin-based shell that surrounds a liquid fill. The shell can be made of gelatin, plasticizer (e.g., glycerin and/or sorbitol), modifier, water, color, antioxidant, or flavor. The shell can be made with starch or carrageenan. The outer layer can be enteric coated. In one embodiment, a softgel formulation can include a water or oil soluble fill solution, or suspension of a composition covered by a layer of gelatin.

Solid formulations for oral use may comprise an enteric coating, which may control the location at which a glycan polymer preparation is absorbed in the digestive system. For example, an enteric coating can be designed such that a glycan polymer preparation does not dissolve in the stomach but rather travels to the small intestine, where it dissolves. An enteric coating can be stable at low pH (such as in the stomach) and can dissolve at higher pH (for example, in the small intestine). Material that can be used in enteric coatings includes, for example, alginic acid, cellulose acetate phthalate, plastics, waxes, shellac, and fatty acids (e.g., stearic acid, palmitic acid).

Formulations for oral use may also be presented in a liquid dosage from. Liquid preparations can be in the form of, for example, aqueous or oily suspensions, solutions, emulsions syrups or elixirs, or can be presented as a dry product for reconstitution with water or other suitable vehicle before use. Such liquid preparations can contain conventional additives, such as suspending agents, for example sorbitol, methyl cellulose, glucose syrup, gelatin, hydroxyethyl cellulose, carboxymethyl cellulose, aluminum stearate gel or hydrogenated edible fats, emulsifying agents, for example lecithin, sorbitan monooleate, acacia; nonaqueous vehicles (which can include edible oils), for example almond oil, oily esters such as glycerine, propylene glycol, or ethyl alcohol; preservatives, for example methyl or propyl p-hydoxybenzoate or sorbic acid, and, if desired, conventional flavoring or coloring agents. In some embodiments, liquid formulations can comprise, for example, an agent in water-in-solution and/or suspension form; and a vehicle comprising polyethoxylated castor oil, alcohol, and/or a polyoxyethylated sorbitan mono-oleate with or without flavoring. Each dosage form may comprise an effective amount of a glycan polymer and can optionally comprise pharmaceutically inert agents, such as conventional excipients, vehicles, fillers, binders, disintegrants, pH adjusting substances, buffer, solvents, solubilizing agents, sweeteners, coloring agents, and any other inactive agents that can be included in pharmaceutical dosage forms for administration. Examples of such vehicles and additives can be found in Remington's Pharmaceutical Sciences, 17th edition (1985).

The pharmaceutical compositions provided herein can be in unit-dosage forms or multiple-dosage forms. A unit-dosage form, as used herein, refers to physically discrete unit suitable for administration to human in need thereof. In an embodiment, the unit-dosage form is provided in a package. Each unit-dose can contain a predetermined quantity of an active ingredient(s) sufficient to produce the desired therapeutic effect, in association with other pharmaceutical carriers or excipients. Examples of unit-dosage forms include, but are not limited to, ampoules, syringes, and individually packaged tablets and capsules. Unit-dosage forms can be administered in fractions or multiples thereof. A multiple-dosage form is a plurality of identical unit-dosage forms packaged in a single container, which can be administered in segregated unit-dosage form. Examples of multiple-dosage forms include, but are not limited to, vials, bottles of tablets or capsules, or bottles of pints or gallons. In another embodiment, the multiple dosage forms comprise different pharmaceutically active agents. For example, a multiple dosage form can be provided which comprises a first dosage element comprising a composition comprising a glycan polymer and a second dosage element comprising a prebiotic, a therapeutic agent and/or a probiotic, which can be in a modified release form. In this example a pair of dosage elements can make a single unit dosage. In one embodiment, a kit is provided comprising multiple unit dosages, wherein each unit comprises a first dosage element comprising a composition comprising a glycan polymer preparation and a second dosage element comprising probiotic, a pharmaceutical agent, a prebiotic or a combination thereof, which can be in a modified release form. In another embodiment, the kit further comprises a set of instructions.

In some embodiments, the unit-dosage form comprises between about 1 mg to about 100 g of the glycan polymer preparation (e.g., a glycan polymer disclosed herein). For example, the unit-dosage form may comprise about 50 mg to about 50 g, about 500 mg to about 50 g, about 5 g to about 50 g, about 100 mg to about 100 g, about 1 g to about 100 g, about 10 g to about 100 g, about 1 g to about 10 g, about 1 g to about 20 g, about 1 g to about 30 g, about 1 g to about 40 g, about 1 g to about 50 g, about 1 g to about 60 g, about 1 g to about 70 g, about 1 g to about 80 g, about 1 g to about 90 g, about 1 g to about 100 g, about 1 g to about 150 g, about 1 g to about 200 g of the glycan polymer.

In other embodiments, the unit-dosage form comprises between about 0.001 mL to about 1000 mL of the glycan polymer (e.g., a glycan polymer disclosed herein). For example, the unit-dosage form may comprise about 0.001 mL to about 950 mL, about 0.005 mL to about 900 mL, about 0.01 mL to about 850 mL, about 0.05 mL to about 800 mL, about 0.075 mL to about 750 mL, about 0.1 mL to about 700 mL, about 0.25 mL to about 650 mL, about 0.5 mL to about 600 mL, about 0.75 mL to about 550 mL, about 1 mL to about 500 mL, about 2.5 mL to about 450 mL, about 5 mL to about 400 mL, about 7.5 mL to about 350 mL, about 10 mL to about 300 mL, about 12.5 mL to about 250 mL, about 15 mL to about 200 mL, about 17.5 mL to about 150 mL, about 20 mL to about 100 mL, or about 25 mL to about 75 mL of the glycan polymer.

In certain embodiments, the unit-dosage form comprises about 0.001 mL to about 10 mL, about 0.005 mL to about 7.5 mL, about 0.01 mL to about 5 mL, about 0.05 mL to about 2.5 mL, about 0.1 mL to about 1 mL, about 0.25 mL to about 1 mL, or about 0.5 mL to about 1 mL of the glycan polymer. In other embodiments, the unit-dosage form comprises about 0.01 mL to about 10 mL, about 0.025 mL to about 7.5 mL, about 0.05 mL to about 5 mL, or about 0.1 mL to about 2.5 mL of the glycan polymer. In other embodiments, the unit-dosage form comprises about 0.1 mL to about 10 mL, about 0.25 mL to about 7.5 mL, about 0.5 mL to about 5 mL, about 0.5 mL to about 2.5 mL, or about 0.5 mL to about 1 mL of the glycan polymer.

In some embodiments, the unit-dosage form, e.g., a tablet, capsule (e.g., a hard capsule, push-fit capsule, or soft capsule), or softgel, has a body length of between about 0.1 inches to about 1.5 inches (e.g., about 0.5 inches and about 1 inch), or about 5 mm to about 50 mm (e.g., about 10 mm to about 25 mm). In some embodiments, the unit-dosage form. e.g., a tablet, capsule (e.g., a hard capsule, push-fit capsule, or soft capsule), or softgel, has an external diameter of about 0.05 inches to about 1 inch (e.g., about 0.1 inches to about 0.5 inches), or about 1 mm to about 25 mm (e.g., about 5 mm to about 10 mm).

Each unit-dosage form of the glycan polymer may have a caloric value of between about 0.01 kcal and about 1000 kcal. For example, the unit-dosage form may have a caloric value of about 0.01 kcal to about 100 kcal, about 0.05 kcal to about 50 kcal, about 0.1 kcal to about 10 kcal, about 0.25 kcal to about 2.5 kcal, about 0.5 kcal to about 5 kcal, about 0.75 kcal to about 7.5 kcal, about 1 kcal to 10 kcal, about 5 kcal to about 50 kcal, or about 10 kcal to about 100 kcal. In certain embodiments, the unit-dosage form of the glycan polymer has a caloric value of between 10 kcal to about 500 kcal. In certain embodiments, the unit-dosage form of the glycan polymer has a caloric value of between 1 kcal to about 100 kcal. In certain embodiments, the unit-dosage form of the glycan polymer has a caloric value of between 0.1 kcal to about 10 kcal.

In still other embodiments, the unit-dosage form may have a caloric value of about 0.001 kcal to about 10 kcal, about 0.005 kcal to about 10 kcal, about 0.01 kcal to about 10 kcal, about 0.025 kcal to about 25 kcal, about 0.05 kcal to about 50 kcal, about 0.075 kcal to about 75 kcal, about 0.1 kcal to 100 kcal, about 0.25 kcal to about 10 kcal, about 0.5 kcal to about 5 kcal, about 0.25 kcal to about 25 kcal, or about 0.1 kcal to about 1 kcal.

The unit-dosage form of the glycan polymer may be formulated to dissolve in an aqueous solution (e.g., water, milk, juice, and the like) and is orally administered as a beverage, syrup, solution, or suspension. For example, the unit-form dosage of the glycan polymer may comprise a cube, packet, lozenge, pill, tablet, capsule, candy, powder, elixir, or concentrated syrup formulated for dissolving into an aqueous solution prior to oral administration. In other embodiments, the unit-dosage form of the glycan polymer may comprise a cube, packet, lozenge, pill, tablet, capsule, candy, powder, elixir, or concentrated syrup formulated to dissolve in vivo, e.g., in the mouth, stomach, intestine, or colon of the subject upon oral administration.

In some embodiments, the glycan polymer preparation is administered enterically. This preferentially includes oral administration, or by an oral or nasal tube (including nasogastric, nasojejunal, oral gastric, or oral jejunal). In other embodiments, administration includes rectal administration (including enema, suppository, or colonoscopy).

The dosage forms described herein can be manufactured using processes that are known to those of skill in the art. For example, for the manufacture of tablets, an effective amount of a prebiotic can be dispersed uniformly in one or more excipients or additives, for example, using high shear granulation, low shear granulation, fluid bed granulation, or by blending for direct compression. Excipients and additives include diluents, binders, disintegrants, dispersants, lubricants, glidants, stabilizers, surfactants, antiadherents, sorbents, sweeteners, and colorants, or a combination thereof. Diluents, also termed fillers, can be used to increase the bulk of a tablet so that a practical size is provided for compression. Non-limiting examples of diluents include lactose, cellulose, microcrystalline cellulose, mannitol, dry starch, hydrolyzed starches, powdered sugar, talc, sodium chloride, silicon dioxide, titanium oxide, dicalcium phosphate dihydrate, calcium sulfate, calcium carbonate, alumina and kaolin. Binders can impart cohesive qualities to a tablet formulation and can be used to help a tablet remain intact after compression. Non-limiting examples of suitable binders include starch (including corn starch and pregelatinized starch), gelatin, sugars (e.g., glucose, dextrose, sucrose, lactose and sorbitol), celluloses, polyethylene glycol, alginic acid, dextrin, casein, methyl cellulose, waxes, natural and synthetic gums, e.g., acacia, tragacanth, sodium alginate, gum arabic, xantan gum, and synthetic polymers such as polymethacrylates, polyvinyl alcohols, hydroxypropylcellulose, and polyvinylpyrrolidone. Lubricants can also facilitate tablet manufacture; non-limiting examples thereof include magnesium stearate, calcium stearate, stearic acid, glyceryl behenate, and polyethylene glycol. Disintegrants can facilitate tablet disintegration after administration, and non-limiting examples thereof include starches, alginic acid, crosslinked polymers such as, e.g., crosslinked polyvinylpyrrolidone, croscarmellose sodium, potassium or sodium starch glycolate, clays, celluloses (e.g., carboxymethylcelluloses (e.g., carboxymethylcellulose (CMC), CMC-Na, CMC-Ca)), starches, gums and the like. Non-limiting examples of suitable glidants include silicon dioxide, talc, and the like. Stabilizers can inhibit or retard drug decomposition reactions, including oxidative reactions. Surfactants can also include and can be anionic, cationic, amphoteric or nonionic. Exemplary sweeteners may include stevia extract, aspartame, sucrose, alitame, saccharin, and the like. If desired, the tablets can also comprise nontoxic auxiliary substances such as pH buffering agents, preservatives, e.g., antioxidants, wetting or emulsifying agents, solubilizing agents, coating agents, flavoring agents (e.g., mint, cherry, anise, peach, apricot, licorice, raspberry, vanilla), and the like. Additional excipients and additives may include aluminum acetate, benzyl alcohol, butyl paraben, butylated hydroxy toluene, calcium disodium EDTA, calcium hydrogen phosphate dihydrate, dibasic calcium phosphate, tribasic calcium phosphate, candelilla wax, carnuba wax, castor oil hydrogenated, cetylpyridine chloride, citric acid, colloidal silicone dioxide, copolyvidone, corn starch, cysteine HCl, dimethicone, disodium hydrogen phosphate, erythrosine sodium, ethyl cellulose, gelatin, glycerin, glyceryl monooleate, glyceryl monostearate, glycine, HPMC pthalate, hydroxypropylcellulose, hydroxyl propyl methyl cellulose, hypromellose, iron oxide red or ferric oxide, iron oxide yellow, iron oxide or ferric oxide, magnesium carbonate, magnesium oxide, magnesium stearate, methionine, methacrylic acid copolymer, methyl paraben, silicified microcrystalline cellulose, mineral oil, phosphoric acid, plain calcium phosphate, anhydrous calcium phosphate, polaxamer 407, polaxamer 188, plain polaxamer, polyethylene oxide, polyoxyl40 stearate, polysorbate 80, potassium bicarbonate, potassium sorbate, potato starch, povidone, propylene glycol, propylene paraben, propyl paraben, retinyl palmitate, saccharin sodium, selenium, silica, silica gel, fumed silica, sodium benzoate, sodium carbonate, sodium citrate dihydrate, sodium crossmellose, sodium lauryl sulfate, sodium metabisulfite, sodium propionate, sodium starch, sodium starch glycolate, sodium stearyl fumarate, sorbic acid, sorbitol, sorbitan monooleate, pregelatinized starch, succinic acid, triacetin, triethyl citrate, vegetable stearin, vitamin A, vitamin E, vitamin C, or a combination thereof. The amounts of these excipients and additives can be properly selected based on their relation to other components and properties of the preparation and production method.

Immediate-release formulations of an effective amount of a glycan polymer preparation can comprise one or more combinations of excipients that allow for a rapid release of a pharmaceutically active agent (such as from 1 minute to 1 hour after administration).Controlled-release formulations (also referred to as sustained release (SR), extended-release (ER, XR, or XL), time-release or timed-release, controlled-release (CR), or continuous-release) refer to the release of a glycan polymer preparation from a dosage form at a particular desired point in time after the dosage form is administered to a subject.

In one embodiment a controlled release dosage form begins its release and continues that release over an extended period of time. Release can occur beginning almost immediately or can be sustained. Release can be constant, can increase or decrease over time, can be pulsed, can be continuous or intermittent, and the like. In one embodiment, a controlled release dosage refers to the release of an agent from a composition or dosage form in which the agent is released according to a desired profile over an extended period of time. In one aspect, controlled-release refers to delayed release of an agent from a composition or dosage form in which the agent is released according to a desired profile in which the release occurs after a period of time.

Pharmaceutical carriers or vehicles suitable for administration of the compounds provided herein include all such carriers known to those skilled in the art to be suitable for the particular mode of administration. In addition, the compositions can one or more components that do not impair the desired action, or with components that supplement the desired action, or have another action.

In a further aspect, the dosage form can be an effervescent dosage form. Effervescent means that the dosage form, when mixed with liquid, including water and saliva, evolves a gas. Some effervescent agents (or effervescent couple) evolve gas by means of a chemical reaction which takes place upon exposure of the effervescent disintegration agent to water or to saliva in the mouth. This reaction can be the result of the reaction of a soluble acid source and an alkali monocarbonate or carbonate source. The reaction of these two general compounds produces carbon dioxide gas upon contact with water or saliva. An effervescent couple (or the individual acid and base separately) can be coated with a solvent protective or enteric coating to prevent premature reaction. Such a couple can also be mixed with previously lyophilized particles (such as a glycan polymer). The acid sources can be any which are safe for human consumption and can generally include food acids, acid and hydrite antacids such as, for example: citric, tartaric, amalic, fumeric, adipic, and succinics. Carbonate sources include dry solid carbonate and bicarbonate salt such as sodium bicarbonate, sodium carbonate, potassium bicarbonate and potassium carbonate, magnesium carbonate and the like. Reactants which evolve oxygen or other gasses and which are safe for human consumption are also included. In one embodiment citric acid and sodium bicarbonate are used.

In another aspect, the dosage form can be in a candy form (e.g., matrix), such as a lollipop or lozenge. In one embodiment an effective amount of a glycan polymer is dispersed within a candy matrix. In one embodiment the candy matrix comprises one or more sugars (such as dextrose or sucrose). In another embodiment the candy matrix is a sugar-free matrix. The choice of a particular candy matrix is subject to wide variation. Conventional sweeteners (e.g., sucrose), sugar alcohols suitable for use with diabetic patients (e.g., sorbitol or mannitol), or other sweeteners (e.g., sweeteners described herein) may be employed. The candy base can be very soft and fast dissolving or can be hard and slower dissolving. Various forms will have advantages in different situations.

A candy mass composition comprising an effective amount of the glycan polymer can be orally administered to a subject in need thereof so that an effective amount of the glycan polymer will be released into the subject's mouth as the candy mass dissolves and is swallowed. A subject in need thereof includes a human adult or child.

The dosage forms described herein can also take the form of pharmaceutical particles manufactured by a variety of methods, including but not limited to high-pressure homogenization, wet or dry ball milling, or small particle precipitation (e.g., nGimat's NanoSpray). Other methods useful to make a suitable powder formulation are the preparation of a solution of active ingredients and excipients, followed by precipitation, filtration, and pulverization, or followed by removal of the solvent by freeze-drying, followed by pulverization of the powder to the desired particle size. In one embodiment, the pharmaceutical particles have a final size of 3-1000 microns, such as at most 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000 microns. In another embodiment, the pharmaceutical particles have a final size of 10-500 microns. In another embodiment, the pharmaceutical particles have a final size of 50-600 microns. In another embodiment, the pharmaceutical particles have a final size of 100-800 microns.

In another aspect, the disclosure provides a method of making a unit-dosage form described herein, comprising providing a glycan polymer (e.g., a glycan polymer described herein); formulating the glycan polymer into a unit-dosage form (e.g., a unit-dosage form described herein), packaging the unit-dosage form, labelling the packaged unit-dosage form, and/or selling or offering for sale the packaged and labeled unit-dosage form.

The unit-dosage forms described herein may also be processed. In one embodiment, the processing comprises one or more of: processing the dosage form into a pharmaceutical composition, e.g., formulating, combining with a second component, e.g., an excipient or buffer; portioning into smaller or larger aliquots; disposing into a container, e.g., a gas or liquid tight container; packaging; associating with a label; shipping or moving to a different location. In one embodiment, the processing comprises one or more of: classifying, selecting, accepting or discarding, releasing or withholding, processing into a pharmaceutical composition, shipping, moving to a different location, formulating, labeling, packaging, releasing into commerce, or selling or offering for sale, depending on whether the predetermined threshold is met. In some embodiments, the processed dosage forms comprise a glycan polymer described herein.

In some embodiments, the processing comprises one or more of: processing the dosage form into a pharmaceutical composition, e.g., formulating, combining with a second component, e.g., an excipient or buffer; portioning into smaller or larger aliquots; disposing into a container, e.g., a gas or liquid tight container; packaging; associating with a label; shipping or moving to a different location. In one embodiment, the processing comprises one or more of: classifying, selecting, accepting or discarding, releasing or withholding, processing into a pharmaceutical composition, shipping, moving to a different location, formulating, labeling, packaging, releasing into commerce, or selling or offering for sale, depending on the determination.

In another embodiment, an oral dosage form is provided comprising a glycan polymer preparation, wherein the oral dosage form is a syrup. The syrup can comprise about 1%, 5%, 10%, 15%, 20%, 25%, 30%, 35%, 40%, 45%, 50%, 55%, 60%, 65%, 70%, 75%, 80%, or 85% solid. The syrup can comprise about 15%, 20%, 25%, 30%, 35%, 40%, 45%, or 50% liquid, for example, water. The solid can comprise a glycan polymer preparation. The solid can be, for example, about 1-96%, 10-96%, 20-96%, 30-96%, 40-96%, 50-96%, 60-96%, 70-96%, 80-96%, or 90-96% glycan polymer preparation. In another embodiment, a glycan polymer preparation is formulated as a viscous fluid.

In one embodiment, the composition comprises a foaming component, a neutralizing component, or a water-insoluble dietary fiber. A foaming component can be at least one member selected from the group consisting of sodium hydrogencarbonate, sodium carbonate, and calcium carbonate. In one embodiment a neutralizing component can be at least one member selected from the group consisting of citric acid, L-tartaric acid, fumaric acid, L-ascorbic acid, DL-malic acid, acetic acid, lactic acid, and anhydrous citric acid. In one embodiment a water-insoluble dietary fiber can be at least one member selected from the group consisting of crystalline cellulose, wheat bran, oat bran, cone fiber, soy fiber, and beet fiber. The formulation can contain a sucrose fatty acid ester, powder sugar, fruit juice powder, and/or flavoring material.

In some embodiments, the dosage forms are formulated to release the pharmaceutical compositions comprising glycan polymer preparations in a specific region(s) of the GI tract, such as the small or the large intestine. In some embodiments, the dosage forms are formulated to release the pharmaceutical compositions comprising glycan polymer preparations in a specific region(s) of the GI tract, such as the cecum, ascending colon, transverse colon, descending colon, sigmoid colon, and/or rectum.

In some embodiments, the dosage form for the glycan polymer preparations described herein is an enzyme-responsive delivery system. For example, trypsin responsive polymers can be made using hydrogels that are crosslinked by peptides that are degraded by trypsin. Trypsin is active in the small intestine. Trypsin-responsive delivery systems can be used to target delivery of the glycan polymer preparations to the small intestine. In another example, enzyme-digestible hydrogels consisting of poly(vinyl pyrrolidone) crosslinked with albumin are degraded in the presence of pepsin.

In some embodiments, the dosage form for the glycan polymer preparations described herein is a delivery device that enables prolonged retention at a specific site in the GI tract. For example, a gastroretentive delivery system enables prolonged release of the glycan polymer preparations to the stomach. Gastroretentive delivery may be used for the glycan polymer preparations that modulate bacteria in the stomach or in the upper small intestine.

In some embodiments, the dosage form for the glycan polymer preparations described herein is a mucoadhesive delivery system that adheres to the mucosal surfaces of the stomach. They are typically composed of polymers with numerous hydrogen-bonding groups, e.g., cross-linked polyacrylic acids, sodium carboxymethyl cellulose, sodium alginate, carrageenan, Carbopol 934P, or thiolated polycarbophil.

In some embodiments, the dosage form for the glycan polymer preparations described herein is an expanding delivery system that rapidly increases in size in the stomach, which slows its passage through the pylorus. Such systems include systems that unfold in the stomach. For example, geometric shapes such as tetrahedrons, rings, disks, etc. can be packed into a gelatin capsule. When the capsule dissolves, the shape unfolds. The systems can be composed of one or more erodible polymer (e.g., hydroxypropyl cellulose), one or more nonerodible polymer (e.g., polyolefins, polyamides, polyurethanes). The glycan polymer may then be dispersed within the polymer matrix. The retention times can be fine-tuned by the polymer blend. Alternatively, devices made out of elastic polymers that are stable in the acidic pH of the stomach but dissolve in the neutral/alkaline conditions further along the GI tract can be used. Such polymer formulations can prevent intestinal obstruction when the device exits the stomach. Supramolecular polymer gels crosslinked by hydrogen bonds between carboxyl groups may also be used, e.g. composed of poly(acryloyl 6-aminocaproic acid) (PA6ACA) and poly(methacrylic acid-co-ethyl acrylate) (EUDRAGIT L 100-55). Other systems include swellable excipients, such as collagen sponges. For example, a hydrogel matrix (e.g. a swellable core: polyvinyl pyrrolidone XL, Carbopol 934P, calcium carbonate) swells 2-50 times in the stomach. Superporous hydrogel composites swell to hundreds of times their original volume in a few minutes. Some systems exploit gas generation to achieve expansion, e.g. carbon dioxide-generating, expandable systems that are surrounded by a hydrophilic membrane.

In some embodiments, the dosage form for the glycan polymer preparations described herein is a density-controlled delivery system. These systems are designed to either float or sink in gastric fluids, which delays their emptying from the stomach. For example, high-density systems enable the device to settle to the bottom of the stomach, below the pylorus, and thus avoid stomach emptying. Other systems are low-density/floating systems. Such devices may, e.g., comprise entrapped air in hollow chambers or may incorporate low-density materials like fats, oils, or foam powder. Low density may be achieved through swelling, e.g. hydrocolloid containing capsules dissolve upon contacting gastric fluid and the hydrocolloids swell to form a mucous body. Alternative polymers include: chitosans, sodium alginate, and glycerol monooleate matrix. Low density may be achieved through gas generation. For example, tablets loaded with carbonate and optionally citric acid generate carbon dioxide after contact with acidic aqueous media. The carbon dioxide generated is entrapped within the gelling hydrocolloid causing the system to float. Hydrocolloids include hydroxypropyl methylcellulose and Carbopol 934P.

In some embodiments, the dosage form for the glycan polymer preparations described herein employs a design to retain a device in the small or large intestine. The location-specific nature of the device is provided by a specific triggering method, e.g. pH, enzyme, etc. These include systems designed for mucoadhesion and also microneedle pills. Microneedle pills comprise a drug reservoir spiked with microneedles that is encapsulated in a pH-responsive coating. When the pill reaches the desired location in the GI tract and the coating dissolves, the microneedles enable the pill to become stuck to the lining of the GI tract. In other embodiments, the microneedle pills comprise a capsule that consists of two chemical compartments filled with citric acid and sodium bicarbonate, respectively. As the pill dissolves in the digestive system, barriers between the two substances erode, allowing them to mix and create a chemical reaction that pushes micro-needles of saccharides through the outer layer of the capsule and into the lining of the small intestine. The saccharide needles can be filled with drugs that are delivered into nearby blood vessels as the saccharide is absorbed.

In some embodiments, the dosage form for the glycan polymer preparations described herein employs a pH sensitive polymer coating. For example, pH-dependent polymers (bi- or tri-phasic) can be insoluble at low pH levels (e.g. acid resistance in the stomach, pH 1-2) and become increasingly soluble as pH rises, e.g. to about 5.5-6.2 in the duodenum, to about pH 5.7 in the ascending colon, to about pH 6.4 in the cecum, to about pH 6.6 in the transverse colon, to about pH 7.0 in the descending colon, to about 7.2-7.5 in the ileum, or to about pH 7.5 in the distal small intestine. In one example, TARGIT™ technology may be used for site-specific delivery of the glycan polymer preparations in the gastrointestinal (GI) tract. The system employs pH-sensitive coatings onto injection-moulded starch capsules to target the terminal ileum and colon.

In some embodiments, the dosage form for the glycan polymer preparations described herein is a delayed release system or time-controlled release system. Such systems usually employ enteric coatings that may be combined with pH sensitive and time release functions. For example, ETP (enteric coated time-release press coated) tablets may be used that are composed of three components: a glycan polymer-containing core tablet (rapid release function), a press-coated, swellable hydrophobic polymer layer (e.g. hydroxypropyl cellulose layer (HPC), and a time release function. The duration of lag phase can be controlled either by weight or composition of polymer layer and an enteric coating layer (acid resistance function).

In some embodiments, the dosage form for the glycan polymer preparations described herein employs Eudragit® enteric coatings of tablets and capsules. Other suitable synthetic polymers include: Shellac, ethyl cellulose, cellulose acetate phthalate, hydroxypropylmethyl cellulose, polyvinyl acetate phthalate and poly glutamic acid coatings, such as poly-γ-glutamic acid (y-PGA). These coatings combine both mucoadhesive and pH-dependent release strategies. To enhance colon targeted delivery Eudragits® are methacrylic co-polymers with varying side group compositions that alter the pH at which they are soluble. For example, for Eudragit®-coated systems no significant drug release occurs in the stomach (e.g. at pH 1.4) and in the small intestine (e.g. at pH 6.3), while significant drug release can be seen at pH 7.8 in the ileocaecal region.

In some embodiments, the dosage form for the glycan polymer preparations described herein is a microbial-triggered system, such as a polysaccharide-based delivery system. Polysaccharide based delivery systems contain biodegradable and mucoadhesive polymer coatings, including coatings of chitosan and pectin. Other suitable natural polymers include, e.g., guar gum, inulin, cyclodextrin, dextran, amylase, chondrotin sulphate, and locust bean gum. These delivery systems can be used to target the glycan polymer to the small intestine. Coatings with naturally occurring polysaccharides like guar gum, xanthan gum, chitosan, alginates, etc. are degraded by colonic gut microbiota, e.g. enzymes such as, xylosidase, arabinosidase, galactosidase etc. For example, CODES™ technology may be used to deliver the glycan polymer preparations. This system combines the polysaccharide coating with a pH-sensitive coating. In some embodiments, the system consists of a core tablet coated with three layers of polymer coatings: The outer coating is composed of Eudragit L. This coating gets dissolved in the duodenum and exposes the next coating. The next coating is composed of Eudragit E. This layer allows the release of lactulose present in the inner core. The lactulose gets metabolized into short chain fatty acids that lower the surrounding pH where the Eudragit E layer dissolves. The dissolving of Eudragit E results in the exposure of the glycan polymer. The bacteria present in the colon are responsible for the degradation of polysaccharides that are released from the core tablet. The degradation of polysaccharides may result in organic acids formation that lowers the pH of the contents surrounding the tablet.

In some embodiments, the dosage form for the glycan polymer preparations described herein is a pressure-controlled delivery system. The system employs the fact that higher pressures are encountered in the colon than in the small intestine. For example, for ethylcellulose systems that are insoluble in water, the release of glycan polymers occurs following disintegration of a water-insoluble polymer capsule as a result of pressure in the lumen of the colon. The release profile may be adjusted by varying the thickness of the ethylcellulose, the capsule size and/or density of the capsule.

In some embodiments, the dosage form for the glycan polymer preparations described herein is a pulsatile colon targeted delivery system. For example, the system can be a pulsincap system. The capsule which is employed comprises a plug that is placed in the capsule that controls the release of the glycan polymer. A swellable hydrogel (e.g. hydroxyl propyl methyl cellulose (HPMC), poly methyl methacrylate or polyvinyl acetate) seals the drug content. When the capsule gets in contact with a fluid the plug is pushed off from the capsule and the glycan polymer is released. The release profile can be controlled by varying the length and/or point of intersection of the plug with the capsule body. Another system is a port system. The capsule body is enclosed in a semi-permeable membrane. The insoluble plug consists of an osmotically active agent and the glycan polymer. When the capsule gets in contact with a fluid the semi-permeable membrane permits inflow of the fluid which increases pressure in the capsule body. This leads to an expelling of the plug and release of the glycan polymer.

In some embodiments, the dosage form for the glycan polymer preparations described herein is an osmotically controlled colon targeted delivery system. An exemplary system, OROS-CT, consists of osmotic units (up to 5 or 6 push pull units) encapsulated in a hard gelatin capsule. The push pull units are bilayered with outer enteric impermeable membrane and inner semi-permeable membrane. The internal, central part of the push pull consists of the drug layer and push layer. The glycan polymer is released through the semi-permeable membrane. The capsule body enclosing the push pull units is dissolved immediately after administration. In the GI tract the enteric impermeable membrane prevents water absorption. The enteric coating is dissolved in small intestine (higher pH, >7), water enters the unit through the semi-permeable membrane causing push layer to swell and force out the glycan polymer.

In some embodiments, the dosage form for the glycan polymer preparations described herein is “smart pill” which can be used to release the glycan polymer just before reaching the ileocecal valve.

In some embodiments, the dosage form for the glycan polymer preparations described herein is a rectally administered formulation. For example, enemas introduce a glycan polymer preparation in liquid formulation into the rectum. The volume administered is typically less than 10 mL. Suppositories introduce a glycan polymer preparation into the rectum. Suppositories are solid dosage forms that melt or dissolve when inserted into the rectum, releasing the glycan polymers. Typical excipients for suppository formulations include cocoa butter, polyethylene glycols, and agar.

Dosage forms

The glycan compositions described herein may be formulated into any suitable dosage form, e.g. for oral or enteral administration or formulated for injection. Suitable dosage forms for use in the methods and compositions described herein can be found in WO 2016/122889, WO 2016/172657, and WO 2016/172658, which in their entirety, is hereby incorporated by reference.

The dosage forms described herein can be manufactured using processes that are known to those of skill in the art. The dosage form may be suitable for any route of administration, including orally or parenterally, such as intravenously, intramuscularly, subcutaneously, intraorbitally, intracapsularly, intraperitoneally, intrarectally, intracisternally, intratumorally, intravasally, intradermally or by passive or facilitated absorption through the skin.

The dosage form may be a packet, such as any individual container that contains a glycan composition in the form of, e.g., a liquid (wash/rinse), a solid, a gel, a cream, an ointment, a powder, a tablet, a pill, a capsule, a lozenge, a suppository, a depository, a single-use applicator, a softgel or medical device (e.g. a syringe). For example, provided is also an article of manufacture, such as a container comprising a unit dosage form of the glycan composition, and a label containing instructions for use of such glycan composition.

The compositions provided herein can be in unit-dosage forms or multiple-dosage forms. A unit-dosage form, as used herein, refers to physically discrete unit suitable for administration to human in need thereof. In an embodiment, the unit-dosage form is provided in a package. Each unit-dose can contain a predetermined quantity of an active ingredient(s) sufficient to produce the desired therapeutic effect, in association with other pharmaceutical carriers or excipients. Examples of unit-dosage forms include ampoules, syringes, and individually packaged tablets and capsules. Unit-dosage forms can be administered in fractions or multiples thereof. A multiple-dosage form is a plurality of identical unit-dosage forms packaged in a single container, which can be administered in segregated unit-dosage form.

Kits

Kits also are contemplated. For example, a kit can comprise unit dosage forms of the glycan polymer preparation, and a package insert containing instructions for use of the glycan polymer in treatment of a gastrointestinal disorder or condition. The kits include a glycan polymer preparation in suitable packaging for use by a subject in need thereof. Any of the compositions described herein can be packaged in the form of a kit. A kit can contain an amount of a glycan polymer preparation (optionally additionally comprising a prebiotic substance, a probiotic bacterium, and/or a second therapeutic agent) sufficient for an entire course of treatment, or for a portion of a course of treatment. Doses of a glycan polymer preparation can be individually packaged, or the glycan polymer preparation can be provided in bulk, or combinations thereof. Thus, in one embodiment, a kit provides, in suitable packaging, individual doses of a glycan polymer preparation that correspond to dosing points in a treatment regimen, wherein the doses are packaged in one or more packets.

In one embodiment, the glycan polymer preparation can be provided in bulk in a single container, or in two, three, four, five, or more than five containers. For example, \each container may contain enough of a glycan polymer preparation for a particular week of a treatment program that runs for a month. If more than one bulk container is provided, the bulk containers can be suitably packaged together to provide sufficient glycan polymer preparation for all or a portion of a treatment period. The container or containers can be labeled with a label indicating information useful to the subject in need thereof or the physician performing the treatment protocol, such as, e.g. dosing schedules.

The glycan polymer preparation can be packaged with other suitable substances, such as probiotic bacteria, prebiotic substances or other substances, as described herein. The other substance or substances can be packaged separately from the glycan polymer preparation, or mixed with the glycan polymer preparation, or combinations thereof. Thus, in one embodiment, kits include a dosage form containing all the ingredients intended to be used in a course of treatment or a portion of a course of treatment, e.g., a glycan polymer preparation and optionally buffers, excipients, etc., a probiotic, prebiotic or a polymer agent. In one embodiment, a glycan polymer preparation is packaged in one package or set of packages, and additional components, such as probiotic bacteria, prebiotics, and therapeutic agents are packaged separately from the glycan polymer preparation.

Kits can further include written materials, such as instructions, expected results, testimonials, explanations, warnings, clinical data, information for health professionals, and the like. In one embodiment, the kits contain a label or other information indicating that the kit is only for use under the direction of a health professional. The container can further include scoops, syringes, bottles, cups, applicators or other measuring or serving devices.

Medical Food

Also provided herein are preparations of glycan polymers formulated as a medical food. Any glycan polymer preparation described herein may be formulated as a medical food as well as pharmaceutical compositions that comprise glycan polymer preparations.

A medical food is defined in section 5(b)(3) of the Orphan Drug Act (21 U.S.C. 360ee(b)(3)). Medical food is formulated to be consumed (oral intake) or administered enterally (e.g. feeding/nasogastric tube) under medical supervision, e.g. by a physician. It is intended for the specific dietary management of a disease or condition, such as, e.g. dysbiosis or a GI-tract disease. Medical foods as used herein do not include food that is merely recommended by a physician as part of an overall diet to manage the symptoms or reduce the risk of a disease or condition. Medical foods comprising a preparation of glycan polymers are foods that are synthetic (e.g., formulated and/or processed products, such as, being formulated for the partial or exclusive feeding of a patient by oral intake or enteral feeding by tube) and not naturally occurring foodstuff used in a natural state.

In some embodiments, the subject has limited or impaired capacity to ingest, digest, absorb, or metabolize ordinary foodstuffs or certain nutrients. In other embodiments, the subject has other special medically determined nutrient requirements, the dietary management of which cannot be achieved by the modification of the normal diet alone. Medical foods comprising a preparation of glycan polymers are administered to a subject in need thereof under medical supervision (which may be active and ongoing) and usually, the subject receives instructions on the use of the medical food. Medical foods may comprise one or more food additives, color additives, GRAS excipients and other agents or substances suitable for medical foods. Medical food preparations may be nutritionally complete or incomplete formulas.

Dietary Supplements

Any glycan polymer preparation described herein may be formulated as a dietary supplement, e.g, for use in a method described herein. Dietary supplements are regulated under the Dietary Supplement Health and Education Act (DSHEA) of 1994. A dietary supplement is a product taken by mouth that contains a “dietary ingredient” intended to supplement the diet. The “dietary ingredients” in these products may include, in addition to a glycan polymer preparation described herein, one or more of: vitamins, minerals, herbs or other botanicals, amino acids, and substances such as enzymes, organ tissues, glandulars, and metabolites. Dietary supplements can also be extracts or concentrates and may be found in many forms such as tablets, capsules, softgels, gelcaps, liquids, or powders. They can also be in other forms, such as a bar, but if they are, information on their label must not represent the product as a conventional food or a sole item of a meal or diet. DSHEA requires that every supplement be labeled a dietary supplement and not as a general food.

Food Ingredient

Any glycan polymer preparation described herein may be formulated as a food ingredient or food additive, e.g, for use in a method described herein. Food ingredients may be generally recognized as safe (GRAS) or may require FDA authorization. Glycan polymer preparations can be added to any desireable food, e.g. beverages (incl., e.g., fruit juices), dairy products (e.g., milk, yogurt, cheese), cereals (any grain products), bread, spreads, etc.

A glycan preparation may be formulated as a food. The term “food” as defined in the Federal Food, Drug and Cosmetic Act (21 U.S.C. Section 321(a)(f)) refers to articles used for food or drink for man or other animals, chewing gum, and articles used for components of any such article. Food is formulated to be consumed (oral intake). Foods may comprise, in addition to a glycan preparation, one or more food additives, color additives, GRAS excipients and other agents or substances suitable for foods. Food preparations may be nutritionally complete or incomplete formulas.

### Methods of Modulating Microbial Taxa

The compounds and compositions provided herein may be used in methods to modulate a bacterial taxa (e.g. 1, 2, 3, 4, 5 or more taxa) present in the microbiota of a subject. In some embodiments, modulation comprises a change in the structure of the microbiota, such as a change in the relative composition of a taxa or a change in the relative abundance of a taxa, e.g., relative to another taxa or relative to what would be observed in the absence of the modulation. In other embodiments, modulation comprises a change in a function of the microbiota, such as a change in gene expression, level of a gene product (e.g., RNA or protein), or metabolic output of the microbiota, or a change in a functional pathway of the host (e.g, a change in gene expression, level of a gene product, or metabolic output of a host cell or host process). Methods of modulating microbial taxa disclosed in WO 2016/122889 and WO 2016/172657 which are hereby incorporated by reference, are suitable for use in methods described herein.

The methods describe herein include administering to a subject a composition described herein, e.g., comprising a glycan composition described herein, in an amount effective to modulate taxa. In some embodiments, the abundance of a bacterial taxa may increase relative to other taxa (or relative from one point in time to another) when the composition is administered and the increase can be at least a 5%, 10%, 25% 50%, 75%, 100%, 250%, 500%, 750% increase or at least a 1000% increase. The abundance of a bacterial taxa may also decrease relative to other taxa (or relative from one point in time to another) when the composition is administered and the decrease can be at least a 5%, 10%, 25% 50%, 75%, 85%, 90%, 95%, 96%, 97%, 98%, 99% decrease, or at least a 99.9% decrease. Administration of the composition can modulate the abundance of the desired and/or non-desired bacterial taxa in the subject's gastrointestinal microbiota.

In some embodiments, the composition described herein, e.g., comprising a glycan composition described herein, modulates (e.g., increases or decreases) the growth of one or more bacterium, such as, e.g., those that belong to genera Bacteroides, Odoribacter, Parabacteroides, Alistipes, Blautia, Clostridium, Coprococcus, Dorea, Eubacterium, Lachnospira, Roseburia, Ruminococcus, Faecalibacterium, Oscillospira, and Subdoligranulum which can be found in the GI tract. In some embodiments, the composition described herein, e.g., comprising a glycan composition described herein, modulates (e.g., increases or decreases) the growth of one or more bacterium, such as, e.g., of the genus Akkermansia, Anaerofilum, Bacteroides, Blautia, Bifidobacterium, Butyrivibrio, Clostridium, Coprococcus , Dialister, Dorea, Fusobacterium, Eubacterium, Faecalibacterium, Lachnospira, Lactobacillus, Phascolarctobacterium, Peptococcus, Peptostreptococcus, Prevotella, Roseburia, Ruminococcus, and Streptococcus, and/or one or more of the species Akkermansia municiphilia, Christensenella minuta, Clostridium coccoides, Clostridium leptum, Clostridium scindens, Dialister invisus, Eubacterium rectal, Eubacterium eligens, Faecalibacterium prausnitzii, Streptococcus salivarius, and Streptococcus thermophilus.

In some embodiments, the composition described herein, e.g., comprising a glycan composition described herein modulates (e.g., increases or decreases) the growth of at least two bacterial taxa selected from Prevotella, Akkermansia, Bacteroides, Clostridium (Erysipelotrichaceae), Clostridium (Clostridiaceae), Bifidobacterium, Aggregatibacter, Clostridium (Peptostreptococcaveae), Parabacteroides, Lactobacillus, and Enterococcus.

In some embodiments, a composition described herein, e.g., comprising a glycan composition described herein, modulates (e.g., increases or decreases) the growth of one or more bacterial taxa residing in the GI tract, such as, e.g., those that belong to genera Bacteroides, Odoribacter, Parabacteroides, Alistipes, Blautia, Clostridium, Coprococcus, Dorea, Eubacterium, Lachnospira, Roseburia, Ruminococcus, Faecalibacterium, Oscillospira, and Subdoligranulum which can be found in the GI tract. In some embodiments, a composition described herein, e.g., comprising a glycan composition described herein, modulates (e.g., increases or decreases) the growth of one or more bacterial taxa, such as those that are thought to be associated with a healthy gastrointestinal state, e.g., one or more of the genus Akkermansia, Anaerofilum, Bacteroides, Blautia, Bifidobacterium, Butyrivibrio, Clostridium, Coprococcus , Dialister, Dorea, Fusobacterium, Eubacterium, Faecalibacterium, Lachnospira, Lactobacillus, Phascolarctobacterium, Peptococcus, Peptostreptococcus, Prevotella, Roseburia, Ruminococcus, and Streptococcus, and/or one or more of the species Akkermansia municiphilia, Christensenella minuta, Clostridium coccoides, Clostridium leptum, Clostridium scindens, Dialister invisus, Eubacterium rectal, Eubacterium eligens, Faecalibacterium prausnitzii, Streptococcus salivarius, and Streptococcus thermophilus. In some embodiments, the composition described herein, e.g., comprising a glycan composition described herein, modulates (e.g., increases or decreases) the growth of one or more bacterial taxa, such as taxa of the phylum Verrucomicrobia, e.g., those of the genus Akkermansia.

In some embodiments, a composition described herein, e.g., comprising a glycan composition described herein, modulates (e.g., increases or decreases) the growth of one or more bacterial taxa predominantly residing in the small intestine. For example, the composition described herein, e.g., comprising a glycan composition described herein, modulates one or more (2, 3, 4, 5, 6, 7, 8, 9, 10 or more) bacterial taxa that reside predominantly in the small intestine, such as, e.g. Actinobacteria, Firmicutes (Bacilli, Clostridia), and Proteobacteria (Alphaproteobacteria, Betaproteobacteria). In some embodiments, a composition described herein, e.g., comprising a glycan composition described herein, modulates one or more (2, 3, 4, 5, 6, 7, 8, 9, 10 or more) bacterial taxa that reside predominantly in the small intestine selected from the genera: Cryocola, Mycobacterium, Enterococcus, Lactococcus, Streptococcus, Turicibacter, Blautia, Coprococcus, Holdemania, Pseudoramibacter Eubacterium, Agrobacterium, Sphingomonas, Achromobacter, Burkholderia, and Ralstonia.

In some embodiments, a composition described herein, e.g., comprising a glycan composition described herein, modulates (e.g., increases or decreases) the growth of one or more bacterial taxa predominantly residing in the large intestine. For example, a composition described herein, e.g., comprising a glycan composition described herein, modulates one or more (2, 3, 4, 5, 6, 7, 8, 9, 10 or more) bacterial taxa that reside predominantly in the large intestine, such as, e.g. Bacteroidetes, Firmicutes (Clostridia), Verrucomicrobia, and Proteobacteria (Deltaproteobacteria). In some embodiments, a composition described herein, e.g., comprising a glycan composition described herein, modulates one or more (2, 3, 4, 5, 6, 7, 8, 9, 10 or more) bacterial taxa that reside predominantly in the large intestine selected from the genera: Bacteroides, Butyricimonas, Odoribacter, Parabacteroides, Prevotella, Anaerotruncus, Phascolarctobacterium, Ruminococcus, Bilophila, and Akkermansia.

In some embodiments, the composition described herein, e.g., comprising a glycan composition described herein, modulates (e.g., increases or decreases) the growth of one or more bacterial taxa predominantly residing in the cecum, such as, e.g., Actinobacteria, Bacteroides, Bacilli, Clostridia, Mollicutes, Alpha Proteobacteria, and Verrucomicrobia.

In some embodiments, a composition described herein, e.g., comprising a glycan composition described herein, modulates (e.g., increases or decreases) the growth of one or more bacterial taxa predominantly residing in the ascending colon, such as, e.g. Actinobacteria, Bacteroides, Bacilli, Clostridia, Fusobacteria, Beta Proteobacteria, Delta/Epsilon Proteobacteria, Gamma Proteobacteria, and Verrucomicrobia.

In some embodiments, the composition described herein, e.g., comprising a glycan composition described herein, modulates (e.g., increases or decreases) the growth of one or more bacterial taxa predominantly residing in the traverse colon, such as, e.g. Actinobacteria, Bacteroides, Clostridia, Mollicutes, Fusobacteria, and Gamma Proteobacteria.

In some embodiments, a composition described herein, e.g., comprising a glycan composition described herein, modulates (e.g., increases or decreases) the growth of one or more bacterial taxa predominantly residing in the descending colon, such as, e.g., Bacteroides, Clostridia, Mollicutes, Fusobacteria, Delta/Epsilon Proteobacteria and Verrucomicrobia.

In some embodiments, a composition described herein, e.g., comprising a glycan composition described herein, modulates (e.g., increases or decreases) the growth of one or more bacterial taxa predominantly residing in the sigmoid colon, such as, e.g. Actinobacteria, Bacteroides, Bacilli, Clostridia, Mollicutes, Alpha Proteobacteria, Beta Proteobacteria, and Verrucomicrobia.

In some embodiments, a composition described herein, e.g., comprising a glycan composition described herein, modulates (e.g., increases or decreases) the growth of one or more bacterial taxa predominantly residing in the rectum, such as, e.g. Bacteroides, Clostridia, Mollicutes, Alpha Probacteria, Gamma Proteobacteria, and Verrucomicrobia.

In some embodiments, a composition described herein, e.g., comprising a glycan composition described herein, modulates (e.g., stimulate/increase or suppress/decrease) the growth of one or more (e.g., 1, 2, 3, 4, 5, 6, 7, 8, 9, 10, or more) bacterial taxa of genera including, e.g. Alistipes, Akkermansia, Anaerofilum, Bacteroides, Blautia, Bifidobacterium, Butyrivibrio, Clostridium, Coprococcus, Dialister, Dorea, Fusobacterium, Eubacterium, Faecalibacterium, Lachnospira, Lactobacillus, Odoribacter, Oscillospira, Parabacteroides, Phascolarctobacterium, Peptococcus, Peptostreptococcus, Prevotella, Roseburia, Ruminococcus, and Streptococcus and Subdoligranulum.

In some embodiments, a composition described herein, e.g., comprising a glycan composition described herein, modulates (e.g., stimulate/increase or suppress/decrease) the growth of one or more (e.g., 1, 2, 3, 4, 5, 6, 7, 8, 9, 10, or more) microbial taxa of genera Akkermansia, Anaerofilum, Bacteroides, Blautia, Bifidobacterium, Butyrivibrio, Clostridium, Coprococcus, Dialister, Dorea, Fusobacterium, Eubacterium, Faecalibacterium, Lachnospira, Lactobacillus, Phascolarctobacterium, Peptococcus, Peptostreptococcus, Prevotella, Roseburia, Ruminococcus, and Streptococcus and of the species Akkermansia municiphilia, Christensenella minuta, Clostridium coccoides, Clostridium leptum, Clostridium scindens, Dialister invisus, Eubacterium rectal, Eubacterium eligens, Faecalibacterium prausnitzii, Streptococcus salivarius, and Streptococcus thermophilus.

In some embodiments, a composition described herein, e.g., comprising a glycan composition described herein, modulates (e.g. substantially increase or substantially decrease) the growth (and the total number) of (or substantially increase or substantially decrease the relative representation/abundance in the total (gastrointestinal) community) of one or more of (e.g. 1, 2, 3, 4, 5, 6, 7, 8, 9, 10, or more) bacterial taxa listed in Tables 1-3 or 4-6.

In some embodiments, a composition described herein, e.g., comprising a glycan composition described herein, substantially increases the growth, e.g. the total number or the relative representation/abundance in the total (gastrointestinal) community) of one or more of (e.g. 1, 2, 3, 4, 5, 6, 7, 8, 9, 10, or more) bacterial taxa listed in Tables 1-3 or 4-6.

In some embodiments, a composition described herein, e.g., comprising a glycan composition described herein, substantially decreases the growth, e.g. the total number or the relative representation/abundance in the total (gastrointestinal) community) of one or more of (e.g. 1, 2, 3, 4, 5, 6, 7, 8, 9, 10, or more) bacterial taxa listed in Tables 1-3 or 4-6.

In some embodiments, a composition described herein, e.g., comprising a glycan composition described herein, substantially increases and decreases the growth, e.g. the total number or the relative representation/abundance in the total (gastrointestinal) community) of one or more of (e.g. 1, 2, 3, 4, 5, 6, 7, 8, 9, 10, or more) bacterial taxa listed in Tables 1-3 or 4-6.

In certain embodiments, the ratio of certain bacterial taxa or their relative abundance may be shifted. Such shifts may be measured with respect to the ratio present in the subject prior to administration of the pharmaceutical glycan composition, or to a control group not taking the pharmaceutical glycan composition.

### Proteomic Analysis of Microbial Populations

Preparations of glycan compositions may be selected based on their ability to modulate the expression of microbial proteins, e.g., enzymes, associated with the processing of exogenous substances as described, e.g., in Tables 1-3. Suitable methods for proteomic analysis of microbial populations can be found in WO 2016/122889 and WO 2016/172657, which are hereby incorporated by reference. In some embodiments, proteomic analysis can be performed following protocols described in e.g., Cordwell, Exploring and exploiting bacterial proteomes, Methods in Molecular Biology, 2004, 266:115.

Identification of Microbial (e.g. Bacterial) Constituents

Microbial modulation (e.g., of representation/abundance of a taxa) by the glycan compositions described herein, e.g., occurring in vivo in the GI tract can be analyzed using any number of methods known in the art and described herein. Suitable methods can be found in WO WO 2016/122889, WO 2016/172657, and WO 2016/172658, which are hereby incorporated by reference. In some embodiments, quantitative PCR (qPCR) can be used as a method to determine whether a glycan composition can result in a shift of the population of bacteria in the GI tract.

In some embodiments, microbial constituents can be identified by characterizing the DNA sequence of microbial 16S small subunit ribosomal RNA gene (16S rRNA gene). In other embodiments, a microbial composition can be identified by characterizing nucleotide markers or genes, in particular highly conserved genes (e.g., “house-keeping” genes), or a combination thereof, or whole genome shotgun sequence (WGS).

### Administration to a Subject

The glycan compsitions, pharmaceutical compositions and therapeutic agents described herein can be administered to a subject in need thereof by various routes (e.g., systemically or locally) including, for example, orally or parenterally, such as intravenously, intramuscularly, subcutaneously, intraorbitally, intracapsularly, intraperitoneally, intrarectally, intracisternally, intratumorally, intravasally, intradermally or by passive or facilitated absorption through the skin. The therapeutic agents can be administered locally to the site of a pathologic condition, for example, intravenously or intra-arterially into a blood vessel supplying a tumor. In some embodiments, the glycan composition is administered enterically. This includes oral administration, or by an oral or nasal tube (including nasogastric, nasojejunal, oral gastric, or oral jejunal). In other embodiments, administration includes rectal administration (including enema, suppository, or colonoscopy). Methods of administering to a subject sutiable for use with methods and compositions described herein can be found in WO 2016/122889, WO 2016/172657, and WO 2016/172658, which in their entirety, are hereby incorporated by reference.

Active compounds and pharmaceutical agents, e.g., prebiotic substances, probiotic bacteria or drugs, may be administered separately, e.g., prior to, concurrent with or after administration of the glycan compositions and not as a part of the pharmaceutical composition or medical food or dietary supplement (e.g. as a co-formulation) of glycan compositions. In some embodiments, pharmaceutical compositions or medical foods comprising preparations of glycan compositions are administered in combination with a recommended or prescribed diet, e.g. a diet that is rich in probiotic and/or prebiotic-containing foods, such as it may be determined by a physician or other healthcare professional.

### Methods of Treating

Provided herein are methods of treating a disease, disorder, condition, or pathological condition comprising administering to a subject in need thereof a glycan composition, e.g., a glycan composition described herein. Also provided herein are exogenous substances (e.g., pharmaceutical agents) for use in any method described herein. The exogenous substance can be included in a composition comprising the glycan composition or can be administered/formulated separately. In embodiments, the pharmaceutical agent is used to treat a disease, disorder, condition, or pathological condition described herein.

Diseases and disorders that can be treated with methods and compositions described herein can be found in WO 2016/122889, WO 2016/172657, and WO 2016/172658, which in their entirety, is hereby incorporated by reference. Exemplary diseases, disorders, conditions, or pathological conditions can include but are not limited to: a proliferative disease (e.g., cancer), a dysbiosis, an infectious disease, a metabolic disease, a neurodegenerative disease, an allergy, and the like.

Diseases, disorders, and conditions that can be treated with methods described herein include: pain, migraines, arthritis, cancer, colon and rectum cancer, bacterial infection, viral infection, HIV, hepatitis, hepatitis C, fungal infection, nematode infection, hookworm infection, osteoporosis, pain related to cancer, diabetes, blood sugar imbalance, seizures, panic disorder, anxiety, heart conditions, heart failure, arrhythmia, high blood pressure, angina, chest pain, thrombocytopenia, aplastic anemia, Parkinson's disease and Parkinson's like symptoms, diarrhea, high cholesterol and triglyceride levels, ADHD, recreational drug use, insomnia, ulcers, gastroesophageal reflux disease (GERD), heart burn, drug toxicity (e.g., 5-fluorouracil-induced gastrointestinal toxicity), schizophrenia, bipolar disorder, irritability caused by autism, constipation, and epilepsy.

In some embodiments, the pharmaceutical composition comprising a glycan composition is administered prior to, concomitant with or after administration of the (e.g. anti-cancer) drug or non-drug (e.g., anti-cancer) treatment, administration of which induces the symptoms.

In one embodiment, a method of lowering toxicity of a drug treatment (e.g., an anti-cancer drug treatment) in a subject is provided. The method includes: a) administering a pharmaceutical composition comprising a glycan composition to a subject who has received the drug treatment; b) administering the drug treatment to a subject who has been treated with a pharmaceutical composition comprising a glycan composition; or c) administering a pharmaceutical composition comprising a glycan composition and administering the drug treatment, to a subject, thereby treating the subject. In some embodiments, the toxicity is dose-limiting toxicity. In some embodiments, the method increases the tolerance of the subject to drug treatment, e.g. an anti-cancer drug treatment.

In some embodiments, dose limiting toxicity prevents subjects from being treated with the maximal efficacious dose of a drug. As one example of dose-limiting toxicity, diarrhea can be caused by the chemotherapy drugs irinotecan and 5-fluoruracil. In some embodiments, glycan composition is administered to treat dose limiting toxicity, e.g., to increase the dose that is tolerated by the subject. In some embodiments, tolerability is increased by limiting one or more digestive abnormalities associated with the respective efficacious drug dose.

All publications, patents, and patent applications cited or referenced in this specification are herein incorporated by reference to the same extent as if each independent publication or patent publication was specifically and individually indicated to be incorporated by reference.

### EXAMPLES

The invention is further illustrated by the following examples. The examples are provided for illustrative purposes only and are not to be construed as limiting the scope or content of the invention in any way. The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); Green & Sambrook et al., Molecular Cloning: A Laboratory Manual, 4th Edition (Cold Spring Harbor Laboratory Press, 2012); Colowick & Kaplan, Methods In Enzymology (Academic Press); Remington: The Science and Practice of Pharmacy, 22nd Edition (Pharmaceutical Press, 2012); Sundberg & Carey, Advanced Organic Chemistry: Parts A and B, 5th Edition (Springer, 2007).

### Example 1

### Effect of Glycans on Microbial Populations Ex Vivo

To determine the desired composition of glycans, bacterial cultures were grown in the presence of candidate glycans and assayed for their growth, community composition (e.g., by 16S rRNA gene sequencing), production of metabolites, and phenotypic or transcriptomic properties. Desired glycans were selected based on their ability to elicit desired properties within the bacterial culture. Bacterial cultures include monocultures, mixed cultures, cultures isolated from humans or laboratory animal models, cultures isolated from a human or laboratory animal model and spiked with an isolate or collection of isolates, or cultures isolated from a human or laboratory animal model and depleted of a collection of species (for example, by application of an antibiotic). This assay can be performed in the presence of antibiotics or other test compounds. The results obtained from the in vitro assays are compared with those obtained after treating humans with glycans or administering the glycans to a laboratory animal establishing the in vitro—in vivo correlation of results.

### Example 2

### Growth of Beta-Glucuronidase-Associated Strains, Non-Associated Strains, and Other Gut Commensals on Several Glycans

An in vitro assay was performed to assess the ability of various bacterial strains, including commensals of the gastrointestinal tract, to utilize different glycans as growth substrates. This assay was designed to assess the ability of selected glycans to promote the growth of microbiota associated with beta-glucuronidase production, those not associated with beta-glucuronidase production, and other gut commensals. Lactobacillus gasseri was handled aerobically, and all other bacterial strains were handled at all steps in an anaerobic chamber (AS-580, Anaerobe Systems) featuring a palladium catalyst. The chamber was initially made anaerobic by purging with an anaerobic gas mixture of 5% hydrogen, 5% carbon dioxide and 90% nitrogen and subsequently maintained in an anaerobic state using this same anaerobic gas mixture. Anaerobicity of the chamber was confirmed daily using Oxoid anaerobic indicator strips that change color in the presence of oxygen. All culture media, assay plates, other reagents and plastic consumables were pre-reduced in the anaerobic chamber for 24-48 hours prior to contact with bacteria. Glycans glu33gal33fuc33, gal100, glu50gal50, man80glu20, man60glu40, man80gal20, glu100, man100, man52glu29gal19, man66gal33, xyl75ara25, glu80man20 and glu60man40, a commercially available control, FOS (Nutraflora FOS; NOW Foods, Bloomingdale Ill.), and dextrose were prepared at 5% w/v in water, filter-sterilized and added to Costar 3370 assay plates for a final concentration of 0.5% w/v in the assay, with each glycan assayed in 2-4 non-adjacent wells and dextrose and water supplied as positive and negative controls.

Bacterial isolates were obtained from the American Type Culture Collection (ATCC) and Leibniz Institute DSMZ-German Institute of Microorganisms and Cell Cultures (DSMZ). Cultures of strains Ruminococcus obeum 0ATCC 29714 “ROB.74”, Bacteroides caccae ATCC 43185 “BCA.26”, Bacteroides thetaiotaomicron ATCC 29741 “BTH.8”, Bacteroides cellulosilyticus DSM 14838 “BCE.71”, Parabacteroides distasonis ATCC 8503 “PDI.6”, Bacteroides vulgatus ATCC 8482 “BVU.10”, Clostridium scindens ATCC 35704 “CSC.32”, Dorea formicigenerans ATCC 27755 “DFO.36”, and the Bifidobacteria Bifidobacterium longum ATCC 15707 “BL0.16” and Bifidobacterium longum DSM 20088 “BL0.83”, were grown anaerobically in Chopped Meat Glucose broth (CMG, Anaerobe Systems), a pre-reduced enriched medium including lean ground beef, enzymatic digest of casein, yeast extract, potassium phosphate, dextrose, cysteine, hemin and Vitamin K1, for 18-48 hours at 37° C. Cultures of Lactobacillus gasseri ATCC 33323 “LGA.44” were grown aerobically in CMG for 18-48 hours at 37° C. Inocula were prepared by determining the optical density of each culture at 600 nM (0D600) in a Costar 3370 polystyrene 96-well flat-bottom assay plate using a Biotek Synergy 2 plate reader with Gen5 2.0 All-In-One Microplate Reader Software according to manufacturer's protocol, and diluting the cells to OD600 0.01 final in defined and semi-defined media that were prepared without sugars. B. vulgatus, D. formicigenerans, P. distasonis, and B. longum isolates were tested in 900 mg/L sodium chloride, 26 mg/L calcium chloride dihydrate, 20 mg/L magnesium chloride hexahydrate, 10 mg/L manganese chloride tetrahydrate, 40 mg/L ammonium sulfate, 4 mg/L iron sulfate heptahydrate, 1 mg/L cobalt chloride hexahydrate, 300 mg/L potassium phosphate dibasic, 1.5 g/L sodium phosphate dibasic, 5 g/L soidum bicarbonate, 0.125 mg/L biotin, 1 mg/L pyridoxine, 1 m/L pantothenate, 75 mg/L histidine, 75 mg/L glycine, 75 mg/L tryptophan, 150 mg/L arginine, 150 mg/L methionine, 150 mg/L threonine, 225 mg/L valine, 225 mg/L isoleucine, 300 mg/L leucine, 400 mg/L cysteine, and 450 mg/L proline (Theriot CM et al. Nat Commun. 2014; 5:3114), supplemented with 0-5% (v/v) CMG. B. thetaiotaomicron, B. caccae and B. cellulosyliticus were tested in 100 mM potassium phosphate buffer (pH 7.2), 15 mM sodium chloride, 8.5 mM ammonium sulfate, 4 mM L-cysteine, 1.9 μM hematin, 200 μM L-histidine, 100 μM magnesium chloride, 1.4 μM iron sulfate heptahydrate, 50 μM calcium chloride, 1 μg/mL vitamin K3 and 5 ng/mL vitamin B12 (Martens EC et al. Cell Host & Microbe 2008; 4, 447-457). L. gasseri, C. scindens and R. obeum were tested in 10 g/L tryptone peptone, 5 g/L yeast extract, 0.5 g/L L-cysteine hydrochloride, 0.1 M potassium phosphate buffer pH 7.2, 1 μg/mL vitamin K3, 0.08% w/v calcium chloride, 0.4 μg/mL iron sulfate heptahydrate, 1 μg/mL resazurin, 1.2 μg/mL hematin, 0.2 mM histidine, 0.05% Tween 80, 0.5% meat extract (Sigma), 1% trace mineral supplement (ATCC), 1% vitamin supplement (ATCC), 0.017% v/v acetic acid, 0.001% v/v isovaleric acid, 0.2% v/v propionic acid and 0.2% v/v N-butyric acid (Romano KA et al. mBio 2015; 6(2):e02481-14) supplemented with 0-5% CMG. Bacteria were exposed to glycans ara50gal50, glu33gal33fuc33, glu50gal50, gal100, glu100, xyl100, ara100, ara60xyl40, glu80man20, glu60man40, man52glu29gal19, man100, xyl75ara25, commercial FOS and dextrose at a final concentration of 0.5% w/v in 96-well microplates, 200 μL final volume per well, at 37° C. for 18-48 hours, anaerobically. OD600 measurements for each isolate at the end of the incubation period were obtained using a Biotek Synergy2 reader with Gen5 2.0 software according to manufacturer's specifications.

Measurements were normalized by dividing the OD600 readings of the isolate on test glycans by the average OD600 of the isolate in medium supplemented with 0.5% w/v dextrose to facilitate comparison of glycan utilization by strains that grow within significantly different OD600 ranges. Table 8 summarizes the results obtained.

TABLE 8
Growth of beta-glucuronidase-associated strains, non-associated
strains, and other gut commensals on glycans.

| | Gut Commensals, Average Normalized Growth Values | | | | | | | | | | |
|---|---|---|---|---|---|---|---|---|---|---|---|
| | β-Glucuronidase-Associated | | | | Non-Associated | | | Other | | | |
| ID | LGA.44 | ROB.74 | BLO.16 | BLO.83 | BTH.8 | BVU.10 | DFO.36 | BCA.26 | BCE.71 | CSC.32 | PDI.6 |
| 1 | − | − | − | − | ++ | + | + | ++ | + | − | ++ |
| 2 | − | − | − | − | + | − | − | +++ | + | + | ++ |
| 3 | − | − | − | − | ++ | − | − | − | − | − | + |
| 4 | + | − | − | − | +++ | − | − | + | + | + | +++ |
| 5 | + | − | − | − | ++ | + | + | +++ | ++ | − | +++ |
| 6 | + | − | − | − | +++ | + | − | ++ | ++ | − | +++ |
| 7 | + | − | − | − | ++ | − | + | ++ | ++ | − | ++ |
| 8 | + | − | − | − | ++ | − | − | + | + | − | +++ |
| 9 | + | + | − | − | +++ | + | + | +++ | ++ | − | +++ |
| 10 | + | + | − | − | +++ | + | − | +++ | ++ | − | +++ |
| 11 | − | − | − | − | + | − | − | + | + | − | − |
| FOS | − | +++ | +++ | +++ | ++ | +++ | + | +++ | +++ | + | +++ |
| 12 | ++ | ++ | ++ | + | +++ | ++ | +++ | +++ | +++ | + | +++ |
| 13 | ++ | + | + | − | +++ | ++ | ++ | ++ | ++ | + | +++ |

L. gasseri, R. obeum and B. longum belong to the same species or genera as reported β-glucuronidase producers and thus are associated with β-glucuronidase production (Russell, W. M. and Klaenhammer, T. R. Identification and cloning of gusA, encoding a new β-glucuronidase from Lactobacillus gasseri ADH. Appl Environ. Microbiol. 2001; Beaud et al, Genetic characterization of the β-glucuronidase enzyme from a human intestinal bacterium, Ruminococcus gnavus. Microbiology 2005; Roy, D & Ward, P. Rapid detection of Bifidobacterium dentium by enzymatic hydrolysis of (3-glucuronide substrates. J Food Protect. 1992.). B. thetaiotaomicron and B. vulgatus reportedly have no detectable β-glucuronidase activity in vitro (Dabek et al, Distribution of β-glucosidase activity and of β-glucuronidase gene gus in human colonic bacteria. FEMS Microbiol Ecol 2008), and no β-glucuronidase is found in the NCBI protein database for Dorea species, including D. formicigenerans; consequently, these 3 isolates are considered to be non-associated with β-glucuronidase production.

In the assay, a number of glycans supported growth of non-β-glucuronidase associated strains and other gut commensals better than β-glucuronidase associated strains, producing relatively low average normalized growth values with the β-glucuronidase associated strains.

Glu33gal33fuc33 and gal100 did not support growth of any of the β-glucuronidase associated strains in the assay but supported growth of 5-6 non-βglucuronidase associated and other gut commensal strains with average normalized growth values greater than 0.15. In the assay, glu50gal50, man80glu20, man60glu40, man80gal20, glu100, man100, man52glu29gal19 and man66gal33 supported normalized growth values of at least 0.3 with the non-β-glucuronidase associated strain B. thetaiotaomicron and with 1-3 other gut commensals, while they supported no relatively weak to no growth of β-glucuronidase producers, with average normalized growth values <0.3 for L. gasseri and <0.15 for R. obeum and B. longum.

Bacterial β-glucuronidases are associated with toxicity of some drugs. For example, microbial β-glucuronidases in the gut convert a non-toxic form of the cancer drug irinotecan to a form that exhibits toxicity toward intestinal epithelial cells (Spanogiannopoulos P. et al. The microbial pharmacists within us: a metagenomics view of xenobiotic metabolism. Nature Reviews Microbiology vol 14, May 2016). Administering a glycan that selectively supports growth of non-β-glucuronidase associated strains to a patient undergoing irinotecan chemotherapy may reduce the relative abundance of β-glucuronidase-producing strains in the gut and thereby reduce irinotecan-associated diarrhea.

TABLE 9
Key to Example 2 Glycans
Key to Glycans

| glycan # | composition |
|---|---|
| 1 | glu33gal33fuc33 |
| 2 | gal100 |
| 3 | glu50gal50 |
| 4 | man80glu20 |
| 5 | man60glu40 |
| 6 | Man80gal20 |
| 7 | glu100 |
| 8 | man100 |
| 9 | man52glu29gal19 |
| 10 | Man66gal33 |
| 11 | xyl75ara25 |
| 12 | glu80man20 |
| 13 | glu60man40 |

TABLE 10
Key to Example 2 Symbols

| Symbol | NGV |
|---|---|
| − | <0.15 |
| + | 0.15-0.3 |
| ++ | 0.3-0.6 |
| +++ | >0.6 |

### Example 3

### Glycan-Supported Growth of Bacteria Associated with Phytoestrogen Metabolism

An in vitro assay was performed to assess the ability of various bacterial strains, including commensals of the gastrointestinal tract, to utilize different glycans as growth substrates. This assay was designed to assess the ability of selected glycans to promote the growth of microbiota associated with phytoestrogen metabolism, which has been associated with protective effects against breast cancer. Bacterial strains were handled at all steps in an anaerobic chamber (AS-580, Anaerobe Systems) featuring a palladium catalyst. The chamber was initially made anaerobic by purging with an anaerobic gas mixture of 5% hydrogen, 5% carbon dioxide and 90% nitrogen and subsequently maintained in an anaerobic state using this same anaerobic gas mixture. Anaerobicity of the chamber was confirmed daily using Oxoid anaerobic indicator strips that change color in the presence of oxygen. All culture media, assay plates, other reagents and plastic consumables were pre-reduced in the anaerobic chamber for 24-48 hours prior to contact with bacteria. Glycans glu80man20, glu60man40, glu100, gal100, man80glu20, glu33gal33fuc33, man60glu40, man80gal20, man66gal33, man100, man52glu29gal19, xyl75ara25 and xyl100 were prepared at 5% w/v in water, filter-sterilized and added to Costar 3370 assay plates for a final concentration of 0.5% w/v in the assay, with each glycan assayed in two non-adjacent wells and dextrose and water supplied as positive and negative controls.

Bacterial isolates were obtained from the American Type Culture Collection (ATCC) and Leibniz Institute DSMZ-German Institute of Microorganisms and Cell Cultures (DSMZ). Cultures of Blautia producta ATCC 27340 “BPR.22”, Clostridium scindens ATCC 35704 “CSC.32”, Enterococcus faecium ATCC 700221 “EFM.66” and the Bifidobacteria Bifidobacterium longum ATCC 15707 “BL0.16” and Bifidobacterium longum DSM 20088 “BL0.83”, were grown anaerobically in Chopped Meat Glucose broth (CMG, Anaerobe Systems), a pre-reduced enriched medium including lean ground beef, enzymatic digest of casein, yeast extract, potassium phosphate, dextrose, cysteine, hemin and Vitamin K1, for 18-48 hours at 37° C. Inocula were prepared by determining the optical density of each culture at 600 nM (OD₆₀₀) in a Costar 3370 polystyrene 96-well flat-bottom assay plate using a Biotek Synergy 2 plate reader with Gen5 2.0 All-In-One Microplate Reader Software according to manufacturer's protocol and diluting the cells to OD₆₀₀ 0.01 final in defined and semi-defined media that were prepared without sugars. B. producta, E. faecium and B. longum isolates were tested in 900 mg/L sodium chloride, 26 mg/L calcium chloride dihydrate, 20 mg/L magnesium chloride hexahydrate, 10 mg/L manganese chloride tetrahydrate, 40 mg/L ammonium sulfate, 4 mg/L iron sulfate heptahydrate, 1 mg/L cobalt chloride hexahydrate, 300 mg/L potassium phosphate dibasic, 1.5 g/L sodium phosphate dibasic, 5 g/L sodium bicarbonate, 0.125 mg/L biotin, 1 mg/L pyridoxine, 1 m/L pantothenate, 75 mg/L histidine, 75 mg/L glycine, 75 mg/L tryptophan, 150 mg/L arginine, 150 mg/L methionine, 150 mg/L threonine, 225 mg/L valine, 225 mg/L isoleucine, 300 mg/L leucine, 400 mg/L cysteine, and 450 mg/L proline (Theriot CM et al. Nat Commun. 2014; 5:3114), supplemented with 0-5% (v/v) CMG. C. scindens was tested in 10 g/L tryptone peptone, 5 g/L yeast extract, 0.5 g/L L-cysteine hydrochloride, 0.1 M potassium phosphate buffer pH 7.2, 1μg/mL vitamin K3, 0.08% w/v calcium chloride, 0.4 μg/mL iron sulfate heptahydrate, 1 μg/mL resazurin, 1.2 μg/mL hematin, 0.2 mM histidine, 0.05% Tween 80, 0.5% meat extract (Sigma), 1% trace mineral supplement (ATCC), 1% vitamin supplement (ATCC), 0.017% v/v acetic acid, 0.001% v/v isovaleric acid, 0.2% v/v propionic acid and 0.2% v/v N-butyric acid (Romano K A et al. mBio 2015; 6(2):e02481-14). Bacteria were exposed to glu80man20, glu60man40, glu100, gal100, man80glu20, glu33gal33fuc33, man60glu40, man80gal20, man66gal33, man100, man52glu29gal19, xyl75ara25, xyl100 and dextrose at a final concentration of 0.5% w/v in 96-well microplates, 200 ᵢ.t.L final volume per well, at 37° C. for 18-48 hours, anaerobically. OD₆₀₀ measurements for each isolate at the end of the incubation period were obtained using a Biotek Synergy2 reader with Gen5 2.0 software according to manufacturer's specifications. Measurements were normalized by dividing the OD₆₀₀ readings of the isolate on test glycans by the average OD₆₀₀ of the isolate in medium supplemented with 0.5% w/v dextrose to facilitate comparison of glycan utilization by strains that grow within significantly different OD₆₀₀ ranges. Table 11 summarizes the results obtained.

TABLE 11
Glycan-supported growth of bacteria associated
with phytoestrogen metabolism

| | Phytoestrogen Metabolism-Associated Bacteria, | | | | |
|---|---|---|---|---|---|
| | Average Normalized Growth | | | | |
| glycan # | BPR.22 | EFM.66 | CSC.32 | BLO.16 | BLO.83 |
| 1 | +++ | +++ | + | ++ | + |
| 2 | +++ | ++ | + | + | − |
| 3 | +++ | ++ | − | + | + |
| 4 | +++ | + | + | − | − |
| 5 | +++ | + | + | − | − |
| 6 | +++ | + | − | − | − |
| 7 | +++ | + | − | − | − |
| 8 | +++ | + | − | − | − |
| 9 | +++ | + | − | − | − |
| 10 | +++ | + | − | − | − |
| 11 | +++ | + | − | − | − |
| 12 | +++ | − | − | − | − |
| 13 | ++ | − | − | − | − |

TABLE 12
Key to Example 3 Symbols

| Symbol | NGV |
|---|---|
| − | <0.15 |
| + | 0.15-0.3 |
| ++ | 0.3-0.6 |
| +++ | >0.6 |

Bifidobacterium species and E. faecium have been reported to metabolize the phytoestrogen daidzin, an isoflavone, to equol. B. producta, C. scindens and Enterococcus faecalis have been reported to metabolize plant lignans, a different class of phytoestrogens, to enterodiol and enterolactone (Spanogiannopoulos P. et al. The microbial pharmacists within us: a metagenomics view of xenobiotic metabolism. Nature Reviews Microbiology vol 14, May 2016). B. longum, B. producta, C. scindens and E. faecalis thus are associated with metabolism of phytoestrogens.

In the assay, different glycans supported different levels of growth of different numbers of these bacterial strains associated with phytoestrogen metabolism. In the assay, glu80man20, glu60man40 and glu100 supported growth of 4-5 of 5 strains, producing average normalized growth values of at least 0.15. Glu100 and gal100 supported growth of 3 strains, with average normalized growth values of at least 0.15 in the assay. Glu33gal33fuc33, man60glu40, man80gal20, man66gal33, man100 and man52glu29gal19 supported growth of 2 strains in the assay, and xyl75ara25 and xyl100 supported growth of 1 strain in the assay.

Metabolism of phytoestrogens such as plant lignans and isoflavones by bacteria in the gut to molecules that bind estrogen receptors may have protective effects against breast cancer (Mabrook, H. B et al. Lignan transformation by gut bacteria lowers tumor burden in a gnotobiotic rat model of breast cancer. Carcinogenesis 33, 203-208 (2012)). Administration of glycans that promote the growth of bacterial species associated with phytoestrogen metabolism thus may elicit protective effects against breast cancer and/or other types of cancer in humans.

TABLE 13
Key to Example 3 Glycans
Key to glycans

| glycan # | composition |
|---|---|
| 1 | glu80man20 |
| 2 | glu60man40 |
| 3 | glu100 |
| 4 | gal100 |
| 5 | man80glu20 |
| 6 | glu33gal33fuc33 |
| 7 | man60glu40 |
| 8 | man80gal20 |
| 9 | man66gal33 |
| 10 | man100 |
| 11 | man52glu29gal19 |
| 12 | xyl75ara25 |
| 13 | xyl100 |

### Example 4

### Modification of Exogenous Substances by Microbiota

Untargeted metabolomics was performed on 30-50 mg of cecal contents from mice fed either a High Fat diet (Research Diets D12492) or High Fat diet+glycan using Metabolon's LC-MS based DiscoveryHD4 platform.

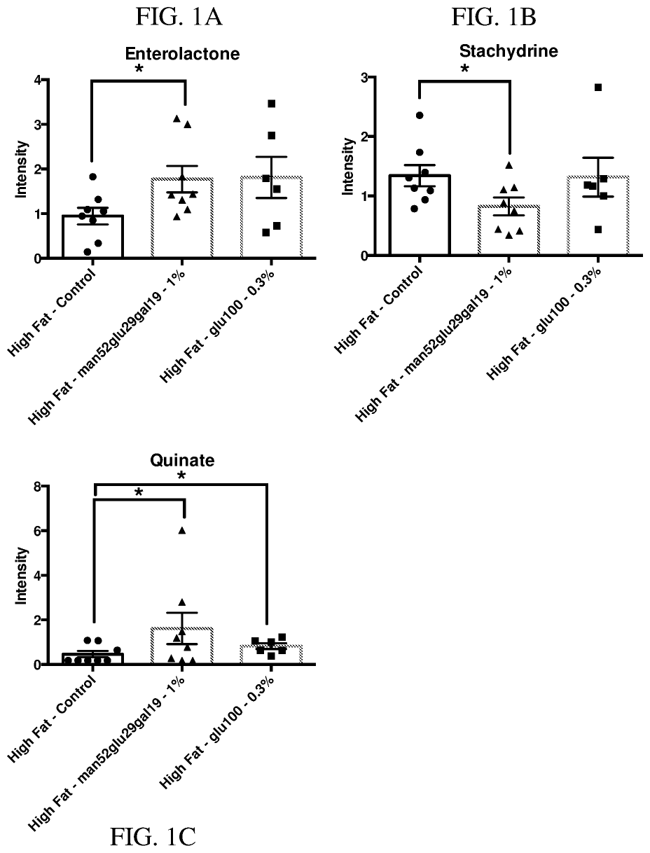

Three exogenous substances, enterolactone, stachydrine, and quinate, were identified that were modified by the microbiota with the addition of man52glu29gal19 at 1% in the drinking water. See FIGS. 1A, 1B, and 1C.

Enterolactone, a mammalian lignin formed by the action of intestinal bacteria from plant lignin precursors present in the diet, was increased in animals treated with man52glu29gal19. It is believed that enterolactone may possess beneficial health effects in humans. Reduction in enterolactone has been associated with a number of human pathologies. For example, it has been demonstrated that individuals with Type 2 Diabetes have significant reduction of urinary enterolactone when compared to healthy controls (Sun et al., Diabetes Care, 2014). Enterolactone has also been shown to have some anticarcinogenic action as the administration of enterolactone has been shown to inhibit or delay the growth of experimental mammary cancer (Saarinen et al., Molecular Nutrition & Food Research, 2013). In epidemiological studies, lower concentrations of enterolactone have been observed in breast cancer patients compared to healthy controls, which may suggest that enterolactone is anti-carcinogenic. Enterolactone and lignans may also be protective of cardiovascular disease.

In addition, stachydrine was reduced with the addition of man52glu29gal 19 at 1% in the drinking water. Stachydrine is also called proline betaine. Proline betaine is a glycine betaine analogue found in many citrus foods. Stachydrine has been shown to be a marker of citrus intake in the diet (Guertin et al., AJCN, 2014). Elevated levels of proline betaine in human urine are found after the consumption of citrus fruits and juices.

Further, quinate was found to increase with the addition of either glu100 or man52glu29gal19 to a high fat diet. Quinate is an abundant plant product that is utilized as a carbon source for a number of microorganisms (Teramoto, et al., Appl. Environ. Microbiol., 2009). These data suggest that microbial turnover of various exogenous substances by microbial taxa (such as those residing in the gastrointestinal tract, e.g., of a human subject) can be modulated by administering a glycan composition (such as described herein) to a subject in an amount effective to modulate (e.g., increase or decrease) the level of the exogenous substance in the subject.

### Example 5

### Modulation of Beta-Glucuronidase Levels in the Gastrointestinal Tract of Human Subjects and Mouse Model by Glycans for the Reduction/Prevention of Irinotecan Toxicity

Patients with histologically or cytologically confirmed colorectal adenocarcinoma are randomized to receive a glycan treatment or placebo for one week prior to administration of a chemotherapeutic regimen consisting of FOLFIRI [folinic acid (leucovorin) 400 mg/m\hat{ }2 by vein (IV) Day 1; 5-FU 400 mg/m\hat{ }2 IV injection Day 1 immediately followed by 2.4 g/m\hat{ }2 IV over 46 hours over Days 1-3; Irinotecan 180 mg/m\hat{ }2 IV on Day 1]. Primary treatment outcome will be the proportion of patients on glycan or placebo that experience Grade 2 or greater diarrhea in the week following the FOLFIRI regimen. A first secondary outcome will be the proportion of patients on glycan or placebo that experience Grade 3 or greater diarrhea in the week following the FOLFIRI regimen. A second secondary outcome will be the proportion of patients on glycan or placebo that require an antidiarrheal treatment (such as Loperamide) in the week following the FOLFIRI regimen. The glycan treatment is expected to increase in the gastrointestinal tract of patients the growth of bacteria that do not express (β-Glucuronidase relative to bacteria that do express (β-Glucuronidase, and thus will proportionally decrease the concentration of (β-Glucuronidase enzymes in the gastrointestinal tract that can activate the toxic metabolite of irinotecan (SN38). For example, glycan 1 is expected to increase the growth of gut commensal bacterial strains, BTH. 8, BVU. 10, and DFO. 36, while not increasing the growth of LGA. 44, ROB. 74, BLO. 16, and BLO. 83 (see Example 2 and Table 8).

A proof of concept experiment was conducted to elucidate the effects of glycans on chemotherapy-induced toxicity in a mouse model using irinotecan chemotherapeutic.

In this study, 150 male BALB/c mice (Charles River Laboratories) were randomized into 13 mice per group, for 7 groups, 3 groups of 18 mice, and 1 group of 5 mice and were group housed. The mice were allowed to acclimate for five days following arrival. One group of 5 mice received irinotecan. All mice received special diet, with monitoring of daily body weights starting at day −7.

Mice received oral gavage for 14 days with deionized water or glycan composition. Starting on Day −6, animals in glycan-treated groups began treatment with glycan compositions or FOS at 6 g/kg/day by oral gavage; treatment with glycans continued through Day 7. During this same period, animals in the control group received water. All groups (apart from the control group not receiving irinotecan) received a single dose of irinotecan on day 0 at a dose of 250 mg/kg intraperitoneal injection. At the time of irinotecan dosing to prevent transient diarrhea, atropine was given by subcutaneous injection at 0.03mg/kg.

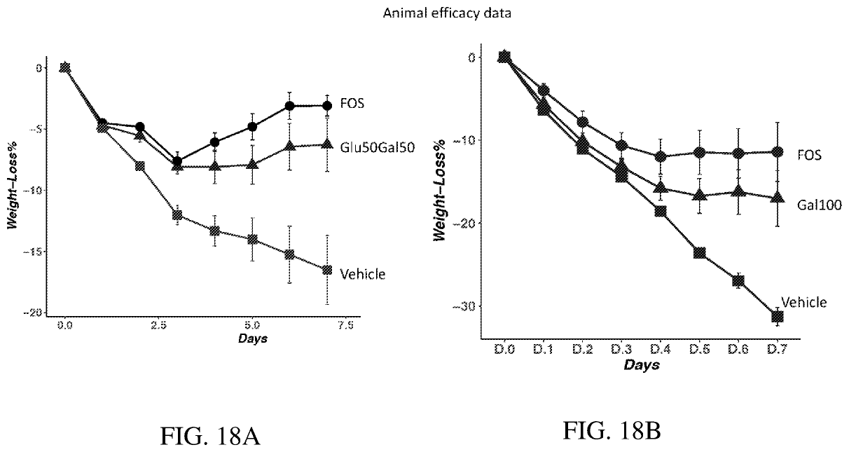

Daily, mice were weighed and monitored for survival. On day -1, 5 mice from 4 groups were euthanized with cecal and colonic contents collected for 16S sequencing and short-chain fatty acid assessment. Feces collected on days −6, −1, 1, 3, 5 and 7 and stored at −80° C. On day 7 remaining animals were euthanized for blood and tissue collection. FIGS. 18A and 18B show that mice fed FOS, Glu50Gal50, or Gal100 lost less weight over the time period of the experiment than mice that were not fed a glycan composition. The reduction in weight loss is a sign that the animals were healthier and encountered less toxicity from irinotecan metabolism. These data suggest that the glycans may shift the microbial community in the animals in a way that limits the production of toxic irinotecan products/intermediates by the microbial processing of the exogenous substance (as described above) in the animal, thereby limiting the overall toxicity of the drug.

### Example 6

### Modulation of Phytoestrogen Metabolism-Associated Bacteria by Glycans for the Treatment of Breast Cancer

Metabolism of phytoestrogens such as plant lignans and isoflavones by bacteria in the gut to molecules that bind estrogen receptors may have protective effects against breast cancer (Mabrook, H. B et al. Lignin transformation by gut bacteria lowers tumor burden in a gnotobiotic rat model of breast cancer. Carcinogenesis 33, 203-208 (2012)). Administration of glycans increases the relative growth of phytoestrogen metabolism-associated bacteria, such as BPR.22 and EFM.66 (see Example 3 and Table 11), and thus will increase the concentration of phytoestrogens and elicit protective effects against breast cancer and/or other types of cancer in humans. Women who are at high risk for breast cancer are randomized to receive glycan treatment or placebo for 12 months. Inclusion criteria are any of: i) Five year Gail risk >1.7%, ii) Known BRCA1/BRCA2 mutation carrier, iii) Family history consistent with hereditary breast cancer, iv) Prior biopsy exhibiting atypical hyperplasia or lobular carcinoma in situ (LCIS), or v) History of invasive breast cancer or ductal carcinoma in situ (DCIS) and have completed standard therapy including tamoxifen/aromatase inhibitor or will not be treated with tamoxifen/aromatase inhibitor. After 1 year of glycan or placebo treatment, patients are assessed for: i) reduced magnetic resonance imaging (MRI) volume (equivalent to 3-dimensional mammographic density) in high-risk women or those with invasive breast cancer or DCIS who are supplemented daily with glycan compared to placebo (microcrystalline cellulose), ii) decreased cell proliferation and apoptosis, as measured by Ki67 and caspase 3 staining, respectively, of breast epithelial cells with glycan treatment compared to placebo, iii) decreased intermediate molecular marker expression of estrogen receptor alpha (ER alpha) and ER beta in women treated with glycans compared to the placebo.

### Example 7

### Modulation of Phytoestrogen Metabolism-Associated Bacteria by Glycans for the Treatment of Type 2 Diabetes

The microbiome affects the development and progression of type 2 diabetes. Patients with type 2 diabetes have been found to have altered levels of microbial metabolites circulation as measured by urine. For example, the concentration of the microbially-produced lignan enterolactone is inversely correlated with the risk of type 2 diabetes (Sun et al. Diabetes Care 37.5(2014):1287-95). Increasing the concentration of molecules such as enterolactone may improve the outcome of patients with type 2 diabetes. Glycans can increase the production of enterolactone from the diet of mice as shown in FIGS. 1A-1C.

Patients with type 2 diabetes can be treated with glycans to increase the concentration of enterolactone in circulation compared to a placebo control treatment. Patients are included based on at least one of the following criteria: 1) an elevated glucose concentration (fasting plasma glucose ≥7.0 mmol/L, random plasma glucose ≥11.1 mmol/L, or plasma glucose ≥11.1 mmol/L after an oral glucose load) and at least one symptom related to diabetes; 2) no symptoms, but elevated glucose concentrations on two separate occasions; or 3) treatment with insulin or oral hypoglycemic medication. Patients are treated with a glycan or a placebo control for 6 months. At the end of 6 months, glycan treatment is expected to increase the concentration of enterolactone in the patient's urine as assessed via electrospray ionization orbitrap liquid chromatography-mass spectrometry compared to the placebo. Furthermore, at the end of 6 months, glycan treatment is expected to decrease fasting plasma glucose or glucose after an oral load compared to patients treated with placebo.

### Example 8

### Protocol for Examples 9-21

Examples 9-21 were carried out according to the following protocol:

An ex vivo assay was designed to determine if glycans can be used to modulate a complex community of microorganisms and if the glycans produced consistent effects across communities obtained from multiple (twelve) subjects. Fecal samples and slurries were handled in an anaerobic chamber (AS-580, Anaerobe Systems) featuring a palladium catalyst. Glycans ara100, gal100, glu60man40, glu100, glu50gal50, gal33man33ara33, man75gal25, glu33gal33man34, glu33gal33Ara33, man100, man52glu29gal19 and commercially available glycans lactulose (Alfa Aesar) and FOS (Nutraflora FOS; NOW Foods, Bloomingdale IL) were included in the study. The fecal sample donations obtained from 12 subjects were stored at −80° C. To prepare working stocks the fecal samples were transferred into the anaerobic chamber and allowed to thaw. The fecal samples were prepared to 20% w/v in phosphate buffered saline (PBS) pH 7.4 (P0261, Teknova Inc., Hollister, Calif.), 15% glycerol and stored at −80° C. The 20% w/v fecal slurry+15% glycerol was centrifuged at 2,000×g, supernatant was removed, and the pellet was suspended in PBS pH 7.4 to 1% w/v fecal slurry. Prepared 1% w/v fecal slurry were exposed to the studied glycans at a final concentration of 0.5% w/v in 96-well deep well microplates, 500 μL final volume per well, at 37° C. for 18 hours, anaerobically. Genomic DNA was extracted from the fecal samples and variable region 4 of the 16S rRNA gene was amplified and sequenced (Earth Microbiome Project protocol www.earthmicrobiome.org/emp-standard-protocols/16s/and Caporaso J G et al. 2012. Ultra-high-throughput microbial community analysis on the Illumina HiSeq and MiSeq platforms. ISME J.). Operational Taxonomic Units (OTUs) were generated by aligning 16S rRNA sequences at 97% identity. Microbial communities were compared to each other using UniFrac distance metric (Lozupone C. et al., Appl. Environ. Microbiol. December 2005 vol. 71 no. 12 8228-8235).

### Example 9

### Modulation of Cardiac Glycoside (e.g., Digoxin) Metabolism-Associated Bacteria by Glycans

The gut actinobacterium, Eggerthella lenta, has been previously shown to inactivate cardiac drug digoxin (see, e.g., Haiser et al. 2014. Gut Microbes: 5(2):233-238). The association with digoxin inactivation is limited to strains of Eggerthella lenta possessing the cardiac glycoyl reductase(cgr) operon, but suppressing the levels of Eggerthella lenta in patients under digoxin treatment might prevent or decrease inactivation of the digoxin drug.

Ex vivo assays showed that several glycans significantly reduced the levels of Eggerthella lenta (FIG. 7). All the samples with glycan preparations showed statistically significant (wilcoxon rank sum test—p-value <0.05) reduction in the relative composition of Eggerthella lenta when compared to the controls at 45 hours post inoculation. Four glycans also showed higher reduction than the naturally occurring fructose oligosaccharide (FOS).

### Example 10

### Modulation of Sulfonamide (e.g., Sulfasalazine) Metabolism-Associated Bacteria by Glycans

Microbial azoreductase, found in Bacteroides sp., Enteroccocus faecalis, and Lactobacillus sp., is known to transform sulfasalazine, which is used to treat rheumatoid arthritis, into its active form. Increasing the levels or activity of microbial azoreductase expressing microbes may increase the effectiveness of sulfasalazine treatment.

Ex vivo assays showed that several glycans significantly increase the levels of Bacteroides sp. and Enteroccaceae/Enterococcus (FIGS. 2 and 3).

In this assay, man100, glu100, man75gal25, man52glu29gal19, glu50gal50, ara100, FOS, gal100, and glu60man40 appeared to increase the levels of Bacteroides sp. and/or Enteroccaceae/Enterococcus. In addition, man100 increased the levels of Lactobacillus sp.

### Example 11

### Modulation of SN-38 Glucuronide Metabolism-Associated Bacteria by Glycans

Microbial beta-glucuronidase, found in Proteobacteria, Firmicutes, and Actinobacteria, is known to modify SN-38 glucuronide, which is used to treat cancer (e.g., colorectal cancer) and has the side-effect of diarrhea, by removing a sugar moiety, creating a compound that is toxic to intestinal epithelial cells. Decreasing the levels or activity of microbial beta-glucuronidase expressing microbes may increase the effectiveness of SN-38 glucuronidine treatment and/or decrease/prevent side effects.

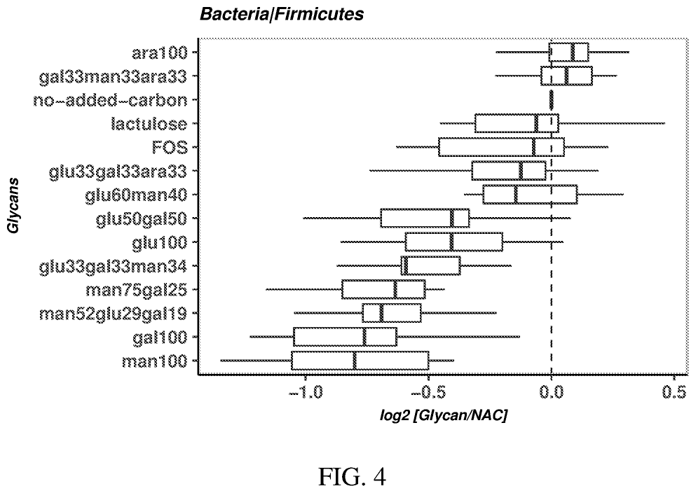

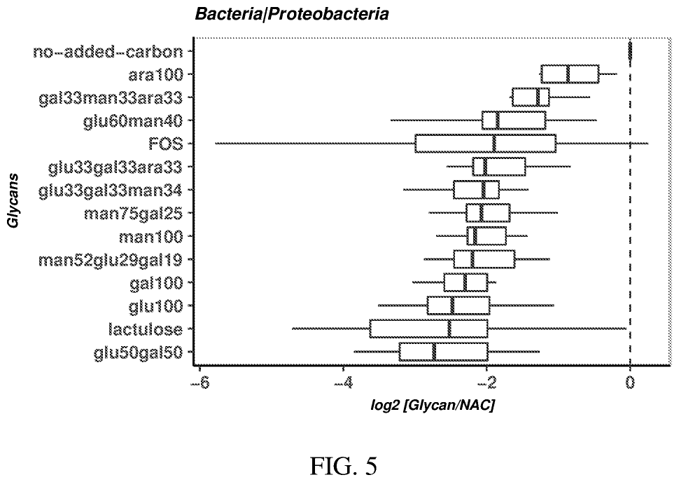

Ex vivo assays showed that several glycans significantly decrease the levels of Firmicutes, Proteobacteria, and Actinobacteria (FIGS. 4-6).

In this assay, man100, gal100, man52glu29gal19, man75gal25, glu33gal33man34, glu100, glu50gal50, lactulose, glu33gal33ara33, FOS, glu60man40, and ara100 appeared to decrease the levels of Firmicutes, Proteobacteria, and/or Actinobacteria.

### Example 12

### Modulation of NSAID (e.g., Tyrosine and Phenylalanine) Metabolism-Associated Bacteria by Glycans

A microbial enzyme, found in Firmicutes (e.g., Clostridium difficile), Bacteroidetes, Actinobacteria, and/or Fusobacteria, is known to modify tyrosine and/or phenylalanine into p-cresol, which competes with acetaminophen for SULT1A1. Decreasing the levels or activity of microbes expressing said microbial enzyme may increase the effectiveness of acetaminophen and/or reduce drug-induced toxicity.

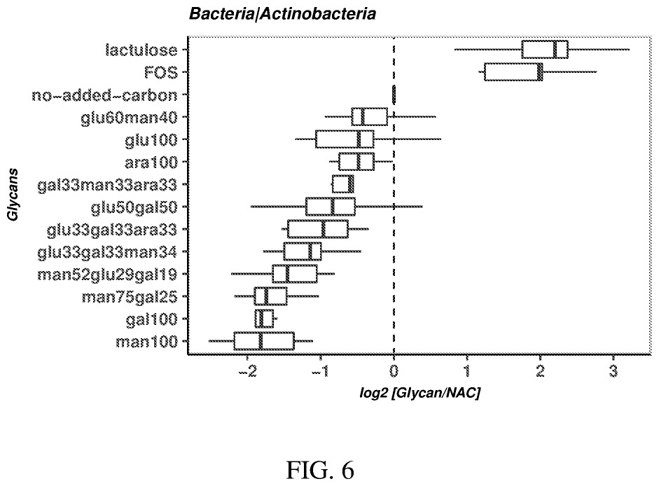

Ex vivo assays showed that several glycans significantly decrease the levels of Firmicutes and Actinobacteria (FIGS. 4 and 6).

In this assay, man100, gal100, man52glu29gal19, man75gal25, glu33gal33man34, glu33gal33ara33, and gal33man33ara33 appeared to decrease the levels of Firmicutes and/or Actinobacteria.

### Example 13

### Modulation of Polyphenol (e.g., Ellagitannin) Metabolism-Associated Bacteria by Glycans

A microbial enzyme found in Actinobacteria and Coriobacteriaceae/Gordonibacter is known to hydrolyze ellagitannin to ellagic acid.

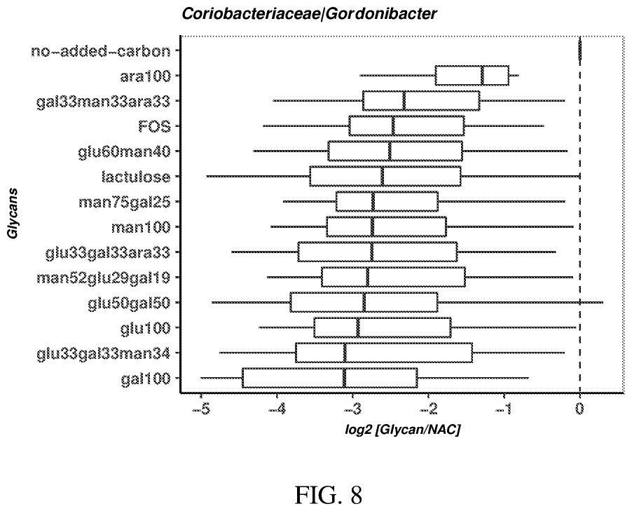

Ex vivo assays showed that several glycans significantly decrease the levels of Actinobacteria and Coriobacteriaceae/Gordonibacter (FIGS. 6 and 8). In this assay, man100, gal100, man75gal25, man52glu29gal19, glu33gal33man34, glu33gal33ara33, glu50gal50, glu100, lactulose, glu60man40, FOS, and ara100 appeared to decrease the levels of Actinobacteria and/or Coriobacteriaceae/Gordonibacter.

### Example 14

### Modulation of Phytoestrogen Metabolism-Associated Bacteria by Glycans

A microbial enzyme, found in Actinobacteria, Bacteroidetes, and Firmicutes is known to metabolize phytoestrogen to molecules that bind estrogen receptors and may have protective effects against breast cancer. Increasing the levels or activity of microbes expressing said microbial enzyme may have a preventative effect on cancer, e.g., breast cancer, or treat cancer, e.g., breast cancer.

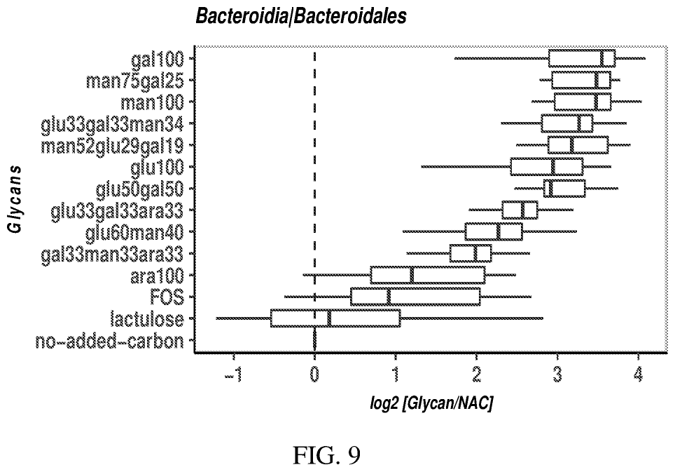

Ex vivo assays showed that several glycans significantly increase the levels of Bacteroidetes (FIG. 9).

In this assay, gal100, man75gal25, man100, glu33gal33man34, man52glu29gal19, glu100, glu50gal50, glu33gal33ara33, glu60man40, and gal33man33ara33 appeared to increase the levels of Bacteroidetes.

### Example 15

### Modulation of Polyphenol/Isoflavone (e.g., Daidzein) Metabolism-Associated Bacteria by Glycans

A microbial enzyme, found in Enterococcus faecium, Lactobacillus mucosae, Bifidobacterium sp., and Eggerthella sp., is known to metabolize daidzin into equol, which binds estrogen receptor-beta and may be associated with lower risk/incidence of breast cancer. Increasing the levels or activity of microbes expressing said microbial enzyme may increase the have a preventative effect on cancer, e.g., breast cancer, or treat cancer, e.g., breast cancer.

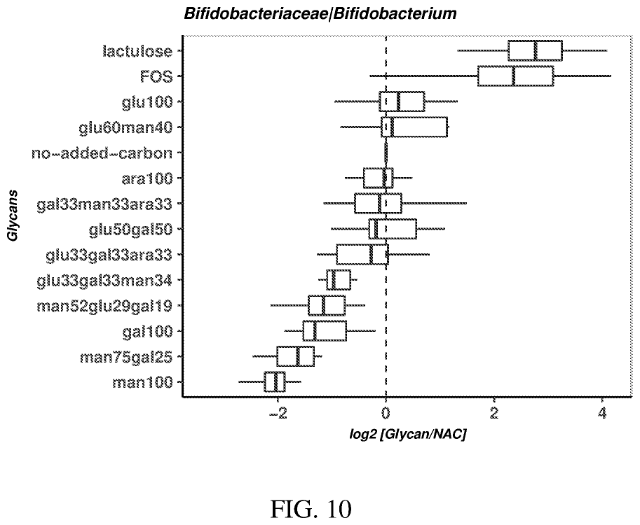

Ex vivo assays showed that several glycans significantly increase the levels of Enterococcaceae/Enterococcus and Bifidobacteriaceae/Bifidobacterium (FIGS. 3 and 10).

In this assay, glu60man40, gal33man33ara33, man75gal25, glu50gal50, man100, FOS, and lactulose appeared to increase the levels Enterococcaceae/Enterococcus and/or Bifidobacteriaceae/Bifidobacterium.

### Example 16

### Modulation of Phytoestrogen/Polyphenol (e.g., Lignan Metabolism-Associated Bacteria by Glycans

A microbial enzyme, found in E. faecalis, E. lenta, Blautia product, Eubacterium limosum, Clostridium scindens, Lactonifactor longoviformis, Clostridium saccharogumia, and P. product, is known to metabolize lignan, e.g., pinoresinol and secoisolariciresinol, to enterodiol and enterolactone, which may be protective against breast cancer. Increasing the levels or activity of microbes expressing said microbial enzyme may increase the have a preventative effect on cancer, e.g., breast cancer, or treat cancer, e.g., breast cancer.

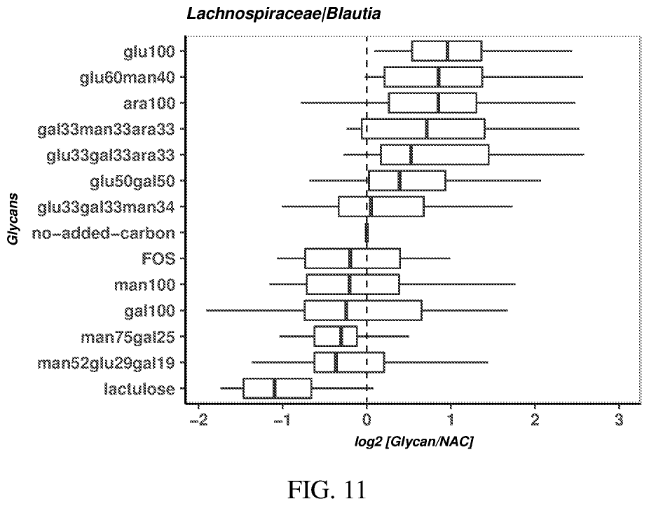

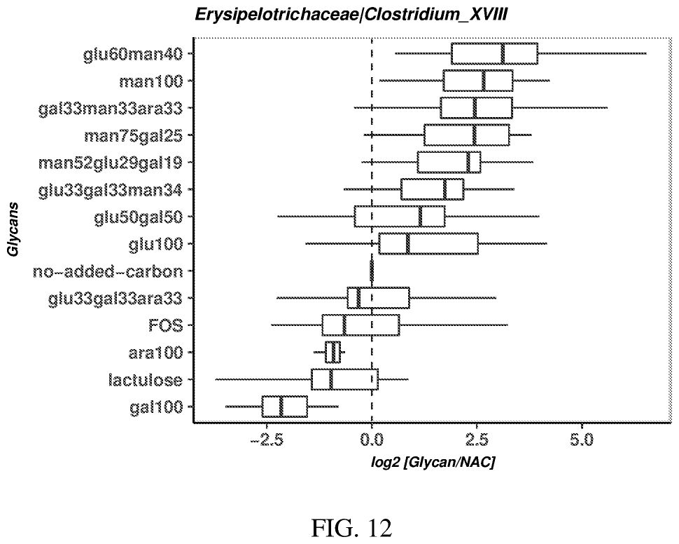

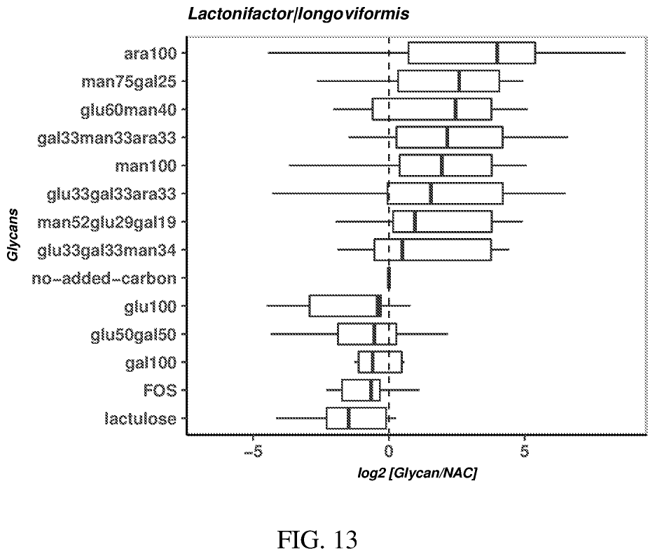

Ex vivo assays showed that several glycans significantly increase the levels of Lachnospiraceae/Blautia, Eryspelotrichaceae/Clostridium XVIII, and/or Lactonifactor/longoviformis (FIGS. 11-13).

In this assay, glu100, glu60man40, ara100, gal33man33ara33, glu33gal33ara33, glu50gal50, man75gal25, man52glu29gal19, and glu33gal33man34 appear to increase the levels of Lachnospiraceae/Blautia, Eryspelotrichaceae/Clostridium XVIII, and/or Lactonifactor/longoviformis.

### Example 17

### Modulation of Heterocyclic Amine (HCA)/Polycyclic Aromatic Hydrocarbon (PAH) Metabolism-Associated Bacteria by Glycans

Microbial beta-glucuronidase, found in bacteria that carry the uidA gene, e.g., Escherichia coli, is known to reactivate glucuronidated substrate, e.g., heterocyclic amines, (detoxified by hepatic glucuronidation), by removing the conjugate, generating a toxic compound. Decreasing the levels or activity of microbial beta-glucuronidase expressing microbes may decrease the generation of toxic, e.g., carcinogenic, compounds, which may have a preventative effect on cancer.

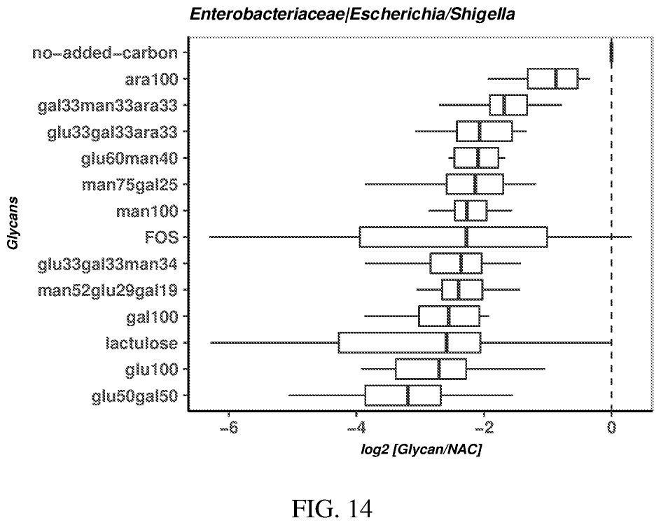

Ex vivo assays showed that several glycans significantly decrease the levels of Enterobacteriaceae/Escherichia/Shigella microbes (FIG. 14).

In this assay, ara100, gal33man33ara33, glu33gal33ara33, glu60man40, man75gal25, man100, FOS, glu33gal33man34, man52glu29gal19, gal100, lactulose, glu100, and glu50gal50 appeared to decrease the levels of Enterobacteriaceae/Escherichia/Shigella microbes.

### Example 18

### Modulation of Non-Caloric Artificial Sweetener Metabolism-Associated Bacteria by Glycans

A microbial enzyme, found in Enterococcus, Clostridium, Corynebacterium, Campylobacter, and Escherichia, is known to convert sweetener to a compound that can be toxic, e.g., converts cyclamate to cyclohexylamine, which can be toxic. Decreasing the levels or activity of microbes expressing said microbial enzyme may decrease the generation of toxic compounds.

Ex vivo assays showed that several glycans significantly decrease the levels of Enterococcaceae/Enterococcus and Enterobacteriaceae/Escherichia/Shigella microbes (FIGS. 3 and 14).

In this assay, ara100, gal33man33ara33, glu33gal33ara33, glu60man40, man75gal25, man100, FOS, glu33gal33man34, man52glu29gal19, gal100, lactulose, glu100, and glu50gal50 appeared to decrease the levels of Enterococcaceae/Enterococcus and/or Enterobacteriaceae/Escherichia/Shigella microbes.

### Example 19

### Modulation of Nucleoside Analog (e.g., Sorivudine) Metabolism-Associated Bacteria by Glycans

Microbial phosphorylase, e.g., thymidine phosphorylase or uridine phosphorylase, found in Enterobacteria, e.g., K. pneumoniae, is known to deglycosylate sorivudine, inactivating it. Sorivudine is used to treat viral infection (e.g. herpes simplex virus 1 and varicella-zoster virus). Decreasing the levels or activity of microbes expressing said microbial phosphorylase may decrease the inactivation of sorivudine and increase the effectiveness of sorivudine treatment against viral infections.

Ex vivo assays showed that several glycans significantly decrease the levels of Enterobacteriales/enterobacteriaceae microbes (FIG. 15).

In this assay, ara100, gal33man33ara33, glu60man40, FOS, man75gal25, glu33gal33man34, glu33gal33ara33, man52glu29gal19, man100, gal100, glu100, lactulose, and glu50gal50 appeared to decrease the levels of Enterobacteriales/Enterobacteriaceae microbes.

### Example 20

### Modulation of Immunotherapeutic Antigen (e.g., CTLA4 Inhibitor) Metabolism-Associated Bacteria by Glycans

A microbial enzyme found in Bacteroides, e.g., Bacteroides thetaiotaomicron and/or Bacteroides fragilis, is known to modify immunotherapeutic antigens. Increasing the levels or activity of microbes expressing said microbial enzyme may increase the effectiveness of CTLA4 inhibitor, e.g., to treat cancer.

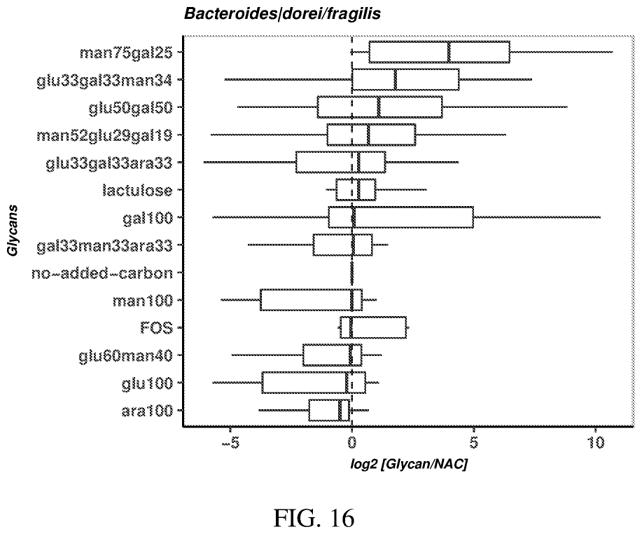

Ex vivo assays showed that several glycans significantly increase the levels of Bacteroides/dorei/fragilis and Bacteroidaceae/Bacteroides microbes (FIGS. 16 and 2).

In this assay, man72gal25, glu33gal33man34, glu50gal50, man100, glu100, man52glu29gal19, ara100, FOS, gal100, and glu60man40 appeared to increase the levels of Bacteroides/dorei/fragilis and Bacteroidaceae/Bacteroides microbes.

### Example 21

### Modulation of Drug Additive (e.g., Emulsifying Agent) Metabolism-Associated Bacteria by Glycans

A microbial enzyme found in Bacteroidetes (e.g., Bacteroidales) and mucolytic bacteria such as Ruminococcus gnavus, is known to cause metabolic and inflammatory response to emulsifying agents. Decreasing the levels or activity of microbes expressing said microbial enzyme may increase the effectiveness of emulsifying agents, e.g, carboxymethylcellulose or polysorbate-80, e.g., to decrease risk/incidence of metabolic syndrome and/or inflammation.

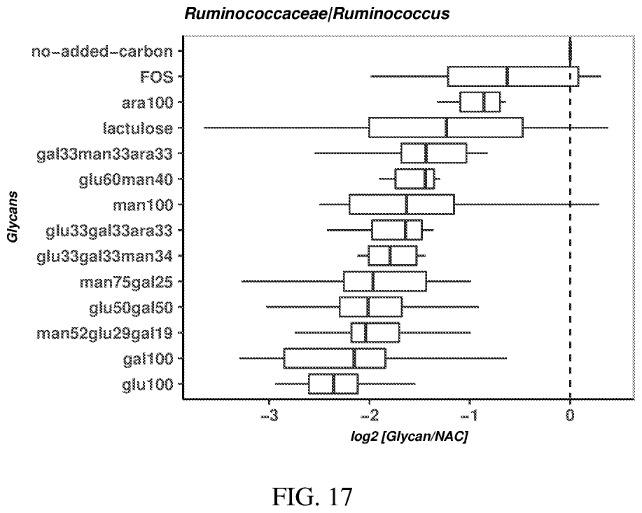

Ex vivo assays showed that several glycans significantly decrease the levels of Ruminococcaceae/Ruminococcus microbes (FIG. 17).

In this assay, ara100, lactulose, gal33man33ara33, glu60man40, man100, glu33gal33ara33, glu33gal33man34, man75gal25, glu50gal50, man52glu29gal19, gal100, and glu100 appeared to decrease the levels of Ruminococcaceae/Ruminococcus microbes.

### Example 22

### Glycan Preparations

To a round bottom flask equipped with an overhead stirrer and a jacketed short-path condenser was added one or more mono- or disaccharides along with 3-20% (or, alternatively, 0.1-15%) by dry weight of one or more of the catalysts e.g. acid, ionic, ionic/acid containing catalysts such as, e.g. described in U.S. Pat. No. 9,079,171 and WO 2016/007778, which are incorporated herein by reference in their entirety. Water or another compatible solvent (zero to 10 equiv.) was added to the dry mixture and the slurry was combined at approximately 100 rpm using a paddle sized to match the contours of the selected round bottom flask as closely as possible. The mixture was then heated to 80-185° C. Once the solids achieved a molten state, the vessel was placed under 10-1000 mbar vacuum pressure. Alternatively, the reaction may be performed without use of a vacuum. The reaction was stirred for 30 minutes to 8 hours, constantly removing water from the reaction. Reaction progress was monitored by HPLC. When sufficient oligomerization had occurred, the stirrer was shut off, the reaction was cooled to room temperature and vented to atmospheric pressure, and the product, either as a solid or syrup, was dissolved in a volume of water sufficient to create a solution of approximately 50 Brix (grams sugar per 100 g solution)(e.g., 50% solids). Once dissolution was complete, solid catalyst was removed by filtration and the oligomer solution was concentrated to approximately 50-75 Brix (e.g., 50-75% solids) by rotary evaporation. In cases in which an organic solvent has been used, water immiscible solvents can be removed by biphasic extraction and water miscible solvents can be removed by rotary evaporation concomitant to the concentration step.

Among others, the following glycans were made in multiple batches and tested in various assays described herein:

Single glycan unit (homo-glycans): ara100, fru100, gal100, galA100, glcNac100, glu100, gluA100, Lglu100, man100, rha100, xyl100.

Two glycan units (hetero-glycans): Ara60Xyl40, Ara80Xyl20, Gal20Ara80, Gal20Xyl80, Gal40Ara60, Gal40Man60, Gal40Xyl60, Gal57Glu43, Gal60Ara40, Gal60Man40, Gal60Xyl40, Gal80Ara20, Gal80Man20, Gal80Xyl20, Glu20Ara80, Glu20Xyl80, Glu40Ara60, Glu40Gal60, Glu40Xyl60, Glu50Gal50, Glu50Lglu50, Glu60Ara40, Glu60Gal20Man20, Glu60Gal40, Glu60Man40, Glu60Xyl40, Glu66Fru33, Glu75Gala25, Glu75GluA25, Glu75GluN25, Glu80Ara20, Glu80Gal20, Glu80Lglu20, Glu80Man20, Glu80Xyl20, Glu90LGlu10, Man20Ara80, Man20Xyl80, Man40Ara60, Man40Xyl60, Man60Ara40, Man60Glu40, Man60Xyl40, Man75Gal25, Man80Ara20, Man80Gal20, Man80Glu21, Man80Xyl20, Xyl60Ara40, Xyl75Ara25, Xyl80Ara20, and the hybrid glycans glu90sor10 and glu90gly10.

Three glycan units (hetero-glycans): Gal5Xyl5Ara90, Gal5Xyl90Ara5, Gal10Xyl10Ara80, Gal10Xyl45Ara45, Gal10Xyl80Ara10, Gal20Xyl20Ara60, Gal20Xyl40Ara40, Gal20Xyl60Ara20, Gal30Xyl30Ara40, Gal30Xyl40Ara30, Gal33Man33Ara33, Gal33Man33Xyl33, Gal33Xyl33Ara33, Gal45Xyl10Ara45, Gal45Xyl45Ara10, Gal50Glu25Fru25, Gal40Xyl20Ara40, Gal40Xyl30Ara30, Gal40Xyl40Ara20, Gal60Xyl20Ara20, Gal80Xyl10Ara10, Gal90Xyl5Ara5, Glu5Gal5Man90, Glu5Gal90Man5, Glu5Xyl5Ara90, Glu5Xyl90Ara5, Glu10Gal10Man80, Glu10Gal45Man45, Glu10Gal80Man10, Glu10Xyl10Ara80, Glu10Xyl45Ara45, Glu10Xyl80Ara10, Glu20Gal20Man60, Glu20Gal40Man40, Glu20Gal60Man20, Glu20Gal80, Glu20Xyl20Ara60, Glu20Xyl40Ara40, Glu20Xyl60Ara20, Glu30Gal30Man40, Glu30Gal40Man30, Glu30Xyl30Ara40, Glu30Xyl40Ara30, Glu33Gal33Ara33, Glu33Gal33Fuc33, Glu33Gal33Man33, Glu33Gal33Xyl33, Glu33Man33Ara33, Glu33Man33Xyl33, Glu33Xyl33Ara33, Glu40Gal20Man40, Glu40Gal30Man30, Glu40Gal40Man20, Glu40Xyl20Ara40, Glu40Xyl30Ara30, Glu40Xyl40Ara20, Glu45Gal10Man45, Glu45Gal45Man10, Glu45Xyl10Ara45, Glu45Xyl45Ara10, Glu60Xyl20Ara20, Glu75GluNAc25, Glu80Gal10Man10, Glu80Xyl10Ara10, Glu90Gal5Man5, Glu90Xyl5Ara5, Man33Xyl33Ara33, Man52Glu29Gal19.

Four glycan units (hetero-glycans): Gal25Man25Xyl25Ara25, Glu25Gal25Man25Ara25, Glu25Gal25Man25Xyl25, Glu25Gal25Xyl25Ara25, Glu25Man25Xyl25Ara25.

Five glycan units (hetero-glycans): Glu20Gal20Man20Xyl20Ara20.

Glycans are described by a three- to six-letter code representing the monomeric sugar component followed by a number out of one hundred reflecting the percentage of the material that monomer constitutes. Thus, ‘glu100’ is ascribed to a glycan generated from a100% D-glucose (glycan unit) input and ‘glu50gal50’ is ascribed to a glycan generated from 50% D-glucose and 50% D-galactose (glycan units) input or, alternatively from a lactose dimer (glycan unit) input. As used herein: xyl=D-xylose; ara=L-arabinose; gal=D-galactose; glu=D-glucose; rha=L-rhamnose; fuc=L-fucose; man=D-mannose; sor=D-sorbitol; gly=D-glycerol; neu=NAc-neuraminic acid; Lglu=L-glucose; gluA=D-glucuronic acid; gluN=D-glucosamine; gluNAc=N-acetyl-D-glucosamine; galA=D-galacturonic acid; 3-Bn=benzyl; 3-Obn=3-benzyloxy; 6-TBDPS=6-tert-butyldiphenylsilyl; galnac=N-acetyl galactosamine; rib=D-ribose; Sor=sorbitol.

### Example 23

### Purification

Oligo- and polysaccharides were dissolved in deionized water to a final concentration of 25-50 Brix (e.g., 25-50% solids). The material was then exposed to at least 2 mass equivalents of Dowex Monosphere 88 ion exchange resin. Exposure may occur by swirling in a flask at 120-170 rpm or by filtration through a wet slurry packed column as long as the residence time is sufficient for the solution to achieve a final pH between 3 and 5. The oligomer solution was isolated by filtration (as in the case of swirled reactions) or elution (as in the case of column filtration) and the process was repeated with Dowex Monosphere 77 ion exchange resin in an analogous fashion until the solution pH was above 5.5. Finally, the solution was exposed to Dowex Optipore SD-2 Adsorbent decolorizing resin until the solution was sufficiently clarified and filtered through a 0.2 micron filter to remove residual resin and resin fines. The final solution was then concentrated to 50-85 Brix by rotary evaporation or to a solid by lyophilization.

### Example 24

### High-Throughput Preparation at Small Scale

The oligomers and polymers were synthesized in a parallel fashion in 24-, 48-, or 96-well plates or similarly sized arrays of 1 dram vials housed in aluminum heating blocks. In this example, all liquid transfers were handled by a programmable robot or manually using calibrated pipettes. To each vial or well was added 20-100% by dry weight of one or more catalysts e.g. acid, ionic, ionic/acid containing catalysts such as, e.g. described in U.S. Pat. No. 9,079,171 and WO 2016/007778. The plate or heating block was placed uncovered in a vacuum oven heated to 50 to 150° C. under a vacuum of 10-800 mbar. The oven vacuum pump was protected by a two-stage condenser consisting of a recirculating chiller trap followed by a dry ice/acetone trap. The plates or blocks are heated for 30 minutes to 6 hours under elevated temperature and reduced pressure without stirring. After a pre-established period of time, the oven was vented to atmospheric pressure, the plates or blocks were cooled to room temperature, and each well or vial was diluted to approximately 50 Brix (e.g., 50% solids) with deionized water. The solid-phase extraction steps described in Example 2 were performed by elution through sequential wet-packed columns in which the eluent from each column flows immediately into the top of the next column at a rate between 2 and 6 bed volumes/hour using a peristaltic pump or other suitable small pump. The column stack was then rinsed with deionized water and the combined effluents are concentrated by lyophilization to isolate solid powders with residual water content of 1-10% by mass.

### Example 25

### Removal of Low Molecular Weight Species

Oligomers or polymers were modified so as to remove low molecular weight species. In one embodiment the separation was achieved by osmotic separation. Approximately 45 cm of 1.0 kD MWCO Biotech CE dialysis tubing (31 mm flat width) from Spectrum Labs was placed into deionized water and soaked for 10 minutes, then one end was sealed with a dialysis tubing clip. A 25 Brix (e.g., 25% solids) solution of 8 grams dry oligosaccharide was sterile filtered and sealed into the tube with a second clip along with a few mL of air to permit the tube to float. The filled tube was then placed in a 3 gallon tank of deionized water which was stirred with sufficient force to induce slow swirling of the sealed tubes. After 8 hours, the water in the tank was replaced and the tube was allowed to stir for an additional 16 hours. Once the dialysis was complete and the material had a DP2+ yield greater than 95% and a DP3+ yield greater than 90%, the dilute solution was sterile filtered and concentrated in vacuo to a final concentration of approximately 65 Brix (e.g., 65% solids) or lyophilized to a solid with a residual moisture between 1 and 10%.

In a second embodiment the separation was achieved by tangential flow filtration (TFF). In this case, 100 mL of 25 Brix (e.g., 25% solids) glycan sample dissolved in deionized water and sterile filtered was placed into the feed bottle of a Spectrum Labs KrosFlo Research IIi TFF system that was prepared according to the manufacturer's recommendation. The sample was then diafiltered through a 1 kD mPES MidiKros hollow-fiber filter at a transmembrane pressure of 25 psig. HPLC samples of the feed stock taken every 0.5 diafiltration volumes were used to determine when the material had a DP2+ yield greater than 95% and a DP3+ yield greater than 90% at which point the solution was sterile filtered and concentrated in vacuo to a 65 Brix (e.g., 65% solids) syrup or lyophilized to a solid with residual water content of 1-10% by mass.

In a third embodiment the separation was achieved by ethanol precipitation. In this case, 100 mL of 25 Brix (e.g., 25% solids) glycan sample was poured into a vigorously stirred beaker containing 900 mL of pure, USP-grade ethanol at a rate no higher than 10 mL/minute. Once the addition was complete, the precipitated solids were subjected to stirring for an additional 15 minutes at or slightly below room temperature. The precipitated solids were isolated by filtration through a fine frit sintered glass funnel under an atmosphere of nitrogen to prevent hydration and gumming. The solids were rinsed once with ethanol, then dissolved in water to a final concentration of 25 Brix (e.g., 25% solids) and reconcentrated to >65 (e.g., >65% solids) Brix. This syrup was then diluted back to 25 (e.g., 25% solids) Brix and concentrated once more to ensure removal of residual ethanol.

### Example 26

### Methods for Analyzing Preparations

Measurement of Concentration by Liquid Refractometry

This experiment was designed to quantitate the amount of glycan in any given aqueous solution. A Mettler-Toledo Refracto 30GS portable sugar refractometer was calibrated using high-purity reverse-osmosis deionized water. Several drops of the glycan solution were filtered through a 0.2 micron syringe filter directly onto the lens of the refractometer. The measurement was taken at room temperature and reported as Brix (e.g., % solids). The glycans were routinely concentrated to 50, 60, 70, or 75 Brix (e.g., % solids) without obvious solidification or crystallization at 23° C. Brix (e.g., % solids) can then be converted to solubility assuming a specific density of water equal to 1.0 g/mL. Thus, 75 Brix (100 grams of solution consisting of 75 grams of glycan and 25 grams of water) equals an aqueous solubility of 3.0 g/mL. As a comparison, the aqueous solubility of D-glucose is reported to be 0.909 g/mL (48 Brix (e.g., 48% solids)) at 25° C. by Sigma-Aldrich.

Monomeric Composition by Hydrolysis and GC-MS

This experiment was designed to quantitate the ratio of monomer content within a given oligosaccharide. Glycosyl composition analysis was performed by combined gas chromatography/mass spectrometry (GC/MS) of the per-O-trimethylsilyl (TMS) derivatives of the monosaccharide methyl glycosides produced from the sample by acidic methanolysis as described previously by Santander et al. (2013) Microbiology 159:1471. Between 100 and 200 1.tg of sample were lyophilized into a suitable test tube. Inositol (20 μg) was added to the sample as an internal standard, then the sample was heated to 80° C. in 1M HCl/methanol for 18 hours. The resulting monosaccharides were then re-acetylated using pyridine and acetic anhydride in MeOH, and per-O-trimethylsilylated with Tri-Sil (Pierce) at 80° C. for 30 minutes. GC/MS analysis of the TMS methyl glycosides was performed on an Agilent 7890A GC interfaced to a 5975C MSD, using a Supelco Equity-1 fused silica capillary column (30 m×0.25 mm ID). Each peak was assigned to a component sugar based upon comparison to known standards and integration of the respective peaks allowed clean calculation of the relative percentage of monomers within an exemplified glycan. In all enumerated glycans, conditions can be routinely identified in which the monomer composition of a given oligosaccharide matched the input ratio within experimental error and the output composition matched the input composition within the precision of the measurement.

Molecular Weight Distribution by Size-Exclusion Chromatography (SEC)

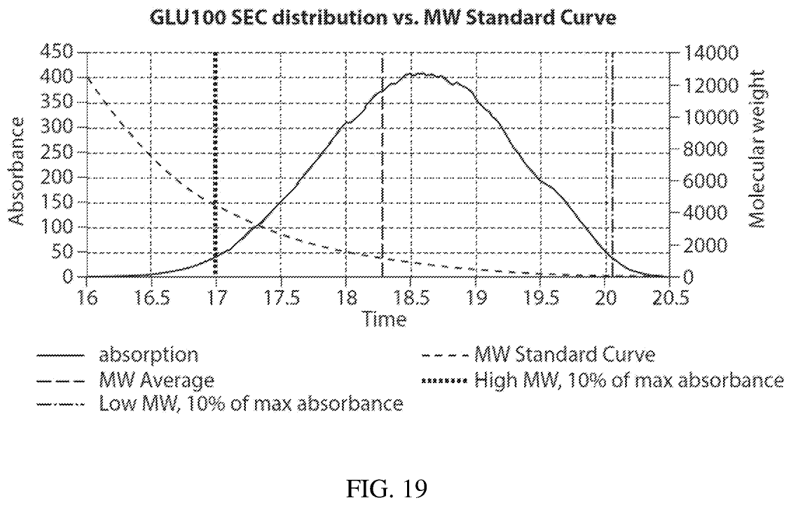

This experiment was designed to quantitate the distribution of molecular weights within a given oligosaccharide. The measurement was made by HPLC using the method described in Monograph of United States Pharmacopeia, 38(6) In-Process Revision: Heparin Sodium (USP37-NF32). Separations were achieved on an Agilent 1200 HPLC system via a GE superpose 12 column using 50 mM ammonium acetate as an eluent at 1.0 mL/min flow rate and an ELSD detector. The column temperature was set at 30° C. and dextran (1 kD, 5 kD, 10 kD weight) were used to draw a standard curve. A 2 mg/ml solution of the samples was prepared and passed through a 0.45 μm spin filter, followed by 40 μl injections into the HPLC. A third-order polynomial curve was constructed based on the logarithmic molecular weights and elution volumes of the listed standards. The weight-average molecular weight (Mw), the number average molecular weight (Mn), and the polydispersity index (PDI) for the sample were calculated by comparison to the standard curve. FIG. 19 shows the curve generated during the SEC evaluation of a glu100 sample in which the average molecular weight was determined to be 1212 g/mol or approximately DP7. The upper end of molecular weight of the material as defined by the point of the curve at 10% of maximum absorption leading the curve was determined to be 4559 g/mol or approximately DP28. The lower end of molecular weight of the material as defined by 10% of the maximum absorption trailing the curve was determined to be 200 g/mol or approximately DP1. Similar analysis of a glu50gal50 sample showed a MW, high mass, and low mass of 1195 g/mol (˜DP7), 4331 g/mol (˜DP27), and 221 g/mol (˜DP1) respectively.

Alternatively, the average molecular weight (Mw) and polydispersity index (PDI) may be measured or calculated, respectively, by injecting a sample with a concentration of 1-50 mg/mL onto an Agilent 1100 HPLC equipped with two 7.8×300 mm Agilent PL aquagel-OH 20, 30, or 40 columns as described in Monograph of United States Pharmacopeia, 38(6) In-Process Revision: Heparin Sodium (USP37-NF32). Separations were achieved using 0.2M sodium nitrate as an eluent at 0.90 mL/min flow rate and an RID detector. The column temperature was set at 50° C. and pullulan (glucose, 1.5 kD, 6 kD, 12 kD, 20 kD, 50 kD weight) was used to draw a standard curve. A 3rd order polynomial curve was constructed based on the logarithmic molecular weights and elution volumes of the listed standards. The weight-average molecular weight (Mw), the number average molecular weight (Mn), and the polydispersity index (PDI) for the sample were calculated by comparison to the standard curve.

Molecular Weight Distribution by Ion-Affinity Chromatography (IAC)

The proportion of glycan with DP greater than or equal to 2 (DP2+) and 3(DP3+) may be measured by ion-affinity chromatography. A sample of glycan was diluted out to 50-100 mg/mL and 10 0_, of this solution was injected onto an Agilent 1260 BioPure HPLC equipped with a 7.8×300 mm BioRad Aminex HPX-42A column and RI detector. Using pure HPLC-grade water as an eluent, the sample was eluted at 0.6 mL/min through an 80° C. column and an RI detector maintained at 50° C. The peaks representing DP1-6 are assigned by comparison to reference standards and integrated using the Agilent ChemStation software. Peaks are typically integrated as DP1, DP2, DP3, DP4-7, and DP8+. The DP that is achievable by the reaction described in Example 1 varies from monomer to monomer although it is consistent across batches if the procedure is followed. For example, across 17 batches of glu100, DP2+values ranged from 77-93% and DP3+ values ranged from 80-90%. Conversely, across 6 batches of ara100, DP2+ values ranged from 63-78% and DP3+ values ranged from 48-71%. Mixtures of monomers behaved as averages of the individual components.

Alpha-/Beta-Distribution by 2D NMR

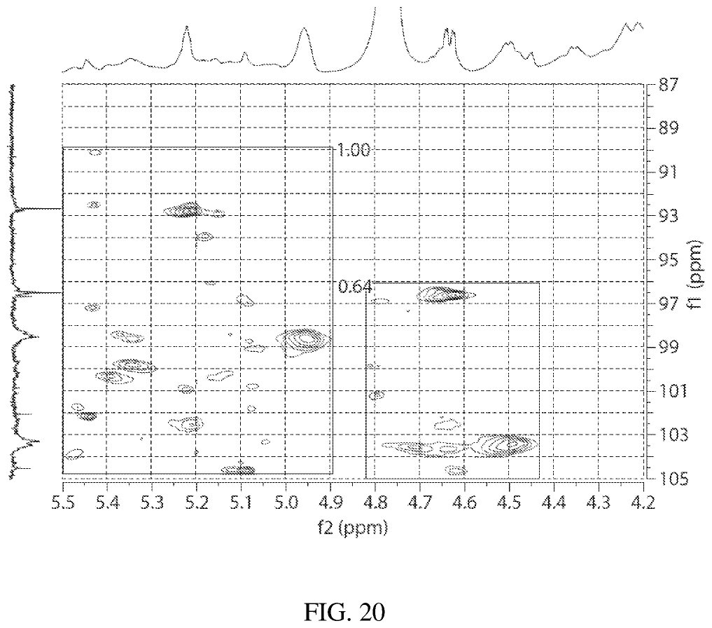

This experiment was designed to quantitate the ratio of alpha- and beta-glycosidic bonds within a given sample by two-dimensional NMR. Approximately 150 mg of 65 Brix oligosaccharide solution was dried to stable mass in a vacuum oven at 45-95° C. under 400 mbar pressure. The sample was subjected to two cycles of dissolution in D2O and drying to remove residual H2O. Once dried, the sample was dissolved in 750 μL D2O with 0.1% acetone, placed into a 3 mm NMR tube, and analyzed in a Bruker Avance-III operating at 500.13MHz 1H (125.77 MHz 13C) equipped with a Bruker BBFO probe operating at 21.1° C. The sample was analyzed using a heteroatomic single quantum coherence pulse sequence (HSQC) using the standard Bruker pulse sequence. Anomeric protons between 4-6 ppm (1H) and 80-120 ppm (13C) were assigned by analogy to glucose as reported in Roslund, et al. (2008) Carbohydrate Res. 343:101-112. Spectra were referenced to the internal acetone signal: 1H-2.22 ppm; 13C-30.8 ppm. Isomers were quantitated by integration of their respective peaks using the MNova software package from Mestrelab Research (Santiago de Compostela, Spain). FIG. 20 shows the anomeric region of a representative spectrum. Over 300 samples have been assayed in this fashion and Table 2 lists the distribution across a sample of combinations of monomers showing the alpha-/beta-ratio to be as high as 4:1 as in the case of rha100 and as low as 1:1 as in the case of glu50gal50.

TABLE 7
Distribution of alpha- and beta-bonds
across batches and types of glycans

| glycans | alpha-bonds (%) | beta-bonds (%) | alpha/beta ratio |
|---|---|---|---|
| Glu100 | 58 | 42 | 1.4 |
| | 61 | 39 | 1.6 |
| | 64 | 36 | 1.8 |
| | 64 | 36 | 1.8 |
| | 62 | 38 | 1.6 |
| | 61 | 39 | 1.6 |
| | 62 | 38 | 1.6 |
| | 63 | 37 | 1.7 |
| | 60 | 40 | 1.5 |
| | 65 | 35 | 1.9 |
| | 65 | 35 | 1.9 |
| | 60 | 40 | 1.5 |
| Gal100 | 60 | 40 | 1.5 |
| Gal33man33ara33 | 79 | 21 | 3.8 |
| | 75 | 25 | 3.0 |
| Glu50gal50 | 50 | 50 | 1.0 |
| | 56 | 44 | 1.3 |
| | 61 | 39 | 1.6 |
| | 65 | 35 | 1.9 |
| Glu33gal33fuc33 | 55 | 45 | 1.2 |
| Man100 | 57 | 43 | 1.3 |
| Man52glu29gal19 | 76 | 24 | 3.2 |
| Ara100 | 67 | 33 | 2.0 |
| Rha100 | 80 | 20 | 4.0 |
| Xyl100 | 57 | 43 | 1.3 |
| | 59 | 41 | 1.4 |
| Xyl75gal25 | 56 | 44 | 1.5 |

### Identification of Composition by NMR

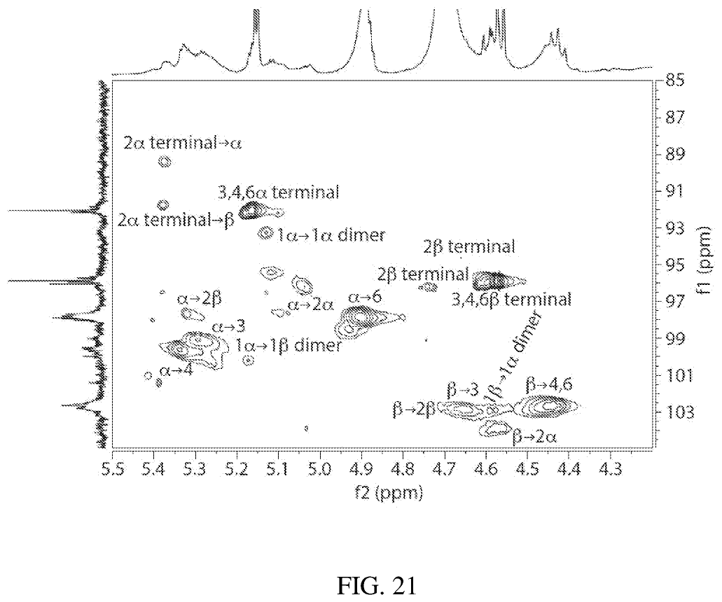

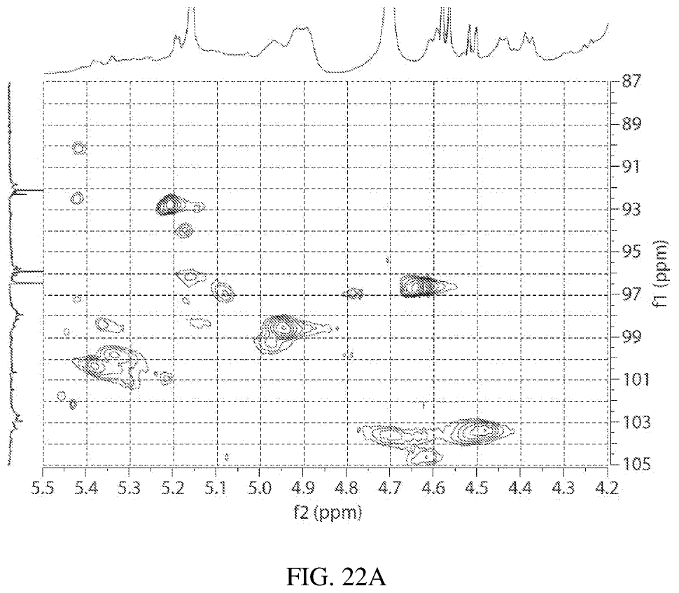

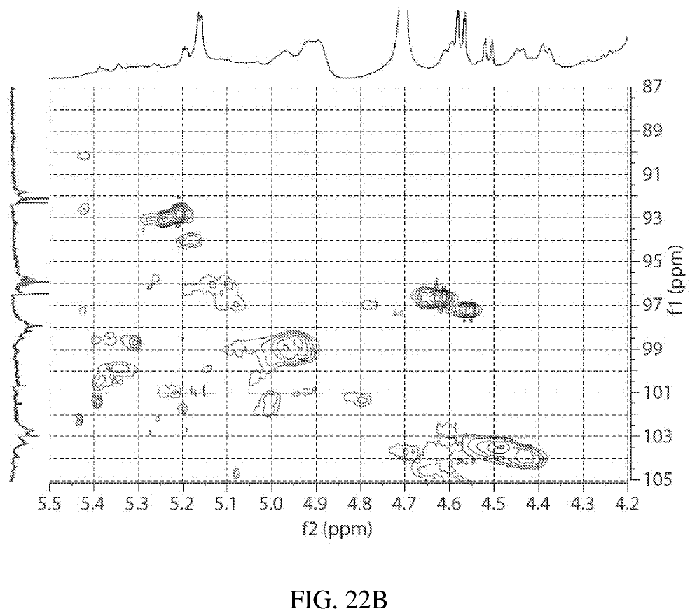

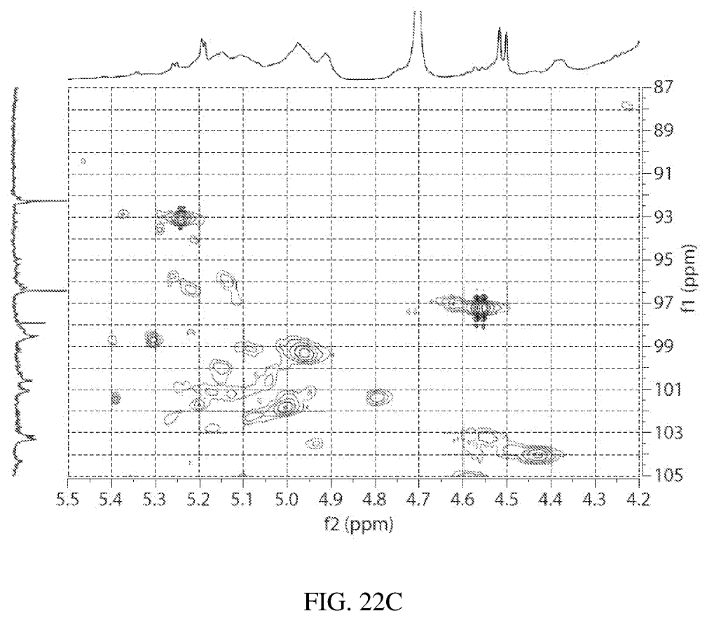

This experiment was designed to identify the composition of a glycan by 2D-NMR identification of the constituent monomers. Approximately 150 mg of 65 Brix oligosaccharide solution was dried to stable mass in a vacuum oven at 45-95° C. under 400 mbar pressure. The sample was subjected to two cycles of dissolution in D2O and drying to remove residual H₂O. Once dried, the sample was dissolved in 750 μL D2O with 0.1% acetone, placed into a 3 mm NMR tube, and analyzed in a Bruker Avance-III operating at 500.13 MHz 1H (125.77 MHz 13C) equipped with a Bruker BBFO probe operating at 70° C. The sample was analyzed using a heteroatomic single quantum coherence pulse sequence (HSQC) using the standard Bruker pulse sequence. The anomeric region of each glycan spectra derived from a single sugar monomer was then examined for peaks representing specific glycosidic bonds characteristic to that monomer. For any given glycan, the HSQC spectra allow the identification of peaks that are unique to specific regio- and stereochemical bond arrangement. For example, FIG. 21 shows a partial assignment of the spectra of a glu100 preparation demonstrating how these peaks may be used to identify specific glycosidic regio- and stereo-chemistries. Due to the spin-isolated nature of single carbohydrate rings within polysaccharides, the HSQC spectra of a glycan with more than one monomer is predicted to be represented by the sum of the HSQC peaks of each of its constituent sugars. Therefore, each constituent monomer has unique HSQC peaks that will appear in any glycan that contains that monomer irrespective of other constituent monomers and furthermore, the monomers used to synthesize a glycan can be determined by identifying the fingerprint peaks unique to each constituent monomer. For example, FIG. 22B shows that the HSQC spectra of glu50gal50 is a hybrid of the spectra of glu100 (FIG. 22A) and gal100 (FIG. 22C). Table 3 lists the fingerprint peaks for selected glycan units.

TABLE 8
Diagnostic HSQC peaks for each component sugar.

| Monomer | 1H shift | 13C shift | Monomer | 1H shift | 13C shift |
|---|---|---|---|---|---|
| Glucose | 5.42 | 92.5 | Xylose | 5.18 | 93.0 |
| | 5.21 | 92.8 | | 5.10 | 94.3 |
| | 5.18 | 93.9 | | 5.34 | 98.2 |
| | 5.08 | 97.0 | | 5.31 | 99.6 |
| | 5.36 | 98.4 | | 5.11 | 100.8 |
| | 5.34 | 99.8 | | 4.91 | 99.4 |
| | 5.38 | 100.3 | | 4.56 | 97.3 |
| | 4.95 | 98.6 | | 4.64 | 104.2 |
| | 4.62 | 96.6 | | 4.54 | 103.4 |
| | 4.70 | 103.6 | | 4.44 | 102.6 |
| | 4.49 | 103.4 | | 4.44 | 104.1 |
| Galactose | 5.37 | 92.9 | Arabinose | 5.22 | 93.2 |
| | 5.24 | 93.1 | | 5.13 | 93.2 |
| | 5.14 | 96.0 | | 5.29 | 96.0 |
| | 4.96 | 99.3 | | 5.26 | 97.2 |
| | 5.31 | 98.7 | | 5.12 | 96.6 |
| | 5.39 | 101.4 | | 5.18 | 99.6 |
| | 5.00 | 101.8 | | 5.06 | 99.2 |
| | 4.80 | 101.3 | | 4.99 | 100.0 |
| | 4.63 | 97.0 | | 5.26 | 101.9 |
| | 4.56 | 97.2 | | 5.06 | 102.1 |
| | 4.53 | 103.1 | | 4.55 | 97.4 |
| | 4.43 | 104.1 | | 4.54 | 105.2 |
| Fucose | 5.18 | 92.9 | | 4.50 | 105.5 |
| | 5.33 | 92.4 | | 4.38 | 103.9 |
| | 5.04 | 96.3 | Rhamnose | 5.21 | 93.2 |
| | 4.90 | 99.7 | | 5.10 | 94.5 |
| | 4.52 | 97.0 | | 4.85 | 94.1 |
| | 4.39 | 103.6 | | 5.01 | 95.8 |
| Mannose | 5.37 | 93.0 | | 5.35 | 100.5 |
| | 5.16 | 94.6 | | 5.15 | 102.2 |
| | 4.88 | 94.2 | | 5.04 | 102.9 |
| | 5.39 | 101.7 | | 4.78 | 97.9 |
| | 5.24 | 101.9 | | 4.71 | 99.0 |
| | 5.13 | 102.8 | | 4.72 | 101.0 |
| | 5.03 | 102.7 | | | |
| | 5.24 | 105.6 | | | |
| | 5.09 | 108.0 | | | |
| | 4.88 | 94.2 | | | |
| | 4.89 | 100.0 | | | |
| | 4.70 | 101.1 | | | |

At least 5 peaks appeared for each glycan unit used as a starting material in the synthesis of glycans containing 3 or fewer distinct glycan units. The HSQC spectra of glycans containing 4 or more distinct glycan units have at least 4 peaks for each constituent glycan unit.

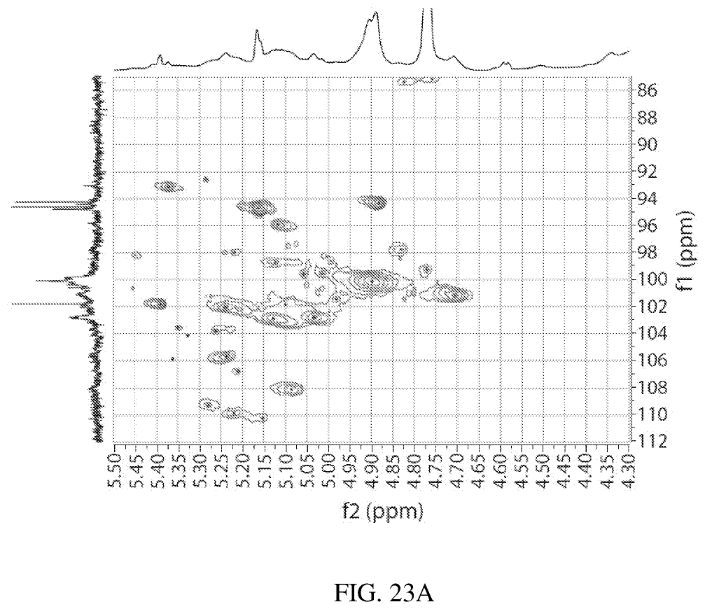

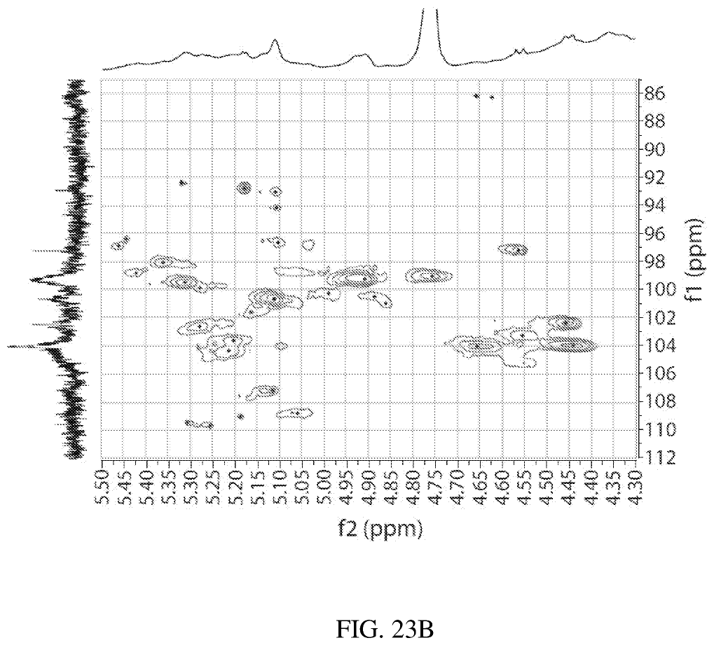

FIGS. 23A and 23B show the HSQC spectra for man100 and xyl100, respectively.

Glycosidic Linkage Analysis

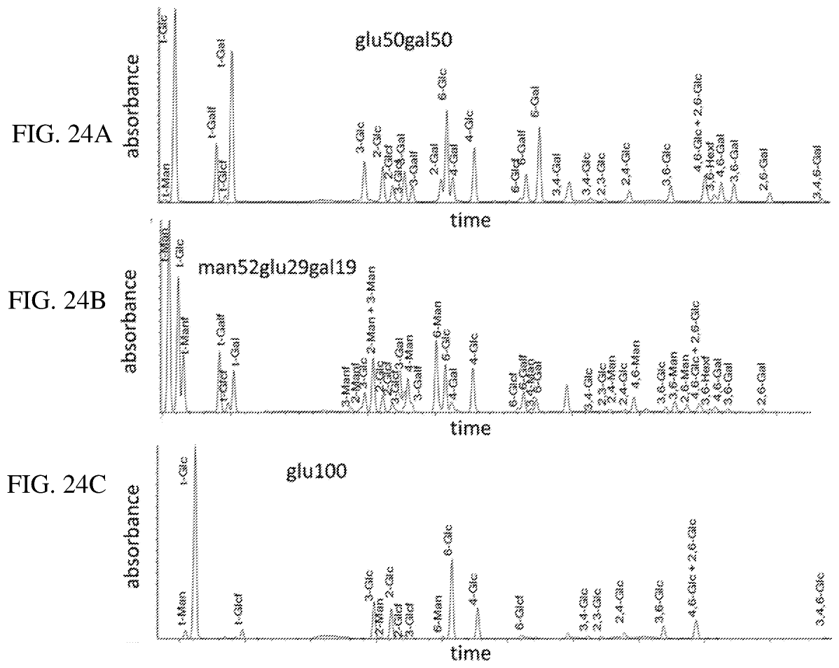

This experiment was designed to quantitate the distribution of glycosidic regioisomers (branching) within a given oligosaccharide. For glycosyl linkage analysis, the samples were permethylated, depolymerized, reduced, and acetylated; and the resultant partially methylated alditol acetates (PMAAs) analyzed by gas chromatography-mass spectrometry (GC-MS) as described by Heiss et al (2009) Carbohydr. Res. 344:915. The samples were suspended in 200 μl of dimethyl sulfoxide and left to stir for 1 day. Permethylation was affected by two rounds of treatment with sodium hydroxide (15 min) and methyl iodide (45 min). The aqueous solution was hydrolyzed by addition of 2M trifluoroacetic acid and heating to 121° C. for 2 hours. Solids were isolated in vacuo and acetylated in acetic acid/trifluoroacetic acid. The resulting PMAAs were analyzed on an Agilent 7890A GC interfaced to a 5975C MSD (mass selective detector, electron impact ionization mode); separation was performed on a 30 m Supelco SP-2331 bonded phase fused silica capillary column. FIGS. 24A-24C show three representative GC spectra from this analysis. These analyses show that the glycans had at least 0.1%, 0.2%, 0.5%, 1%, 2%, 5%, 10% or more of the 1,2-glycoside bond type, e.g. ara100=3.8%, gal100=7.2%; at least 0.1%, 0.2%, 0.5%, 1%, 2%, 5%, 10% or more of the 1,3-glycoside bond type, e.g. 3-bn-glu100=41.7%, glu50gal50=10.4%; at least 0.1%, 0.2%, 0.5%, 1%, 2%, 5%, 10% or more of the 1,4-glycoside bond type, e.g. glu50gal50=5.9%, gal33man33ara33=10.1%; and at least 0.1%, 0.2%, 0.5%, 1%, 2%, 5%, 10%, 15%, 20%, 25% or more of the 1,6-glycoside bond type, e.g. gal33man33ara33=13.4%, glu100=25.4%. The materials also contained at least 5%, 10%, 15%, 20%, 25%, 30%, 40%, 50%, or more of the branched bond types (including but not limited to 1,3,6-; 1,4,6-; or 1,2,4-glycosides, e.g. Table 14), a degree of branching (DB) of at least 0.05. Degree of branching is defined as the average number of branched monomers relative to total number of monomer units. For example, a glu100 glycan polymer in which 20% of the glucose monomer units contain glycosidic linkages to three or more other glucose monomers would have a DB of 0.20. The glycans also have about 3-12% of the monomeric units in the furanose form. A glycan originating from a single monomer consisted of at least 12 distinct non-terminal substitution patterns. A glycan originating from two monomers consisted of at least 18 distinct non-terminal substitution patterns, e.g. glu-1,2-glu; glu-1,2-gal; gal-1,2-glu; gal-1,2-gal; glu-1,2(glu),6-glu; glu-1,3-glu; glu-1,3-gal; etc. A glycan originating from three or more monomers consisted of at least 24 distinct non-terminal substitution patterns.

TABLE 14
A sample of degree of branching (DB) measurements; sample selected
from 54 different preparations characterized as described herein.

% branched monomers

| | highest | lowest |
|---|---|---|
| composition | measure | measure |
| glu100 | 40.4 | 10.4 |

| | |
|---|---|
| glu80man20 | 16.1 |
| glu60man40 | 16.4 |
| man80glu20 | 18.6 |
| man60glu40 | 20.5 |

| | | |
|---|---|---|
| glu50gal50 | 22.4 | 12.6 |

| | |
|---|---|
| gal100 | 22.2 |
| glu33gal33fuc33 | 41.8 |
| ara100 | 16.6 |
| xyl100 | 63.2 |
| xyl75ara25 | 26.9 |

| | | |
|---|---|---|
| man52glu29gal19 | 22.7 | 9.8 |

| | |
|---|---|
| man100 | 40.0 |

TABLE 10
A selection of different glycan preparations for use in the methods disclosed herein.

| | | | | | | | | alpha/beta | | | | | | |
|---|---|---|---|---|---|---|---|---|---|---|---|---|---|---|
| | total molar incidence | | | | Misc glycoside | | | ratio by | | | | | | |
| | of a bond (%) | | | | sums (%) | | | HSQC | | | | | | |
| | to- | to- | to- | to- | total | total | total | NMR | | SEC data | | | | |
| | tal | tal | tal | tal | branch- | fura- | terminal | % | % | DP | | | | |
| Glycan | 1,2 | 1,3 | 1,4 | 1,6 | ing | nose | sugars | alpha | beta | 2+ % | Mw | Mn | PD | DPn |
| Glu5Gal5Man90-2 | 19% | 15% | 22% | 43% | 25.9 | 12 | 34.9 | 80% | 20% | 98% | 1842 | 946 | 1.95 | 11.26 |
| Glu10Gal10Man80-1 | 15% | 16% | 24% | 45% | 22.6 | 6.7 | 33.1 | 81% | 19% | 98.60% | 1978 | 1021 | 1.94 | 12.1 |
| Glu20Gal20Man20Xyl20Ara20-1 | 16% | 18% | 32% | 34% | 25.1 | 33.1 | 6.85 | 87% | 13% | 100% | 1278 | 935 | 1.37 | 7.78 |
| Glu20Gal20Man20Xyl20Ara20-2 | 16% | 19% | 16% | 48% | 4.8 | 35.3 | 1.68 | 63% | 37% | 100% | 1845 | 1000 | 1.85 | 11.28 |
| Gal33Man33Ara33-8 | 17% | 26% | 23% | 34% | 25.5 | 27.5 | 32.7 | 87% | 13% | 98% | 1527 | 834 | 1.83 | 9.31 |
| Gal57Glu43-1 | 4% | 7% | 73% | 16% | 2 | 2.7 | 50.9 | 33% | 67% | 94% | 374 | 349 | 1.07 | 2.20 |
| Glu100-87 | 1% | 3% | 93% | 4% | 0 | 0 | 34.7 | 69% | 31% | 100% | 416 | 399 | 1.04 | 2.46 |
| Gal57Glu43-2 | 2% | 2% | 1% | 94% | 1.3 | 1.5 | 46.6 | 65% | 35% | 98% | 390 | 374 | 1.04 | 2.3 |
| Glu50Gal50-11 | 15% | 20% | 20% | 45% | 14.8 | 12.2 | 38.3 | 64% | 36% | 91% | 1456 | 675 | 2.16 | 8.88 |
| Glu50Gal50-32 | 15% | 16% | 26% | 43% | 13.1 | 17.9 | 45.2 | 66% | 34% | 96% | 1114 | 790 | 1.41 | 6.77 |
| Glu50Gal50-14 | 13% | 17% | 25% | 44% | 13.5 | 22.4 | 43.3 | 70% | 30% | | | | | |
| Glu50Gal50-27 | 15% | 20% | 22% | 43% | 19.5 | 9.6 | 29.5 | 61% | 39% | 99% | 1776 | 945 | 1.88 | 10.85 |
| Glu50Gal50-23 | 17% | 20% | 20% | 44% | 19.2 | 17.2 | 35.5 | 71% | 29% | 99% | 1497 | 855 | 1.75 | 9.13 |
| Glu50Gal50-2 | 16% | 21% | 18% | 45% | 19.4 | 15.6 | 35.5 | 65% | 35% | | 1931 | 936 | 2.06 | 11.8 |
| Glu100-129 | 20% | 19% | 16% | 46% | 19.1 | 5.3 | 36.3 | 62% | 38% | 99% | 1411 | 1712 | 1.21 | 7.84 |
| Glu100-136 | 19% | 20% | 16% | 46% | 19.6 | 4.7 | 34.8 | 64% | 36% | 99% | 1577 | 1834 | 1.16 | 8.76 |
| Glu100-17 | 19% | 20% | 15% | 47% | 19.7 | 3.1 | 31.6 | 61% | 39% | 98% | 1523 | 1797 | 1.18 | 8.46 |
| Glu100-64 | 19% | 21% | 15% | 46% | 19.6 | 3.3 | 34.6 | 62% | 38% | 98% | 1620 | 1871 | 1.15 | 9.00 |
| Glu100-76 | 18% | 19% | 15% | 47% | 18.5 | 3.8 | 33.4 | 62% | 38% | 99% | 1410 | 1702 | 1.21 | 7.83 |
| Glu100-131 | 18% | 18% | 17% | 46% | 16.4 | 7.4 | 39.2 | 61% | 39% | 98% | 1200 | 1520 | 1.27 | 6.67 |
| Glu100-83 | 19% | 20% | 18% | 44% | 22.2 | 8.7 | 34.5 | 64% | 36% | 99% | 1605 | 1849 | 1.15 | 8.92 |
| Glu100-139 | 19% | 20% | 15% | 46% | 19.4 | 4.5 | 34.5 | 64% | 36% | 98% | 1542 | 1819 | 1.18 | 8.57 |
| Glu100-84 | 19% | 20% | 15% | 46% | 19 | 3.5 | 32.6 | 62% | 38% | 99% | 1431 | 1726 | 1.21 | 7.95 |
| Glu100-74 | 19% | 19% | 17% | 45% | 22.2 | 6.7 | 27.9 | 61% | 39% | 98% | 1387 | 1697 | 1.22 | 7.71 |
| Glu100-98 | 19% | 19% | 18% | 45% | 18.5 | 6.9 | 36.4 | 62% | 38% | 98% | 1383 | 1690 | 1.22 | 7.68 |
| Glu100-141 | 18% | 24% | 16% | 41% | 40.4 | 3.7 | 16.3 | 63% | 37% | 99% | 1673 | 1898 | 1.13 | 9.29 |
| Glu100-29 | 19% | 18% | 16% | 46% | 19.5 | 3.8 | 30 | 60% | 40% | 98% | 1311 | 1624 | 1.24 | 7.28 |
| Glu100-18 | 20% | 21% | 15% | 45% | 27.5 | 3.4 | 18.9 | 65% | 35% | 99% | 1748 | 1946 | 1.11 | 9.71 |
| Glu100-99 | 18% | 20% | 16% | 45% | 20.1 | 6.5 | 35.4 | 64% | 36% | 99% | 1641 | 1876 | 1.14 | 9.12 |
| Glu100-72 | 19% | 20% | 17% | 44% | 22.2 | 6.3 | 32.2 | 64% | 36% | 99% | 1716 | 1929 | 1.12 | 9.54 |
| Glu100-82 | 18% | 21% | 17% | 44% | 22 | 6.4 | 30.6 | 65% | 35% | 99% | 1711 | 1927 | 1.13 | 9.50 |
| Glu100-130 | 18% | 21% | 17% | 44% | 21.9 | 5.2 | 32.9 | 63% | 37% | 99% | 1781 | 1967 | 1.10 | 9.90 |
| Glu100-78 | 18% | 20% | 17% | 44% | 21.6 | 4.5 | 32 | 63% | 37% | 99% | 1719 | 1926 | 1.12 | 9.55 |
| Glu100-66 | 19% | 20% | 17% | 44% | 22 | 6.6 | 31.1 | 62% | 38% | 98% | 1472 | 1763 | 1.20 | 8.18 |
| Glu100-89 | 18% | 19% | 16% | 48% | 18.6 | 6.7 | 35.9 | 61% | 39% | 98% | 1326 | 1638 | 1.23 | 7.37 |
| Glu100-133 | 17% | 18% | 18% | 46% | 20.1 | 11.1 | 35.8 | 65% | 35% | 97% | 1224 | 1567 | 1.28 | 6.80 |
| Glu100-68 | 18% | 19% | 17% | 46% | 18.7 | 7.4 | 36.3 | 60% | 40% | 98% | 1394 | 1701 | 1.22 | 7.74 |
| Glu100-90 | 19% | 20% | 16% | 45% | 16.8 | 4.2 | 38.8 | 51% | 49% | 96% | 982 | 674 | 1.46 | 5.90 |
| Glu100-94 | 19% | 19% | 14% | 47% | 17.7 | 3.1 | 35.1 | 54% | 46% | 100% | 1369 | 978 | 1.40 | 8.30 |
| Glu100-5 | 19% | 19% | 14% | 48% | 16.3 | 3 | 36.6 | 57% | 43% | 100% | 1226 | 902 | 1.36 | 7.40 |
| 3-Obn Glu100-1 | 14% | 5% | 31% | 50% | 34.4 | 5.5 | 5.8 | 66% | 34% | 100% | 1014 | 486 | 2.09 | 6.15 |
| Gal100-30 | 16% | 19% | 24% | 41% | 17.2 | 32.6 | 30.4 | 74% | 26% | | | | | |
| Glu33Gal33Fuc33-3 | 15% | 30% | 29% | 27% | 41.8 | 15.2 | 22.5 | 65% | 35% | | | | | |
| Ara100-12 | 26% | 42% | 32% | NA | 16.6 | 36.7 | 23.1 | 74% | 26% | | | | | |
| Xyl100-8 | 19% | 35% | 46% | NA | 63.2 | 3.8 | 0.3 | 70% | 30% | | | | | |
| Xyl75Ara25-3 | 25% | 32% | 43% | NA | 26.9 | 18.7 | 23.5 | 69% | 31% | | | | | |
| Glu80Man20-2 | 15% | 19% | 21% | 45% | 16.1 | 4.6 | 34 | 68% | 32% | | | | | |
| Glu60Man40-5 | 10% | 24% | 23% | 43% | 16.4 | 2.1 | 28.3 | 79% | 21% | | | | | |
| Man80Glu20-2 | 8% | 25% | 17% | 50% | 18.6 | 1.8 | 30.9 | 87% | 13% | | | | | |
| Man60Glu40-2 | 8% | 22% | 26% | 43% | 20.5 | 3.7 | 28.6 | 73% | 27% | | | | | |
| Man52Glu29Gal19-2 | 12% | 19% | 27% | 42% | 8.4 | 19 | 5 | 77% | 23% | | | | | |
| Man52Glu29Gal19-3 | 8% | 18% | 31% | 44% | 23.6 | 26.6 | 6.8 | 82% | 18% | | | | | |
| Man100-17 | 12% | 27% | 25% | 36% | 40 | 9.5 | 19.4 | 57% | 43% | | | | | |

Branched (DB > 0) glu-100 was used and analyzed in all the Examples.

### Example 27

### Glycans Affect Growth of Strains Expressing Enzymes

Microbial and enzyme composition data from 300 synthesized glycans were used to identify which glycans would reduce strains that express the enzymes N-acetyl transferase, beta glucuronidase, thymidine phosphorylase, uridine phosphorylase, bile acid CoA hydrolase, and urease. Glycans were tested in fecal samples to determine the level of enzyme produced in a fecal slurry after incubation for 45 hours. The fecal samples were prepared to 20% w/v in phosphate buffered saline (PBS) pH 7.4 (P0261, Teknova Inc., Hollister, Calif.), 15% glycerol and stored at −80° C. The 20% w/v fecal slurry+15% glycerol was centrifuged at 2,000×g, supernatant was removed, and the pellet was suspended in PBS pH 7.4 to 1% w/v fecal slurry. Prepared 1% w/v fecal slurry were exposed to the studied glycans at a final concentration of 0.5% w/v in 96-well deep well microplates, 500 μL final volume per well, at 37° C. for 18 hours, anaerobically. Genomic DNA was extracted from the fecal samples and variable region 4 of the 16S rRNA gene was amplified and sequenced (Earth Microbiome Project protocol and Caporaso JG et al. The ISME Journal (2012) 6, 1621-1624). Operational Taxonomic Units (OTUs) were generated by aligning 16S rRNA sequences at 97% identity. Microbial communities were compared to each other using UniFrac distance metric (Lozupone C. et al., Appl. Environ. Microbiol. December 2005 vol. 71 no. 12 8228-8235).

Following incubation, cells were pelleted by centrifugation at 3,716×g for 10 minutes and the supernatant was stored at −80° C. or on dry ice until it was analyzed. Chromatographic analysis of N-acetyl transferase concentration was carried out on samples using an Agilent 7890B system with a flame ionization detector (FID) (Agilent Technologies, Santa Clara, Calif.). A high-resolution gas chromatography capillary column 30 m×0.25 mm coated with 0.25 um film thickness was used (DB-FFAP) for the volatile acids (Agilent Technologies). Nitrogen was used as the carrier gas. The oven temperature was 145° C. and the FID and injection port was set to 225° C. The injected sample volume was 1 uL and the run time for each analysis was 12 minutes. Chromatograms and data integration was carried out using the OpenLab ChemStation software (Agilent Technologies).

Average fold change of enzyme expression for each glycan was compared to water for the ability to reduce or increase expression of microbial enzymes among the bacterial strains of the fecal slurries. A number of glycans tested demonstrated a greater reduction or increase of enzyme in microbial strains compared to water (FIG. 25A-25F). Glycans Glu5Gal5Man90 (e.g., Glu5Gal5Man90-1, Glu5Gal5Man90-2), Man80Ara20 (e.g., Man80Ara20-1), Gal33Man33Xyl33 (e.g., Gal33Man33Xyl33-1), Man80Xyl20 (e.g., Man80Xyl20-1), Glu10Gal 1 0Man80 (e.g., Glu10Gal 1 0Man80-1, Glu10Gal10Man80-2), Glu10Gal45Man45 (e.g., Glu10Gal45Man45-1, Glu10Gal45Man45-2), Glu30Gal40Man30 (e.g., Glu30Gal40Man30-1), Glu20Gal40Man40 (e.g., Glu20Gal40Man40-1, Glu20Gal40Man40-2), Glu10Gal80Man10 (e.g., Glu10Gal80Man10-1), Glu50Gal50 (e.g., Glu50Gal50-32, Glu50Gal50-3), Gal100 (e.g., Gal100-2, Gal100-3, Gal100-10), Glu45Gal10Man45 (e.g., Glu45Gal10Man45-1, Glu45Gal10Man45-2), Glu20Gal20Man60 (e.g., Glu20Gal20Man60-2), and Glu40Gal20Man40 (e.g., Glu40Gal20Man40-1) all induced a greater reduction of N-acetyl transferase expression in microbial strains than water (FIG. 25A).

Glu100 (e.g., Glu100-132) demonstrated a greater reduction of beta-glucuronidase in microbial strains compared to water (FIG. 25B). Glycans glu100 (e.g., Glu100-87, Glu100-132, Glu100-77, Glu100-100, Glu100-85, Glu100-129, Glu100-141, Glu100-33, Glu100-29, Glu100-66, Glu100-17, Glu100-64, Glu100-138, Glu100-135, Glu100-21, Glu100-76, Glu100-131, Glu100-139, and Glu100-84) all induced a greater reduction of thymidine phosphorylase expression in microbial strains than water (FIG. 25C). Glycans glu100 (e.g., Glu100-85 and Glu100-138) induced a greater reduction of uridine phosphorylase expression in microbial strains than water (FIG. 25D).

Glycans gal100 (e.g., Gal100-3 and Gal100-10) induced a greater reduction of bile acid CoA hydrolase expression in microbial strains than water (FIG. 25E). Glycan glu100 (e.g., Glu100-132) induced a greater increase of urease expression in microbial strains than water (FIG. 25F). This example demonstrates that exposing microbial strains to glycans can affect the expression of different bacterial enzymes. Glycans were able to increase (e.g., urease) or decrease (e.g., N-acetyltransferase, beta-glucuronidase, thymidine phosphorylase, uridine phosphorylase, and bile acid CoA hydrolase) expression of microbial enzymes. cl Equivalents and Scope

This application refers to various issued patents, published patent applications, journal articles, and other publications, each of which is incorporated herein by reference in its entirety, and in the form of any pages, sections or subject matter referred to, is hereby incorporated by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present invention, as defined in the following claims.

We claim:

1. A method for increasing drug activity in a subject, wherein the drug comprises a cardiac glycoside, the method comprising:
a) administering a glycan composition in an amount effective and for a time sufficient to increase the drug activity in the subject;
b) administering a glycan composition in an amount effective and for a time sufficient to increase drug activity in the subject, and wherein at the time of administration of the glycan composition, the subject comprises a level of the drug that, in the presence of the administered glycan composition, provides a therapeutic effect;
c) administering the drug, wherein at the time of administration of the drug, the subject has already been administered the glycan composition in an amount effective and for a time sufficient to increase the drug activity in the subject;
d) administering the drug in an amount effective and for a time sufficient to increase the drug activity in the subject, wherein subject has been determined to be in need of the glycan composition, e.g., to increase the activity of the drug; or
administering the drug and the glycan composition to the subject, in amounts effective and for times sufficient to increase the drug activity in the subject, wherein administration of the drug and the glycan composition overlap;
wherein:
i) the glycan preparation comprises glycan polymers that comprise glucose, galactose, arabinose, mannose, fructose, xylose, fucose, or rhamnose glycan units;
ii) the average degree of branching (DB) of the glycan polymers in the glycan preparation is 0, between 0.01 and 0.6, between 0.05 and 0.5, between 0.1 and 0.4, or between 0.15 and 0.4;
iii) at least 50% (at least 60%, 65%, 70%, 75%, 80%, or 85%, or less than 50%) of the glycan polymers in the glycan preparation have a degree of polymerization (DP) of at least 3 and less than 30 glycan units, at least 3 and less than 10 glycan units, at least 5 and less than 25 glycan units, or at least 10 and less than 35 glycan units;
iv) the average DP (mean DP) of the glycan preparation is between about 5 and 8, between about 8 and 13, between about 13 and 25, between about 5 and 15, between about 5 and 20, or between about 5-15;
v) the ratio of alpha- to beta-glycosidic bonds present in the glycan polymers of the glycan preparation is 0, or between about 0.8:1 to about 5:1, between about 1:1 to about 5:1, between about 1:1 to about 3:1, between about 3:2 to about 2:1, or between about 3:2 to about 3:1,
vi) the glycan preparation comprises between 15 mol % and 75 mol % (between 20 mol % and 60 mol %, between 25 mol % and 50 mol %, or between 30 mol % and 45 mol %) 1,6 glycosidic bonds;
vii) the glycan preparation comprises between 1 mol % and 40 mol % (between 1 mol % and 30 mol %, between 5 mol % and 25 mol %, between 10 mol % and 20 mol %) of each at least one, two, or three of 1,2; 1,3; and 1,4 glycosidic bonds;
viii) the glycan preparation has a final solubility limit in water of at least about 50 (at least about 60, 70, at least about 75, or less than 50) Brix at 23° C.; or
ix) the glycan preparation has a dietary fiber content of at least 50% (at least 60%, 70%, 80%, or at least 90%, or less than 50%), and
x) any combination of two, three, four, five, six, seven, eight, or nine of i), ii), iii), iv), v), vi), vii), viii), and ix).

2. The method of claim 1, wherein the drug and the glycan preparation are administered to the subject for treating heart failure.

3. The method of claim 1 or 2, wherein the drug comprises digoxin, digitoxin, convallotoxin, antiarin, or oleandrin.

4. The method of any of claims 1-3, wherein the subject exhibits symptoms associated with heart failure, including one or more of shortness of breath, excessive tiredness, and leg swelling.

5. The method of any of claims 1-4, wherein the subject has conditions associated with heart failure, including one or more of obesity, kidney failure, anemia, liver problems, and thyroid disease.

6. The method of any of claims 1-5, wherein the subject has findings associated with heart failure, including one or more of cardiomegaly, vascular redistribution, Kerley lines, arrhythmia, ischemic heart disease, ventricular hypertrophy, conduction delay/abnormality, elevated B-natriuretic peptide, and reduced ejection fraction.

7. The method of any of claims 1-6, wherein the subject has a visibly enlarged heart relative to a subject without heart failure.

8. The method of any of claims 1-7, wherein the subject has an elevated B-natriuretic peptide level relative to a subject without heart failure.

9. The method of any of claims 1-9, wherein the subject has a reduced ejection fraction compared to a subject without heart failure.

10. The method of claim 9, wherein the subject has an ejection fraction below 50%.

11. The method of any of claims 1-6, wherein the subject is determined to have heart failure through any one or more of blood tests (e.g., measuring: brain natriuretic peptide, electrolytes, liver function, kidney function, thyroid function, complete blood count (CBC), and/or C-reactive protein), ultrasound (e.g., echocardiography), x-ray imaging, and electrocardiogram.

12. The method of any of claims 1-11, further comprising treating the subject, e.g., a human subject, with an additional therapy for treating heart failure or a heart failure associated condition, e.g., as a combination therapy.

13. The method of claim 12, wherein the additional therapy is administered in combination with the glycan preparation, e.g., administered concurrently with administration of the glycan preparation or administered sequentially with administration of the glycan preparation (e.g., prior to administration of the glycan preparation or after administration of the glycan preparation).

14. The method of either of claim 12 or 13, wherein the additional therapy is for treating heart failure.

15. The method of any of claims 12-14, wherein the additional therapy is selected from one or more of: pharmaceutical compositions, automatic implantable cardioconverter defibrillator (AICD), cardiac contractility modulation (CCM) (e.g., ventricular assist device (VAD)), or heart transplantation.

16. The method of any of claims 12-14, wherein the additional therapy comprises pharmaceutical compositions.

17. The method of claim 16, wherein the pharmaceutical composition is selected from one or more of: angiotensin converting enzyme inhibitor (ACEI), angiotensin receptor blocker (ARB), beta blocker, combination of hydralazine and long-acting nitrate (e.g., isosorbide dinitrate), aldosterone antagonist, mineralocorticoid antagonist (e.g., spironolactone), and diuretic (e.g., loop diuretics, thiazide-like diuretics, and potassium-sparing diuretic).

18. A method for increasing drug activity in a subject, wherein the drug comprises a sulfonamide, the method comprising:
a) administering a glycan composition in an amount effective and for a time sufficient to increase the drug activity in the subject;
b) administering a glycan composition in an amount effective and for a time sufficient to increase drug activity in the subject, and wherein at the time of administration of the glycan composition, the subject comprises a level of the drug that, in the presence of the administered glycan composition, provides a therapeutic effect;
c) administering the drug, wherein at the time of administration of the drug, the subject has already been administered the glycan composition in an amount effective and for a time sufficient to increase the drug activity in the subject;
d) administering the drug in an amount effective and for a time sufficient to increase the drug activity in the subject, wherein subject has been determined to be in need of the glycan composition, e.g., to increase the activity of the drug; or
e) administering the drug and the glycan composition to the subject, in amounts effective and for times sufficient to increase the drug activity in the subject, wherein administration of the drug and the glycan composition overlap;
wherein:
i) the glycan preparation comprises glycan polymers that comprise glucose, galactose, arabinose, mannose, fructose, xylose, fucose, or rhamnose glycan units;
ii) the average degree of branching (DB) of the glycan polymers in the glycan preparation is 0, between 0.01 and 0.6, between 0.05 and 0.5, between 0.1 and 0.4, or between 0.15 and 0.4;
iii) at least 50% (at least 60%, 65%, 70%, 75%, 80%, or 85%, or less than 50%) of the glycan polymers in the glycan preparation have a degree of polymerization (DP) of at least 3 and less than 30 glycan units, at least 3 and less than 10 glycan units, at least 5 and less than 25 glycan units, or at least 10 and less than 35 glycan units;
iv) the average DP (mean DP) of the glycan preparation is between about 5 and 8, between about 8 and 13, between about 13 and 25, between about 5 and 15, between about 5 and 20, or between about 5-15;
v) the ratio of alpha- to beta-glycosidic bonds present in the glycan polymers of the glycan preparation is 0, or between about 0.8:1 to about 5:1, between about 1:1 to about 5:1, between about 1:1 to about 3:1, between about 3:2 to about 2:1, or between about 3:2 to about 3:1,
vi) the glycan preparation comprises between 15 mol % and 75 mol % (between 20 mol % and 60 mol %, between 25 mol % and 50 mol %, or between 30 mol % and 45 mol %) 1,6 glycosidic bonds;
vii) the glycan preparation comprises between 1 mol % and 40 mol % (between 1 mol % and 30 mol %, between 5 mol % and 25 mol %, between 10 mol % and 20 mol %) of each at least one, two, or three of 1,2; 1,3; and 1,4 glycosidic bonds;
viii) the glycan preparation has a final solubility limit in water of at least about 50 (at least about 60, 70, at least about 75, or less than 50) Brix at 23° C.; or
ix) the glycan preparation has a dietary fiber content of at least 50% (at least 60%, 70%, 80%, or at least 90%, or less than 50%), and
x) any combination of two, three, four, five, six, seven, eight, or nine of i), ii), iii), iv), v), vi), vii), viii), and ix).

19. The method of claim 18, wherein the drug and the glycan preparation are administered to the subject for treating a disease selected from Diabetes mellitus, convulsions, Hepatitis C, HIV, inflammation, cardiac arrythmia, hypertension, glaucoma, bacterial infections, Ebola virus infections, Hepatitis B, pulmonary hypertension, migraine, erectile dysfunction, benign prostated hyperplasia.

20. The method of claim 18 or 19, wherein the drug is selected from
a) Sulfonylureas inlcuding Acetohexamide, Carbutamide, Chlorpropamide, Glibenclamide (glyburide), Glibornuride, Gliclazide, Glyclopyramide, Glimepiride, Glipizide, Gliquidone, Glisoxepide, Tolazamide, or Tolbutamide. Sulfonamides used to treat convulsions include Ethoxzolamide, Sultiame, Topiramate, and Zonisamide,
b) Ethoxzolamide, Sultiame, Topiramate, and Zonisamide,
c) Asunaprevir (or other NS3/4A protease inhibitor), Beclabuvir (or other NS5B RNA polymerase inhibitor), Dasabuvir, Grazoprevir, Paritaprevir, and Simeprevir
d) Amprenavir (or other HIV protease inhibitor), Darunavir, Delavirdine (or other non-nucleoside reverse transcriptase inhibitor), Fosamprenavir, and Tipranavir. Nucleoside analogue sulfonamides may also be used to treat HIV. Sulfonamides used to treat HIV alsoinclude deoxyadenosine analogues: didanosine (ddl), vidarabine (antiviral); deoxycytidine analogues: emtricitabine (FTC), lamivudine (3TC), zalcitabine (ddC); guanosine and deoxyguanosine analogues: abacavir; and thymidine and deoxythymidine analogues: stavudine (d4T), and zidovudine (azidothymidine, or AZT),
e) Apricoxib (COX-2 inhibitor), Celecoxib (COX-2 inhibitor), Parecoxib (COX-2 inhibitor), and Sulfasalazine (anti-inflammatory agent and a DMARD),
f) Dofetilide (class III antiarrhythmic), Dorzolamide (anti-glaucoma carbonic anhydrase inhibitor), Dronedarone (class III antiarrhythmic), and Ibutilide (class III antiarrhythmic). In some embodiments, cardiac glycoside sulfonamides used to treat arrhythmias include digoxin, digitoxin, convallotoxin, antiarin, and oleandrin,
g) Acetazolamide, Bumetanide, Chlorthalidone, Clopamide, Furosemide, Hydrochlorothiazide, Indapamide, Mefruside, Metolazone, and Xipamide,
h) Brinzolamide (carbonic anhydrase inhibitor for glaucoma), Dorzolamide (anti-glaucoma carbonic anhydrase inhibitor) and Acetazolamide,
i) Sulfafurazole, Sulfacetamide, Sulfadiazine, Sulfadimidine, Sulfafurazole (sulfisoxazole), Sulfisomidine (sulfaisodimidine), Sulfadoxine, Sulfamethoxazole, Sulfamoxole, Sulfanitran, Sulfadimethoxine, Sulfamethoxypyridazine, Sulfametoxydiazine, Sulfadoxine, Sulfametopyrazine, and Terephtyl,
j) adenosine analogs, e.g., BCX4430,
k) deoxycytidine analogues, e.g. lamivudine (3TC); guanosine and deoxyguanosine analogues, e.g. entecavir; and thymidine and deoxythymidine analogues, e.g. telbivudine,
l) Bosentan and Udenafil,
m) Sumatriptan,
n) Udenafil, or
o) Tamsulosin and Udenafil.

21. The method of claim 20, wherein the drug and the glycan preparation are administered to the subject for treating a disease and wherein the
a) the disease is Diabetes mellitus and the drug is selected from a),
b) the disease is convulsions and and the drug is selected from b),
c) the disease is Hepatitis C and the drug is selected from c),
d) the disease is HIV and the drug is selected from d)
e) the disease is inflammation and the drug is selected from e),
f) the disease is arrythmia and the drug is selected from f),
g) the disease is hypertension and the drug is selected from g),
h) the disease is glaucoma and the drug is selected from h),
i) the disease is bacterial infections and the drug is selected from i),
j) the disease is Ebola virus infections and the drug is selected from j),
k) the disease is Hepatitis B and the drug is selected from k),
l) the disease is pulmonary hypertension and the drug is selected from kl),
m) the disease is migraine and the drug is selected from m),
n) the disease is erectile dysfunction and the drug is selected from n), or
o) the disease is benign prostated hyperplasia and the drug is selected from o).

22. The method of any of claims 18-21, wherein the subject exhibits symptoms associated with:
a) diabetes mellitus, including one or more of polydispia, polyuria, unexplained weight loss, polyphagia, fatigue, neuropathy, and unhealed sores;
b) convulsion, including one or more of blackout, confusion, drooling, loss of bowel control, loss of bladder control, sudden shaking, uncontrolled muscle spasm, and temporary cessation of breathing;
c) Hepatitis C, including one or more of fever, dark urine, abdominal pain, yellow tinged skin, decreased appetite, fatigue, nausea, muscle pain, joint pain, and weight loss; d) Human Immunodeficiency Virus (HIV), including one or more of fever, large tender lymph nodes, sweats (e.g., night sweats), chills, weakness, unintended weight loss, throat inflammation, rash, headache, diarrhea, mouth sores, and genital sores;
e) inflammation, including one or more of heat, pain, redness, swelling, and loss of function;
f) cardiac arrhythmia, including one or more of heart palpitations, lightheadedness, syncope, shortness of breath, lower blood pressure, and chest pain;
g) hypertension, including one or more of elevated resting blood pressure and thickening of heart muscle;
h) glaucoma, including one or more of eye pain, vision loss, blurred vision, mid-dilated pupil, red eye, and nausea;
i) bacterial infection, including one or more of fatigue, pain, loss of appetite, weight loss, fevers, night sweats, chills, aches, abscess, diarrhea, runny nose, coughing, and skin rash;
j) Ebola virus infection, including one or more of fever, sore throat, muscular pain, joint pain, abdominal pain, headache, vomiting, diarrhea, rash, weakness, decreased appetite, feeling tired, internal bleeding, and external bleeding;
k) Hepatitis B, including one or more of loss of appetite, nausea, vomiting, body aches, fever, dark urine, tiredness, jaundice, and itchy skin;
l) pulmonary hypertension, including one or more of shortness of breath, fatigue, chest pain, palpitations, abdominal pain, poor appetite, lightheadedness, syncope, swelling (e.g., swelling in legs and/or swelling in ankles), and cyanosis;
m) migraine, including one or more of recurrent headaches, nausea, vomiting, photophobia, phonophobia, sensitivity to smell, neck pain, vertigo, weakness, tingling in limbs, cognitive symptoms, and irritability;
n) erectile dysfunction, including impotence; or
o) benign prostate hyperplasia (e.g., prostate enlargement), including one or more of abdominal pain, dysuria, urinary hesitancy, urinary intermittency, nocturia, continuous feeling of full bladder, frequent urination, acute urinary retention, slow urine flow, and bladder outlet obstruction.

23. The method of any of claims 18-22, wherein the subject has conditions associated with:
a) diabetes mellitus, including one or more of heart disease, kidney failure, strokes, diabetic retinopathy, and poor blood flow in the limbs;
b) convulsion, including one or more of head trauma, hypoglycemia, fever, stroke, and lack of oxygen to the brain;
c) Hepatitis C, including one or more of liver disease, cirrhosis, liver cancer, liver failure, and dilated blood vessels in the esophagus and stomach;
d) HIV, including one or more of toxoplasmosis (e.g., toxoplasmosis of the brain), candidiasis (e.g., candidiasis of the esophagus, trachea, bronchi, or lungs), Kaposi's sarcoma, Burkitt's lymphoma, primary central nervous system lymphoma, cervical cancer, opportunistic infections, cachexia, and pneumocystis pneumonia;
e) inflammation, including one or more of cancer, allergic reaction, myopathy, atherosclerosis, and ischemic heart disease;
f) cardiac arrhythmia, including one or more of stroke, heart failure, cardiac arrest, and sudden cardiac death;
g) hypertension, including one or more of obesity, coronary artery disease, stroke, heart failure, atrial fibrillation, peripheral vascular disease, vision loss, chronic kidney disease, heart disease, and dementia;
h) glaucoma, including one or more of high blood pressure, migraines, and obesity;
i) bacterial infection, including one or more of respiratory system infection, diarrheal disease, and cutaneous abscess;
j) Ebola virus infection, including one or more of abnormal blood clotting, disseminated intravascular coagulation (DIC), and hemorrhagic fever;
k) Hepatitis B, including one or more of liver damage, cirrhosis, liver cancer, serum-sickness-like syndrome, acute necrotizing vasculitis (e.g., polyarteritis nodose), and membranous glomerulonephritis;
l) pulmonary hypertension, including one or more of lung blood clots, COPD, and sleep apnea;
n) erectile dysfunction, including one or more of diabetes, cardiovascular disease, hormonal insufficiency (e.g., hypogonadism), trauma, coronary artery disease, and peripheral vascular disease; or
o) benign prostate hyperplasia, including one or more of urinary tract infection, bladder stone, chronic kidney problems, prostate cancer, kidney disease, and diabetes.

24. The method of any of claims 18-23, wherein the subject has findings associated with:
a) diabetes mellitus, including one or more of high blood sugar, insulin resistance, and lack of insulin;
b) convulsion, including one or more of structural problems inside the brain and nervous system infection;
c) Hepatitis C, including one or more of antibodies to Hepatitis C Virus (HCV), elevated liver enzymes, lymphocytes in the parenchyma, lymphoid follicles in the portal triad, and bile duct changes;
d) HIV, including one or more of CD4+ T cell count below 200 cells per μL, positive antibodies to HIV-1 and/or HIV-2, and p24 antigen;
e) inflammation, including one or more of increased presence of plasma and leukocytes at sites of injury, increased presence of mononuclear cells at sites of inflammation, and increased swelling;
f) cardiac arrhythmia, including one or more of irregular heartbeat, tachycardia, and bradycardia;
g) hypertension, including one or more of thickening of heart muscle (e.g., left ventricular hypertrophy), heart enlargement, heart damage, consistent systolic blood pressure above 139 mmHg, and consistent diastolic blood pressure measurement above 89 mmHg;
h) glaucoma, including one or more of very high intraocular pressure (e.g., >30 mmHg), damaged optic nerve, and vision loss;
i) bacterial infection, including one or more of fever, diarrhea, and abscess;
j) Ebola virus infection, including one or more of decreased liver function, decreased kidney function, low platelet count, elevated alanine aminotransferase, elevated aspartate aminotransferase, prolonged prothrombin time, and decreased white blood cell count followed by increased white blood cell count;
k) Hepatitis B, including one or more of elevated serum alanine aminotransferase, liver inflammation, Hepatitis B Virus (HBV) DNA, Hepatitis B surface antigen (HBsAg), and Hepatitis B e antigen (HBeAg);
l) pulmonary hypertension, including one or more of PAP at least 25 mm Hg at rest, and PAP >25 mm Hg with pulmonary arterial occlusion pressure <15 mm Hg;
m) migraines, including one or more of nausea, vomiting, photophobia, phonophobia;
n) erectile dysfunction, including one or more of impotence; or
o) benign prostate hyperplasia, including elevated prostate specific antigen (PSA).

25. The method of any of claims 18-24, wherein the subject has:
a) a fasting plasma glucose greater than or equal to 7.0 mmol/l (126 mg/dl); plasma glucose greater than or equal to 11.1 mmol/l (200 mg/dl) two hours after an oral dose of glucose; reduced insulin levels relative to a subject without diabetes mellitus; glycated hemoglobin (HbA 1c) of greater than or equal to 48 mmol/mol; high C-peptide levels relative to a subject without diabetes mellitus type 2; and low C-peptide levels relative to a subject without diabetes mellitus type 1;
b) one or more of: structural problems inside the brain relative to a subject without convulsions;
c) one or more of: antibodies to Hepatitis C Virus (HCV), elevated liver enzymes relative to a subject without HCV, lymphocytes in the parenchyma relative to a subject without HCV, lymphoid follicles in the portal triad relative to a subject without HCV, and bile duct changes relative to a subject without HCV;
d) one or more of: CD4+ T cell count below 200 cells per μL relative to a subject without HIV, positive antibodies to HIV-1 and/or HIV-2, and presence of p24 antigen;
e) one or more of: visibly increased swelling at a site relative to a site without inflammation, increased presence of plasma and leukocytes at sites of injury relative to a subject without inflammation, increased presence of mononuclear cells at sites of inflammation compared to a subject without inflammation;
f) one or more of: an irregular heartbeat relative to a subject without cardiac arrhythmia, heartbeat faster than 100 beats per minute, heartbeat slower than 60 beats per minute;
g) one or more of: visibly enlarged heart relative to a subject without hypertension, elevated thickening of heart muscle (e.g., left ventricular hypertrophy) relative to a subject without hypertension, heart damage compared to a subject without hypertension, consistent systolic blood pressure above 139 mmHg, consistent diastolic blood pressure measurement above 89 mmHg;
h) one or more of: high intraocular pressure relative to a subject without glaucoma, optic nerve damage relative to a subject without glaucoma, vision loss compared to a subject without glaucoma, intraocular pressure above 30 mmHg;
i) one or more of: fever relative to a subject without bacterial infection, diarrhea relative to a subject without bacterial infection, and abscess relative to a subject without bacterial infection;
j) one or more of: decreased liver function relative to a subject without Ebola virus infection, decreased kidney function relative to a subject without Ebola virus infection, low platelet count relative to a subject without Ebola virus infection, elevated alanine aminotransferase level relative to a subject without Ebola virus infection, elevated aspartate aminotransferase level relative to a subject without Ebola virus infection, prolonged prothrombin time relative to a subject without Ebola virus infection, decreased white blood cell count followed by increased white blood cell count;
k) one or more of: elevated serum alanine aminotransferase relative to a subject without Hepatitis B, elevated liver inflammation relative to a subject without Hepatitis B, Hepatitis B Virus (HBV) DNA, Hepatitis B surface antigen (HBsAg), Hepatitis B e antigen (HBeAg);
l) one or more of: PAP at least 25 mm Hg at rest, PAP≥25 mm Hg with pulmonary arterial occlusion pressure≤15 mm Hg;
m) recurrent headaches;
n) impotence; or
o) elevated prostate specific antigen (PSA) relative to a subject without benign prostate hyperplasia.

26. The method of any of claims 18-24, wherein the subject is determined to have:
a) diabetes mellitus through any one or more of fasting glucose test, glucose tolerance test, measuring glycated hemoglobin (HbA1c), and measuring C-peptide level;
b) convulsion through any one or more of blood tests (e.g., measuring: glucose, electrolytes, calcium), lumbar puncture, CT scan, MRI scan, and electroencephalography;
c) Hepatitis C through any one or more of blood tests (e.g., detecting antibodies to HCV), recombinant immunoblot assay, quantitative HCV RNA polymerase chain reaction (PCR), and biopsy;
d) HIV through any one or more of enzyme-linked immunosorbent assay (ELISA), immunoassay combination test, western blot, polymerase chain reaction (PCR), immunofluorescence assay (IFA), and nucleic acid testing (NAT);
e) inflammation through any one or more of physical examination (e.g., examining joint symptoms), blood tests, and x-ray imaging;
f) cardiac arrhythmia through any one or more of auscultation of a heartbeat, feeling peripheral pulse, electrocardiography, and transesophageal atrial stimulation;
g) hypertension through any one or more of chest X-ray, electrocardiogram, measurement of systolic blood pressure, and measurement of diastolic blood pressure;
h) glaucoma through any one or more of tonometry, gonioscopy, ophthalmoscopy, perimetry, pachymetry, nerve fiver analysis, optical coherence tomography, scanning laser polarimetry, and scanning laser ophthalmoscopy;
i) bacterial infection through any one or more of microbial culture, microscopy (e.g., Gram stain, acid-fast stain), x-rays, CAT scan, PET scan, NMR, Gram stain, acid-fast stain, biochemical tests, serological methods (e.g., immunoassay), and polymerase chain reaction (PCR);
j) Ebola virus infection through any one or more of cell culture, microscopy (e.g., electron microscopy), enzyme-linked immunosorbsent assay (ELISA), and polymerase chain reaction (e.g., rRT-PCR);
k) Hepatitis B through any one or more of serum test, blood test, and polymerase chain reaction (PCR) test;
l) pulmonary hypertension through any one or more of echocardiography, electrocardiography, x-rays, computed tomography (CT) scan, and pulmonary function tests (e.g., carbon monoxide and arterial blood gas measurements);
m) migraine based on presence of pulsating headaches;
n) erectile dysfunction through any one or more of duplex ultrasound, bulbocavernous reflex test, penile biothesiometry, dynamic infusion cavernosometry, cavernosography, digital subtraction angiography, and MRI; or
o) benign prostate hyperplasia through any one or more of digital rectal exam, urinalysis, kidney function test, prostate specific antigen screening, and ultrasound.

27. The method of any of claims 18-26, further comprising treating the subject, e.g., a human subject, with an additional therapy for:
a) treating diabetes mellitus or a diabetes mellitus-associated condition, e.g., as a combination therapy;
b) convulsion or a convulsion associated condition, e.g., as a combination therapy;
c) Hepatitis C or a Hepatitis C associated condition, e.g., as a combination therapy;
d) HIV or a HIV associated condition, e.g., as a combination therapy;
e) inflammation or an inflammation associated condition, e.g., as a combination therapy;
f) cardiac arrhythmia or a cardiac arrhythmia associated condition, e.g., as a combination therapy;
g) hypertension or a hypertension associated condition, e.g., as a combination therapy;
h) glaucoma or a glaucoma associated condition, e.g., as a combination therapy;
i) bacterial infection or a bacterial infection associated condition, e.g., as a combination therapy;
j) Ebola virus infection or an Ebola virus infection associated condition, e.g., as a combination therapy;
k) Hepatitis B or a Hepatitis B associated condition, e.g., as a combination therapy;
l) pulmonary hypertension or a pulmonary hypertension associated condition, e.g., as a combination therapy;
m) migraine or a migraine associated condition, e.g., as a combination therapy;
n) erectile dysfunction or an erectile dysfunction associated condition, e.g., as a combination therapy; or
o) benign prostate hyperplasia or a benign prostate hyperplasia associated condition, e.g., as a combination therapy.

28. The method of claim 27, wherein the additional therapy is administered in combination with the glycan preparation, e.g., administered concurrently with administration of the glycan preparation or administered sequentially with administration of the glycan preparation (e.g., prior to administration of the glycan preparation or after administration of the glycan preparation).

29. The method of either of claim 27 or 28, wherein the additional therapy is for treating:
a) diabetes mellitus,
b) convulsion,
c) Hepatitis C,
d) HIV,
e) inflammation,
f) cardiac arrhythmia,
g) hypertension,
h) glaucoma,
i) bacterial infection,
j) Ebola virus,
) Hepatitis B,
l) pulmonary hypertension,
m) migraine,
n) erectile dysfunction, or
o) benign prostate hyperplasia.

30. The method of any of claims 27-29, wherein the additional therapy is selected from one or more of:
a) pharmaceutical compositions for diabetes;
b) pharmaceutical compositions for convulsion;
c) pharmaceutical compositions; liver transplantation, and liver transplantation with ribavirin and pegylated interferon for Hepatitis C;
d) pharmaceutical compositions for HIV;
e) pharmaceutical compositions, physical therapy, and devices to reduce stress on joints (e.g., braces, splints, canes) for inflammation;
f) pharmaceutical compositions, implantable cardioverter-defibrillator (ICD), and pacemaker for cardiac arrhythmia;
g) pharmaceutical compositions for hypertension;
h) pharmaceutical compositions, laser therapy (e.g., argon laser trabeculoplasty, selective laser trabeculoplasty, Nd:YAG laser peripheral irdotomy), or surgery (e.g., canaloplasty, trabeculectomy, non-penetrating deep sclerectomy) for glaucoma;
i) pharmaceutical compositions or surgery for bacterial infection;
j) pharmaceutical compositions for Ebola virus infection;
k) pharmaceutical compositions for Hepatitis B;
l) pharmaceutical compositions, oxygen therapy, atrial septostomy lung transplantation, or pulmonary thromboendarterectomy;
m) pharmaceutical compositions for migraine;
n) pharmaceutical compositions, vacuum erection device, or penile implant for erectile dysfunction; or
o) pharmaceutical compositions, catheterization (e.g., intermittent urinary catheterization), open prostatectomy, transurethral resection of the prostate, transurethral incision of the prostate, or photoselective vaporization of the prostate for benign prostate hyperplasia.

31. The method of any of claims 27-29, wherein the additional therapy is pharmaceutical compositions.

32. The method of claim 31, wherein the pharmaceutical composition is selected from one or more of:
a) metformin, insulin, thiazolidinedione, dipeptidyl peptidase-4 inhibitor, SGLT2 inhibitor, and glucagon-like peptide-1 analog for diabetes;
b) benzodiazepine, barbiturate, and propofol for convulsion;
c) ledipasvir, sofosbuvir, velpatasvir, elbasvir, daclatasvir, and voxilaprevir for Hepatitis C;
d) maraviroc, enfuvirtide, tenofovir, etravirine, rilpivirine, elvitegravir, dolutegravir, raltegravir, lopinavir, nelfinavir, indinavir, ritonavir, atazanavir, bevirimat, vivecon, and combinations thereof for HIV;
e) anti-inflammatory pain reliever (e.g., non-steroidal anti-inflammatory drug (NSAID), aspirin, ibuprofen, Celebrex), corticosteroid (e.g., prednisone), chemotherapeutic, biologic therapy, and narcotic pain reliever for inflammation;
f) warfarin, heparin, and anti-platelet drug (e.g., aspirin) for cardiac arrythmia;
g) thiazide diuretic, calcium channel blocker, angiotensin converting enzyme inhibitor, and angiotensin receptor blocker for hypertension;
h) prostaglandin analog (e.g., latanoprost, bimatoprost, travoprost), beta adrenergic receptor analog (e.g., timolol, levobunolol, betaxolol), alpha2-adrenergic agonist (e.g., brimonidine, apraclonidine), alpha agonist (e.g., epinephrine), miotic agent (e.g., parasympathomimetics (e.g., pilocarpine), and acetylcholinesterase inhibitor (e.g., echothiophate) for glaucoma;
i) a penicillin, a cephalosporin, an aminoglycoside, a macrolide, a quinolone, a tetracycline, and a metronidazole for bacterial infection;
k) adefovir, tenofovir, interferon alpha-2a, and PEGylated interferon alpha-2a for Hepatitis B;
l) epoprostenol, treprostinil, iloprost, ambrisentan, macitentan, sildenafil, remodulin, prostacyclin, prostaglandin I2, tadalafil, endothelin antagonist, prostanoid, phosphodiesterase inhibitor, endothelin receptor antagonist, calcium channel blocker, and diuretic for pulmonary hypertension;
m) ibuprofen, paracetamol (e.g., acetaminophen), metoprolol, valproate, topiramate, ketorolac, ergotamine, dihydroergotamine, metoclopramide, lidocaine, haloperidol, and dexamethasone for migraine;
n) sildenafil, vardenafil, tadalafil, alprostadil (e.g., alprostadil with permeation enhancer DDAIP), papaverine, phentolamine, and prostaglandin E1; or
o) alpha blocker (e.g., alfuzosin, doxazosin, silodosin, and terazosin), 5α-reductase inhibitor (e.g., finasteride and dutasteride), antimuscarinic (e.g., tolterodine), and phosphodiesterase-5 inhibitor (e.g., sildenafil and tadalafil).

33. A method for increasing drug activity in a subject, wherein the drug comprises a nucleoside analogue, the method comprising:
a) administering a glycan composition in an amount effective and for a time sufficient to increase the drug activity in the subject;
b) administering a glycan composition in an amount effective and for a time sufficient to increase drug activity in the subject, and wherein at the time of administration of the glycan composition, the subject comprises a level of the drug that, in the presence of the administered glycan composition, provides a therapeutic effect;
c) administering the drug, wherein at the time of administration of the drug, the subject has already been administered the glycan composition in an amount effective and for a time sufficient to increase the drug activity in the subject;
d) administering the drug in an amount effective and for a time sufficient to increase the drug activity in the subject, wherein subject has been determined to be in need of the glycan composition, e.g., to increase the activity of the drug; or
e) administering the drug and the glycan composition to the subject, in amounts effective and for times sufficient to increase the drug activity in the subject, wherein administration of the drug and the glycan composition overlap;
wherein:
i) the glycan preparation comprises glycan polymers that comprise glucose, galactose, arabinose, mannose, fructose, xylose, fucose, or rhamnose glycan units;
ii) the average degree of branching (DB) of the glycan polymers in the glycan preparation is 0, between 0.01 and 0.6, between 0.05 and 0.5, between 0.1 and 0.4, or between 0.15 and 0.4;
iii) at least 50% (at least 60%, 65%, 70%, 75%, 80%, or 85%, or less than 50%) of the glycan polymers in the glycan preparation have a degree of polymerization (DP) of at least 3 and less than 30 glycan units, at least 3 and less than 10 glycan units, at least 5 and less than 25 glycan units, or at least 10 and less than 35 glycan units;
iv) the average DP (mean DP) of the glycan preparation is between about 5 and 8, between about 8 and 13, between about 13 and 25, between about 5 and 15, between about 5 and 20, or between about 5-15;
v) the ratio of alpha- to beta-glycosidic bonds present in the glycan polymers of the glycan preparation is 0, or between about 0.8:1 to about 5:1, between about 1:1 to about 5:1, between about 1:1 to about 3:1, between about 3:2 to about 2:1, or between about 3:2 to about 3:1,
vi) the glycan preparation comprises between 15 mol % and 75 mol % (between 20 mol % and 60 mol %, between 25 mol % and 50 mol %, or between 30 mol % and 45 mol %) 1,6 glycosidic bonds;
vii) the glycan preparation comprises between 1 mol % and 40 mol % (between 1 mol % and 30 mol %, between 5 mol % and 25 mol %, between 10 mol % and 20 mol %) of each at least one, two, or three of 1,2; 1,3; and 1,4 glycosidic bonds;
viii) the glycan preparation has a final solubility limit in water of at least about 50 (at least about 60, 70, at least about 75, or less than 50) Brix at 23° C.; or
ix) the glycan preparation has a dietary fiber content of at least 50% (at least 60%, 70%, 80%, or at least 90%, or less than 50%), and
x) any combination of two, three, four, five, six, seven, eight, or nine of i), ii), iii), iv), v), vi), vii), viii), and ix).

34. The method of claim 33, wherein the drug and the glycan preparation are administered to the subject for treating cancer.

35. The method of claim 33 or 34, wherein the drug comprises a nucleoside analogue selected from the group consisting of deoxycytidine analogues, e.g. cytarabine, gemcitabine; pyrimidine analogues, e.g. 5-Fluorouracil (5FU), Floxuridine (FUDR), Cytarabine (Cytosine arabinoside), 6-azauracil (6-AU); and purine analogues, e.g. Mercaptopurine, Thiopurines, Fludarabine, Pentostatin.

36. The method of any of claims 33-35, wherein the subject exhibits symptoms associated with cancer, including one or more of abnormal cell growth, lump, abnormal bleeding, prolonged cough, unexplained weight loss, and change in bowel movements.

37. The method of any of claims 33-36, wherein the subject has findings associated with cancer, including one or more of genetic mutations, fusion genes, and numerical chromosome changes relative to a subject without cancer.

38. The method of any of claims 33-37, wherein the subject is determined to have cancer through any one or more of biopsy, blood test, x-ray imaging, CT scan, endoscopy and immunohistochemistry.

39. The method of any of claims 33-38, further comprising treating the subject, e.g., a human subject, with an additional therapy for treating cancer or a cancer associated condition, e.g., as a combination therapy.

40. The method of claim 39, wherein the additional therapy is administered in combination with the glycan preparation, e.g., administered concurrently with administration of the glycan preparation or administered sequentially with administration of the glycan preparation (e.g., prior to administration of the glycan preparation or after administration of the glycan preparation).

41. The method of either of claim 39 or 40, wherein the additional therapy is for treating cancer.

42. The method of any of claims 39-41, wherein the additional therapy is selected from one or more of: chemotherapy, radiation therapy, laser therapy, surgery, antibody, or adoptive cell transfer.

43. The method of claim 33, wherein the drug and the glycan preparation are administered to the subject for treating viral infection (e.g., Herpes Simplex Virus Type 1 and/or Varicella Zoster Virus).

44. The method of claim 33 or 43, wherein the drug comprises a nucleoside analogue selected from the group consisting of sorivudine.

45. The method of any of claim 33 or 43-44, wherein the subject exhibits symptoms aasocited with viral infection (e.g., Herpes Simplex Virus Type 1 and/or Varicella Zoster Virus), including one or more of lesions of the skin (e.g., rashes) and lesions in the skin or mucous membranes of the mouth, lips, nose, or genitals.

46. The method of any of claim 33 or 43-45, wherein the subject is determined to have viral infection (e.g., Herpes Simplex Virus Type 1 and/or Varicella Zoster Virus) through any one or more of detection of lesions, viral DNA, viral proteins, and antibodies to viral proteins.

47. The method of any of claim 33 or 43-46, wherein the subject has conditions associated with viral infection (e.g., Herpes Simplex Virus Type 1 and/or Varicella Zoster Virus), including one or more of Alzheimer's disease, Chickenpox (e.g., varicella), Shingles, encephalitis, pneumonia (e.g., viral or secondary bacterial), bronchitis (e.g., viral or secondary bacterial), postherpetic neuralgia, Mollaret's meningitis, zoster multiplex, brain and/or nerve inflammation, and Reye's syndrome.

48. The method of any of claim 33 or 43-47, further comprising treating the subject, e.g., a human subject, with an additional therapy for treating viral infection (e.g., Herpes Simplex Virus Type 1 and/or Varicella Zoster Virus) or a virall infection associated condition, e.g., as a combination therapy.

49. The method of claim 48, wherein the additional therapy is administered in combination with the glycan preparation, e.g., administered concurrently with administration of the glycan preparation or administered sequentially with administration of the glycan preparation (e.g., prior to administration of the glycan preparation or after administration of the glycan preparation).

50. The method of either of claim 48 or 49, wherein the additional therapy is for treating viral infection (e.g., Herpes Simplex Virus Type 1 and/or Varicella Zoster Virus).

51. The method of any of claims 48-50, wherein the additional therapy is pharmaceutical compositions.

52. The method of claim 51, wherein the pharmaceutical composition is selected from one or more of: aciclovir (e.g., acyclovir), famciclovir, valaciclovir, zoster-immune globulin(ZIG), vidarabine, and VZV immune globulin.

53. A method for increasing drug activity in a subject, wherein the drug comprises an aminosalicylate, the method comprising:
a) administering a glycan composition in an amount effective and for a time sufficient to increase the drug activity in the subject;
b) administering a glycan composition in an amount effective and for a time sufficient to increase drug activity in the subject, and wherein at the time of administration of the glycan composition, the subject comprises a level of the drug that, in the presence of the administered glycan composition, provides a therapeutic effect;
c) administering the drug, wherein at the time of administration of the drug, the subject has already been administered the glycan composition in an amount effective and for a time sufficient to increase the drug activity in the subject;
d) administering the drug in an amount effective and for a time sufficient to increase the drug activity in the subject, wherein subject has been determined to be in need of the glycan composition, e.g., to increase the activity of the drug; or
e) administering the drug and the glycan composition to the subject, in amounts effective and for times sufficient to increase the drug activity in the subject, wherein administration of the drug and the glycan composition overlap;
wherein:
i) the glycan preparation comprises glycan polymers that comprise glucose, galactose, arabinose, mannose, fructose, xylose, fucose, or rhamnose glycan units;
ii) the average degree of branching (DB) of the glycan polymers in the glycan preparation is 0, between 0.01 and 0.6, between 0.05 and 0.5, between 0.1 and 0.4, or between 0.15 and 0.4;
iii) at least 50% (at least 60%, 65%, 70%, 75%, 80%, or 85%, or less than 50%) of the glycan polymers in the glycan preparation have a degree of polymerization (DP) of at least 3 and less than 30 glycan units, at least 3 and less than 10 glycan units, at least 5 and less than 25 glycan units, or at least 10 and less than 35 glycan units;
iv) the average DP (mean DP) of the glycan preparation is between about 5 and 8, between about 8 and 13, between about 13 and 25, between about 5 and 15, between about 5 and 20, or between about 5-15;
v) the ratio of alpha- to beta-glycosidic bonds present in the glycan polymers of the glycan preparation is 0, or between about 0.8:1 to about 5:1, between about 1:1 to about 5:1, between about 1:1 to about 3:1, between about 3:2 to about 2:1, or between about 3:2 to about 3:1,
vi) the glycan preparation comprises between 15 mol % and 75 mol % (between 20 mol % and 60 mol %, between 25 mol % and 50 mol %, or between 30 mol % and 45 mol %) 1,6 glycosidic bonds;
vii) the glycan preparation comprises between 1 mol % and 40 mol % (between 1 mol % and 30 mol %, between 5 mol % and 25 mol %, between 10 mol % and 20 mol %) of each at least one, two, or three of 1,2; 1,3; and 1,4 glycosidic bonds;
viii) the glycan preparation has a final solubility limit in water of at least about 50 (at least about 60, 70, at least about 75, or less than 50) Brix at 23° C.; or ix) the glycan preparation has a dietary fiber content of at least 50% (at least 60%, 70%, 80%, or at least 90%, or less than 50%), and
x) any combination of two, three, four, five, six, seven, eight, or nine of i), ii), iii), iv), v), vi), vii), viii), and ix).

54. The method of claim 53, wherein the drug and the glycan preparation are administered to the subject for treating ulcerative colitis or Crohn's disease.

55. The method of claim 53 or 54, wherein aminosalicylate is selected from 4-Aminosalicylic acid, Balsalazide, Olsalazine, Sulfasalazine, or Mesalazine (5-Aminosalicylic acid).

56. The method of any of claims 53-55, wherein the subject exhibits symptoms associated with ulcerative colitis, including one or more of ulcers (e.g., ulcers of the colon and rectum), inflammation (e.g., inflammation of the colon and rectum), abdominal pain, diarrhea (e.g., diarrhea mixed with blood and mucus), weight loss, painful bowel movements, anemia, fever, and increased bowel movements.

57. The method of any of claims 53-56, wherein the subject has conditions associated with ulcerative colitis, including one or more of megacolon, inflammation (e.g., inflammation of the eye, joints, or liver), hypokalemia, hypomagnesemia, pre-renal failure, and colon cancer.

58. The method of any of claims 53-57, wherein the subject has findings associated with ulcerative colitis, including one or more of elevated erythrocyte sedimentation rate, elevated C-reactive protein level, loss of vascular appearance of the colon erythema (e.g., redness of the intestinal mucosa), superficial ulceration, and pseudopolyps.

59. The method of any of claims 53-58, wherein the subject has elevated erythrocyte sedimentation rate relative to a subject without ulcerative colitis.

60. The method of any of claims 53-58, wherein the subject has an elevated C-reactive protein level relative to a subject without ulcerative colitis.

61. The method of any of claims 53-58, wherein the subject is determined to have ulcerative colitis through any one or more of blood test, electrolyte analysis, liver function test, x-ray imaging, biopsy, and endoscopy (e.g., sigmoidoscopy).

62. The method of any of claims 54-61, further comprising treating the subject, e.g., a human subject, with an additional therapy for treating ulcerative colitis or an ulcerative colitis associated condition, e.g., as a combination therapy.

63. The method of claim 62, wherein the additional therapy is administered in combination with the glycan preparation, e.g., administered concurrently with administration of the glycan preparation or administered sequentially with administration of the glycan preparation (e.g., prior to administration of the glycan preparation or after administration of the glycan preparation).

64. The method of either of claim 62 or 63, wherein the additional therapy is for treating ulcerative colitis.

65. The method of any of claims 62-64, wherein the additional therapy is selected from one or more of: pharmaceutical compositions or surgery (e.g., surgical removal of the large intestine).

66. The method of any of claims 62-64, wherein the additional therapy comprises pharmaceutical compositions.

67. The method of claim 66, wherein the pharmaceutical composition is selected from one or more of: corticosteroid (e.g., prednisone), immunosuppressant (e.g., azathioprine), budesonide, cyclosporin, fexofenadine, tacrolimus, tofacitinib, vedolizumab, etrolizumab, and TNF inhibitors (e.g., infliximab, adalimumab, and golimumab).

68. The method of any of claims 53-55, wherein the subject exhibits symptoms associated with Crohn's disease, including one or more of abdominal pain, diarrhea, fever, and weight loss.

69. The method of any of claims 53-55, and 68, wherein the subject has conditions associated with Crohn's disease, including one or more of anemia, skin rashes, arthritis, inflammation of the eye, tiredness, gallstones, rheumatologic disease (e.g., seronegative spondyloarthropathy) and bowel cancer.

70. The method of any of claims 53-55, 68, and 69, wherein the subject has findings associated with Crohn's disease, including one or more of fistulae, skin lesion (e.g., erythema nodosum and pyoderma gangrenosum), and patchy distribution of inflammation throughout the colon and ileum but not the rectum.

71. The method of any of claims 53-55, and 68-70, wherein the subject is determined to have Crohn's disease through any one or more of biopsy, CT imaging, MRI imaging, and colonoscopy.

72. The method of any of claims 53-55, and 68-71, further comprising treating the subject, e.g., a human subject, with an additional therapy for treating Crohn's disease or a Crohn's disease associated condition, e.g., as a combination therapy.

73. The method of claim 72, wherein the additional therapy is administered in combination with the glycan preparation, e.g., administered concurrently with administration of the glycan preparation or administered sequentially with administration of the glycan preparation (e.g., prior to administration of the glycan preparation or after administration of the glycan preparation).

74. The method of either of claim 72 or 73, wherein the additional therapy is for treating Crohn's disease.

75. The method of any of claims 72-74, wherein the additional therapy is selected from one or more of: pharmaceutical compositions or surgery (e.g., surgical removal of obstructions, fistula, or abscess).

76. The method of any of claims 72-74, wherein the additional therapy comprises pharmaceutical compositions.

77. The method of claim 76, wherein the pharmaceutical composition is selected from one or more of: prednisone, hydrocortisone, immunomodulator (e.g., azathioprine), methotrexate, natalizumab, ustekinumab, certolizumab, infliximab, and adalimumab.

78. The method of any of claims 1-77, wherein the drug activity is increased relative to a reference level, e.g., increased relative to a reference level, e.g., a preselected level, the baseline level at the time of administration of the glycan composition, or the level that would be seen in the absence of administration of the glycan composition.

79. The method of any of claims 1-78, wherein the drug is administered at a higher dose compared to a reference dose, e.g., the dose of drug administered to a subject not administered the glycan composition or a dosage that was approved by a regulatory agency, e.g., FDA (USA) or EMA (EU), or PMDA (Japan).

80. The method of any of any of claims 1-79, wherein the method comprises modifying, e.g., increasing or decreasing, the prevalence of a microbe or microbial taxa in the subject, e.g., in the gastrointestinal tract of the subject, e.g., by at least 25% (e.g., at least 25%, 30%, 40%, 50%, 60%, 70%, 80%, 90%, 95%, 97%, 98%, 99%, 100%, 1.5-fold, 2-fold, 3-fold, 4-fold, 5-fold, 10-fold, 15-fold, 20-fold, 25-fold, 50-fold, 100-fold, 250-fold, 500-fold, 1000-fold, or more), as compared to a reference level, e.g., the baseline level, level prior to administration, or level in the absence of administration of the glycan composition.

81. The method of any of claims 1-80, wherein the method comprises modifying, e.g., increasing or decreasing, the amount of a substrate, metabolite, or product of the enzyme activity in the subject, e.g., in the GI tract of the subject, e.g., by at least 25% (e.g., at least 25%, 30%, 40%, 50%, 60%, 70%, 80%, 90%, 95%, 97%, 98%, 99%, 100%, 1.5-fold, 2-fold, 3-fold, 4-fold, 5-fold, 10-fold, 15-fold, 20-fold, 25-fold, 50-fold, 100-fold, 250-fold, 500-fold, 1000-fold, or more), as compared to a reference level, e.g., the baseline level, level prior to administration, or level in the absence of administration of the glycan composition.

82. The method of any of claims 1-81, wherein the method comprises modulating (i) hydroxylating, (ii) methylating, (iii) sulfonating, (iv) hydrolyzing, (v) oxidizing, (vi) reducing, (vii) aromatizing, (viii) alkylating, (ix) acylating, (x) phosphorylating, (xi) glycosylating, (xii) sulfating, and/or (xiii) nitrosylating, the drug or a drug metabolite.

83. The method of any of claims 1-82, wherein the subject is a human subject.

84. The method of claim 83, wherein the subject is a newborn (a preterm newborn, a full-term newborn), an infant up to one year of age, a young child (e.g., 1 year to 12 years), a teenager (e.g., 13-19 years), an adult (e.g., 20-64 years), or an elderly adult (e.g., 65 years and older).

85. The method of claim 84 wherein the subject is a newborn (a preterm newborn, a full-term newborn) or an infant up to one year of age.

86. The method of claim 84, wherein the subject is a young child (e.g., 1 year to 12 years).

87. The method of claim 84, wherein the subject is a teenager (e.g., 13-19 years).

88. The method of claim 84, wherein the subject is an adult (e.g., 20-64 years).

89. The method of claim 84, wherein the subject is an elderly adult (e.g., 65 years and older).

90. The method of any of claims 1-89, wherein the method comprises modulating enzyme activity in the gastrointestinal tract of the human subject.

91. The method of any of claims 1-90, wherein the glycan composition further comprises a polyphenol.

92. The method of any of claims 1-91, wherein the glycan composition further comprises a probiotic bacterium or preparation thereof.

93. The method of any of claims 1-92 wherein the glycan composition comprises glycan polymers that comprise at least two distinct glycan units of glucose, galactose, arabinose, mannose, fructose, xylose, fucose, and rhamnose.

94. The method of any of claims 1-93, wherein the glycan composition comprises glycan polymers that comprise at least three distinct glycan units of glucose, galactose, arabinose, mannose, fructose, xylose, fucose, and rhamnose.

95. The method of any of claims 1-93, wherein the glycan composition comprises glycan polymers consisting of one or more of glucose, galactose, arabinose, mannose, fructose, xylose, fucose, or rhamnose glycan units.

96. The method of any of claims 1-95, wherein the glycan composition comprises glycan polymers consisting of one or more of glucose, galactose, mannose, or arabinose glycan units.

97. The method of any of claims 1-95, wherein at least two of the glycosidic bonds independently comprise a 1→2 glycosidic bond, a 1→3 glycosidic bond, a 1→4 glycosidic bond, or a 1→6 glycosidic bond.

98. The method of any of claims 1-95, wherein at least three of the glycosidic bonds independently comprise a 1→2 glycosidic bond, a 1→3 glycosidic bond, a 1→4 glycosidic bond, or a 1→6 glycosidic bond.

99. The method of any of claims 1-98, wherein:
i) the glycan preparation comprises glycan polymers that comprise glucose, galactose, arabinose, mannose, fructose, xylose, fucose, or rhamnose glycan units; and
ii) the average DP (mean DP) of the glycan preparation is between about 3 and 25.

100. The method of any of claims 1-99, wherein at least 50% of the glycan polymers in the glycan preparation have a degree of polymerization (DP) of at least 3 and less than 30 glycan units.

101. The method of any of claims 1-99, wherein at least 60% of the glycan polymers in the glycan preparation have a degree of polymerization (DP) of at least 3 and less than 30 glycan units.

102. The method of any of claims 1-101, wherein at least 60% of the glycan polymers in the glycan preparation have a degree of polymerization (DP) of at least 3 and less than 10 glycan units, at least 5 and less than 25 glycan units, or at least 10 and less than 35 glycan units.

103. The method of any of claims 1-102, wherein the glycan preparation comprises between 15 mol % and 75 mol % 1,6 glycosidic bonds.

104. The method of any of claims 1-102, wherein the glycan preparation comprises between 25 mol % and 50 mol % 1,6 glycosidic bonds.

105. The method of any of claims 1-102, wherein the glycan preparation comprises between 30 mol % and 45 mol % 1,6 glycosidic bonds.

106. The method of any of claims 1-102, wherein the glycan preparation comprises between 1 mol % and 40 mol % of each of 1,2; 1,3; and 1,4 glycosidic bonds.

107. The method of any of claims 1-102, wherein the glycan preparation comprises between 5 mol % and 25 mol % of each of 1,2; 1,3; and 1,4 glycosidic bonds.

108. The method of any of claims 1-102, wherein the glycan preparation comprises between 10 mol % and 20 mol % of each of 1,2; 1,3; and 1,4 glycosidic bonds.

109. The method of any of claims 1-108, wherein the glycan preparation has a final solubility limit in water of at least about 50 Brix at 23° C.

110. The method of any of claims 1-108, wherein the glycan preparation has a final solubility limit in water of at least about 70 Brix at 23° C.

111. The method of any of claims 1-110, wherein the glycan preparation has a dietary fiber content of at least 50%.

112. The method of any of claims 1-110, wherein the glycan preparation has a dietary fiber content of at least 70%.

113. The method of any of claims 1-110, wherein the glycan polymers are not branched (average degree of branching is 0).

114. The method of any of claims 1-113, wherein the average degree of branching (DB) of the glycan polymers in the glycan preparation is between 0.01 and 0.6;

115. The method of any of claims 1-113, wherein the average degree of branching (DB) of the glycan polymers in the glycan preparation is between 0.05 and 0.5 or between 0.15 and 0.4.

116. The method of any of claims 1-115, wherein the average DP (mean DP) of the glycan preparation is between about 5 and 8, between about 8 and 13 or between about 13 and 25.

117. The method of any of claims 1-116, wherein the glycan polymers comprise only alpha-glycosidic bonds.

118. The method of any of claims 1-116, wherein the glycan polymers comprise only beta-glycosidic bonds.

119. The method of any of claims 1-116, wherein the glycan polymers comprise both alpha- and beta-glycosidic bonds.

120. The method of any of claims 1-116, wherein the ratio of alpha- to beta-glycosidic bonds present in the glycan polymers of the glycan preparation is between about 0.8:1 to about 5:1.

121. The method of any of claims 1-116, wherein the ratio of alpha- to beta-glycosidic bonds present in the glycan polymers of the glycan preparation is between about 1:1 to about 3:1, or between about 3:2 to about 3:1.

122. The method of any of claims 1-121, wherein the glycan composition comprises glycan polymers that comprise at least two distinct glycan units of glucose, galactose, arabinose, mannose, fructose, xylose, fucose, and rhamnose.

123. The method of any of claims 1-121, wherein the glycan composition comprises glycan polymers that comprise at least three distinct glycan units of glucose, galactose, arabinose, mannose, fructose, xylose, fucose, and rhamnose.

124. The method of any of claims 1-121, wherein the glycan composition comprises glycan polymers consisting of one or more of glucose, galactose, arabinose, mannose, fructose, xylose, fucose, or rhamnose glycan units.

125. The method of any of claims 1-121, wherein the glycan composition comprises glycan polymers consisting of one or more of glucose, galactose, mannose, or arabinose glycan units.

126. The method of any of claims 1-125, wherein at least two of the glycosidic bonds independently comprise a 1→2 glycosidic bond, a 1→3 glycosidic bond, a 1→4 glycosidic bond, or a 1→6 glycosidic bond.

127. The method of any of claims 1-125, wherein at least three of the glycosidic bonds independently comprise a 1→2 glycosidic bond, a 1→3 glycosidic bond, a 1→4 glycosidic bond, or a 1→6 glycosidic bond.

128. The method of any of claims 1-127, wherein
i) the glycan preparation comprises glycan polymers that comprise glucose, galactose, arabinose, mannose, fructose, xylose, fucose, or rhamnose glycan units;
ii) the average degree of branching (DB) of the glycan polymers in the glycan preparation is between 0.1 and 0.4;
iii) at least 50% of the glycan polymers in the glycan preparation have a degree of polymerization (DP) of at least 3 and less than 35 glycan units;
iv) the average DP (mean DP) of the glycan preparation is between about 5-15;
v) the ratio of alpha- to beta-glycosidic bonds present in the glycan polymers of the glycan preparation is between about 1:1 to about 4:1,
vi) the glycan preparation comprises between 30 mol % and 55 mol % 1,6 glycosidic bonds;
vii) the glycan preparation comprises between 10 mol % and 30 mol % of each at least one, two, or three of 1,2; 1,3; and 1,4 glycosidic bonds;
viii) the glycan preparation has a final solubility limit in water of at least about 50 Brix at 23° C.; or
ix) the glycan preparation has a dietary fiber content of at least 50%,
x) any combination of two, three, four, five, six, seven, eight, or nine of i), ii), iii), iv), v), vi), vii), viii), and ix).

129. The method of claim 53, wherein a Man100, Glu100, Man75Gal25, Man52Glu29Gal19, Glu50Gal50, Ara100, Gal100, or Glu60Man40 glycan composition is used to increase activity of rheumatoid arthritis drugs.

130. The method of claim 33, wherein a Man100, Gal100, Glu100, Man75Gal25, Man52Glu29Gal19, Glu50Gal50, Ara100, Glu33Gal33Ara33, Glu33Gal33Man34, Gal33Man33Ara33, or Glu60Man40 glycan composition is used to increase activity of cancer (e.g., colorectal cancer, or breast cancer) drugs.

131. The method of claim 1, wherein a Man100, Gal100, Glu100, Man75Gal25, Man52Glu29Gal19, Glu50Gal50, Ara100, Glu33Gal33Ara33, Glu33Gal33Man34, Gal33Man33Ara33, or Glu60Man40 glycan composition is used to increase activity of cardiac disease/disorder (e.g., cardiac arrhythmia, heart failure) drugs.

132. The method of claim 18, wherein a Man100, Gal100, Man75Gal25, Man52Glu29Gal19, Glu33Gal33Ara33, Glu33Gal33Man34, or Gal33Man33Ara33 glycan composition is used to increase activity of drugs reducing pain, fever, and/or drug-induced toxicity.

133. The method of claim 18, wherein a Gal100, Man75Gal25, or Ara100 glycan composition is used to increase activity of obesity (e.g., diet-induced obesity) drugs.

134. The method of claim 33, wherein a Ara100, Man100, Gal100, Glu100, Man75Gal25, Man52Glu29Gal19, Glu50Gal50, Glu33Gal33Ara33, Glu33Gal33Man34, Gal33Man33Ara33, or Glu60Man40 glycan composition is used to increase activity of viral infection (e.g., Herpes Simplex Virus Type 1 and/or Varicella Zoster Virus) drugs.

135. The method of claim 81, wherein a Man100, Gal100, Glu100, Man75Gal25, Man52Glu29Gal19, Glu50Gal50, Ara100, Gal100, Glu33Gal33Ara33, Glu33Gal33Man34, or Glu60Man40 glycan composition is used to increase levels of microbial enzymes that metabolize ellagitannin or ellagic acid.

136. The method of claim 81, wherein a Man100, Gal100, Glu100, Man75Gal25, Man52Glu29Gal19, Glu50Gal50, Glu33Gal33Ara33, Glu33Gal33Man34, Gal33Man33Ara33, or Glu60Man40 glycan composition is used to increase levels of microbial enzymes that metabolize phytoestrogen.

137. The method of claim 81, wherein a Ara100, Glu100, Man75Gal25, Man52Glu29Gal19, Glu50Gal50, Glu33Gal33Ara33, Glu33Gal33Man34, Gal33Man33Ara33, or Glu60Man40 glycan composition is used to increase levels of microbial enzymes that metabolize lignan (e.g., pinoresinol and/or secoisolariciresinol).

138. The method of claim 81, wherein a Ara100, Man100, Gal100, Glu100, Man75Gal25, Man52Glu29Gal19, Glu50Gal50, Glu33Gal33Ara33, Glu33Gal33Man34, Gal33Man33Ara33, or Glu60Man40 glycan composition is used to reduce levels of microbial enzymes that convert sweeteners to toxic compounds (e.g., convert cyclamate to cyclohexylamine).

139. The method of any of claims 1-128, wherein
i) the glycan preparation comprises glycan polymers that comprise glucose, galactose, arabinose, or mannose glycan units;
ii) the average degree of branching (DB) of the glycan polymers in the glycan preparation is between 0.1 and 0.4;
iii) at least 50% of the glycan polymers in the glycan preparation have a degree of polymerization (DP) of at least 3 and less than 35 glycan units;
iv) the average DP (mean DP) of the glycan preparation is between about 5-15
v) the ratio of alpha- to beta-glycosidic bonds present in the glycan polymers of the glycan preparation is between about 1:1 to about 4:1,
vi) the glycan preparation comprises between 30 mol % and 55 mol % 1,6 glycosidic bonds;
vii) the glycan preparation comprises between 10 mol % and 30 mol % of each at least one, two, or three of 1,2; 1,3; and 1,4 glycosidic bonds,
viii) the glycan preparation has a final solubility limit in water of at least about 50 Brix at 23° C.; and
ix) the glycan preparation has a dietary fiber content of at least 50%,

140. The method of claim 139, wherein the glycan composition is a Glu100, Gal100, Ara100, Man100, Glu50Gal50 or Glu60 Man40 glycan composition.

141. The method of claim 140, wherein a Glu100, Glu50Gal50 or Gal100 glycan composition is used for increasing the activity of a nucleoside analogue, an Ara100 glycan composition is used for increasing the activity of a cardiac glycoside, a Man100, Glu60Man40 or Glu100 glycan composition is used for increasing the activity of an aminosalicylate, and a Glu100, Gal100, Ara100, Man100, Glu50Gal50 or Glu60Man40 glycan composition is used for increasing the activity of a sulfonamide.

142. The method of claim 81, wherein a Glu40Gal20Man40, Glu20Gal20Man60, Glu45Gal10Man45, Gal100, Glu50Gal50, Glu10Gal80Man10, Glu20Gal40Man40, Glu30Gal40Man30, Glu10Gal45Man45, Glu10al10Man80, Man80Xyl20, Gal33Man33Xyl33, Man80Ara20, or Glu5Gal5Man90 is used for reducing activity of a N-acetyl transferase, a Glu100 is used for reducing activity of a beta-glucuronidase, a Glu100 is used for reducing activity of a thymidine phosphorylase, a Glu100 is used for reducing activity of a uridine phosphorylase, a Gal 100 is used for reducing activity of a bile acid CoA hydrolase, and a Glu100 is used for increasing activity of a urease.

143. A method for increasing drug activity in a rheumatoid arthritis subject, wherein the drug comprises an aminosalicylate, optionally, wherein the drug comprises a sulfasalazine, the method comprising:
a) administering a glycan composition in an amount effective and for a time sufficient to increase the drug activity in the subject;
b) administering a glycan composition in an amount effective and for a time sufficient to increase drug activity in the subject, and wherein at the time of administration of the glycan composition, the subject comprises a level of the drug that, in the presence of the administered glycan composition, provides a therapeutic effect;
c) administering the drug, wherein at the time of administration of the drug, the subject has already been administered the glycan composition in an amount effective and for a time sufficient to increase the drug activity in the subject;
d) administering the drug in an amount effective and for a time sufficient to increase the drug activity in the subject, wherein subject has been determined to be in need of the glycan composition, e.g., to increase the activity of the drug; or
e) administering the drug and the glycan composition to the subject, in amounts effective and for times sufficient to increase the drug activity in the subject, wherein administration of the drug and the glycan composition overlap;
wherein:
i) the glycan preparation comprises glycan polymers that comprise glucose, galactose, arabinose, or mannose glycan units;
ii) the average degree of branching (DB) of the glycan polymers in the glycan preparation is between 0.05 and 0.7;
iii) at least 50% of the glycan polymers in the glycan preparation have a degree of polymerization (DP) of at least 3 and less than 30 glycan units;
iv) the average DP (mean DP) of the glycan preparation is between about 5 and 13;
v) the ratio of alpha- to beta-glycosidic bonds present in the glycan polymers of the glycan preparation is between about 0.8:1 to about 5:1,
vi) the glycan preparation comprises between 30 mol % and 50 mol % 1,6 glycosidic bonds;
vii) the glycan preparation comprises between 5 mol % and 30 mol % of 1,2 glycosidic bonds; between 10 mol % and 45 mol % of 1,3 glycosidic bonds; and between 10 mol % and 35 mol % of 1,4 glycosidic bonds;
viii) the glycan preparation has a final solubility limit in water of at least about 50 Brix at 23° C.; or
ix) the glycan preparation has a dietary fiber content of at least 50%, and
x) any combination of two, three, four, five, six, seven, eight, or nine of i), ii), iii), iv), v), vi), vii), viii), and ix).

144. A method for increasing drug activity in a cancer (e.g., colorectal cancer, or breast cancer) subject or virally infected (e.g., HSV1, VZV, or both) subject, wherein the drug comprises a nucleoside analogue, optionally, wherein the drug comprises SN-38 glucuronide or sorivudine, the method comprising:
a) administering a glycan composition in an amount effective and for a time sufficient to increase the drug activity in the subject;
b) administering a glycan composition in an amount effective and for a time sufficient to increase drug activity in the subject, and wherein at the time of administration of the glycan composition, the subject comprises a level of the drug that, in the presence of the administered glycan composition, provides a therapeutic effect;
c) administering the drug, wherein at the time of administration of the drug, the subject has already been administered the glycan composition in an amount effective and for a time sufficient to increase the drug activity in the subject;
d) administering the drug in an amount effective and for a time sufficient to increase the drug activity in the subject, wherein subject has been determined to be in need of the glycan composition, e.g., to increase the activity of the drug; or
e) administering the drug and the glycan composition to the subject, in amounts effective and for times sufficient to increase the drug activity in the subject, wherein administration of the drug and the glycan composition overlap;
wherein:
i) the glycan preparation comprises glycan polymers that comprise glucose, galactose, arabinose, or mannose glycan units;
ii) the average degree of branching (DB) of the glycan polymers in the glycan preparation is between 0.05 and 0.7;
iii) at least 50% of the glycan polymers in the glycan preparation have a degree of polymerization (DP) of at least 3 and less than 30 glycan units;
iv) the average DP (mean DP) of the glycan preparation is between about 5 and 13;
v) the ratio of alpha- to beta-glycosidic bonds present in the glycan polymers of the glycan preparation is between about 0.8:1 to about 5:1, vi) the glycan preparation comprises between 30 mol % and 50 mol % 1,6 glycosidic bonds;
vii) the glycan preparation comprises between 5 mol % and 30 mol % of 1,2 glycosidic bonds; between 10 mol % and 45 mol % of 1,3 glycosidic bonds; and between 10 mol % and 35 mol % of 1,4 glycosidic bonds;
viii) the glycan preparation has a final solubility limit in water of at least about 50 Brix at 23° C.; or
ix) the glycan preparation has a dietary fiber content of at least 50%, and
x) any combination of two, three, four, five, six, seven, eight, or nine of i), ii), iii), iv), v), vi), vii), viii), and ix).

145. A method for increasing drug activity in a cardiac disease (e.g., cardiac arrhythmia, heart failure, or both) subject, wherein the drug comprises a cardiac glycoside, optionally, wherein the drug is digoxin, the method comprising:
a) administering a glycan composition in an amount effective and for a time sufficient to increase the drug activity in the subject;
b) administering a glycan composition in an amount effective and for a time sufficient to increase drug activity in the subject, and wherein at the time of administration of the glycan composition, the subject comprises a level of the drug that, in the presence of the administered glycan composition, provides a therapeutic effect;
c) administering the drug, wherein at the time of administration of the drug, the subject has already been administered the glycan composition in an amount effective and for a time sufficient to increase the drug activity in the subject;
d) administering the drug in an amount effective and for a time sufficient to increase the drug activity in the subject, wherein subject has been determined to be in need of the glycan composition, e.g., to increase the activity of the drug; or e) administering the drug and the glycan composition to the subject, in amounts effective and for times sufficient to increase the drug activity in the subject, wherein administration of the drug and the glycan composition overlap;
wherein:
i) the glycan preparation comprises glycan polymers that comprise glucose, galactose, arabinose, or mannose glycan units;
ii) the average degree of branching (DB) of the glycan polymers in the glycan preparation is between 0.05 and 0.6;
iii) at least 50% of the glycan polymers in the glycan preparation have a degree of polymerization (DP) of at least 3 and less than 30 glycan units;
iv) the average DP (mean DP) of the glycan preparation is between about 5 and 13;
v) the ratio of alpha- to beta-glycosidic bonds present in the glycan polymers of the glycan preparation is between about 1.1:1 to about 7:1,
vi) the glycan preparation comprises between 30 mol % and 50 mol % 1,6 glycosidic bonds;
vii) the glycan preparation comprises between 5 mol % and 30 mol % of 1,2 glycosidic bonds; between 10 mol % and 45 mol % of 1,3 glycosidic bonds; and between 10 mol % and 35 mol % of 1,4 glycosidic bonds;
viii) the glycan preparation has a final solubility limit in water of at least about 50 Brix at 23° C.; or
ix) the glycan preparation has a dietary fiber content of at least 50%, and
x) any combination of two, three, four, five, six, seven, eight, or nine of i), ii), iii), iv), v), vi), vii), viii), and ix).

146. A method for increasing drug activity in a subject experiencing fever, pain, drug-induced toxicity, or any combination thereof, wherein the drug comprises a sulfonamide, optionally, wherein the drug is acetaminophen, the method comprising:
a) administering a glycan composition in an amount effective and for a time sufficient to increase the drug activity in the subject;
b) administering a glycan composition in an amount effective and for a time sufficient to increase drug activity in the subject, and wherein at the time of administration of the glycan composition, the subject comprises a level of the drug that, in the presence of the administered glycan composition, provides a therapeutic effect;
c) administering the drug, wherein at the time of administration of the drug, the subject has already been administered the glycan composition in an amount effective and for a time sufficient to increase the drug activity in the subject;
d) administering the drug in an amount effective and for a time sufficient to increase the drug activity in the subject, wherein subject has been determined to be in need of the glycan composition, e.g., to increase the activity of the drug; or
e) administering the drug and the glycan composition to the subject, in amounts effective and for times sufficient to increase the drug activity in the subject, wherein administration of the drug and the glycan composition overlap;
wherein:
i) the glycan preparation comprises glycan polymers that comprise glucose, galactose, arabinose, or mannose glycan units;
ii) the average degree of branching (DB) of the glycan polymers in the glycan preparation is between 0.05 and 0.6;
iii) at least 50% of the glycan polymers in the glycan preparation have a degree of polymerization (DP) of at least 3 and less than 30 glycan units;
iv) the average DP (mean DP) of the glycan preparation is between about 5 and 11;
v) the ratio of alpha- to beta-glycosidic bonds present in the glycan polymers of the glycan preparation is between about 1.1:1 to about 7:1,
vi) the glycan preparation comprises between 30 mol % and 50 mol % 1,6 glycosidic bonds;
vii) the glycan preparation comprises between 5 mol % and 30 mol % of 1,2 glycosidic bonds; between 10 mol % and 45 mol % of 1,3 glycosidic bonds; and between 10 mol % and 35 mol % of 1,4 glycosidic bonds;
viii) the glycan preparation has a final solubility limit in water of at least about 50 Brix at 23° C.; or
ix) the glycan preparation has a dietary fiber content of at least 50%, and x) any combination of two, three, four, five, six, seven, eight, or nine of i), ii), iii), iv), v), vi), vii), viii), and ix).

147. A method for increasing metabolite activity in an obese subject (e.g., diet-induced obesity), wherein the metabolite comprises a taurine-conjugated bile acid, the method comprising:
a) administering a glycan composition in an amount effective and for a time sufficient to increase the metabolite activity in the subject;
b) administering a glycan composition in an amount effective and for a time sufficient to increase metabolite activity in the subject, and wherein at the time of administration of the glycan composition, the subject comprises a level of the metabolite that, in the presence of the administered glycan composition, provides a therapeutic effect;
c) administering the metabolite, wherein at the time of administration of the metabolite, the subject has already been administered the glycan composition in an amount effective and for a time sufficient to increase the metabolite activity in the subject;
d) administering the metabolite in an amount effective and for a time sufficient to increase the metabolite activity in the subject, wherein subject has been determined to be in need of the glycan composition, e.g., to increase the activity of the metabolite; or
e) administering the metabolite and the glycan composition to the subject, in amounts effective and for times sufficient to increase the metabolite activity in the subject, wherein administration of the metabolite and the glycan composition overlap;
wherein:
i) the glycan preparation comprises glycan polymers that comprise galactose, arabinose, or mannose glycan units;
ii) the average degree of branching (DB) of the glycan polymers in the glycan preparation is between 0.1 and 0.6;
iii) at least 50% of the glycan polymers in the glycan preparation have a degree of polymerization (DP) of at least 3 and less than 30 glycan units;
iv) the average DP (mean DP) of the glycan preparation is between about 5 and 13;
v) the ratio of alpha- to beta-glycosidic bonds present in the glycan polymers of the glycan preparation is between about 1.1:1 to about 6:1,
vi) the glycan preparation comprises between 30 mol % and 50 mol % 1,6 glycosidic bonds;
vii) the glycan preparation comprises between 5 mol % and 30 mol % of 1,2 glycosidic bonds; between 10 mol % and 45 mol % of 1,3 glycosidic bonds; and between 10 mol % and 35 mol % of 1,4 glycosidic bonds;
viii) the glycan preparation has a final solubility limit in water of at least about 50 Brix at 23° C.; or
ix) the glycan preparation has a dietary fiber content of at least 50%, and
x) any combination of two, three, four, five, six, seven, eight, or nine of i), ii), iii), iv), v), vi), vii), viii), and ix).

148. A method for reducing metabolite activity in a subject, wherein the metabolite comprises a heterocyclic amine, the method comprising:
a) administering a glycan composition in an amount effective and for a time sufficient to reduce the metabolite activity in the subject;
b) administering a glycan composition in an amount effective and for a time sufficient to reduce metabolite activity in the subject, and wherein at the time of administration of the glycan composition, the subject comprises a level of the metabolite that, in the presence of the administered glycan composition, provides a therapeutic effect;
c) administering an enzyme inhibitor (e.g., metabolite scavenger), wherein at the time of administration of the enzyme inhibitor (e.g., metabolite scavenger), the subject has already been administered the glycan composition in an amount effective and for a time sufficient to reduce the metabolite activity in the subject;
d) administering the enzyme inhibitor (e.g., metabolite scavenger) in an amount effective and for a time sufficient to reduce the metabolite activity in the subject, wherein subject has been determined to be in need of the glycan composition, e.g., to reduce the activity of the metabolite;
or
e) administering the enzyme inhibitor (e.g., metabolite scavenger) and the glycan composition to the subject, in amounts effective and for times sufficient to reduce the metabolite activity in the subject, wherein administration of the enzyme inhibitor (e.g., metabolite scavenger) and the glycan composition overlap;
wherein: i) the glycan preparation comprises glycan polymers that comprise glucose, galactose, arabinose, or mannose glycan units;
ii) the average degree of branching (DB) of the glycan polymers in the glycan preparation is between 0.05 and 0.7;
iii) at least 50% of the glycan polymers in the glycan preparation have a degree of polymerization (DP) of at least 3 and less than 30 glycan units;
iv) the average DP (mean DP) of the glycan preparation is between about 5 and 13;
v) the ratio of alpha- to beta-glycosidic bonds present in the glycan polymers of the glycan preparation is between about 0.8:1 to about 7:1,
vi) the glycan preparation comprises between 30 mol % and 50 mol % 1,6 glycosidic bonds;
vii) the glycan preparation comprises between 5 mol % and 30 mol % of 1,2 glycosidic bonds; between 10 mol % and 45 mol % of 1,3 glycosidic bonds; and between 10 mol % and 35 mol % of 1,4 glycosidic bonds;
viii) the glycan preparation has a final solubility limit in water of at least about 50 Brix at 23° C.; or
ix) the glycan preparation has a dietary fiber content of at least 50%, and
x) any combination of two, three, four, five, six, seven, eight, or nine of i), ii), iii), iv), v), vi), vii), viii), and ix).

149. A method for increasing the activity of ellagitannin or elagic acid metabolites (e.g., urolithin) in a subject, wherein the method comprises:
a) administering a glycan composition in an amount effective and for a time sufficient to increase the metabolite activity in the subject;
b) administering a glycan composition in an amount effective and for a time sufficient to increase activity in the subject, and wherein at the time of administration of the glycan composition, the subject comprises a level of metabolite that, in the presence of the administered glycan composition, provides a therapeutic effect; or
c) administering the metabolite, wherein at the time of administration of the metabolite, the subject has already been administered the glycan composition in an amount effective and for a time sufficient to increase the metabolite activity in the subject;
d) administering the metabolite in an amount effective and for a time sufficient to increase the metabolite activity in the subject, wherein subject has been determined to be in need of the glycan composition, e.g., to increase the activity of the metabolite; or
e) administering the metabolite and the glycan composition to the subject, in amounts effective and for times sufficient to increase the metabolite activity in the subject, wherein administration of the metabolite and the glycan composition overlap;
wherein:
i) the glycan preparation comprises glycan polymers that comprise glucose, galactose, arabinose, or mannose glycan units;
ii) the average degree of branching (DB) of the glycan polymers in the glycan preparation is between 0.05 and 0.7;
iii) at least 50% of the glycan polymers in the glycan preparation have a degree of polymerization (DP) of at least 3 and less than 30 glycan units;
iv) the average DP (mean DP) of the glycan preparation is between about 5 and 13;
v) the ratio of alpha- to beta-glycosidic bonds present in the glycan polymers of the glycan preparation is between about 0.8:1 to about 5:1,
vi) the glycan preparation comprises between 30 mol % and 50 mol % 1,6 glycosidic bonds;
vii) the glycan preparation comprises between 5 mol % and 30 mol % of 1,2 glycosidic bonds; between 10 mol % and 45 mol % of 1,3 glycosidic bonds; and between 10 mol % and 35 mol % of 1,4 glycosidic bonds;
viii) the glycan preparation has a final solubility limit in water of at least about 50 Brix at 23° C.; or
ix) the glycan preparation has a dietary fiber content of at least 50%, and
x) any combination of two, three, four, five, six, seven, eight, or nine of i), ii), iii), iv), v), vi), vii), viii), and ix).

150. A method for increasing activity of phytoestrogen, optionally, wherein the phytoestrogen is lignan, in a subject, wherein the method comprises:
a) administering a glycan composition in an amount effective and for a time sufficient to increase the activity in the subject;
b) administering a glycan composition in an amount effective and for a time sufficient to increase activity in the subject, and wherein at the time of administration of the glycan composition, the subject comprises a level of phytoestrogen that, in the presence of the administered glycan composition, provides a therapeutic effect;
c) administering the phytoestrogen, wherein at the time of administration of the phytoestrogen, the subject has already been administered the glycan composition in an amount effective and for a time sufficient to increase the phytoestrogen activity in the subject;
d) administering the phytoestrogen in an amount effective and for a time sufficient to increase the phytoestrogen activity in the subject, wherein subject has been determined to be in need of the glycan composition, e.g., to increase the activity of the phytoestrogen; or
e) administering the phytoestrogen and the glycan composition to the subject, in amounts effective and for times sufficient to increase the phytoestrogen activity in the subject, wherein administration of the phytoestrogen and the glycan composition overlap;
wherein:
i) the glycan preparation comprises glycan polymers that comprise glucose, galactose, arabinose, or mannose glycan units;
ii) the average degree of branching (DB) of the glycan polymers in the glycan preparation is between 0.05 and 0.7;
iii) at least 50% of the glycan polymers in the glycan preparation have a degree of polymerization (DP) of at least 3 and less than 30 glycan units;
iv) the average DP (mean DP) of the glycan preparation is between about 5 and 13;
v) the ratio of alpha- to beta-glycosidic bonds present in the glycan polymers of the glycan preparation is between about 0.8:1 to about 5:1,
vi) the glycan preparation comprises between 30 mol % and 50 mol % 1,6 glycosidic bonds;
vii) the glycan preparation comprises between 5 mol % and 30 mol % of 1,2 glycosidic bonds; between 10 mol % and 45 mol % of 1,3 glycosidic bonds; and between 10 mol % and 35 mol % of 1,4 glycosidic bonds;
viii) the glycan preparation has a final solubility limit in water of at least about 50 Brix at 23° C.; or
ix) the glycan preparation has a dietary fiber content of at least 50%, and
x) any combination of two, three, four, five, six, seven, eight, or nine of i), ii), iii), iv), v), vi), vii), viii), and ix).

151. A method for reducing activity of enzymes which convert sweeteners to toxic compounds (e.g., converting cyclamate to cyclohexylamine) in a subject, wherein the method comprises:
a) administering a glycan composition in an amount effective and for a time sufficient to reduce the activity of the enzyme in the subject;
b) administering a glycan composition in an amount effective and for a time sufficient to reduce activity in the subject, and wherein at the time of administration of the glycan composition, the subject comprises a level of the enzyme that, in the presence of the administered glycan composition, provides a non-toxic effect;
c) administering an enzyme inhibitor, wherein at the time of administration of the enzyme inhibitor, the subject has already been administered the glycan composition in an amount effective and for a time sufficient to reduce the enzyme activity in the subject;
d) administering the enzyme inhibitor in an amount effective and for a time sufficient to reduce the enzyme activity in the subject, wherein subject has been determined to be in need of the glycan composition, e.g., to reduce the activity of the enzyme; or e) administering the enzyme inhibitor and the glycan composition to the subject, in amounts effective and for times sufficient to reduce the enzyme activity in the subject, wherein administration of the enzyme inhibitor and the glycan composition overlap;
wherein:
i) the glycan preparation comprises glycan polymers that comprise glucose, galactose, arabinose, or mannose glycan units;
ii) the average degree of branching (DB) of the glycan polymers in the glycan preparation is between 0.05 and 0.7;
iii) at least 50% of the glycan polymers in the glycan preparation have a degree of polymerization (DP) of at least 3 and less than 30 glycan units;
iv) the average DP (mean DP) of the glycan preparation is between about 5 and 13;
v) the ratio of alpha- to beta-glycosidic bonds present in the glycan polymers of the glycan preparation is between about 0.8:1 to about 5:1, p1 vi) the glycan preparation comprises between 30 mol % and 50 mol % 1,6 glycosidic bonds;
vii) the glycan preparation comprises between 5 mol % and 30 mol % of 1,2 glycosidic bonds; between 10 mol % and 45 mol % of 1,3 glycosidic bonds; and between 10 mol % and 35 mol % of 1,4 glycosidic bonds;
viii) the glycan preparation has a final solubility limit in water of at least about 50 Brix at 23° C.; or
ix) the glycan preparation has a dietary fiber content of at least 50%, and
x) any combination of two, three, four, five, six, seven, eight, or nine of i), ii), iii), iv), v), vi), vii), viii), and ix).

152. The method of any of claims 1-146, wherein prior to administration of the glycan preparation and the drug the subject is assessed for the composition of its microbiome to determine whether the patient would benefit from administration of the glycan composition by modulating the abundance of microorganisms capable of increasing the activity of the respective drug.

153. The method of any of claim 1-146 or 152, wherein the glycan composition is replaced with FOS.

154. The method of any of claim 1-146, 152, or 153, wherein the glycan composition is replaced with lactulose.

155. A glycan composition disclosed herein.

156. The method of any of claim 1-146 or 152-155, further comprising administering the drug to the subject.

157. The method of any of claim 1-146 or 152-156, wherein the drug and the glycan composition are administered simultaneously.

158. The method of any of claim 1-146 or 152-157, wherein determining that the subject is in need of the glycan composition comprises acquiring information about the subject's need for the glycan composition, e.g., to increase the activity of the drug or decrease the toxicity of the drug.

159. The method of any of claim 1-146 or 152-158, further comprising acquiring information about the subject's need for the glycan composition e.g., to increase the activity of the drug or decrease the toxicity of the drug.

160. The method of claim 159, further comprising selecting and/or administering the glycan composition to the subject responsive to the information acquired.

161. The method of any of claim 1-146 or 152-160, wherein the glycan composition is administered responsive to the subject reaching a non-therapeutic event.

162. The method of any of claim 1-146 or 152-161, wherein determining that the subject is in need of the glycan composition comprises acquiring information about whether or not the subject reached a non-therapeutic event.

163. The method of any of claim 1-146 or 152-162, further comprising acquiring information about whether or not the subject reached a non-therapeutic event.

164. The method of either of claim 162 or 163, wherein the glycan composition is administered responsive to the information acquired about the non-therapeutic event.

165. The method of any of claim 1-146 or 152-164, wherein the subject has been administered the drug within 1, 5, 10, 15, or 30 days prior to administration of the glycan composition, e.g., an initial dose of the glycan composition.

166. The method of any of claim 1-146 or 152-165, wherein the subject is administered the drug within 1, 5, 10, 15, or 30 days after administration of the glycan composition, e.g., an initial dose of the glycan composition.

167. The method of any of claim 1-146 or 152-166, wherein the glycan composition is administered responsive to achieving a level of the drug in the subject, e.g., a level of the drug that provides a therapeutic effect, e.g., in the presence of the glycan composition.

168. The method of any of claim 1-146 or 152-167, wherein administering, e.g., the drug and/or glycan composition, comprises self-administering.

169. The method of any of claim 1-146 or 152-168, further comprising monitoring for the presence or absence of non-therapeutic events in a subject who has been administered the drug.
